SEQUENCE LISTING (118 sequences)

SEQ ID NO: 1 - DNA, 1833 bp, synthetic construct
  misc_feature 1..1833: note = synthetic construct
  source 1..1833: mol_type = other DNA
catatgaccattgatctcggagtttcactcacgtcgcaggccggcggcctgcaaggcatcgacctcaagagcatggatatccagactctcatggtgtatgtgcagggtcgtcgcgccgaactcctcacggctcaaatgcagacccaggccgaagtggtgcagaaggccaatgaacgcatggcgcagctcaacgaggtcctgtccgcgctgtcccgggccaaggccgagtttccgcccaatccgaagccgggcgacaccatcccgggctgggacaaccagaaggtcagccggatcgaggttcctctcaatgatgcgctgcgcgctgccggcctgacgggcatgttcgaagcgcgcgatggccaagtgaccgcccccggcggccggggtacgcaggtcgtgaacggcacgggcgtcatggccggttccacgacctataaggaactcgaaagtgcctacaccaccgtaaaggggatgctggatacggcgtccaatacgcaacagatggacatgatcaggctgcaggccgccagcaacaagcgcaacgaggctttcgaggtcatgaccaacaccgagaagcggcgcagcgacctgaacagttccatcaccaacaacatgcgcaagcttatgaccgtcatgagtacgaccatatccacagccccgagcggcgccgcgcttgcgccgtctcgcatagatatgcgggcaccggagcccgggagtgccggcgaaggcgccggcatcctggcgccggtgacgacgctggctctggcggcgggccggccggcttttccagcgtcaccgtcgctgcgcaccgcgcccgtcctggatccgccagtgcgcgatctcagccccgccgacttggccgacctgctgcgcgtcttgcgatccagggcggtggacgggcagttggccacggcgcgcgagaacctgcaggacgcgcaagtcaaggcgaagcagaacacccaggcccagctcgacaagctggacgcatggtttcggaaggccgaagaggccgagagcaagggatggctgagcaaggtgttcggctggatcggcaaggtgctggcggtcgtggcatcggccctggcggtgggctttgccgccgtcgccagcgtggccaccggcgcggcggccacacccatgctgctgctcagcggcatggcactggtcagcgccgtgacatcgctggccgaccagatatcgcaagaggcgggaggcccgcctatcagcctgggcgggtttctctccgggctggccggacgtctgctgacagcgttgggggtggatcagtcgcaggccgaccaaattgccaagatcgtcgccggcctggccgtgcccgtcgtcttgctgatcgaaccccagatgctgggcgaaatggcgcaaggcgtggccaggctggctggcgccagcgatgccaccgcggggtacatagccatggcgatgtccatcgtggcggcgatcgcggtcgccgcgatcaatgccgccggtacagccggcgcgggtagcgcttcggcgatcaagggggcctgggatcgggccgccgcggtagccacccaggtccttcaagggggtacggcagtggcgcaaggcggcgtcggcgtgtcgatggcagtcgatcgcaaacaggccgatctcctggtcgccgacaaggcggatctggcggcgagcctgacaaaactgcgggcggccatggagcgtgaggcggacgatatcaagaagatcctggctcaattcgacgaggcctatcacatgatcgcgaagatgatcagcgatatggcgagtacgcacagccaggtcagcgccaacctcgggcggcgccaggcggtgtagctcgag

SEQ ID NO: 2 - protein, 607 aa, synthetic construct
  REGION 1..607: note = synthetic construct
MTIDLGVSLTSQAGGLQGIDLKSMDIQTLMVYVQGRRAELLTAQMQTQAEVVQKANERMAQLNEVLSALSRAKAEFPPNPKPGDTIPGWDNQKVSRIEVPLNDALRAAGLTGMFEARDGQVTAPGGRGTQVVNGTGVMAGSTTYKELESAYTTVKGMLDTASNTQQMDMIRLQAASNKRNEAFEVMTNTEKRRSDLNSSITNNMRKLMTVMSTTISTAPSGAALAPSRIDMRAPEPGSAGEGAGILAPVTTLALAAGRPAFPASPSLRTAPVLDPPVRDLSPADLADLLRVLRSRAVDGQLATARENLQDAQVKAKQNTQAQLDKLDAWFRKAEEAESKGWLSKVFGWIGKVLAVVASALAVGFAAVASVATGAAATPMLLLSGMALVSAVTSLADQISQEAGGPPISLGGFLSGLAGRLLTALGVDQSQADQIAKIVAGLAVPVVLLIEPQMLGEMAQGVARLAGASDATAGYIAMAMSIVAAIAVAAINAAGTAGAGSASAIKGAWDRAAAVATQVLQGGTAVAQGGVGVSMAVDRKQADLLVADKADLAASLTKLRAAMEREADDIKKILAQFDEAYHMIAKMISDMASTHSQVSANLGRRQAV

SEQ ID NO: 3 - DNA, 618 bp, Bordetella spp
  source 1..618: mol_type = genomic DNA
catatgaccattgatctcggagtttcactcacgtcgcaggccggcggcctgcaaggcatcgacctcaagagcatggatatccagactctcatggtgtatgtgcagggtcgtcgcgccgaactcctcacggctcaaatgcagacccaggccgaagtggtgcagaaggccaatgaacgcatggcgcagctcaacgaggtcctgtccgcgctgtcccgggccaaggccgagtttccgcccaatccgaagccgggcgacaccatcccgggctgggacaaccagaaggtcagccggatcgaggttcctctcaatgatgcgctgcgcgctgccggcctgacgggcatgttcgaagcgcgcgatggccaagtgaccgcccccggcggccggggtacgcaggtcgtgaacggcacgggcgtcatggccggttccacgacctataaggaactcgaaagtgcctacaccaccgtaaaggggatgctggatacggcgtccaatacgcaacagatggacatgatcaggctgcaggccgccagcaacaagcgcaacgaggctttcgaggtcatgaccaacaccgagaagcggcgcagcgacctgaacagttccatcaccaacaacatgcgc

SEQ ID NO: 4 - protein, 205 aa, Bordetella spp
MTIDLGVSLTSQAGGLQGIDLKSMDIQTLMVYVQGRRAELLTAQMQTQAEVVQKANERMAQLNEVLSALSRAKAEFPPNPKPGDTIPGWDNQKVSRIEVPLNDALRAAGLTGMFEARDGQVTAPGGRGTQVVNGTGVMAGSTTYKELESAYTTVKGMLDTASNTQQMDMIRLQAASNKRNEAFEVMTNTEKRRSDLNSSITNNMR

SEQ ID NO: 5 - DNA, 1209 bp, Bordetella spp
  source 1..1209: mol_type = genomic DNA
atgaccgtcatgagtacgaccatatccacagccccgagcggcgccgcgcttgcgccgtctcgcatagatatgcgggcaccggagcccgggagtgccggcgaaggcgccggcatcctggcgccggtgacgacgctggctctggcggcgggccggccggcttttccagcgtcaccgtcgctgcgcaccgcgcccgtcctggatccgccagtgcgcgatctcagccccgccgacttggccgacctgctgcgcgtcttgcgatccagggcggtggacgggcagttggccacggcgcgcgagaacctgcaggacgcgcaagtcaaggcgaagcagaacacccaggcccagctcgacaagctggacgcatggtttcggaaggccgaagaggccgagagcaagggatggctgagcaaggtgttcggctggatcggcaaggtgctggcggtcgtggcatcggccctggcggtgggctttgccgccgtcgccagcgtggccaccggcgcggcggccacacccatgctgctgctcagcggcatggcactggtcagcgccgtgacatcgctggccgaccagatatcgcaagaggcgggaggcccgcctatcagcctgggcgggtttctctccgggctggccggacgtctgctgacagcgttgggggtggatcagtcgcaggccgaccaaattgccaagatcgtcgccggcctggccgtgcccgtcgtcttgctgatcgaaccccagatgctgggcgaaatggcgcaaggcgtggccaggctggctggcgccagcgatgccaccgcggggtacatagccatggcgatgtccatcgtggcggcgatcgcggtcgccgcgatcaatgccgccggtacagccggcgcgggtagcgcttcggcgatcaagggggcctgggatcgggccgccgcggtagccacccaggtccttcaagggggtacggcagtggcgcaaggcggcgtcggcgtgtcgatggcagtcgatcgcaaacaggccgatctcctggtcgccgacaaggcggatctggcggcgagcctgacaaaactgcgggcggccatggagcgtgaggcggacgatatcaagaagatcctggctcaattcgacgaggcctatcacatgatcgcgaagatgatcagcgatatggcgagtacgcacagccaggtcagcgccaacctcgggcggcgccaggcggtgtagctcgag

SEQ ID NO: 6 - protein, 400 aa, Bordetella spp
MTVMSTTISTAPSGAALAPSRIDMRAPEPGSAGEGAGILAPVTTLALAAGRPAFPASPSLRTAPVLDPPVRDLSPADLADLLRVLRSRAVDGQLATARENLQDAQVKAKQNTQAQLDKLDAWFRKAEEAESKGWLSKVFGWIGKVLAVVASALAVGFAAVASVATGAAATPMLLLSGMALVSAVTSLADQISQEAGGPPISLGGFLSGLAGRLLTALGVDQSQADQIAKIVAGLAVPVVLLIEPQMLGEMAQGVARLAGASDATAGYIAMAMSIVAAIAVAAINAAGTAGAGSASAIKGAWDRAAAVATQVLQGGTAVAQGGVGVSMAVDRKQADLLVADKADLAASLTKLRAAMEREADDIKKILAQFDEAYHMIAKMISDMASTHSQVSANLGRRQAV

SEQ ID NO: 7 - DNA, 534 bp, synthetic construct
  misc_feature 1..534: note = synthetic construct
  source 1..534: mol_type = other DNA
atgggcagcagccatcaccatcatcaccacagccaggatccgatgccaaagtcagccgagcagggcggctccccggcgtcagcttcgcatgaggcgttgcgccatattctcgacgcaggcgcttcgatgggcagcttgcaggggttggacgaggtgcaacagcaggcgttgtacgcgatcgctcatggcgcctacgaacagggccgctatgccgacgcgttgaaaatgttctgcctgctggtcgcgtgcgatccgctggaagcccgttatctgctggccctgggcgccgcggcccaggagctggggctgtacgagcatgccttgcagcaatacgcggccgcggcggctttgcagttggactcccccaggcccctgttgcatggcgccgagtgcctgtatgcgttgggtcgtcgccgcgacgccctggatacgctcgacatggtgcttgagttgtgcgggtcgccggagcatgcggccctgcgcgaacgggccgagtcgctgcgcaggagctatgcacgtgccgactgaaagctt

SEQ ID NO: 8 - protein, 175 aa, synthetic construct
  REGION 1..175: note = synthetic construct
MGSSHHHHHHSQDPMPKSAEQGGSPASASHEALRHILDAGASMGSLQGLDEVQQQALYAIAHGAYEQGRYADALKMFCLLVACDPLEARYLLALGAAAQELGLYEHALQQYAAAAALQLDSPRPLLHGAECLYALGRRRDALDTLDMVLELCGSPEHAALRERAESLRRSYARAD

SEQ ID NO: 9 - protein, 161 aa, synthetic construct
  REGION 1..161: note = synthetic construct
MPKSAEQGGSPASASHEALRHILDAGASMGSLQGLDEVQQQALYAIAHGAYEQGRYADALKMFCLLVACDPLEARYLLALGAAAQELGLYEHALQQYAAAAALQLDSPRPLLHGAECLYALGRRRDALDTLDMVLELCGSPEHAALRERAESLRRSYARAD

SEQ ID NO: 10 - DNA, 544 bp, synthetic construct
  misc_feature 1..544: note = synthetic construct
  source 1..544: mol_type = other DNA
ccatgggcagcagccatcatcatcatcatcacagcagcggcctggtgccgcgcggcagccatatgctcgagatgtctttaaatatcaccgaaaatgaaagcatctctactgcagtaattgatgcaattaactctggcgctacactgaaagatattaatgcaattcctgatgatatgatggatgacatttattcatatgcttatgacttttacaacaaaggaagaatagaggaagctgaagttttcttcaggtttttatgtatatacgacttttacaatatagactacattatgggactcgcagctatttatcagataaaagaacagttccaacaagcagcagacctttatgctgtcgcttttgcattaggaaaaaatgactatacaccagtattccatactggacaatgccagcttcggttgaaagcccccttaaaagctaaagagtgcttcgaactcgtaattcaacacagcaatgatgaaaaattaaaaataaaagcacaatcatacttggacgcaattcaggatatcaaggagtaggatcc

SEQ ID NO: 11 - protein, 155 aa, synthetic construct
  REGION 1..155: note = synthetic construct
MSLNITENESISTAVIDAINSGATLKDINAIPDDMMDDIYSYAYDFYNKGRIEEAEVFFRFLCIYDFYNVDYIMGLAAIYQIKEQFQQAADLYAVAFALGKNDYTPVFHTGQCQLRLKAPLKAKECFELVIQHSNDEKLKIKAQSYLDAIQDIKE

SEQ ID NO: 12 - DNA, 585 bp, Escherichia coli
  source 1..585: mol_type = genomic DNA
catatggacaatggcgatcgtttataccgtgccgactcgcgtcccccagatgagattaaacgtagcggtgggttaatgccacgtgggcacaatgagtattttgaccgtggaacacagatgaacattaacctttacgatcatgcccgtgggacccagaccgggtttgtccgttatgatgacgggtatgttagtacgagtttgtccttacgctccgcacaccttgcgggacaaagtattttatcaggctacagcacatattacatttatgtgatcgccactgccccaaacatgttcaatgtgaacgatgtgttgggggtttacagcccccatccatatgaacaagaagtctcggcccttggggggatcccatatagccagatttatggttggtaccgcgtaaattttggtgtgattgatgaacgtttgcatcgtaaccgtgaataccgcgatcgctactaccgtaacttgaacattgcacctgccgaggacggctatcgtttagcgggattcccacccgatcatcaggcgtggcgtgaggaaccgtggatccatcacgcccctcaggggtgcgggaacagtagtcgc

SEQ ID NO: 13 - protein, 194 aa, Escherichia coli
MDNGDRLYRADSRPPDEIKRSGGLMPRGHNEYFDRGTQMNINLYDHARGTQTGFVRYDDGYVSTSLSLRSAHLAGQSILSGYSTYYIYVIATAPNMFNVNDVLGVYSPHPYEQEVSALGGIPYSQIYGWYRVNFGVIDERLHRNREYRDRYYRNLNIAPAEDGYRLAGFPPDHQAWREEPWIHHAPQGCGNSSR

SEQ ID NO: 14 - protein, 5 aa, synthetic construct
  REGION 1..5: note = synthetic construct
GSAAS

SEQ ID NO: 15 - DNA, 3351 bp, synthetic construct
  misc_feature 1..3351: note = synthetic construct
  source 1..3351: mol_type = other DNA
catatggacaatggcgatcgtttataccgtgccgactcgcgtcccccagatgagattaaacgtagcggtgggttaatgccacgtgggcacaatgagtattttgaccgtggaacacagatgaacattaacctttacgatcatgcccgtgggacccagaccgggtttgtccgttatgatgacgggtatgttagtacgagtttgtccttacgctccgcacaccttgcgggacaaagtattttatcaggctacagcacatattacatttatgtgatcgccactgccccaaacatgttcaatgtgaacgatgtgttgggggtttacagcccccatccatatgaacaagaagtctcggcccttggggggatcccatatagccagatttatggttggtaccgcgtaaattttggtgtgattgatgaacgtttgcatcgtaaccgtgaataccgcgatcgctactaccgtaacttgaacattgcacctgccgaggacggctatcgtttagcgggattcccacccgatcatcaggcgtggcgtgaggaaccgtggatccatcacgcccctcaggggtgcgggaacagtagtcgcgggtccgcggcatccatgaatataacaactctgactaatagtatttccacctcatcattcagtccaaacaataccaacggttcatcaaccgaaacagttaattctgatataaaaacaacgaccagttctcatcctgtaagttcccttactatgctcaacgacacccttcataatatcagaacaacaaatcaggcattaaagaaagagctttcacaaaaaacgttgactaaaacatcgctagaagaaatagcattacattcatctcagattagcatggatgtaaataaatccgctcaactattggatattctttccaggaacgaatatccaattaataaagacgcaagagaattattacattcagccccgaaagaagccgagcttgatggagatcaaatgatatctcatagagaactgtgggctaaaattgcaaactccatcaatgatattaatgaacagtatctgaaagtatatgaacatgccgttagttcatatactcaaatgtatcaagattttagcgctgttctttccagtcttgccggctggatctctcccggaggtaacgacggaaactccgtgaaattacaagtcaactcgcttaaaaaggcattggaagaactcaaggaaaaatataaagataaaccgctatatccagcaaataatactgttagtcaggaacaagcaaataaatggcttacagaattaggtggaacaatcggcaaggtatctcaaaaaaacgggggatatgttgtcagtataaacatgaccccaatagacaatatgttaaaaagcttagataatctaggtggaaatggcgaggttgtgctagataatgcaaaatatcaggcatggaatgccggattctctgccgaagatgaaacaatgaaaaataatcttcaaactttagttcaaaaatacagtaatgccaatagtatttttgataatttagtaaaggttttgagtagtacaataagctcatgtacagatacagataaactttttctccatttcctcgagatgcataatgtaagcaccacaaccactggttttcctcttgccaaaatattgacttccactgagcttggagacaatactatccaagctgcaaatgatgcagctaacaaattattttctcttacaattgctgatcttactgctaaccaaaatattaatacaactaatgcacactcaacttcaaatatattaatccctgaacttaaagcaccaaagtcattaaatgcaagttcccaactaacgcttttaattggaaaccttattcaaatactcggtgaaaaatctttaactgcattaacaaataaaattactgcttggaagtcccagcaacaggcaagacagcaaaaaaacctagaattctccgataaaattaacactcttctatctgaaactgaaggactaaccagagactatgaaaaacaaattaataaactaaaaaacgcagattctaaaataaaagacctagaaaataaaattaaccaaattcaaacaagattatcgaacctcgatccagagtcaccagaaaagaaaaaattaagccgggaagaaatacaactcactatcaaaaaagacgcagcagttaaagacaggacattgattgagcagaaaaccctgtcaattcatagcaaacttacagataaatcaatgcaactcgaaaaagaaatagactctttttctgcattttcaaacacagcatctgctgaacagctatcaacccagcagaaatcattaaccggacttgccagtgttactcaattgatggcaacctttattcaactagttggaaaaaataatgaagaatctttaaaaaatgatctggctctattccagtctctccaagaatcaagaaaaactgaaatggagagaaaatctgatgagtatgctgctgaagtacgtaaagcagaagaactcaacagagtaatgggttgtgttgggaaaatacttggggcacttttaactatcgttagtgttgttgcagcagctttttctggaggagcctctctagcactggcagctgttggtttagctcttatggttacggatgctatagtacaagcagcgaccggcaattccttcatggaacaagccctgaatccgatcatgaaagcagtcattgaacccttaatcaaactcctttcagatgcatttacaaaaatgctcgaaggcttgggcgtcgactcgaaaaaagccaaaatgattggctctattctgggggcaatcgcaggcgctcttgtcctagttgcagcagtcgttctcgtagccactgttggtaaacaggcagcagcaaaacttgcagaaaatattggcaaaataataggtaaaaccctcacagaccttataccaaagtttctcaagaatttttcttctcaactggacgatttaatcactaatgctgttgccagattaaataaatttcttggtgcagcgggtgatgaagtaatatccaaacaaattatttccacccatttaaaccaagcagttttattaggagaaagtgttaactctgccacacaagcgggaggaagtgtcgcttctgctgttttccagaacagcgcgtcgacaaatctagcagacctgacattatcgaaatatcaagttgaacaactgtcaaaatatatcagtgaagcaatagaaaaattcggccaattgcaggaagtaattgcagatctattagcctcaatgtccaactctcaggctaatagaactgatgttgcaaaagcaattttgcaacaaactactgcttgaggatcc

SEQ ID NO: 16 - protein, 1081 aa, synthetic construct
  REGION 1..1081: note = synthetic construct
MDNGDRLYRADSRPPDEIKRSGGLMPRGHNEYFDRGTQMNINLYDHARGTQTGFVRYDDGYVSTSLSLRSAHLAGQSILSGYSTYYIYVIATAPNMFNVNDVLGVYSPHPYEQEVSALGGIPYSQIYGWYRVNFGVIDERLHRNREYRDRYYRNLNIAPAEDGYRLAGFPPDHQAWREEPWIHHAPQGCGNSSRGSAASMNITTLTNSISTSSFSPNNTNGSSTETVNSDIKTTTSSHPSSLTMLNDTLHNIRTTNQALKKELSQKTLRNEYPINKDARELLHSAPKEAELDGDQMISHRELWAKIANSINDINEQYLKVYEHAVSSYTQMYQDFSAVLSSLAGWISPGGNDGNSVKLQVNSLKKALEELKEKYKDKPLYPANNTVSQEQANKWLTELGGTIGKVSQKNGGYVVSINMTPIDNMLKSLDNLGGNGEVVLDNAKYQAWNGFSAEDETMKNNLQTLVQKYSNANSIFDNLVKVLSSTISSCTDTDKLFLHFLEMHNVSTTTTGFPLAKILTSTELGDNTIQAANDAANKLFSLTIADLTANQNINTTNAHSTSNILIPELKAPKSLNASSQLTLLIGNLIQILGEKSLTALTNKITAWKSQQQARQQKNLEFSDKINTLLSETEGLTRDYEKQINKLKNADSKIKDLENKINQIQTRLSNLDPESPEKKKLSREEIQLTIKKDAAVKDRTLIEQKTLSIHSKLTDKSMQLEKEIDSFSAFSNTASAEQLSTQQKSLTGLASVTQLMATFIQLVGKNNEESLKNDLALFQSLQESRKTEMERKSDEYAAEVRKAEELNRVMGCVGKILGALLTIVSVVAAAFSGGASLALAAVGLALMVTDAIVQAATGNSFMEQALNPIMKAVIEPLIKLLSDAFTKMLEGLGVDSKKAKMIGSILGAIAGALVLVAAVVLVATVGKQAAAKLAENIGKIIGKTLTDLIPKFLKNFSSQLDDLITNAVARLNKFLGAAGDEVISKQIISTHLNQAVLLGESVNSATQAGGSVASAVFQNSASTNLADLTLSKYQVEQLSKYISEAIEKFGQLQEVIADLLASMSNSQANRTDVAKAILQQTTA

SEQ ID NO: 17 - DNA, 2415 bp, synthetic construct
  misc_feature 1..2415: note = synthetic construct
  source 1..2415: mol_type = other DNA
catatggacaatggcgatcgtttataccgtgccgactcgcgtcccccagatgagattaaacgtagcggtgggttaatgccacgtgggcacaatgagtattttgaccgtggaacacagatgaacattaacctttacgatcatgcccgtgggacccagaccgggtttgtccgttatgatgacgggtatgttagtacgagtttgtccttacgctccgcacaccttgcgggacaaagtattttatcaggctacagcacatattacatttatgtgatcgccactgccccaaacatgttcaatgtgaacgatgtgttgggggtttacagcccccatccatatgaacaagaagtctcggcccttggggggatcccatatagccagatttatggttggtaccgcgtaaattttggtgtgattgatgaacgtttgcatcgtaaccgtgaataccgcgatcgctactaccgtaacttgaacattgcacctgccgaggacggctatcgtttagcgggattcccacccgatcatcaggcgtggcgtgaggaaccgtggatccatcacgcccctcaggggtgcgggaacagtagtcgcatgaccattgatctcggagtttcactcacgtcgcaggccggcggcctgcaaggcatcgacctcaagagcatggatatccagactctcatggtgtatgtgcagggtcgtcgcgccgaactcctcacggctcaaatgcagacccaggccgaagtggtgcagaaggccaatgaacgcatggcgcagctcaacgaggtcctgtccgcgctgtcccgggccaaggccgagtttccgcccaatccgaagccgggcgacaccatcccgggctgggacaaccagaaggtcagccggatcgaggttcctctcaatgatgcgctgcgcgctgccggcctgacgggcatgttcgaagcgcgcgatggccaagtgaccgcccccggcggccggggtacgcaggtcgtgaacggcacgggcgtcatggccggttccacgacctataaggaactcgaaagtgcctacaccaccgtaaaggggatgctggatacggcgtccaatacgcaacagatggacatgatcaggctgcaggccgccagcaacaagcgcaacgaggctttcgaggtcatgaccaacaccgagaagcggcgcagcgacctgaacagttccatcaccaacaacatgcgcaagcttatgaccgtcatgagtacgaccatatccacagccccgagcggcgccgcgcttgcgccgtctcgcatagatatgcgggcaccggagcccgggagtgccggcgaaggcgccggcatcctggcgccggtgacgacgctggctctggcggcgggccggccggcttttccagcgtcaccgtcgctgcgcaccgcgcccgtcctggatccgccagtgcgcgatctcagccccgccgacttggccgacctgctgcgcgtcttgcgatccagggcggtggacgggcagttggccacggcgcgcgagaacctgcaggacgcgcaagtcaaggcgaagcagaacacccaggcccagctcgacaagctggacgcatggtttcggaaggccgaagaggccgagagcaagggatggctgagcaaggtgttcggctggatcggcaaggtgctggcggtcgtggcatcggccctggcggtgggctttgccgccgtcgccagcgtggccaccggcgcggcggccacacccatgctgctgctcagcggcatggcactggtcagcgccgtgacatcgctggccgaccagatatcgcaagaggcgggaggcccgcctatcagcctgggcgggtttctctccgggctggccggacgtctgctgacagcgttgggggtggatcagtcgcaggccgaccaaattgccaagatcgtcgccggcctggccgtgcccgtcgtcttgctgatcgaaccccagatgctgggcgaaatggcgcaaggcgtggccaggctggctggcgccagcgatgccaccgcggggtacatagccatggcgatgtccatcgtggcggcgatcgcggtcgccgcgatcaatgccgccggtacagccggcgcgggtagcgcttcggcgatcaagggggcctgggatcgggccgccgcggtagccacccaggtccttcaagggggtacggcagtggcgcaaggcggcgtcggcgtgtcgatggcagtcgatcgcaaacaggccgatctcctggtcgccgacaaggcggatctggcggcgagcctgacaaaactgcgggcggccatggagcgtgaggcggacgatatcaagaagatcctggctcaattcgacgaggcctatcacatgatcgcgaagatgatcagcgatatggcgagtacgcacagccaggtcagcgccaacctcgggcggcgccaggcggtgtagctcgag

SEQ ID NO: 18 - protein, 801 aa, synthetic construct
  REGION 1..801: note = synthetic construct
MDNGDRLYRADSRPPDEIKRSGGLMPRGHNEYFDRGTQMNINLYDHARGTQTGFVRYDDGYVSTSLSLRSAHLAGQSILSGYSTYYIYVIATAPNMFNVNDVLGVYSPHPYEQEVSALGGIPYSQIYGWYRVNFGVIDERLHRNREYRDRYYRNLNIAPAEDGYRLAGFPPDHQAWREEPWIHHAPQGCGNSSRMTIDLGVSLTSQAGGLQGIDLKSMDIQTLMVYVQGRRAELLTAQMQTQAEVVQKANERMAQLNEVLSALSRAKAEFPPNPKPGDTIPGWDNQKVSRIEVPLNDALRAAGLTGMFEARDGQVTAPGGRGTQVVNGTGVMAGSTTYKELESAYTTVKGMLDTASNTQQMDMIRLQAASNKRNEAFEVMTNTEKRRSDLNSSITNNMRKLMTVMSTTISTAPSGAALAPSRIDMRAPEPGSAGEGAGILAPVTTLALAAGRPAFPASPSLRTAPVLDPPVRDLSPADLADLLRVLRSRAVDGQLATARENLQDAQVKAKQNTQAQLDKLDAWFRKAEEAESKGWLSKVFGWIGKVLAVVASALAVGFAAVASVATGAAATPMLLLSGMALVSAVTSLADQISQEAGGPPISLGGFLSGLAGRLLTALGVDQSQADQIAKIVAGLAVPVVLLIEPQMLGEMAQGVARLAGASDATAGYIAMAMSIVAAIAVAAINAAGTAGAGSASAIKGAWDRAAAVATQVLQGGTAVAQGGVGVSMAVDRKQADLLVADKADLAASLTKLRAAMEREADDIKKILAQFDEAYHMIAKMISDMASTHSQVSANLGRRQAV

SEQ ID NO: 19 - DNA, 566 bp, synthetic construct
  misc_feature 1..566: note = synthetic construct
  source 1..566: mol_type = other DNA
atgggcagcagccatcaccatcatcaccacagccaggatccgatgacgcaacgcgacgtgaacatagacgacatcgaggcgcaggaaatggcggcggcgctgctggacgcggtccagaacggcgcgacgctgaaggacctgcatcaggtgccgcaggacctgatggacggcatctatgcgttcgcgtaccgcttctaccagcaggggcggctcgacgacgcggaggtgttcttccgctttctgcgcatctacgacttctacaacgccgaatacgcgatggggctcgcggcggtgtgccagttgaagaaggagtacgcgcgggcgatcgatctgtatgcactcgcgtattcgctgtcgaaggacgaccaccggccgatgttccacaccggccaatgccatctgctgatgggcaaggcggcgctcgcgcggcgctgcttcggcatcgtcgtcgagcgctcgcgcgacgagcgcctcgcgcagaaggcgcagtcctatctcgacgggctcgacgaagtgggcgccgacgcggcgcccgcatccgccgggaacgaccactgagcggccgc

SEQ ID NO: 20 - protein, 185 aa, synthetic construct
  REGION 1..185: note = synthetic construct
MGSSHHHHHHSQDPMTQRDVNIDDIEAQEMAAALLDAVQNGATLKDLHQVPQDLMDGIYAFAYRFYQQGRLDDAEVFFRFLRIYDFYNAEYAMGLAAVCQLKKEYARAIDLYALAYSLSKDDHRPMFHTGQCHLLMGKAALARRCFGIVVERSRDERLAQKAQSYLDGLDEVGADAAPASAGNDH

SEQ ID NO: 21 - DNA, 930 bp, Burkholderia spp
  source 1..930: mol_type = genomic DNA
catatgaacatgcacgtggacatgggtcgtgcgctgaccgttcgtgattggccggcgctggaggcgctggcgaaaaccatgccggcggatgcgggtgcgcgtgcgatgaccgatgatgacctgcgtgcggcgggtgtggaccgtcgtgttccggagcagaagctgggtgcggcgattgatgaattcgcgagcctgcgtctgccggatcgtatcgacggtcgtttcgtggatggccgtcgtgcgaacctgaccgtttttgatgatgcgcgtgttgcggttcgtggtcatgcgcgtgcgcaacgtaacctgctggagcgtctggagaccgaactgctgggtggcaccctggataccgcgggtgacgaaggtggcattcagccggacccgatcctgcaaggcctggtggatgttatcggtcagggcaaaagcgatattgacgcgtacgcgaccatcgtggaaggtctgaccaagtattttcaaagcgtggcggacgttatgagcaaactgcaggattacattagcgcgaaggatgacaaaaacatgaagatcgacggtggcaagatcaaagcgctgattcagcaagtgatcgaccacctgccgaccatgcagctgccgaagggtgcggatattgcgcgttggcgtaaagagctgggcgacgcggttagcatcagcgatagcggtgtggttaccattaacccggacaaactgatcaagatgcgtgatagcctgccgccggatggcaccgtttgggataccgcgcgttaccaagcgtggaacaccgcgttcagcggtcagaaaggccagcatccggaacgtcgtgcggatgcgcgtcgtaaatatagccaccagaacagcaactttgataacctggtgaaggttctgagcggtgcgattagcaccctgaccgacacccagagctatctgcaaatc

SEQ ID NO: 22 - protein, 309 aa, Burkholderia spp
MNMHVDMGRALTVRDWPALEALAKTMPADAGARAMTDDDLRAAGVDRRVPEQKLGAAIDEFASLRLPDRIDGRFVDGRRANLTVFDDARVAVRGHARAQRNLLERLETELLGGTLDTAGDEGGIQPDPILQGLVDVIGQGKSDIDAYATIVEGLTKYFQSVADVMSKLQDYISAKDDKNMKIDGGKIKALIQQVIDHLPTMQLPKGADIARWRKELGDAVSISDSGVVTINPDKLIKMRDSLPPDGTVWDTARYQAWNTAFSGQKGQHPERRADARRKYSHQNSNFDNLVKVLSGAISTLTDTQSYLQI

SEQ ID NO: 23 - DNA, 1869 bp, Burkholderia spp
  source 1..1869: mol_type = genomic DNA
atgagcagcggtgttcaaggtggcccggcggcgaacgcgaacgcgtaccagacccacccgctgcgtgatgcggcgagcgcgctgggcaccctgagcccgcaggcgtatgtggatgtggttagcgcggcgcaacgtaacttcctggagcgtatgagccaactggcgagcgaacagtgcgatgcgcaaccggcggcgcatgatgcgcgtctggatgatcgtccggcgctgcgtgcgccgcaggaacgtgacgcgccgccgctgggtgcgagcgataccggtagccgtgcgagcggtgcggcgaaactgaccgagctgctgggtgtgctgatgagcgttattagcgcgagcagcctggacgaactgaagcaacgtagcgatatctggaaccagatgagcaaagcggcgcaagacaacctgagccgtctgagcgatgcgtttcagcgtgcgaccgacgaggcgaaagcggcggcggatgcggcggaacaggcggcggcggcggcgaagcaagcgggtgcggacgcgaaagcggcggatgcggcggtggatgcggcgcaaaaacgttacgatgacgcggttaagcagggcctgccggatgaccgtctgcaaagcctgaaagcggcgctggagcaggcgcgtcagcaagcgggtgatgcgcatggtcgtgcggatgcgctgcaggcggatgcgaccaagaaactggacgcggcgagcgcgctggcgacccaagcgcgtgcgtgcgaacagcaagtggatgacgcggttaaccaggcgacccagcaatatggtgcgagcgcgagcctgcgtaccccgcaaagcccgcgtctgagcggtgcggcggagctgaccgcggtgctgggcaagctgcaggaactgattagcagcggcaacgttaaagagctggaaagcaagcagaaactgttcaccgagatgcaagcgaagcgtgaggcggaactgcaaaagaaaagcgacgaatatcaggcgcaagtgaagaaagcggaggaaatgcagaaaacgatgggttgcatcggcaagattgtgggttgggttattaccgcggttagctttgcggcggcggcgtttaccggtggcgcgagcctggcgctggcggcggtgggcctggcgctggcggttggtgacgagattagccgtgcgaccaccggtgtgagcttcatggacaagctgatgcagccggttatggatgcgatcctgaaaccgctgatggagatgattagcagcctgatcaccaaggcgctggttgcgtgcggcgttgatcagcaaaaagcggaactggcgggtgcgattctgggtgcggttgttaccggtgtggcgctggttgcggcggcgtttgttggtgcgagcgcggtgaaagcggttgcgagcaaggttatcgacgcgatggcgggtcagctgaccaagctgatggatagcgcgattggcaaaatgctggtgcaactgatcgagaaattcagcgaaaagagcggtctgcaggcgctgggtagccgtaccgcgaccgcgatgacccgtatgcgtcgtgcgattggcgttgaggcgaaggaagacggtatgctgctggcgaaccgttttgaaaaagcgggcaccgtgatgaacgttggtaaccaagtgagccaagcggcgggtggcattgtggttggcgttgagcgtgcgaaagcgatgggtctgctggcggatgtgaaagaagcgatgtatgacatcaagctgctgggtgatctgctgaaacaggcggtggacgcgtttgcggagcacaaccgtgttctggcgcaactgatgcagcaaatgagcgatgcgggcgaaatgcagaccagcaccggcaagctgatcctgcgtaacgcgcgtgcggtttaaggatcc

SEQ ID NO: 24 - protein, 620 aa, Burkholderia spp
MSSGVQGGPAANANAYQTHPLRDAASALGTLSPQAYVDVVSAAQRNFLERMSQLASEQCDAQPAAHDARLDDRPALRAPQERDAPPLGASDTGSRASGAAKLTELLGVLMSVISASSLDELKQRSDIWNQMSKAAQDNLSRLSDAFQRATDEAKAAADAAEQAAAAAKQAGADAKAADAAVDAAQKRYDDAVKQGLPDDRLQSLKAALEQARQQAGDAHGRADALQADATKKLDAASALATQARACEQQVDDAVNQATQQYGASASLRTPQSPRLSGAAELTAVLGKLQELISSGNVKELESKQKLFTEMQAKREAELQKKSDEYQAQVKKAEEMQKTMGCIGKIVGWVITAVSFAAAAFTGGASLALAAVGLALAVGDEISRATTGVSFMDKLMQPVMDAILKPLMEMISSLITKALVACGVDQQKAELAGAILGAVVTGVALVAAAFVGASAVKAVASKVIDAMAGQLTKLMDSAIGKMLVQLIEKFSEKSGLQALGSRTATAMTRMRRAIGVEAKEDGMLLANRFEKAGTVMNVGNQVSQAAGGIVVGVERAKAMGLLADVKEAMYDIKLLGDLLKQAVDAFAEHNRVLAQLMQQMSDAGEMQTSTGKLILRNARAV

SEQ ID NO: 25 - DNA, 2805 bp, synthetic construct
  misc_feature 1..2805: note = synthetic construct
  source 1..2805: mol_type = other DNA
catatgaacatgcacgtggacatgggtcgtgcgctgaccgttcgtgattggccggcgctggaggcgctggcgaaaaccatgccggcggatgcgggtgcgcgtgcgatgaccgatgatgacctgcgtgcggcgggtgtggaccgtcgtgttccggagcagaagctgggtgcggcgattgatgaattcgcgagcctgcgtctgccggatcgtatcgacggtcgtttcgtggatggccgtcgtgcgaacctgaccgtttttgatgatgcgcgtgttgcggttcgtggtcatgcgcgtgcgcaacgtaacctgctggagcgtctggagaccgaactgctgggtggcaccctggataccgcgggtgacgaaggtggcattcagccggacccgatcctgcaaggcctggtggatgttatcggtcagggcaaaagcgatattgacgcgtacgcgaccatcgtggaaggtctgaccaagtattttcaaagcgtggcggacgttatgagcaaactgcaggattacattagcgcgaaggatgacaaaaacatgaagatcgacggtggcaagatcaaagcgctgattcagcaagtgatcgaccacctgccgaccatgcagctgccgaagggtgcggatattgcgcgttggcgtaaagagctgggcgacgcggttagcatcagcgatagcggtgtggttaccattaacccggacaaactgatcaagatgcgtgatagcctgccgccggatggcaccgtttgggataccgcgcgttaccaagcgtggaacaccgcgttcagcggtcagaaaggccagcatccggaacgtcgtgcggatgcgcgtcgtaaatatagccaccagaacagcaactttgataacctggtgaaggttctgagcggtgcgattagcaccctgaccgacacccagagctatctgcaaatcaagcttatgagcagcggtgttcaaggtggcccggcggcgaacgcgaacgcgtaccagacccacccgctgcgtgatgcggcgagcgcgctgggcaccctgagcccgcaggcgtatgtggatgtggttagcgcggcgcaacgtaacttcctggagcgtatgagccaactggcgagcgaacagtgcgatgcgcaaccggcggcgcatgatgcgcgtctggatgatcgtccggcgctgcgtgcgccgcaggaacgtgacgcgccgccgctgggtgcgagcgataccggtagccgtgcgagcggtgcggcgaaactgaccgagctgctgggtgtgctgatgagcgttattagcgcgagcagcctggacgaactgaagcaacgtagcgatatctggaaccagatgagcaaagcggcgcaagacaacctgagccgtctgagcgatgcgtttcagcgtgcgaccgacgaggcgaaagcggcggcggatgcggcggaacaggcggcggcggcggcgaagcaagcgggtgcggacgcgaaagcggcggatgcggcggtggatgcggcgcaaaaacgttacgatgacgcggttaagcagggcctgccggatgaccgtctgcaaagcctgaaagcggcgctggagcaggcgcgtcagcaagcgggtgatgcgcatggtcgtgcggatgcgctgcaggcggatgcgaccaagaaactggacgcggcgagcgcgctggcgacccaagcgcgtgcgtgcgaacagcaagtggatgacgcggttaaccaggcgacccagcaatatggtgcgagcgcgagcctgcgtaccccgcaaagcccgcgtctgagcggtgcggcggagctgaccgcggtgctgggcaagctgcaggaactgattagcagcggcaacgttaaagagctggaaagcaagcagaaactgttcaccgagatgcaagcgaagcgtgaggcggaactgcaaaagaaaagcgacgaatatcaggcgcaagtgaagaaagcggaggaaatgcagaaaacgatgggttgcatcggcaagattgtgggttgggttattaccgcggttagctttgcggcggcggcgtttaccggtggcgcgagcctggcgctggcggcggtgggcctggcgctggcggttggtgacgagattagccgtgcgaccaccggtgtgagcttcatggacaagctgatgcagccggttatggatgcgatcctgaaaccgctgatggagatgattagcagcctgatcaccaaggcgctggttgcgtgcggcgttgatcagcaaaaagcggaactggcgggtgcgattctgggtgcggttgttaccggtgtggcgctggttgcggcggcgtttgttggtgcgagcgcggtgaaagcggttgcgagcaaggttatcgacgcgatggcgggtcagctgaccaagctgatggatagcgcgattggcaaaatgctggtgcaactgatcgagaaattcagcgaaaagagcggtctgcaggcgctgggtagccgtaccgcgaccgcgatgacccgtatgcgtcgtgcgattggcgttgaggcgaaggaagacggtatgctgctggcgaaccgttttgaaaaagcgggcaccgtgatgaacgttggtaaccaagtgagccaagcggcgggtggcattgtggttggcgttgagcgtgcgaaagcgatgggtctgctggcggatgtgaaagaagcgatgtatgacatcaagctgctgggtgatctgctgaaacaggcggtggacgcgtttgcggagcacaaccgtgttctggcgcaactgatgcagcaaatgagcgatgcgggcgaaatgcagaccagcaccggcaagctgatcctgcgtaacgcgcgtgcggtttaaggatcc

SEQ ID NO: 26 - protein, 931 aa, synthetic construct
  REGION 1..931: note = synthetic construct
MNMHVDMGRALTVRDWPALEALAKTMPADAGARAMTDDDLRAAGVDRRVPEQKLGAAIDEFASLRLPDRIDGRFVDGRRANLTVFDDARVAVRGHARAQRNLLERLETELLGGTLDTAGDEGGIQPDPILQGLVDVIGQGKSDIDAYATIVEGLTKYFQSVADVMSKLQDYISAKDDKNMKIDGGKIKALIQQVIDHLPTMQLPKGADIARWRKELGDAVSISDSGVVTINPDKLIKMRDSLPPDGTVWDTARYQAWNTAFSGQKGQHPERRADARRKYSHQNSNFDNLVKVLSGAISTLTDTQSYLQIKLMSSGVQGGPAANANAYQTHPLRDAASALGTLSPQAYVDVVSAAQRNFLERMSQLASEQCDAQPAAHDARLDDRPALRAPQERDAPPLGASDTGSRASGAAKLTELLGVLMSVISASSLDELKQRSDIWNQMSKAAQDNLSRLSDAFQRATDEAKAAADAAEQAAAAAKQAGADAKAADAAVDAAQKRYDDAVKQGLPDDRLQSLKAALEQARQQAGDAHGRADALQADATKKLDAASALATQARACEQQVDDAVNQATQQYGASASLRTPQSPRLSGAAELTAVLGKLQELISSGNVKELESKQKLFTEMQAKREAELQKKSDEYQAQVKKAEEMQKTMGCIGKIVGWVITAVSFAAAAFTGGASLALAAVGLALAVGDEISRATTGVSFMDKLMQPVMDAILKPLMEMISSLITKALVACGVDQQKAELAGAILGAVVTGVALVAAAFVGASAVKAVASKVIDAMAGQLTKLMDSAIGKMLVQLIEKFSEKSGLQALGSRTATAMTRMRRAIGVEAKEDGMLLANRFEKAGTVMNVGNQVSQAAGGIVVGVERAKAMGLLADVKEAMYDIKLLGDLLKQAVDAFAEHNRVLAQLMQQMSDAGEMQTSTGKLILRNARAV

SEQ ID NO: 27 - DNA, 3390 bp, synthetic construct
  misc_feature 1..3390: note = synthetic construct
  source 1..3390: mol_type = other DNA
catatggacaatggcgatcgtttataccgtgccgactcgcgtcccccagatgagattaaacgtagcggtgggttaatgccacgtgggcacaatgagtattttgaccgtggaacacagatgaacattaacctttacgatcatgcccgtgggacccagaccgggtttgtccgttatgatgacgggtatgttagtacgagtttgtccttacgctccgcacaccttgcgggacaaagtattttatcaggctacagcacatattacatttatgtgatcgccactgccccaaacatgttcaatgtgaacgatgtgttgggggtttacagcccccatccatatgaacaagaagtctcggcccttggggggatcccatatagccagatttatggttggtaccgcgtaaattttggtgtgattgatgaacgtttgcatcgtaaccgtgaataccgcgatcgctactaccgtaacttgaacattgcacctgccgaggacggctatcgtttagcgggattcccacccgatcatcaggcgtggcgtgaggaaccgtggatccatcacgcccctcaggggtgcgggaacagtagtcgccatatgaacatgcacgtggacatgggtcgtgcgctgaccgttcgtgattggccggcgctggaggcgctggcgaaaaccatgccggcggatgcgggtgcgcgtgcgatgaccgatgatgacctgcgtgcggcgggtgtggaccgtcgtgttccggagcagaagctgggtgcggcgattgatgaattcgcgagcctgcgtctgccggatcgtatcgacggtcgtttcgtggatggccgtcgtgcgaacctgaccgtttttgatgatgcgcgtgttgcggttcgtggtcatgcgcgtgcgcaacgtaacctgctggagcgtctggagaccgaactgctgggtggcaccctggataccgcgggtgacgaaggtggcattcagccggacccgatcctgcaaggcctggtggatgttatcggtcagggcaaaagcgatattgacgcgtacgcgaccatcgtggaaggtctgaccaagtattttcaaagcgtggcggacgttatgagcaaactgcaggattacattagcgcgaaggatgacaaaaacatgaagatcgacggtggcaagatcaaagcgctgattcagcaagtgatcgaccacctgccgaccatgcagctgccgaagggtgcggatattgcgcgttggcgtaaagagctgggcgacgcggttagcatcagcgatagcggtgtggttaccattaacccggacaaactgatcaagatgcgtgatagcctgccgccggatggcaccgtttgggataccgcgcgttaccaagcgtggaacaccgcgttcagcggtcagaaaggccagcatccggaacgtcgtgcggatgcgcgtcgtaaatatagccaccagaacagcaactttgataacctggtgaaggttctgagcggtgcgattagcaccctgaccgacacccagagctatctgcaaatcaagcttatgagcagcggtgttcaaggtggcccggcggcgaacgcgaacgcgtaccagacccacccgctgcgtgatgcggcgagcgcgctgggcaccctgagcccgcaggcgtatgtggatgtggttagcgcggcgcaacgtaacttcctggagcgtatgagccaactggcgagcgaacagtgcgatgcgcaaccggcggcgcatgatgcgcgtctggatgatcgtccggcgctgcgtgcgccgcaggaacgtgacgcgccgccgctgggtgcgagcgataccggtagccgtgcgagcggtgcggcgaaactgaccgagctgctgggtgtgctgatgagcgttattagcgcgagcagcctggacgaactgaagcaacgtagcgatatctggaaccagatgagcaaagcggcgcaagacaacctgagccgtctgagcgatgcgtttcagcgtgcgaccgacgaggcgaaagcggcggcggatgcggcggaacaggcggcggcggcggcgaagcaagcgggtgcggacgcgaaagcggcggatgcggcggtggatgcggcgcaaaaacgttacgatgacgcggttaagcagggcctgccggatgaccgtctgcaaagcctgaaagcggcgctggagcaggcgcgtcagcaagcgggtgatgcgcatggtcgtgcggatgcgctgcaggcggatgcgaccaagaaactggacgcggcgagcgcgctggcgacccaagcgcgtgcgtgcgaacagcaagtggatgacgcggttaaccaggcgacccagcaatatggtgcgagcgcgagcctgcgtaccccgcaaagcccgcgtctgagcggtgcggcggagctgaccgcggtgctgggcaagctgcaggaactgattagcagcggcaacgttaaagagctggaaagcaagcagaaactgttcaccgagatgcaagcgaagcgtgaggcggaactgcaaaagaaaagcgacgaatatcaggcgcaagtgaagaaagcggaggaaatgcagaaaacgatgggttgcatcggcaagattgtgggttgggttattaccgcggttagctttgcggcggcggcgtttaccggtggcgcgagcctggcgctggcggcggtgggcctggcgctggcggttggtgacgagattagccgtgcgaccaccggtgtgagcttcatggacaagctgatgcagccggttatggatgcgatcctgaaaccgctgatggagatgattagcagcctgatcaccaaggcgctggttgcgtgcggcgttgatcagcaaaaagcggaactggcgggtgcgattctgggtgcggttgttaccggtgtggcgctggttgcggcggcgtttgttggtgcgagcgcggtgaaagcggttgcgagcaaggttatcgacgcgatggcgggtcagctgaccaagctgatggatagcgcgattggcaaaatgctggtgcaactgatcgagaaattcagcgaaaagagcggtctgcaggcgctgggtagccgtaccgcgaccgcgatgacccgtatgcgtcgtgcgattggcgttgaggcgaaggaagacggtatgctgctggcgaaccgttttgaaaaagcgggcaccgtgatgaacgttggtaaccaagtgagccaagcggcgggtggcattgtggttggcgttgagcgtgcgaaagcgatgggtctgctggcggatgtgaaagaagcgatgtatgacatcaagctgctgggtgatctgctgaaacaggcggtggacgcgtttgcggagcacaaccgtgttctggcgcaactgatgcagcaaatgagcgatgcgggcgaaatgcagaccagcaccggcaagctgatcctgcgtaacgcgcgtgcggtttaaggatcc

SEQ ID NO: 28 - protein, 1125 aa, synthetic construct
  REGION 1..1125: note = synthetic construct
MDNGDRLYRADSRPPDEIKRSGGLMPRGHNEYFDRGTQMNINLYDHARGTQTGFVRYDDGYVSTSLSLRSAHLAGQSILSGYSTYYIYVIATAPNMFNVNDVLGVYSPHPYEQEVSALGGIPYSQIYGWYRVNFGVIDERLHRNREYRDRYYRNLNIAPAEDGYRLAGFPPDHQAWREEPWIHHAPQGCGNSSRMNMHVDMGRALTVRDWPALEALAKTMPADAGARAMTDDDLRAAGVDRRVPEQKLGAAIDEFASLRLPDRIDGRFVDGRRANLTVFDDARVAVRGHARAQRNLLERLETELLGGTLDTAGDEGGIQPDPILQGLVDVIGQGKSDIDAYATIVEGLTKYFQSVADVMSKLQDYISAKDDKNMKIDGGKIKALIQQVIDHLPTMQLPKGADIARWRKELGDAVSISDSGVVTINPDKLIKMRDSLPPDGTVWDTARYQAWNTAFSGQKGQHPERRADARRKYSHQNSNFDNLVKVLSGAISTLTDTQSYLQIKLMSSGVQGGPAANANAYQTHPLRDAASALGTLSPQAYVDVVSAAQRNFLERMSQLASEQCDAQPAAHDARLDDRPALRAPQERDAPPLGASDTGSRASGAAKLTELLGVLMSVISASSLDELKQRSDIWNQMSKAAQDNLSRLSDAFQRATDEAKAAADAAEQAAAAAKQAGADAKAADAAVDAAQKRYDDAVKQGLPDDRLQSLKAALEQARQQAGDAHGRADALQADATKKLDAASALATQARACEQQVDDAVNQATQQYGASASLRTPQSPRLSGAAELTAVLGKLQELISSGNVKELESKQKLFTEMQAKREAELQKKSDEYQAQVKKAEEMQKTMGCIGKIVGWVITAVSFAAAAFTGGASLALAAVGLALAVGDEISRATTGVSFMDKLMQPVMDAILKPLMEMISSLITKALVACGVDQQKAELAGAILGAVVTGVALVAAAFVGASAVKAVASKVIDAMAGQLTKLMDSAIGKMLVQLIEKFSEKSGLQALGSRTATAMTRMRRAIGVEAKEDGMLLANRFEKAGTVMNVGNQVSQAAGGIVVGVERAKAMGLLADVKEAMYDIKLLGDLLKQAVDAFAEHNRVLAQLMQQMSDAGEMQTSTGKLILRNARAV

SEQ ID NO: 29 - DNA, 552 bp, synthetic construct
  misc_feature 1..552: note = synthetic construct
  source 1..552: mol_type = other DNA
atgggcagcagccatcaccatcatcaccacagccaggatccgatgaaccagccgaccccttccgacaccgaccagcaacaggcgctggaggccttcctgcgcgacggcggcaccctggcgatgcttcgcggactcagcgaggacaccctggagcagctctatgcgctgggcttcaaccagtaccaggcgggcaagtgggacgacgcgcagaagatcttccaggcactgtgcatgctcgaccactacgacgcccgctactttctcggcctgggcgcctgccgccagtccctcggtctctatgaacaggccctgcagagctacagctacggcgcgctgatggacatcaacgagccgcgctttcccttccatgccgccgagtgccacctgcaactgggtgatctcgacggagccgagagtggcttctactcggcccgggccctggccgcggcacagccggcgcacgaggccctggccgcgcgtgccggcgccatgttggaagccgtaaccgcgagaaaggatcgagcctatgaatccgataacgcttgaaagctt

SEQ ID NO: 30 - protein, 181 aa, synthetic construct
  REGION 1..181: note = synthetic construct
MGSSHHHHHHSQDPMNQPTPSDTDQQQALEAFLRDGGTLAMLRGLSEDTLEQLYALGFNQYQAGKWDDAQKIFQALCMLDHYDARYFLGLGACRQSLGLYEQALQSYSYGALMDINEPRFPFHAAECHLQLGDLDGAESGFYSARALAAAQPAHEALAARAGAMLEAVTARKDRAYESDNA

SEQ ID NO: 31 - DNA, 885 bp, Pseudomonas spp
  source 1..885: mol_type = genomic DNA
catatggaagtcagaaaccttaatgccgctcgcgagctgttcctggacgagctcctggccgcgtcggcggcgcctgccagtgccgagcaggaggaactgctggccctgttgcgcagcgagcggatcgtgctggcccacgccggccagccgctgagcgaggcgcaagtgctcaaggcgctcgcctggttgctcgcggccaatccgtccgcgcctccggggcagggcctcgaggtactccgcgaagtcctgcaggcacgtcggcagcccggtgcgcagtgggatctgcgtgagttcctggtgtcggcctatttcagcctgcacgggcgtctcgacgaggatgtcatcggtgtctacaaggatgtcctgcagacccaggacggcaagcgcaaggcgctgctcgacgagctcaaggcgctgaccgcggagttgaaggtctacagcgtgatccagtcgcagatcaacgccgcgctgtcggccaggcagggcatcaggatcgacgctggcggtatcgatctggtcgaccccacgctatatggctatgccgtcggcgatcccaggtggaaggacagccccgagtatgcgctgctgagcaatctggataccttcagcggcaagctgtcgatcaaggattttctcagcggctcgccgaagcagagcggggaactcaagggcctcagcgatgagtaccccttcgagaaggacaacaacccggtcggcaatttcgccaccacggtgagcgaccgctcgcgtccgctgaacgacaaggtcaacgagaagaccaccctgctcaacgacaccagctcccgctacaactcggcggtcgaggcgctcaaccgcttcatccagaaatacgacagcgtcctgagcgacattctcagcgcgatc

SEQ ID NO: 32 - protein, 294 aa, Pseudomonas spp
MEVRNLNAARELFLDELLAASAAPASAEQEELLALLRSERIVLAHAGQPLSEAQVLKALAWLLAANPSAPPGQGLEVLREVLQARRQPGAQWDLREFLVSAYFSLHGRLDEDVIGVYKDVLQTQDGKRKALLDELKALTAELKVYSVIQSQINAALSARQGIRIDAGGIDLVDPTLYGYAVGDPRWKDSPEYALLSNLDTFSGKLSIKDFLSGSPKQSGELKGLSDEYPFEKDNNPVGNFATTVSDRSRPLNDKVNEKTTLLNDTSSRYNSAVEALNRFIQKYDSVLSDILSAI

SEQ ID NO: 33 - DNA, 1179 bp, Pseudomonas spp
  source 1..1179: mol_type = genomic DNA
atgaacccgattacgctggaacgtgctggtctgccgtatggtgttgccgatgctggtgacatcccggctctgggtcgcccggtcgcacgtgatgtggaaagtctgcgtgttgaacgtctggcagcaccggcagctgcaagcgcatctggcaccggtgtcgctctgacgccgccgtctgcagcaagtcagcaacgtctggaagttgctaaccgcgcggaaattgcctcactggtccaggcagtgggtgaagacgtgggtctggcacgtcaagtggttctggcaggtgcatcgaccctgctgagcgcaggtctgatgtcgccgcaggcgttcgaaattgaactggccaaaatcaccggcgaagttgaaaatcagcagaaaaaactgaaactgacggaaatcgaacaggcccgtaaacagaacctgcaaaaaatggaagataaccagcaaaaaatccgcgaatcggaagaagctgcgaaagaagcgcagaaaagcggcctggccgcaaaaatttttggttggatttctgctatcgcgagtattatcgtgggtgcaatcatggttgcaaccggtgtcggtgctgcagcaggtgcactgatgattgctggcggtgtcatgggtgtcgtgagtcagtccgtgcagcaagcagctgcggatggtctgatctcaaaagaagtgatggaaaaactgggcccggccctgatgggtattgaaatggccgtggcactgctggccgcagttgtctcctttggtggttcagcagttggtggtctggcacgtctgggtgcaaaaatcggcggtaaagctgcggaaatgacggcatccctggcttcaaaagtggcagacctgggcggtaaattcggctctctggcgggccagtcactgtcgcatagcctgaaactgggtgtgcaagtttctgatctgaccctggacgttgcaaacggcgccgcacaggctacgcacagtggttttcaagcgaaagctgcgaatcgtcaggccgatgttcaagaatcccgtgcagacctgaccacgctgcagggtgtcattgaacgtctgaaagaagaactgagccgcatgctggaagcctttcaggaaattatggaacgcatcttcgcaatgctgcaagcgaaaggcgaaaccctgcacaatctgtcttcccgtccggcggctatctgaggatcc

SEQ ID NO: 34 - protein, 390 aa, Pseudomonas spp
MNPITLERAGLPYGVADAGDIPALGRPVARDVESLRVERLAAPAAASASGTGVALTPPSAASQQRLEVANRAEIASLVQAVGEDVGLARQVVLAGASTLLSAGLMSPQAFEIELAKITGEVENQQKKLKLTEIEQARKQNLQKMEDNQQKIRESEEAAKEAQKSGLAAKIFGWISAIASIIVGAIMVATGVGAAAGALMIAGGVMGVVSQSVQQAAADGLISKEVMEKLGPALMGIEMAVALLAAVVSFGGSAVGGLARLGAKIGGKAAEMTASLASKVADLGGKFGSLAGQSLSHSLKLGVQVSDLTLDVANGAAQATHSGFQAKAANRQADVQESRADLTTLQGVIERLKEELSRMLEAFQEIMERIFAMLQAKGETLHNLSSRPAAI

SEQ ID NO: 35 - DNA, 2070 bp, synthetic construct
  misc_feature 1..2070: note = synthetic construct
  source 1..2070: mol_type = other DNA
catatggaagtcagaaaccttaatgccgctcgcgagctgttcctggacgagctcctggccgcgtcggcggcgcctgccagtgccgagcaggaggaactgctggccctgttgcgcagcgagcggatcgtgctggcccacgccggccagccgctgagcgaggcgcaagtgctcaaggcgctcgcctggttgctcgcggccaatccgtccgcgcctccggggcagggcctcgaggtactccgcgaagtcctgcaggcacgtcggcagcccggtgcgcagtgggatctgcgtgagttcctggtgtcggcctatttcagcctgcacgggcgtctcgacgaggatgtcatcggtgtctacaaggatgtcctgcagacccaggacggcaagcgcaaggcgctgctcgacgagctcaaggcgctgaccgcggagttgaaggtctacagcgtgatccagtcgcagatcaacgccgcgctgtcggccaggcagggcatcaggatcgacgctggcggtatcgatctggtcgaccccacgctatatggctatgccgtcggcgatcccaggtggaaggacagccccgagtatgcgctgctgagcaatctggataccttcagcggcaagctgtcgatcaaggattttctcagcggctcgccgaagcagagcggggaactcaagggcctcagcgatgagtaccccttcgagaaggacaacaacccggtcggcaatttcgccaccacggtgagcgaccgctcgcgtccgctgaacgacaaggtcaacgagaagaccaccctgctcaacgacaccagctcccgctacaactcggcggtcgaggcgctcaaccgcttcatccagaaatacgacagcgtcctgagcgacattctcagcgcgatcggatccatgaacccgattacgctggaacgtgctggtctgccgtatggtgttgccgatgctggtgacatcccggctctgggtcgcccggtcgcacgtgatgtggaaagtctgcgtgttgaacgtctggcagcaccggcagctgcaagcgcatctggcaccggtgtcgctctgacgccgccgtctgcagcaagtcagcaacgtctggaagttgctaaccgcgcggaaattgcctcactggtccaggcagtgggtgaagacgtgggtctggcacgtcaagtggttctggcaggtgcatcgaccctgctgagcgcaggtctgatgtcgccgcaggcgttcgaaattgaactggccaaaatcaccggcgaagttgaaaatcagcagaaaaaactgaaactgacggaaatcgaacaggcccgtaaacagaacctgcaaaaaatggaagataaccagcaaaaaatccgcgaatcggaagaagctgcgaaagaagcgcagaaaagcggcctggccgcaaaaatttttggttggatttctgctatcgcgagtattatcgtgggtgcaatcatggttgcaaccggtgtcggtgctgcagcaggtgcactgatgattgctggcggtgtcatgggtgtcgtgagtcagtccgtgcagcaagcagctgcggatggtctgatctcaaaagaagtgatggaaaaactgggcccggccctgatgggtattgaaatggccgtggcactgctggccgcagttgtctcctttggtggttcagcagttggtggtctggcacgtctgggtgcaaaaatcggcggtaaagctgcggaaatgacggcatccctggcttcaaaagtggcagacctgggcggtaaattcggctctctggcgggccagtcactgtcgcatagcctgaaactgggtgtgcaagtttctgatctgaccctggacgttgcaaacggcgccgcacaggctacgcacagtggttttcaagcgaaagctgcgaatcgtcaggccgatgttcaagaatcccgtgcagacctgaccacgctgcagggtgtcattgaacgtctgaaagaagaactgagccgcatgctggaagcctttcaggaaattatggaacgcatcttcgcaatgctgcaagcgaaaggcgaaaccctgcacaatctgtcttcccgtccggcggctatctgaggatcc

SEQ ID NO: 36 - protein, 686 aa, synthetic construct
  REGION 1..686: note = synthetic construct
MEVRNLNAARELFLDELLAASAAPASAEQEELLALLRSERIVLAHAGQPLSEAQVLKALAWLLAANPSAPPGQGLEVLREVLQARRQPGAQWDLREFLVSAYFSLHGRLDEDVIGVYKDVLQTQDGKRKALLDELKALTAELKVYSVIQSQINAALSARQGIRIDAGGIDLVDPTLYGYAVGDPRWKDSPEYALLSNLDTFSGKLSIKDFLSGSPKQSGELKGLSDEYPFEKDNNPVGNFATTVSDRSRPLNDKVNEKTTLLNDTSSRYNSAVEALNRFIQKYDSVLSDILSAIGSMNPITLERAGLPYGVADAGDIPALGRPVARDVESLRVERLAAPAAASASGTGVALTPPSAASQQRLEVANRAEIASLVQAVGEDVGLARQVVLAGASTLLSAGLMSPQAFEIELAKITGEVENQQKKLKLTEIEQARKQNLQKMEDNQQKIRESEEAAKEAQKSGLAAKIFGWISAIASIIVGAIMVATGVGAAAGALMIAGGVMGVVSQSVQQAAADGLISKEVMEKLGPALMGIEMAVALLAAVVSFGGSAVGGLARLGAKIGGKAAEMTASLASKVADLGGKFGSLAGQSLSHSLKLGVQVSDLTLDVANGAAQATHSGFQAKAANRQADVQESRADLTTLQGVIERLKEELSRMLEAFQEIMERIFAMLQAKGETLHNLSSRPAAI

SEQ ID NO: 37 - DNA, 2655 bp, synthetic construct
  misc_feature 1..2655: note = synthetic construct
  source 1..2655: mol_type = other DNA
catatggacaatggcgatcgtttataccgtgccgactcgcgtcccccagatgagattaaacgtagcggtgggttaatgccacgtgggcacaatgagtattttgaccgtggaacacagatgaacattaacctttacgatcatgcccgtgggacccagaccgggtttgtccgttatgatgacgggtatgttagtacgagtttgtccttacgctccgcacaccttgcgggacaaagtattttatcaggctacagcacatattacatttatgtgatcgccactgccccaaacatgttcaatgtgaacgatgtgttgggggtttacagcccccatccatatgaacaagaagtctcggcccttggggggatcccatatagccagatttatggttggtaccgcgtaaattttggtgtgattgatgaacgtttgcatcgtaaccgtgaataccgcgatcgctactaccgtaacttgaacattgcacctgccgaggacggctatcgtttagcgggattcccacccgatcatcaggcgtggcgtgaggaaccgtggatccatcacgcccctcaggggtgcgggaacagtagtcgccatatggaagtcagaaaccttaatgccgctcgcgagctgttcctggacgagctcctggccgcgtcggcggcgcctgccagtgccgagcaggaggaactgctggccctgttgcgcagcgagcggatcgtgctggcccacgccggccagccgctgagcgaggcgcaagtgctcaaggcgctcgcctggttgctcgcggccaatccgtccgcgcctccggggcagggcctcgaggtactccgcgaagtcctgcaggcacgtcggcagcccggtgcgcagtgggatctgcgtgagttcctggtgtcggcctatttcagcctgcacgggcgtctcgacgaggatgtcatcggtgtctacaaggatgtcctgcagacccaggacggcaagcgcaaggcgctgctcgacgagctcaaggcgctgaccgcggagttgaaggtctacagcgtgatccagtcgcagatcaacgccgcgctgtcggccaggcagggcatcaggatcgacgctggcggtatcgatctggtcgaccccacgctatatggctatgccgtcggcgatcccaggtggaaggacagccccgagtatgcgctgctgagcaatctggataccttcagcggcaagctgtcgatcaaggattttctcagcggctcgccgaagcagagcggggaactcaagggcctcagcgatgagtaccccttcgagaaggacaacaacccggtcggcaatttcgccaccacggtgagcgaccgctcgcgtccgctgaacgacaaggtcaacgagaagaccaccctgctcaacgacaccagctcccgctacaactcggcggtcgaggcgctcaaccgcttcatccagaaatacgacagcgtcctgagcgacattctcagcgcgatcggatccatgaacccgattacgctggaacgtgctggtctgccgtatggtgttgccgatgctggtgacatcccggctctgggtcgcccggtcgcacgtgatgtggaaagtctgcgtgttgaacgtctggcagcaccggcagctgcaagcgcatctggcaccggtgtcgctctgacgccgccgtctgcagcaagtcagcaacgtctggaagttgctaaccgcgcggaaattgcctcactggtccaggcagtgggtgaagacgtgggtctggcacgtcaagtggttctggcaggtgcatcgaccctgctgagcgcaggtctgatgtcgccgcaggcgttcgaaattgaactggccaaaatcaccggcgaagttgaaaatcagcagaaaaaactgaaactgacggaaatcgaacaggcccgtaaacagaacctgcaaaaaatggaagataaccagcaaaaaatccgcgaatcggaagaagctgcgaaagaagcgcagaaaagcggcctggccgcaaaaatttttggttggatttctgctatcgcgagtattatcgtgggtgcaatcatggttgcaaccggtgtcggtgctgcagcaggtgcactgatgattgctggcggtgtcatgggtgtcgtgagtcagtccgtgcagcaagcagctgcggatggtctgatctcaaaagaagtgatggaaaaactgggcccggccctgatgggtattgaaatggccgtggcactgctggccgcagttgtctcctttggtggttcagcagttggtggtctggcacgtctgggtgcaaaaatcggcggtaaagctgcggaaatgacggcatccctggcttcaaaagtggcagacctgggcggtaaattcggctctctggcgggccagtcactgtcgcatagcctgaaactgggtgtgcaagtttctgatctgaccctggacgttgcaaacggcgccgcacaggctacgcacagtggttttcaagcgaaagctgcgaatcgtcaggccgatgttcaagaatcccgtgcagacctgaccacgctgcagggtgtcattgaacgtctgaaagaagaactgagccgcatgctggaagcctttcaggaaattatggaacgcatcttcgcaatgctgcaagcgaaaggcgaaaccctgcacaatctgtcttcccgtccggcggctatctgaggatcc

SEQ ID NO: 38 - protein, 880 aa, synthetic construct
  REGION 1..880: note = synthetic construct
MDNGDRLYRADSRPPDEIKRSGGLMPRGHNEYFDRGTQMNINLYDHARGTQTGFVRYDDGYVSTSLSLRSAHLAGQSILSGYSTYYIYVIATAPNMFNVNDVLGVYSPHPYEQEVSALGGIPYSQIYGWYRVNFGVIDERLHRNREYRDRYYRNLNIAPAEDGYRLAGFPPDHQAWREEPWIHHAPQGCGNSSRMEVRNLNAARELFLDELLAASAAPASAEQEELLALLRSERIVLAHAGQPLSEAQVLKALAWLLAANPSAPPGQGLEVLREVLQARRQPGAQWDLREFLVSAYFSLHGRLDEDVIGVYKDVLQTQDGKRKALLDELKALTAELKVYSVIQSQINAALSARQGIRIDAGGIDLVDPTLYGYAVGDPRWKDSPEYALLSNLDTFSGKLSIKDFLSGSPKQSGELKGLSDEYPFEKDNNPVGNFATTVSDRSRPLNDKVNEKTTLLNDTSSRYNSAVEALNRFIQKYDSVLSDILSAIGSMNPITLERAGLPYGVADAGDIPALGRPVARDVESLRVERLAAPAAASASGTGVALTPPSAASQQRLEVANRAEIASLVQAVGEDVGLARQVVLAGASTLLSAGLMSPQAFEIELAKITGEVENQQKKLKLTEIEQARKQNLQKMEDNQQKIRESEEAAKEAQKSGLAAKIFGWISAIASIIVGAIMVATGVGAAAGALMIAGGVMGVVSQSVQQAAADGLISKEVMEKLGPALMGIEMAVALLAAVVSFGGSAVGGLARLGAKIGGKAAEMTASLASKVADLGGKFGSLAGQSLSHSLKLGVQVSDLTLDVANGAAQATHSGFQAKAANRQADVQESRADLTTLQGVIERLKEELSRMLEAFQEIMERIFAMLQAKGETLHNLSSRPAAI

SEQ ID NO: 39 - DNA, 555 bp, synthetic construct
  misc_feature 1..555: note = synthetic construct
  source 1..555: mol_type = other DNA
atgggcagcagccatcaccatcatcaccacagccaggatccgatgcaacaagagacgacagacactcaagaataccagctggcaatggaatccttcctaaaaggagggggaactatcgccatgctcaacgaaatttcaagtgacactttagagcaactctactctcttgcgtttaaccaataccagtcaggaaaatacgaggatgctcacaaggtctttcaagctctctgtgtgctagaccactatgattcacgtttctttttagggctaggcgcttgtcgtcaagccatggggcaatacgacttagcgattcatagctacagctatggcgccataatggatataaaagaacctcgttttccgtttcatgctgccgaatgtttactgcaaaagggagagcttgctgaagcagaaagtggcttgttcttggctcaagagcttatcgcagacaaacctgagtttaaggagctttccacccgagttagctcaatgttagaagcaattaaattgaaaaaggagatggaacatgagtgcgttgataacccatgaaagctt

SEQ ID NO: 40 - protein, 188 aa, synthetic construct
  REGION 1..188: note = synthetic construct
MGSSHHHHHHSSGLVPRGSHMQQETTDTQEYQLAMESFLKGGGTIAMLNEISSDTLEQLYSLAFNQYQSGKYEDAHKVFQALCVLDHYDSRFFLGLGACRQAMGQYDLAIHSYSYGAIMDIKEPRFPFHAAECLLQKGELAEAESGLFLAQELIADKPEFKELSTRVSSMLEAIKLKKEMEHECVDNP

SEQ ID NO: 41 - DNA, 981 bp, Yersinia spp
  source 1..981: mol_type = genomic DNA
catatgattagagcctacgaacaaaacccacaacattttattgaggatctagaaaaagttagggtggaacaacttactggtcatggttcttcagttttagaagaattggttcagttagtcaaagataaaaatatagatatttccattaaatatgatcccagaaaagattcggaggtttttgccaatagagtaattactgatgatatcgaattgctcaagaaaatcctagcttattttctacccgaggatgccattcttaaaggcggtcattatgacaaccaactgcaaaatggcatcaagcgagtaaaagagttccttgaatcatcgccgaatacacaatgggaattgcgggcgttcatggcagtaatgcatttctctttaaccgccgatcgtatcgatgatgatattttgaaagtgattgttgattcaatgaatcatcatggtgatgcccgtagcaagttgcgtgaagaattagctgagcttaccgccgaattaaagatttattcagttattcaagccgaaattaataagcatctgtctagtagtggcaccataaatatccatgataaatccattaatctcatggataaaaatttatatggttatacagatgaagagatttttaaagccagcgcagagtacaaaattctcgagaaaatgcctcaaaccaccattcaggtggatgggagcgagaaaaaaatagtctcgataaaggactttcttggaagtgagaataaaagaaccggggcgttgggtaatctgaaaaactcatactcttataataaagataataatgaattatctcactttgccaccacctgctcggataagtccaggccgctcaacgacttggttagccaaaaaacaactcagctgtctgatattacatcacgttttaattcagctattgaagcactgaaccgtttcattcagaaatatgattcagtgatgcaacgtctgctagatgacacgtctggtaaa

SEQ ID NO: 42 - protein, 326 aa, Yersinia spp
MIRAYEQNPQHFIEDLEKVRVEQLTGHGSSVLEELVQLVKDKNIDISIKYDPRKDSEVFANRVITDDIELLKKILAYFLPEDAILKGGHYDNQLQNGIKRVKEFLESSPNTQWELRAFMAVMHFSLTADRIDDDILKVIVDSMNHHGDARSKLREELAELTAELKIYSVIQAEINKHLSSSGTINIHDKSINLMDKNLYGYTDEEIFKASAEYKILEKMPQTTIQVDGSEKKIVSIKDFLGSENKRTGALGNLKNSYSYNKDNNELSHFATTCSDKSRPLNDLVSQKTTQLSDITSRFNSAIEALNRFIQKYDSVMQRLLDDTSGK

SEQ ID NO: 43 - DNA, 1212 bp, Yersinia spp
  source 1..1212: mol_type = genomic DNA
atgagtgcgttgataacccatgatcgctcaacgccagtaactggaagtctacttccctacgtcgagacaccagcgcccgccccccttcagactcaacaagtcgcgggagaactgaaggataaaaatggtggggtgagttctcagggcgtacagctccctgcaccactagcagtggttgccagccaagtcactgaaggacaacagcaagaaatcactaaattattggagtcggtcacccgcggcacggcaggatctcaactgatatcaaattatgtttcagtgctaacgaattttacgctcgcttcacctgatacatttgagattgagttaggtaagctagtttctaatttagaagaagtacgcaaagacataaaaatcgctgatattcagcgtcttcatgaacaaaacatgaagaaaattgaagagaatcaagagaaaatcaaagaaacagaagagaatgccaagcaagtcaagaaatccggcatggcatcaaagatttttggctggctcagcgccatagcctcagtggttatcggtgccatcatggtggcctcaggggtaggagccgttgccggtgcaatgatgattgcctcaggcgtaattgggatggcgaatatggctgtgaaacaagcggcggaagatggcctgatatcccaagaggcaatgcaagtattagggccgatactcactgcgattgaagtcgcattgactgtagtttcaaccgtaatgacctttggcggttcggcactaaaatgcctggctgatattggcgcaaaactcggtgctaacaccgcaagtcttgctgctaaaggagccgagttttcggccaaagttgcccaaatttcgacaggcatatcaaacactgtcgggaatgcagtgactaaattagggggcagttttggtagtttaacaatgagccatgtaatccgtacaggatcacaggcaacacaagtcgccgttggtgtgggcagcggaataactcagaccatcaataataaaaaacaagctgatttacaacataataacgctgatttggccttgaacaaggcagacatggcagcgttacaaagtattattgaccgactcaaagaagagttatcccatttgtcagagtcacatcaacaagtgatggaactgattttccagatgattaatgcaaaaggtgacatgctgcataatttggccggcagaccccatactgtttaaggtacc

SEQ ID NO: 44 - protein, 401 aa, Yersinia spp
MSALITHDRSTPVTGSLLPYVETPAPAPLQTQQVAGELKDKNGGVSSQGVQLPAPLAVVASQVTEGQQQEITKLLESVTRGTAGSQLISNYVSVLTNFTLASPDTFEIELGKLVSNLEEVRKDIKIADIQRLHEQNMKKIEENQEKIKETEENAKQVKKSGMASKIFGWLSAIASVVIGAIMVASGVGAVAGAMMIASGVIGMANMAVKQAAEDGLISQEAMQVLGPILTAIEVALTVVSTVMTFGGSALKCLADIGAKLGANTASLAAKGAEFSAKVAQISTGISNTVGNAVTKLGGSFGSLTMSHVIRTGSQATQVAVGVGSGITQTINNKKQADLQHNNADLALNKADMAALQSIIDRLKEELSHLSESHQQVMELIFQMINAKGDMLHNLAGRPHTV

SEQ ID NO: 45 - DNA, 2199 bp, synthetic construct
  misc_feature 1..2199: note = synthetic construct
  source 1..2199: mol_type = other DNA
catatgattagagcctacgaacaaaacccacaacattttattgaggatctagaaaaagttagggtggaacaacttactggtcatggttcttcagttttagaagaattggttcagttagtcaaagataaaaatatagatatttccattaaatatgatcccagaaaagattcggaggtttttgccaatagagtaattactgatgatatcgaattgctcaagaaaatcctagcttattttctacccgaggatgccattcttaaaggcggtcattatgacaaccaactgcaaaatggcatcaagcgagtaaaagagttccttgaatcatcgccgaatacacaatgggaattgcgggcgttcatggcagtaatgcatttctctttaaccgccgatcgtatcgatgatgatattttgaaagtgattgttgattcaatgaatcatcatggtgatgcccgtagcaagttgcgtgaagaattagctgagcttaccgccgaattaaagatttattcagttattcaagccgaaattaataagcatctgtctagtagtggcaccataaatatccatgataaatccattaatctcatggataaaaatttatatggttatacagatgaagagatttttaaagccagcgcagagtacaaaattctcgagaaaatgcctcaaaccaccattcaggtggatgggagcgagaaaaaaatagtctcgataaaggactttcttggaagtgagaataaaagaaccggggcgttgggtaatctgaaaaactcatactcttataataaagataataatgaattatctcactttgccaccacctgctcggataagtccaggccgctcaacgacttggttagccaaaaaacaactcagctgtctgatattacatcacgttttaattcagctattgaagcactgaaccgtttcattcagaaatatgattcagtgatgcaacgtctgctagatgacacgtctggtaaaggatccatgagtgcgttgataacccatgatcgctcaacgccagtaactggaagtctacttccctacgtcgagacaccagcgcccgccccccttcagactcaacaagtcgcgggagaactgaaggataaaaatggtggggtgagttctcagggcgtacagctccctgcaccactagcagtggttgccagccaagtcactgaaggacaacagcaagaaatcactaaattattggagtcggtcacccgcggcacggcaggatctcaactgatatcaaattatgtttcagtgctaacgaattttacgctcgcttcacctgatacatttgagattgagttaggtaagctagtttctaatttagaagaagtacgcaaagacataaaaatcgctgatattcagcgtcttcatgaacaaaacatgaagaaaattgaagagaatcaagagaaaatcaaagaaacagaagagaatgccaagcaagtcaagaaatccggcatggcatcaaagatttttggctggctcagcgccatagcctcagtggttatcggtgccatcatggtggcctcaggggtaggagccgttgccggtgcaatgatgattgcctcaggcgtaattgggatggcgaatatggctgtgaaacaagcggcggaagatggcctgatatcccaagaggcaatgcaagtattagggccgatactcactgcgattgaagtcgcattgactgtagtttcaaccgtaatgacctttggcggttcggcactaaaatgcctggctgatattggcgcaaaactcggtgctaacaccgcaagtcttgctgctaaaggagccgagttttcggccaaagttgcccaaatttcgacaggcatatcaaacactgtcgggaatgcagtgactaaattagggggcagttttggtagtttaacaatgagccatgtaatccgtacaggatcacaggcaacacaagtcgccgttggtgtgggcagcggaataactcagaccatcaataataaaaaacaagctgatttacaacataataacgctgatttggccttgaacaaggcagacatggcagcgttacaaagtattattgaccgactcaaagaagagttatcccatttgtcagagtcacatcaacaagtgatggaactgattttccagatgattaatgcaaaaggtgacatgctgcataatttggccggcagaccccatactgtttaaggtacc

SEQ ID NO: 46 - protein, 729 aa, synthetic construct
  REGION 1..729: note = synthetic construct
MIRAYEQNPQHFIEDLEKVRVEQLTGHGSSVLEELVQLVKDKNIDISIKYDPRKDSEVFANRVITDDIELLKKILAYFLPEDAILKGGHYDNQLQNGIKRVKEFLESSPNTQWELRAFMAVMHFSLTADRIDDDILKVIVDSMNHHGDARSKLREELAELTAELKIYSVIQAEINKHLSSSGTINIHDKSINLMDKNLYGYTDEEIFKASAEYKILEKMPQTTIQVDGSEKKIVSIKDFLGSENKRTGALGNLKNSYSYNKDNNELSHFATTCSDKSRPLNDLVSQKTTQLSDITSRFNSAIEALNRFIQKYDSVMQRLLDDTSGKGSMSALITHDRSTPVTGSLLPYVETPAPAPLQTQQVAGELKDKNGGVSSQGVQLPAPLAVVASQVTEGQQQEITKLLESVTRGTAGSQLISNYVSVLTNFTLASPDTFEIELGKLVSNLEEVRKDIKIADIQRLHEQNMKKIEENQEKIKETEENAKQVKKSGMASKIFGWLSAIASVVIGAIMVASGVGAVAGAMMIASGVIGMANMAVKQAAEDGLISQEAMQVLGPILTAIEVALTVVSTVMTFGGSALKCLADIGAKLGANTASLAAKGAEFSAKVAQISTGISNTVGNAVTKLGGSFGSLTMSHVIRTGSQATQVAVGVGSGITQTINNKKQADLQHNNADLALNKADMAALQSIIDRLKEELSHLSESHQQVMELIFQMINAKGDMLHNLAGRPHTV

SEQ ID NO: 47 - DNA, 2784 bp, synthetic construct
  misc_feature 1..2784: note = synthetic construct
  source 1..2784: mol_type = other DNA
catatggacaatggcgatcgtttataccgtgccgactcgcgtcccccagatgagattaaacgtagcggtgggttaatgccacgtgggcacaatgagtattttgaccgtggaacacagatgaacattaacctttacgatcatgcccgtgggacccagaccgggtttgtccgttatgatgacgggtatgttagtacgagtttgtccttacgctccgcacaccttgcgggacaaagtattttatcaggctacagcacatattacatttatgtgatcgccactgccccaaacatgttcaatgtgaacgatgtgttgggggtttacagcccccatccatatgaacaagaagtctcggcccttggggggatcccatatagccagatttatggttggtaccgcgtaaattttggtgtgattgatgaacgtttgcatcgtaaccgtgaataccgcgatcgctactaccgtaacttgaacattgcacctgccgaggacggctatcgtttagcgggattcccacccgatcatcaggcgtggcgtgaggaaccgtggatccatcacgcccctcaggggtgcgggaacagtagtcgccatatgattagagcctacgaacaaaacccacaacattttattgaggatctagaaaaagttagggtggaacaacttactggtcatggttcttcagttttagaagaattggttcagttagtcaaagataaaaatatagatatttccattaaatatgatcccagaaaagattcggaggtttttgccaatagagtaattactgatgatatcgaattgctcaagaaaatcctagcttattttctacccgaggatgccattcttaaaggcggtcattatgacaaccaactgcaaaatggcatcaagcgagtaaaagagttccttgaatcatcgccgaatacacaatgggaattgcgggcgttcatggcagtaatgcatttctctttaaccgccgatcgtatcgatgatgatattttgaaagtgattgttgattcaatgaatcatcatggtgatgcccgtagcaagttgcgtgaagaattagctgagcttaccgccgaattaaagatttattcagttattcaagccgaaattaataagcatctgtctagtagtggcaccataaatatccatgataaatccattaatctcatggataaaaatttatatggttatacagatgaagagatttttaaagccagcgcagagtacaaaattctcgagaaaatgcctcaaaccaccattcaggtggatgggagcgagaaaaaaatagtctcgataaaggactttcttggaagtgagaataaaagaaccggggcgttgggtaatctgaaaaactcatactcttataataaagataataatgaattatctcactttgccaccacctgctcggataagtccaggccgctcaacgacttggttagccaaaaaacaactcagctgtctgatattacatcacgttttaattcagctattgaagcactgaaccgtttcattcagaaatatgattcagtgatgcaacgtctgctagatgacacgtctggtaaaggatccatgagtgcgttgataacccatgatcgctcaacgccagtaactggaagtctacttccctacgtcgagacaccagcgcccgccccccttcagactcaacaagtcgcgggagaactgaaggataaaaatggtggggtgagttctcagggcgtacagctccctgcaccactagcagtggttgccagccaagtcactgaaggacaacagcaagaaatcactaaattattggagtcggtcacccgcggcacggcaggatctcaactgatatcaaattatgtttcagtgctaacgaattttacgctcgcttcacctgatacatttgagattgagttaggtaagctagtttctaatttagaagaagtacgcaaagacataaaaatcgctgatattcagcgtcttcatgaacaaaacatgaagaaaattgaagagaatcaagagaaaatcaaagaaacagaagagaatgccaagcaagtcaagaaatccggcatggcatcaaagatttttggctggctcagcgccatagcctcagtggttatcggtgccatcatggtggcctcaggggtaggagccgttgccggtgcaatgatgattgcctcaggcgtaattgggatggcgaatatggctgtgaaacaagcggcggaagatggcctgatatcccaagaggcaatgcaagtattagggccgatactcactgcgattgaagtcgcattgactgtagtttcaaccgtaatgacctttggcggttcggcactaaaatgcctggctgatattggcgcaaaactcggtgctaacaccgcaagtcttgctgctaaaggagccgagttttcggccaaagttgcccaaatttcgacaggcatatcaaacactgtcgggaatgcagtgactaaattagggggcagttttggtagtttaacaatgagccatgtaatccgtacaggatcacaggcaacacaagtcgccgttggtgtgggcagcggaataactcagaccatcaataataaaaaacaagctgatttacaacataataacgctgatttggccttgaacaaggcagacatggcagcgttacaaagtattattgaccgactcaaagaagagttatcccatttgtcagagtcacatcaacaagtgatggaactgattttccagatgattaatgcaaaaggtgacatgctgcataatttggccggcagaccccatactgtttaaggtacc

SEQ ID NO: 48 - protein, 923 aa, synthetic construct
  REGION 1..923: note = synthetic construct
MDNGDRLYRADSRPPDEIKRSGGLMPRGHNEYFDRGTQMNINLYDHARGTQTGFVRYDDGYVSTSLSLRSAHLAGQSILSGYSTYYIYVIATAPNMFNVNDVLGVYSPHPYEQEVSALGGIPYSQIYGWYRVNFGVIDERLHRNREYRDRYYRNLNIAPAEDGYRLAGFPPDHQAWREEPWIHHAPQGCGNSSRMIRAYEQNPQHFIEDLEKVRVEQLTGHGSSVLEELVQLVKDKNIDISIKYDPRKDSEVFANRVITDDIELLKKILAYFLPEDAILKGGHYDNQLQNGIKRVKEFLESSPNTQWELRAFMAVMHFSLTADRIDDDILKVIVDSMNHHGDARSKLREELAELTAELKIYSVIQAEINKHLSSSGTINIHDKSINLMDKNLYGYTDEEIFKASAEYKILEKMPQTTIQVDGSEKKIVSIKDFLGSENKRTGALGNLKNSYSYNKDNNELSHFATTCSDKSRPLNDLVSQKTTQLSDITSRFNSAIEALNRFIQKYDSVMQRLLDDTSGKGSMSALITHDRSTPVTGSLLPYVETPAPAPLQTQQVAGELKDKNGGVSSQGVQLPAPLAVVASQVTEGQQQEITKLLESVTRGTAGSQLISNYVSVLTNFTLASPDTFEIELGKLVSNLEEVRKDIKIADIQRLHEQNMKKIEENQEKIKETEENAKQVKKSGMASKIFGWLSAIASVVIGAIMVASGVGAVAGAMMIASGVIGMANMAVKQAAEDGLISQEAMQVLGPILTAIEVALTVVSTVMTFGGSALKCLADIGAKLGANTASLAAKGAEFSAKVAQISTGISNTVGNAVTKLGGSFGSLTMSHVIRTGSQATQVAVGVGSGITQTINNKKQADLQHNNADLALNKADMAALQSIIDRLKEELSHLSESHQQVMELIFQMINAKGDMLHNLAGRPHTV

SEQ ID NO: 49 - DNA, 546 bp, synthetic construct
  misc_feature 1..546: note = synthetic construct
  source 1..546: mol_type = other DNA
atgggcagcagccatcaccatcatcaccacagccaggatccgatggactaccagaacaacgtcagcgaagaacgtgttgcggaaatgatttgggatgccgttagtgaaggcgccacgctaaaagacgttcatggaatccctcaagatatgatggacggtttatatgctcatgcttatgagttttataaccagggacgactggatgaagctgagacgttctttcgtttcttatgcatttatgatttttacaatcccgattacaccatgggactggcggcagtatgccaactgaaaaaacaatttcagaaagcatgtgacctttatgcagtagcgtttacgttacttaaaaatgattatcgccccgttttttttaccgggcagtgtcaattattaatgcgtaaggcagcaaaagccagacagtgttttgaacttgtcaatgaacgtactgaagatgagtctctgcgggcaaaagcgttggtctatctggaggcgctaaaaacggcggagacagagcagcacagcgagcaggagaaggagtaaaagctt

SEQ ID NO: 50 - protein, 179 aa, synthetic construct
  REGION 1..179: note = synthetic construct
MGSSHHHHHHSQDPMDYQNNVSEERVAEMIWDAVSEGATLKDVHGIPQDMMDGLYAHAYEFYNQGRLDEAETFFRFLCIYDFYNPDYTMGLAAVCQLKKQFQKACDLYAVAFTLLKNDYRPVFFTGQCQLLMRKAAKARQCFELVNERTEDESLRAKALVYLEALKTAETEQHSEQEKE

SEQ ID NO: 51 - DNA, 1029 bp, Salmonella spp
  source 1..1029: mol_type = genomic DNA
atgcttaatattcaaaattattccgcttctcctcatccggggatcgttgccgaacggccgcagactccctcggcgagcgagcacgtcgagactgccgtggtaccgtctaccacagaacatcgcggtacagatatcatttcattatcgcaggcggctactaaaatccaccaggcacagcagacgctgcagtcaacgccaccgatctctgaagagaataatgacgagcgcacgctggcgcgccagcagttgaccagcagcctgaatgcgctggcgaagtccggcgtgtcattatccgcagaacaaaatgagaacctgcggagcgcgttttctgcgccgacgtcggccttatttagcgcttcgcctatggcgcagccgagaacaaccatttctgatgctgagatttgggatatggtttcccaaaatatatcggcgataggtgacagctatctgggcgtttatgaaaacgttgtcgcagtctataccgatttttatcaggccttcagtgatattctttccaaaatgggaggctggttattaccaggtaaggacggtaataccgttaagctagatgttacctcactcaaaaatgatttaaacagtttagtcaataaatataatcaaataaacagtaataccgttttatttccagcgcagtcaggcagcggcgttaaagtagccactgaagcggaagcgagacagtggctcagtgaattgaatttaccgaatagctgcctgaaatcttatggatccggttatgtcgtcaccgttgatctgacgccattacaaaaaatggttcaggatattgatggtttaggcgcgccgggaaaagactcaaaactcgaaatggataacgccaaatatcaagcctggcagtcgggttttaaagcgcaggaagaaaatatgaaaaccacattacagacgctgacgcaaaaatatagcaatgccaattcattgtacgacaacctggtaaaagtgctgagcagtacgataagtagcagcctggaaaccgccaaaagcttcctgcaagga

SEQ ID NO: 52 - protein, 343 aa, Salmonella spp
MLNIQNYSASPHPGIVAERPQTPSASEHVETAVVPSTTEHRGTDIISLSQAATKIHQAQQTLQSTPPISEENNDERTLARQQLTSSLNALAKSGVSLSAEQNENLRSAFSAPTSALFSASPMAQPRTTISDAEIWDMVSQNISAIGDSYLGVYENVVAVYTDFYQAFSDILSKMGGWLLPGKDGNTVKLDVTSLKNDLNSLVNKYNQINSNTVLFPAQSGSGVKVATEAEARQWLSELNLPNSCLKSYGSGYVVTVDLTPLQKMVQDIDGLGAPGKDSKLEMDNAKYQAWQSGFKAQEENMKTTLQTLTQKYSNANSLYDNLVKVLSSTISSSLETAKSFLQG

SEQ ID NO: 53 - DNA, 1782 bp, Salmonella spp
  source 1..1782: mol_type = genomic DNA
atggtaaatgacgcaagtagcattagccgtagcggatatacccaaaatccgcgcctcgctgaggcggcttttgaaggcgttcgtaagaacacggactttttaaaagcggcggataaagcttttaaagatgtggtggcaacgaaagcgggcgaccttaaagccggaacaaagtccggcgagagcgctattaatacggtgggtctaaagccgcctacggacgccgcccgggaaaaactctccagcgaagggcaattgacattactgcttggcaagttaatgaccctactgggcgatgtttcgctgtctcaactggagtctcgtctggcggtatggcaggcgatgattgagtcacaaaaagagatggggattcaggtatcgaaagaattccagacggctctgggagaggctcaggaggcgacggatctctatgaagccagtatcaaaaagacggataccgccaagagtgtttatgacgctgcgaccaaaaaactgacgcaggcgcaaaataaattgcaatcgctggacccggctgaccccggctatgcacaagctgaagccgcggtagaacaggccggaaaagaagcgacagaggcgaaagaggccttagataaggccacggatgcgacggttaaagcaggcacagacgccaaagcgaaagccgagaaagcggataacattctgaccaaattccagggaacggctaatgccgcctctcagaatcaggtttcccagggtgagcaggataatctgtcaaatgtcgcccgcctcactatgctcatggccatgtttattgagattgtgggcaaaaatacggaagaaagcctgcaaaacgatcttgcgcttttcaacgccttgcaggaagggcgtcaggcggagatggaaaagaaatcggctgaattccaggaagagacgcgcaaagccgaggaaacgaaccgcattatgggatgtatcgggaaagtcctcggcgcgctgctaaccattgtcagcgttgtggccgctgtttttaccggtggggcgagtctggcgctggctgcggtgggacttgcggtaatggtggccgatgaaattgtgaaggcggcgacgggagtgtcgtttattcagcaggcgctaaacccgattatggagcatgtgctgaagccgttaatggagctgattggcaaggcgattaccaaagcgctggaaggattaggcgtcgataagaaaacggcagagatggccggcagcattgttggtgcgattgtcgccgctattgccatggtggcggtcattgtggtggtcgcagttgtcgggaaaggcgcggcggcgaaactgggtaacgcgctgagcaaaatgatgggcgaaacgattaagaagttggtgcctaacgtgctgaaacagttggcgcaaaacggcagcaaactctttacccaggggatgcaacgtattactagcggtctgggtaatgtgggtagcaagatgggcctgcaaacgaatgccttaagtaaagagctggtaggtaataccctaaataaagtggcgttgggcatggaagtcacgaataccgcagcccagtcagccggtggtgttgccgagggcgtatttattaaaaatgccagcgaggcgcttgctgattttatgctcgcccgttttgccatggatcagattcagcagtggcttaaacaatccgtagaaatatttggtgaaaaccagaaggtaacggcggaactgcaaaaagccatgtcttctgcggtacagcaaaatgcggatgcttcgcgttttattctgcgccagagtcgcgcataa

SEQ ID NO: 54 - protein, 593 aa, Salmonella spp
MVNDASSISRSGYTQNPRLAEAAFEGVRKNTDFLKAADKAFKDVVATKAGDLKAGTKSGESAINTVGLKPPTDAAREKLSSEGQLTLLLGKLMTLLGDVSLSQLESRLAVWQAMIESQKEMGIQVSKEFQTALGEAQEATDLYEASIKKTDTAKSVYDAATKKLTQAQNKLQSLDPADPGYAQAEAAVEQAGKEATEAKEALDKATDATVKAGTDAKAKAEKADNILTKFQGTANAASQNQVSQGEQDNLSNVARLTMLMAMFIEIVGKNTEESLQNDLALFNALQEGRQAEMEKKSAEFQEETRKAEETNRIMGCIGKVLGALLTIVSVVAAVFTGGASLALAAVGLAVMVADEIVKAATGVSFIQQALNPIMEHVLKPLMELIGKAITKALEGLGVDKKTAEMAGSIVGAIVAAIAMVAVIVVVAVVGKGAAAKLGNALSKMMGETIKKLVPNVLKQLAQNGSKLFTQGMQRITSGLGNVGSKMGLQTNALSKELVGNTLNKVALGMEVTNTAAQSAGGVAEGVFIKNASEALADFMLARFAMDQIQQWLKQSVEIFGENQKVTAELQKAMSSAVQQNADASRFILRQSRA

SEQ ID NO: 55 - DNA, 2817 bp, synthetic construct
  misc_feature 1..2817: note = synthetic construct
  source 1..2817: mol_type = other DNA
atgcttaatattcaaaattattccgcttctcctcatccggggatcgttgccgaacggccgcagactccctcggcgagcgagcacgtcgagactgccgtggtaccgtctaccacagaacatcgcggtacagatatcatttcattatcgcaggcggctactaaaatccaccaggcacagcagacgctgcagtcaacgccaccgatctctgaagagaataatgacgagcgcacgctggcgcgccagcagttgaccagcagcctgaatgcgctggcgaagtccggcgtgtcattatccgcagaacaaaatgagaacctgcggagcgcgttttctgcgccgacgtcggccttatttagcgcttcgcctatggcgcagccgagaacaaccatttctgatgctgagatttgggatatggtttcccaaaatatatcggcgataggtgacagctatctgggcgtttatgaaaacgttgtcgcagtctataccgatttttatcaggccttcagtgatattctttccaaaatgggaggctggttattaccaggtaaggacggtaataccgttaagctagatgttacctcactcaaaaatgatttaaacagtttagtcaataaatataatcaaataaacagtaataccgttttatttccagcgcagtcaggcagcggcgttaaagtagccactgaagcggaagcgagacagtggctcagtgaattgaatttaccgaatagctgcctgaaatcttatggatccggttatgtcgtcaccgttgatctgacgccattacaaaaaatggttcaggatattgatggtttaggcgcgccgggaaaagactcaaaactcgaaatggataacgccaaatatcaagcctggcagtcgggttttaaagcgcaggaagaaaatatgaaaaccacattacagacgctgacgcaaaaatatagcaatgccaattcattgtacgacaacctggtaaaagtgctgagcagtacgataagtagcagcctggaaaccgccaaaagcttcctgcaaggagtcgacatggtaaatgacgcaagtagcattagccgtagcggatatacccaaaatccgcgcctcgctgaggcggcttttgaaggcgttcgtaagaacacggactttttaaaagcggcggataaagcttttaaagatgtggtggcaacgaaagcgggcgaccttaaagccggaacaaagtccggcgagagcgctattaatacggtgggtctaaagccgcctacggacgccgcccgggaaaaactctccagcgaagggcaattgacattactgcttggcaagttaatgaccctactgggcgatgtttcgctgtctcaactggagtctcgtctggcggtatggcaggcgatgattgagtcacaaaaagagatggggattcaggtatcgaaagaattccagacggctctgggagaggctcaggaggcgacggatctctatgaagccagtatcaaaaagacggataccgccaagagtgtttatgacgctgcgaccaaaaaactgacgcaggcgcaaaataaattgcaatcgctggacccggctgaccccggctatgcacaagctgaagccgcggtagaacaggccggaaaagaagcgacagaggcgaaagaggccttagataaggccacggatgcgacggttaaagcaggcacagacgccaaagcgaaagccgagaaagcggataacattctgaccaaattccagggaacggctaatgccgcctctcagaatcaggtttcccagggtgagcaggataatctgtcaaatgtcgcccgcctcactatgctcatggccatgtttattgagattgtgggcaaaaatacggaagaaagcctgcaaaacgatcttgcgcttttcaacgccttgcaggaagggcgtcaggcggagatggaaaagaaatcggctgaattccaggaagagacgcgcaaagccgaggaaacgaaccgcattatgggatgtatcgggaaagtcctcggcgcgctgctaaccattgtcagcgttgtggccgctgtttttaccggtggggcgagtctggcgctggctgcggtgggacttgcggtaatggtggccgatgaaattgtgaaggcggcgacgggagtgtcgtttattcagcaggcgctaaacccgattatggagcatgtgctgaagccgttaatggagctgattggcaaggcgattaccaaagcgctggaaggattaggcgtcgataagaaaacggcagagatggccggcagcattgttggtgcgattgtcgccgctattgccatggtggcggtcattgtggtggtcgcagttgtcgggaaaggcgcggcggcgaaactgggtaacgcgctgagcaaaatgatgggcgaaacgattaagaagttggtgcctaacgtgctgaaacagttggcgcaaaacggcagcaaactctttacccaggggatgcaacgtattactagcggtctgggtaatgtgggtagcaagatgggcctgcaaacgaatgccttaagtaaagagctggtaggtaataccctaaataaagtggcgttgggcatggaagtcacgaataccgcagcccagtcagccggtggtgttgccgagggcgtatttattaaaaatgccagcgaggcgcttgctgattttatgctcgcccgttttgccatggatcagattcagcagtggcttaaacaatccgtagaaatatttggtgaaaaccagaaggtaacggcggaactgcaaaaagccatgtcttctgcggtacagcaaaatgcggatgcttcgcgttttattctgcgccagagtcgcgcataa

SEQ ID NO: 56 - protein, 938 aa, synthetic construct
  REGION 1..938: note = synthetic construct
MLNIQNYSASPHPGIVAERPQTPSASEHVETAVVPSTTEHRGTDIISLSQAATKIHQAQQTLQSTPPISEENNDERTLARQQLTSSLNALAKSGVSLSAEQNENLRSAFSAPTSALFSASPMAQPRTTISDAEIWDMVSQNISAIGDSYLGVYENVVAVYTDFYQAFSDILSKMGGWLLPGKDGNTVKLDVTSLKNDLNSLVNKYNQINSNTVLFPAQSGSGVKVATEAEARQWLSELNLPNSCLKSYGSGYVVTVDLTPLQKMVQDIDGLGAPGKDSKLEMDNAKYQAWQSGFKAQEENMKTTLQTLTQKYSNANSLYDNLVKVLSSTISSSLETAKSFLQGVDMVNDASSISRSGYTQNPRLAEAAFEGVRKNTDFLKAADKAFKDVVATKAGDLKAGTKSGESAINTVGLKPPTDAAREKLSSEGQLTLLLGKLMTLLGDVSLSQLESRLAVWQAMIESQKEMGIQVSKEFQTALGEAQEATDLYEASIKKTDTAKSVYDAATKKLTQAQNKLQSLDPADPGYAQAEAAVEQAGKEATEAKEALDKATDATVKAGTDAKAKAEKADNILTKFQGTANAASQNQVSQGEQDNLSNVARLTMLMAMFIEIVGKNTEESLQNDLALFNALQEGRQAEMEKKSAEFQEETRKAEETNRIMGCIGKVLGALLTIVSVVAAVFTGGASLALAAVGLAVMVADEIVKAATGVSFIQQALNPIMEHVLKPLMELIGKAITKALEGLGVDKKTAEMAGSIVGAIVAAIAMVAVIVVVAVVGKGAAAKLGNALSKMMGETIKKLVPNVLKQLAQNGSKLFTQGMQRITSGLGNVGSKMGLQTNALSKELVGNTLNKVALGMEVTNTAAQSAGGVAEGVFIKNASEALADFMLARFAMDQIQQWLKQSVEIFGENQKVTAELQKAMSSAVQQNADASRFILRQSRA

SEQ ID NO: 57 - DNA, 3423 bp, synthetic construct
  misc_feature 1..3423: note = synthetic construct
  source 1..3423: mol_type = other DNA
catatggacaatggcgatcgtttataccgtgccgactcgcgtcccccagatgagattaaacgtagcggtgggttaatgccacgtgggcacaatgagtattttgaccgtggaacacagatgaacattaacctttacgatcatgcccgtgggacccagaccgggtttgtccgttatgatgacgggtatgttagtacgagtttgtccttacgctccgcacaccttgcgggacaaagtattttatcaggctacagcacatattacatttatgtgatcgccactgccccaaacatgttcaatgtgaacgatgtgttgggggtttacagcccccatccatatgaacaagaagtctcggcccttggggggatcccatatagccagatttatggttggtaccgcgtaaattttggtgtgattgatgaacgtttgcatcgtaaccgtgaataccgcgatcgctactaccgtaacttgaacattgcacctgccgaggacggctatcgtttagcgggattcccacccgatcatcaggcgtggcgtgaggaaccgtggatccatcacgcccctcaggggtgcgggaacagtagtcgcgggtccgcggcatccatgcttaatattcaaaattattccgcttctcctcatccggggatcgttgccgaacggccgcagactccctcggcgagcgagcacgtcgagactgccgtggtaccgtctaccacagaacatcgcggtacagatatcatttcattatcgcaggcggctactaaaatccaccaggcacagcagacgctgcagtcaacgccaccgatctctgaagagaataatgacgagcgcacgctggcgcgccagcagttgaccagcagcctgaatgcgctggcgaagtccggcgtgtcattatccgcagaacaaaatgagaacctgcggagcgcgttttctgcgccgacgtcggccttatttagcgcttcgcctatggcgcagccgagaacaaccatttctgatgctgagatttgggatatggtttcccaaaatatatcggcgataggtgacagctatctgggcgtttatgaaaacgttgtcgcagtctataccgatttttatcaggccttcagtgatattctttccaaaatgggaggctggttattaccaggtaaggacggtaataccgttaagctagatgttacctcactcaaaaatgatttaaacagtttagtcaataaatataatcaaataaacagtaataccgttttatttccagcgcagtcaggcagcggcgttaaagtagccactgaagcggaagcgagacagtggctcagtgaattgaatttaccgaatagctgcctgaaatcttatggatccggttatgtcgtcaccgttgatctgacgccattacaaaaaatggttcaggatattgatggtttaggcgcgccgggaaaagactcaaaactcgaaatggataacgccaaatatcaagcctggcagtcgggttttaaagcgcaggaagaaaatatgaaaaccacattacagacgctgacgcaaaaatatagcaatgccaattcattgtacgacaacctggtaaaagtgctgagcagtacgataagtagcagcctggaaaccgccaaaagcttcctgcaaggagtcgacatggtaaatgacgcaagtagcattagccgtagcggatatacccaaaatccgcgcctcgctgaggcggcttttgaaggcgttcgtaagaacacggactttttaaaagcggcggataaagcttttaaagatgtggtggcaacgaaagcgggcgaccttaaagccggaacaaagtccggcgagagcgctattaatacggtgggtctaaagccgcctacggacgccgcccgggaaaaactctccagcgaagggcaattgacattactgcttggcaagttaatgaccctactgggcgatgtttcgctgtctcaactggagtctcgtctggcggtatggcaggcgatgattgagtcacaaaaagagatggggattcaggtatcgaaagaattccagacggctctgggagaggctcaggaggcgacggatctctatgaagccagtatcaaaaagacggataccgccaagagtgtttatgacgctgcgaccaaaaaactgacgcaggcgcaaaataaattgcaatcgctggacccggctgaccccggctatgcacaagctgaagccgcggtagaacaggccggaaaagaagcgacagaggcgaaagaggccttagataaggccacggatgcgacggttaaagcaggcacagacgccaaagcgaaagccgagaaagcggataacattctgaccaaattccagggaacggctaatgccgcctctcagaatcaggtttcccagggtgagcaggataatctgtcaaatgtcgcccgcctcactatgctcatggccatgtttattgagattgtgggcaaaaatacggaagaaagcctgcaaaacgatcttgcgcttttcaacgccttgcaggaagggcgtcaggcggagatggaaaagaaatcggctgaattccaggaagagacgcgcaaagccgaggaaacgaaccgcattatgggatgtatcgggaaagtcctcggcgcgctgctaaccattgtcagcgttgtggccgctgtttttaccggtggggcgagtctggcgctggctgcggtgggacttgcggtaatggtggccgatgaaattgtgaaggcggcgacgggagtgtcgtttattcagcaggcgctaaacccgattatggagcatgtgctgaagccgttaatggagctgattggcaaggcgattaccaaagcgctggaaggattaggcgtcgataagaaaacggcagagatggccggcagcattgttggtgcgattgtcgccgctattgccatggtggcggtcattgtggtggtcgcagttgtcgggaaaggcgcggcggcgaaactgggtaacgcgctgagcaaaatgatgggcgaaacgattaagaagttggtgcctaacgtgctgaaacagttggcgcaaaacggcagcaaactctttacccaggggatgcaacgtattactagcggtctgggtaatgtgggtagcaagatgggcctgcaaacgaatgccttaagtaaagagctggtaggtaataccctaaataaagtggcgttgggcatggaagtcacgaataccgcagcccagtcagccggtggtgttgccgagggcgtatttattaaaaatgccagcgaggcgcttgctgattttatgctcgcccgttttgccatggatcagattcagcagtggcttaaacaatccgtagaaatatttggtgaaaaccagaaggtaacggcggaactgcaaaaagccatgtcttctgcggtacagcaaaatgcggatgcttcgcgttttattctgcgccagagtcgcgcataactcgag

SEQ ID NO: 58 - protein, 1137 aa, synthetic construct
  REGION 1..1137: note = synthetic construct
MDNGDRLYRADSRPPDEIKRSGGLMPRGHNEYFDRGTQMNINLYDHARGTQTGFVRYDDGYVSTSLSLRSAHLAGQSILSGYSTYYIYVIATAPNMFNVNDVLGVYSPHPYEQEVSALGGIPYSQIYGWYRVNFGVIDERLHRNREYRDRYYRNLNIAPAEDGYRLAGFPPDHQAWREEPWIHHAPQGCGNSSRGSAASMLNIQNYSASPHPGIVAERPQTPSASEHVETAVVPSTTEHRGTDIISLSQAATKIHQAQQTLQSTPPISEENNDERTLARQQLTSSLNALAKSGVSLSAEQNENLRSAFSAPTSALFSASPMAQPRTTISDAEIWDMVSQNISAIGDSYLGVYENVVAVYTDFYQAFSDILSKMGGWLLPGKDGNTVKLDVTSLKNDLNSLVNKYNQINSNTVLFPAQSGSGVKVATEAEARQWLSELNLPNSCLKSYGSGYVVTVDLTPLQKMVQDIDGLGAPGKDSKLEMDNAKYQAWQSGFKAQEENMKTTLQTLTQKYSNANSLYDNLVKVLSSTISSSLETAKSFLQGVDMVNDASSISRSGYTQNPRLAEAAFEGVRKNTDFLKAADKAFKDVVATKAGDLKAGTKSGESAINTVGLKPPTDAAREKLSSEGQLTLLLGKLMTLLGDVSLSQLESRLAVWQAMIESQKEMGIQVSKEFQTALGEAQEATDLYEASIKKTDTAKSVYDAATKKLTQAQNKLQSLDPADPGYAQAEAAVEQAGKEATEAKEALDKATDATVKAGTDAKAKAEKADNILTKFQGTANAASQNQVSQGEQDNLSNVARLTMLMAMFIEIVGKNTEESLQNDLALFNALQEGRQAEMEKKSAEFQEETRKAEETNRIMGCIGKVLGALLTIVSVVAAVFTGGASLALAAVGLAVMVADEIVKAATGVSFIQQALNPIMEHVLKPLMELIGKAITKALEGLGVDKKTAEMAGSIVGAIVAAIAMVAVIVVVAVVGKGAAAKLGNALSKMMGETIKKLVPNVLKQLAQNGSKLFTQGMQRITSGLGNVGSKMGLQTNALSKELVGNTLNKVALGMEVTNTAAQSAGGVAEGVFIKNASEALADFMLARFAMDQIQQWLKQSVEIFGENQKVTAELQKAMSSAVQQNADASRFILRQSRA

SEQ ID NO: 59 - DNA, 522 bp, synthetic construct
  misc_feature 1..522: note = synthetic construct
  source 1..522: mol_type = other DNA
atgggcagcagccatcaccatcatcaccacagccaggatccgatgaaaaaagacccgaccctacaacaggcacatgacacgatgcggtttttccggcgtggcggctcgctgcgtatgttgttggatgacgatgttacacagccgcttaatactctgtatcgctatgccacgcagcttatggaggtaaaagaattcgccggcgcagcgcgactttttcaattgctgacgatatatgatgcctggtcatttgactactggtttcggttaggggaatgctgccaggctcaaaaacattggggggaagcgatatacgcttatggacgcgcggcacaaattaagattgatgcgccgcaggcgccatgggccgcagcggaatgctatctcgcgtgtgataacgtctgttatgcaatcaaagcgttaaaggccgtggtgcgtatttgcggcgaggtcagtgaacatcaaattctccgacagcgtgcagaaaagatgttacagcaactttctgacaggagctaaaagctt

SEQ ID NO: 60 - protein, 171 aa, synthetic construct
  REGION 1..171: note = synthetic construct
MGSSHHHHHHSQDPMKKDPTLQQAHDTMRFFRRGGSLRMLLDDDVTQPLNTLYRYATQLMEVKEFAGAARLFQLLTIYDAWSFDYWFRLGECCQAQKHWGEAIYAYGRAAQIKIDAPQAPWAAAECYLACDNVCYAIKALKAVVRICGEVSEHQILRQRAEKMLQQLSDRS

SEQ ID NO: 61 - DNA, 588 bp, Salmonella spp
  source 1..588: mol_type = genomic DNA
atgtcttcaggaaacatcttatggggaagtcaaaaccctattgtgtttaaaaatagcttcggcgtcagcaacgctgataccgggagccaggatgacttatcccagcaaaatccgtttgccgaagggtatggtgttttgcttattctccttatggttattcaggctatcgcaaataataaatttattgaagtccagaagaacgctgaacgtgccagaaatacccaggaaaagtcaaatgagatggatgaggtgattgctaaagcagccaaaggggatgctaaaaccaaagaggaggtgcctgaggatgtaattaaatacatgcgtgataatggtattctcatcgatggtatgaccattgatgattatatggctaaatatggcgatcatgggaagctggataaaggtggcctacaggcgatcaaagcggctttggataatgacgccaaccggaataccgatcttatgagtcaggggcagataacaattcaaaaaatgtctcaggagcttaacgctgtccttacccaactgacagggcttatcagtaagtggggggaaatttccagtatgatagcgcagaaaacgtactca

SEQ ID NO: 62 - protein, 196 aa, Salmonella spp
MSSGNILWGSQNPIVFKNSFGVSNADTGSQDDLSQQNPFAEGYGVLLILLMVIQAIANNKFIEVQKNAERARNTQEKSNEMDEVIAKAAKGDAKTKEEVPEDVIKYMRDNGILIDGMTIDDYMAKYGDHGKLDKGGLQAIKAALDNDANRNTDLMSQGQITIQKMSQELNAVLTQLTGLISKWGEISSMIAQKTYS

SEQ ID NO: 63 - DNA, 1455 bp, Salmonella spp
  source 1..1455: mol_type = genomic DNA
atgaatcgaattcacagtaatagcgacagcgccgcaggagtaaccgccttaacacatcatcacttaagcaatgtcagttgcgtttcctcgggttcgctgggaaagcgccagcatcgtgtgaattctacttttggcgatggcaacgccgcgtgtctgctatccgggaaaattagtcttcaggaggcaagcaatgcgttgaagcaactgcttgatgccgtacccggaaatcataagcgtccatcattgcctgactttttgcagaccaatcccgcggttttatcaatgatgatgacgtcattaatactcaacgtctttggtaataacgctcaatcgttatgccaacagcttgagcgggcaactgaggtgcaaaatgcattacgtaataagcaggtaaaggagtatcaggagcagatccagaaagcgatagagcaggaggataaagcgcgtaaagcgggtatttttggcgctatttttgactggattaccggcatatttgaaaccgtgattggcgccttaaaagttgtggaaggttttctgtccggaaatcccgcagaaatggctagcggcgtagcttatatggccgcaggttgtgcaggaatggttaaagccggagccgaaacggcaatgatgtgcggtgctgaccacgatacctgtcaggcaattattgacgtgacaagtaagattcaatttggttgtgaagccgtcgcgctggcactggatgttttccagattggccgtgcttttatggcgacgagaggtttatctggcgcagctgcaaaagtgcttgactccggttttggcgaggaagtggttgagcgtatggtaggtgcaggggaagcagaaatagaggagttggctgaaaagtttggcgaagaagtgagcgaaagtttttccaaacaatttgagccgcttgaacgtgaaatggctatggcgaatgagatggcagaggaggctgccgagttttctcgtaacgtagaaaataatatgacgcgaagcgcgggaaaaagctttacgaaagagggggtgaaagcaatggcaaaagaagcggcaaaagaagccctggaaaaatgtgtgcaagaaggtggaaagttcctgttaaaaaaattccgtaataaagttctcttcaatatgttcaaaaaaatcctgtatgccttactgagggattgttcatttaaaggcttacaggctatcagatgtgcaaccgagggcgccagtcagatgaatactggcatggttaacacagaaaaagcgaagatcgaaaagaaaatagagcaattaataactcagcaacggtttctggatttcataatgcaacaaacagaaaaccagaaaaagatagaacaaaaacgcttagaggagctttataaggggagcggtgccgcgcttagagatgtattagataccattgatcactatagtagcgttcaggcgagaatagctggctatcgcgcttaa

SEQ ID NO: 64 - protein, 484 aa, Salmonella spp
MNRIHSNSDSAAGVTALTHHHLSNVSCVSSGSLGKRQHRVNSTFGDGNAACLLSGKISLQEASNALKQLLDAVPGNHKRPSLPDFLQTNPAVLSMMMTSLILNVFGNNAQSLCQQLERATEVQNALRNKQVKEYQEQIQKAIEQEDKARKAGIFGAIFDWITGIFETVIGALKVVEGFLSGNPAEMASGVAYMAAGCAGMVKAGAETAMMCGADHDTCQAIIDVTSKIQFGCEAVALALDVFQIGRAFMATRGLSGAAAKVLDSGFGEEVVERMVGAGEAEIEELAEKFGEEVSESFSKQFEPLEREMAMANEMAEEAAEFSRNVENNMTRSAGKSFTKEGVKAMAKEAAKEALEKCVQEGGKFLLKKFRNKVLFNMFKKILYALLRDCSFKGLQAIRCATEGASQMNTGMVNTEKAKIEKKIEQLITQQRFLDFIMQQTENQKKIEQKRLEELYKGSGAALRDVLDTIDHYSSVQARIAGYRA

SEQ ID NO: 65 - DNA, 2049 bp, synthetic construct
  misc_feature 1..2049: note = synthetic construct
  source 1..2049: mol_type = other DNA
atgtcttcaggaaacatcttatggggaagtcaaaaccctattgtgtttaaaaatagcttcggcgtcagcaacgctgataccgggagccaggatgacttatcccagcaaaatccgtttgccgaagggtatggtgttttgcttattctccttatggttattcaggctatcgcaaataataaatttattgaagtccagaagaacgctgaacgtgccagaaatacccaggaaaagtcaaatgagatggatgaggtgattgctaaagcagccaaaggggatgctaaaaccaaagaggaggtgcctgaggatgtaattaaatacatgcgtgataatggtattctcatcgatggtatgaccattgatgattatatggctaaatatggcgatcatgggaagctggataaaggtggcctacaggcgatcaaagcggctttggataatgacgccaaccggaataccgatcttatgagtcaggggcagataacaattcaaaaaatgtctcaggagcttaacgctgtccttacccaactgacagggcttatcagtaagtggggggaaatttccagtatgatagcgcagaaaacgtactcagagctcatgaatcgaattcacagtaatagcgacagcgccgcaggagtaaccgccttaacacatcatcacttaagcaatgtcagttgcgtttcctcgggttcgctgggaaagcgccagcatcgtgtgaattctacttttggcgatggcaacgccgcgtgtctgctatccgggaaaattagtcttcaggaggcaagcaatgcgttgaagcaactgcttgatgccgtacccggaaatcataagcgtccatcattgcctgactttttgcagaccaatcccgcggttttatcaatgatgatgacgtcattaatactcaacgtctttggtaataacgctcaatcgttatgccaacagcttgagcgggcaactgaggtgcaaaatgcattacgtaataagcaggtaaaggagtatcaggagcagatccagaaagcgatagagcaggaggataaagcgcgtaaagcgggtatttttggcgctatttttgactggattaccggcatatttgaaaccgtgattggcgccttaaaagttgtggaaggttttctgtccggaaatcccgcagaaatggctagcggcgtagcttatatggccgcaggttgtgcaggaatggttaaagccggagccgaaacggcaatgatgtgcggtgctgaccacgatacctgtcaggcaattattgacgtgacaagtaagattcaatttggttgtgaagccgtcgcgctggcactggatgttttccagattggccgtgcttttatggcgacgagaggtttatctggcgcagctgcaaaagtgcttgactccggttttggcgaggaagtggttgagcgtatggtaggtgcaggggaagcagaaatagaggagttggctgaaaagtttggcgaagaagtgagcgaaagtttttccaaacaatttgagccgcttgaacgtgaaatggctatggcgaatgagatggcagaggaggctgccgagttttctcgtaacgtagaaaataatatgacgcgaagcgcgggaaaaagctttacgaaagagggggtgaaagcaatggcaaaagaagcggcaaaagaagccctggaaaaatgtgtgcaagaaggtggaaagttcctgttaaaaaaattccgtaataaagttctcttcaatatgttcaaaaaaatcctgtatgccttactgagggattgttcatttaaaggcttacaggctatcagatgtgcaaccgagggcgccagtcagatgaatactggcatggttaacacagaaaaagcgaagatcgaaaagaaaatagagcaattaataactcagcaacggtttctggatttcataatgcaacaaacagaaaaccagaaaaagatagaacaaaaacgcttagaggagctttataaggggagcggtgccgcgcttagagatgtattagataccattgatcactatagtagcgttcaggcgagaatagctggctatcgcgcttaa

SEQ ID NO: 66 - protein, 682 aa, synthetic construct
  REGION 1..682: note = synthetic construct
MSSGNILWGSQNPIVFKNSFGVSNADTGSQDDLSQQNPFAEGYGVLLILLMVIQAIANNKFIEVQKNAERARNTQEKSNEMDEVIAKAAKGDAKTKEEVPEDVIKYMRDNGILIDGMTIDDYMAKYGDHGKLDKGGLQAIKAALDNDANRNTDLMSQGQITIQKMSQELNAVLTQLTGLISKWGEISSMIAQKTYSELMNRIHSNSDSAAGVTALTHHHLSNVSCVSSGSLGKRQHRVNSTFGDGNAACLLSGKISLQEASNALKQLLDAVPGNHKRPSLPDFLQTNPAVLSMMMTSLILNVFGNNAQSLCQQLERATEVQNALRNKQVKEYQEQIQKAIEQEDKARKAGIFGAIFDWITGIFETVIGALKVVEGFLSGNPAEMASGVAYMAAGCAGMVKAGAETAMMCGADHDTCQAIIDVTSKIQFGCEAVALALDVFQIGRAFMATRGLSGAAAKVLDSGFGEEVVERMVGAGEAEIEELAEKFGEEVSESFSKQFEPLEREMAMANEMAEEAAEFSRNVENNMTRSAGKSFTKEGVKAMAKEAAKEALEKCVQEGGKFLLKKFRNKVLFNMFKKILYALLRDCSFKGLQAIRCATEGASQMNTGMVNTEKAKIEKKIEQLITQQRFLDFIMQQTENQKKIEQKRLEELYKGSGAALRDVLDTIDHYSSVQARIAGYRA

SEQ ID NO: 67 - DNA, 2655 bp, synthetic construct
  misc_feature 1..2655: note = synthetic construct
  source 1..2655: mol_type = other DNA
catatggacaatggcgatcgtttataccgtgccgactcgcgtcccccagatgagattaaacgtagcggtgggttaatgccacgtgggcacaatgagtattttgaccgtggaacacagatgaacattaacctttacgatcatgcccgtgggacccagaccgggtttgtccgttatgatgacgggtatgttagtacgagtttgtccttacgctccgcacaccttgcgggacaaagtattttatcaggctacagcacatattacatttatgtgatcgccactgccccaaacatgttcaatgtgaacgatgtgttgggggtttacagcccccatccatatgaacaagaagtctcggcccttggggggatcccatatagccagatttatggttggtaccgcgtaaattttggtgtgattgatgaacgtttgcatcgtaaccgtgaataccgcgatcgctactaccgtaacttgaacattgcacctgccgaggacggctatcgtttagcgggattcccacccgatcatcaggcgtggcgtgaggaaccgtggatccatcacgcccctcaggggtgcgggaacagtagtcgcgggtccgcggcatccatgtcttcaggaaacatcttatggggaagtcaaaaccctattgtgtttaaaaatagcttcggcgtcagcaacgctgataccgggagccaggatgacttatcccagcaaaatccgtttgccgaagggtatggtgttttgcttattctccttatggttattcaggctatcgcaaataataaatttattgaagtccagaagaacgctgaacgtgccagaaatacccaggaaaagtcaaatgagatggatgaggtgattgctaaagcagccaaaggggatgctaaaaccaaagaggaggtgcctgaggatgtaattaaatacatgcgtgataatggtattctcatcgatggtatgaccattgatgattatatggctaaatatggcgatcatgggaagctggataaaggtggcctacaggcgatcaaagcggctttggataatgacgccaaccggaataccgatcttatgagtcaggggcagataacaattcaaaaaatgtctcaggagcttaacgctgtccttacccaactgacagggcttatcagtaagtggggggaaatttccagtatgatagcgcagaaaacgtactcagagctcatgaatcgaattcacagtaatagcgacagcgccgcaggagtaaccgccttaacacatcatcacttaagcaatgtcagttgcgtttcctcgggttcgctgggaaagcgccagcatcgtgtgaattctacttttggcgatggcaacgccgcgtgtctgctatccgggaaaattagtcttcaggaggcaagcaatgcgttgaagcaactgcttgatgccgtacccggaaatcataagcgtccatcattgcctgactttttgcagaccaatcccgcggttttatcaatgatgatgacgtcattaatactcaacgtctttggtaataacgctcaatcgttatgccaacagcttgagcgggcaactgaggtgcaaaatgcattacgtaataagcaggtaaaggagtatcaggagcagatccagaaagcgatagagcaggaggataaagcgcgtaaagcgggtatttttggcgctatttttgactggattaccggcatatttgaaaccgtgattggcgccttaaaagttgtggaaggttttctgtccggaaatcccgcagaaatggctagcggcgtagcttatatggccgcaggttgtgcaggaatggttaaagccggagccgaaacggcaatgatgtgcggtgctgaccacgatacctgtcaggcaattattgacgtgacaagtaagattcaatttggttgtgaagccgtcgcgctggcactggatgttttccagattggccgtgcttttatggcgacgagaggtttatctggcgcagctgcaaaagtgcttgactccggttttggcgaggaagtggttgagcgtatggtaggtgcaggggaagcagaaatagaggagttggctgaaaagtttggcgaagaagtgagcgaaagtttttccaaacaatttgagccgcttgaacgtgaaatggctatggcgaatgagatggcagaggaggctgccgagttttctcgtaacgtagaaaataatatgacgcgaagcgcgggaaaaagctttacgaaagagggggtgaaagcaatggcaaaagaagcggcaaaagaagccctggaaaaatgtgtgcaagaaggtggaaagttcctgttaaaaaaattccgtaataaagttctcttcaatatgttcaaaaaaatcctgtatgccttactgagggattgttcatttaaaggcttacaggctatcagatgtgcaaccgagggcgccagtcagatgaatactggcatggttaacacagaaaaagcgaagatcgaaaagaaaatagagcaattaataactcagcaacggtttctggatttcataatgcaacaaacagaaaaccagaaaaagatagaacaaaaacgcttagaggagctttataaggggagcggtgccgcgcttagagatgtattagataccattgatcactatagtagcgttcaggcgagaatagctggctatcgcgcttaactcgag

SEQ ID NO: 68 - protein, 881 aa, synthetic construct
  REGION 1..881: note = synthetic construct
MDNGDRLYRADSRPPDEIKRSGGLMPRGHNEYFDRGTQMNINLYDHARGTQTGFVRYDDGYVSTSLSLRSAHLAGQSILSGYSTYYIYVIATAPNMFNVNDVLGVYSPHPYEQEVSALGGIPYSQIYGWYRVNFGVIDERLHRNREYRDRYYRNLNIAPAEDGYRLAGFPPDHQAWREEPWIHHAPQGCGNSSRGSAASMSSGNILWGSQNPIVFKNSFGVSNADTGSQDDLSQQNPFAEGYGVLLILLMVIQAIANNKFIEVQKNAERARNTQEKSNEMDEVIAKAAKGDAKTKEEVPEDVIKYMRDNGILIDGMTIDDYMAKYGDHGKLDKGGLQAIKAALDNDANRNTDLMSQGQITIQKMSQELNAVLTQLTGLISKWGEISSMIAQKTYSELMNRIHSNSDSAAGVTALTHHHLSNVSCVSSGSLGKRQHRVNSTFGDGNAACLLSGKISLQEASNALKQLLDAVPGNHKRPSLPDFLQTNPAVLSMMMTSLILNVFGNNAQSLCQQLERATEVQNALRNKQVKEYQEQIQKAIEQEDKARKAGIFGAIFDWITGIFETVIGALKVVEGFLSGNPAEMASGVAYMAAGCAGMVKAGAETAMMCGADHDTCQAIIDVTSKIQFGCEAVALALDVFQIGRAFMATRGLSGAAAKVLDSGFGEEVVERMVGAGEAEIEELAEKFGEEVSESFSKQFEPLEREMAMANEMAEEAAEFSRNVENNMTRSAGKSFTKEGVKAMAKEAAKEALEKCVQEGGKFLLKKFRNKVLFNMFKKILYALLRDCSFKGLQAIRCATEGASQMNTGMVNTEKAKIEKKIEQLITQQRFLDFIMQQTENQKKIEQKRLEELYKGSGAALRDVLDTIDHYSSVQARIAGYRA

SEQ ID NO: 69 - DNA, 1254 bp, synthetic construct
  misc_feature 1..1254: note = synthetic construct
  source 1..1254: mol_type = other DNA
atgggcagcagccatcatcatcatcatcacagcagcggcctggtgccgcgcggcagccatatggacaatggcgatcgtttataccgtgccgactcgcgtcccccagatgagattaaacgtagcggtgggttaatgccacgtgggcacaatgagtattttgaccgtggaacacagatgaacattaacctttacgatcatgcccgtgggacccagaccgggtttgtccgttatgatgacgggtatgttagtacgagtttgtccttacgctccgcacaccttgcgggacaaagtattttatcaggctacagcacatattacatttatgtgatcgccactgccccaaacatgttcaatgtgaacgatgtgttgggggtttacagcccccatccatatgaacaagaagtctcggcccttggggggatcccatatagccagatttatggttggtaccgcgtaaattttggtgtgattgatgaacgtttgcatcgtaaccgtgaataccgcgatcgctactaccgtaacttgaacattgcacctgccgaggacggctatcgtttagcgggattcccacccgatcatcaggcgtggcgtgaggaaccgtggatccatcacgcccctcaggggtgcgggaacagtagtcgcgggtccgcggcatccatgtcttcaggaaacatcttatggggaagtcaaaaccctattgtgtttaaaaatagcttcggcgtcagcaacgctgataccgggagccaggatgacttatcccagcaaaatccgtttgccgaagggtatggtgttttgcttattctccttatggttattcaggctatcgcaaataataaatttattgaagtccagaagaacgctgaacgtgccagaaatacccaggaaaagtcaaatgagatggatgaggtgattgctaaagcagccaaaggggatgctaaaaccaaagaggaggtgcctgaggatgtaattaaatacatgcgtgataatggtattctcatcgatggtatgaccattgatgattatatggctaaatatggcgatcatgggaagctggataaaggtggcctacaggcgatcaaagcggctttggataatgacgccaaccggaataccgatcttatgagtcaggggcagataacaattcaaaaaatgtctcaggagcttaacgctgtccttacccaactgacagggcttatcagtaagtggggggaaatttccagtatgatagcgcagaaaacgtactcataaggatcc

SEQ ID NO: 70 - protein, 415 aa, synthetic construct
  REGION 1..415: note = synthetic construct
MGSSHHHHHHSSGLVPRGSHMDNGDRLYRADSRPPDEIKRSGGLMPRGHNEYFDRGTQMNINLYDHARGTQTGFVRYDDGYVSTSLSLRSAHLAGQSILSGYSTYYIYVIATAPNMFNVNDVLGVYSPHPYEQEVSALGGIPYSQIYGWYRVNFGVIDERLHRNREYRDRYYRNLNIAPAEDGYRLAGFPPDHQAWREEPWIHHAPQGCGNSSRGSAASMSSGNILWGSQNPIVFKNSFGVSNADTGSQDDLSQQNPFAEGYGVLLILLMVIQAIANNKFIEVQKNAERARNTQEKSNEMDEVIAKAAKGDAKTKEEVPEDVIKYMRDNGILIDGMTIDDYMAKYGDHGKLDKGGLQAIKAALDNDANRNTDLMSQGQITIQKMSQELNAVLTQLTGLISKWGEISSMIAQKTYS

SEQ ID NO: 71 - DNA, 749 bp, synthetic construct
  misc_feature 1..749: note = synthetic construct
  source 1..749: mol_type = other DNA
atgggcagcagccatcaccatcatcaccacagccaggatccgatgagcactccatcttctaataattctaaaaaaccttcggcctcttttaataaaaaatcacgtagccgcttggccgagattgctgcacaaaaaaaagcaaaagctgaggatttggaacaaaaatatcctgttcctacggaagaggagacaaaacaagttctcatggacatcctacaggggttaagcaacggattaactcttcagcaaattttaggtctctccgacgtcctccttgaagagatctacaccgtagcatataccttctactcccaagggaaatatcgggaagctatcggtcttttccaaatcttaacagcctccaaacctcaatgctacaaatacatcttaggtcttagctcttgctatcaccagctaaaaatgtatgatgaagccgcttttggtttcttcctagctttcgatgctcaacccgaaaaccccatccctccttactacatcgccgatagcttgatgaagctaaaccaacccgaagaatctcaagacttcctcgatattacgatcgatatgtgtaagaacaagccggaatataaagttcttaaagatcgctgcagcattatgaagcaatctttagatgccgtgctgaaaaaagagaaatctgcaaaaggctctgaaacacaagcctcctctcctaaaaacacaaaagctaaaaaagctgcttctaacaagaaaaaagcaaagtaagcggccgc

SEQ ID NO: 72 - protein, 246 aa, synthetic construct
  REGION 1..246: note = synthetic construct
MGSSHHHHHHSQDPMSTPSSNNSKKPSASFNKKSRSRLAEIAAQKKAKAEDLEQKYPVPTEEETKQVLMDILQGLSNGLTLQQILGLSDVLLEEIYTVAYTFYSQGKYREAIGLFQILTASKPQCYKYILGLSSCYHQLKMYDEAAFGFFLAFDAQPENPIPPYYIADSLMKLNQPEESQDFLDITIDMCKNKPEYKVLKDRCSIMKQSLDAVLKKEKSAKGSETQASSPKNTKAKKAASNKKKAK

SEQ ID NO: 73 - DNA, 441 bp, Chlamydia spp
  source 1..441: mol_type = genomic DNA
aaaagtgagcgtttaaaaaaattagaatcagagcttcatgatcttacccagtggatgcaacttggccttgttcctaaaaaagaaatcgagagacaccaggaagaaatccgtctgctagaaagcaaaatccttgaagagaaagaacgtctacaacttctcaaagaaagcggtgagatcaaagagtacgtaacccctcgaagaactccagctaaaaccatttacccagatggccccagcgtttcagacgttgagtttgtagaatcctcggatacagaagtggatctcgatgccggtgacacaattgagattgacctaggtgatgaggcaagagaagaaagcggaaacgaactcgactactctagtgaagacgatgaggatcctttcagcgatcgcaatcgttggcgccgaggaggcatcatagatcctgacgcgaatgaatgg

SEQ ID NO: 74 - protein, 148 aa, Chlamydia spp
MKSERLKKLESELHDLTQWMQLGLVPKKEIERHQEEIRLLESKILEEKERLQLLKESGEIKEYVTPRRTPAKTIYPDGPSVSDVEFVESSDTEVDLDAGDTIEIDLGDEAREESGNELDYSSEDDEDPFSDRNRWRRGGIIDPDANEW

SEQ ID NO: 75 - DNA, 1464 bp, Chlamydia spp
  source 1..1464: mol_type = genomic DNA
atgagcttgtcatccagcagcagctcggatagttcgaatctgaaaaatgtgttatctcaggtcatcgcgtctacaccacagggggttcctaatgctgacaaattaaccgacaatcaggtaaaacaagtccagcagacccgtcaaaaccgtgatgatctgtccatggagagcgacgtcgcggtggcgggaacagccggaaaagatcgtgctgcgtcggcgtcccagatcgagggacaagagctgattgagcaacagggacttgcggctgggaaagagacggcttctgctgatgctacatcattgacccagtcggcatccaaaggcgcttccagtcagcagtgtattgaggataccagtaagtccctggagctttcttcgctttcgagcctgtcaagcgtagatgcgacacatttgcaggaaatccaatcgatcgtgtcttcagcaatgggcgccaccaacgaattgtcattgacgaacttagagacaccgggattaccaaagccgagtaccactccacgccaggaagttatggagatcagccttgccttagcgaaggccatcactgcattgggtgagagcactcaggctgccttggaaaattttcagtccactcagagtcagtccgcgaacatgaataagatgagtttggaatcccaaggcttgaaaatcgacaaggagcgtgaagaatttaagaaaatgcaggagattcagcaaaagagcggcacaaattcaaccatggatactgtgaataaagttatgattggcgtgacagtggcaattacagtaatctctgttgtttcagcattgtttacctgcggtttgggcttgattggcacagccgctgcgggtgccacagccgccaccgctggggcaacggccgccgccacgaccgctacctctgtgacgaccacagtcgctacccaggtgacgatgcaagcggtggtccaagtcgttaagcaggctattatccaagcagtaaaacgcgccatcgtccaagcgattaaacaggggattaagcaaggcattaaacaagcgatcaaacaggcagtcaaggcaagcgtgaagacacttgccaaaaatgtaggcaagattttcagcgcaggcaagaacgctgtgagtaagtccttcccaaaattgtctaaggtgattaatacacttggttccaaatgggttactcttggcgtgggggcccttacagcggtgccgcagttagtcagtggcattacctcccttcaattgtctgatatgcaaaaagaacttgcacaaatccaaaaggaagtgggtgcacttacggcgcagagtgagatgatgaaagcgtttacactgttctggcagcaagcttcgaaaatcgcggccaaacaaacggaatcaccttcagagacgcaacaacaggcagctaagaccggcgcccagatcgctaaagcgttgtccgccatttcgggtgctttagctgctgctgcttag

SEQ ID NO: 76 - protein, 487 aa, Chlamydia spp
MSLSSSSSSDSSNLKNVLSQVIASTPQGVPNADKLTDNQVKQVQQTRQNRDDLSMESDVAVAGTAGKDRAASASQIEGQELIEQQGLAAGKETASADATSLTQSASKGASSQQCIEDTSKSLELSSLSSLSSVDATHLQEIQSIVSSAMGATNELSLTNLETPGLPKPSTTPRQEVMEISLALAKAITALGESTQAALENFQSTQSQSANMNKMSLESQGLKIDKEREEFKKMQEIQQKSGTNSTMDTVNKVMIGVTVAITVISVVSALFTCGLGLIGTAAAGATAATAGATAAATTATSVTTTVATQVTMQAVVQVVKQAIIQAVKRAIVQAIKQGIKQGIKQAIKQAVKASVKTLAKNVGKIFSAGKNAVSKSFPKLSKVINTLGSKWVTLGVGALTAVPQLVSGITSLQLSDMQKELAQIQKEVGALTAQSEMMKAFTLFWQQASKIAAKQTESPSETQQQAAKTGAQIAKALSAISGALAAAA

SEQ ID NO: 77 - DNA, 1920 bp, synthetic construct
  misc_feature 1..1920: note = synthetic construct
  source 1..1920: mol_type = other DNA
aaaagtgagcgtttaaaaaaattagaatcagagcttcatgatcttacccagtggatgcaacttggccttgttcctaaaaaagaaatcgagagacaccaggaagaaatccgtctgctagaaagcaaaatccttgaagagaaagaacgtctacaacttctcaaagaaagcggtgagatcaaagagtacgtaacccctcgaagaactccagctaaaaccatttacccagatggccccagcgtttcagacgttgagtttgtagaatcctcggatacagaagtggatctcgatgccggtgacacaattgagattgacctaggtgatgaggcaagagaagaaagcggaaacgaactcgactactctagtgaagacgatgaggatcctttcagcgatcgcaatcgttggcgccgaggaggcatcatagatcctgacgcgaatgaatggggttcagctgcttcaatgagcttgtcatccagcagcagctcggatagttcgaatctgaaaaatgtgttatctcaggtcatcgcgtctacaccacagggggttcctaatgctgacaaattaaccgacaatcaggtaaaacaagtccagcagacccgtcaaaaccgtgatgatctgtccatggagagcgacgtcgcggtggcgggaacagccggaaaagatcgtgctgcgtcggcgtcccagatcgagggacaagagctgattgagcaacagggacttgcggctgggaaagagacggcttctgctgatgctacatcattgacccagtcggcatccaaaggcgcttccagtcagcagtgtattgaggataccagtaagtccctggagctttcttcgctttcgagcctgtcaagcgtagatgcgacacatttgcaggaaatccaatcgatcgtgtcttcagcaatgggcgccaccaacgaattgtcattgacgaacttagagacaccgggattaccaaagccgagtaccactccacgccaggaagttatggagatcagccttgccttagcgaaggccatcactgcattgggtgagagcactcaggctgccttggaaaattttcagtccactcagagtcagtccgcgaacatgaataagatgagtttggaatcccaaggcttgaaaatcgacaaggagcgtgaagaatttaagaaaatgcaggagattcagcaaaagagcggcacaaattcaaccatggatactgtgaataaagttatgattggcgtgacagtggcaattacagtaatctctgttgtttcagcattgtttacctgcggtttgggcttgattggcacagccgctgcgggtgccacagccgccaccgctggggcaacggccgccgccacgaccgctacctctgtgacgaccacagtcgctacccaggtgacgatgcaagcggtggtccaagtcgttaagcaggctattatccaagcagtaaaacgcgccatcgtccaagcgattaaacaggggattaagcaaggcattaaacaagcgatcaaacaggcagtcaaggcaagcgtgaagacacttgccaaaaatgtaggcaagattttcagcgcaggcaagaacgctgtgagtaagtccttcccaaaattgtctaaggtgattaatacacttggttccaaatgggttactcttggcgtgggggcccttacagcggtgccgcagttagtcagtggcattacctcccttcaattgtctgatatgcaaaaagaacttgcacaaatccaaaaggaagtgggtgcacttacggcgcagagtgagatgatgaaagcgtttacactgttctggcagcaagcttcgaaaatcgcggccaaacaaacggaatcaccttcagagacgcaacaacaggcagctaagaccggcgcccagatcgctaaagcgttgtccgccatttcgggtgctttagctgctgctgcttag

SEQ ID NO: 78 - protein, 640 aa, synthetic construct
  REGION 1..640: note = synthetic construct
MKSERLKKLESELHDLTQWMQLGLVPKKEIERHQEEIRLLESKILEEKERLQLLKESGEIKEYVTPRRTPAKTIYPDGPSVSDVEFVESSDTEVDLDAGDTIEIDLGDEAREESGNELDYSSEDDEDPFSDRNRWRRGGIIDPDANEWGSAASMSLSSSSSSDSSNLKNVLSQVIASTPQGVPNADKLTDNQVKQVQQTRQNRDDLSMESDVAVAGTAGKDRAASASQIEGQELIEQQGLAAGKETASADATSLTQSASKGASSQQCIEDTSKSLELSSLSSLSSVDATHLQEIQSIVSSAMGATNELSLTNLETPGLPKPSTTPRQEVMEISLALAKAITALGESTQAALENFQSTQSQSANMNKMSLESQGLKIDKEREEFKKMQEIQQKSGTNSTMDTVNKVMIGVTVAITVISVVSALFTCGLGLIGTAAAGATAATAGATAAATTATSVTTTVATQVTMQAVVQVVKQAIIQAVKRAIVQAIKQGIKQGIKQAIKQAVKASVKTLAKNVGKIFSAGKNAVSKSFPKLSKVINTLGSKWVTLGVGALTAVPQLVSGITSLQLSDMQKELAQIQKEVGALTAQSEMMKAFTLFWQQASKIAAKQTESPSETQQQAAKTGAQIAKALSAISGALAAAA

SEQ ID NO: 79 - DNA, 2517 bp, synthetic construct
  misc_feature 1..2517: note = synthetic construct
  source 1..2517: mol_type = other DNA
catatggacaatggcgatcgtttataccgtgccgactcgcgtcccccagatgagattaaacgtagcggtgggttaatgccacgtgggcacaatgagtattttgaccgtggaacacagatgaacattaacctttacgatcatgcccgtgggacccagaccgggtttgtccgttatgatgacgggtatgttagtacgagtttgtccttacgctccgcacaccttgcgggacaaagtattttatcaggctacagcacatattacatttatgtgatcgccactgccccaaacatgttcaatgtgaacgatgtgttgggggtttacagcccccatccatatgaacaagaagtctcggcccttggggggatcccatatagccagatttatggttggtaccgcgtaaattttggtgtgattgatgaacgtttgcatcgtaaccgtgaataccgcgatcgctactaccgtaacttgaacattgcacctgccgaggacggctatcgtttagcgggattcccacccgatcatcaggcgtggcgtgaggaaccgtggatccatcacgcccctcaggggtgcgggaacagtagtcgccatatgaaaagtgagcgtttaaaaaaattagaatcagagcttcatgatcttacccagtggatgcaacttggccttgttcctaaaaaagaaatcgagagacaccaggaagaaatccgtctgctagaaagcaaaatccttgaagagaaagaacgtctacaacttctcaaagaaagcggtgagatcaaagagtacgtaacccctcgaagaactccagctaaaaccatttacccagatggccccagcgtttcagacgttgagtttgtagaatcctcggatacagaagtggatctcgatgccggtgacacaattgagattgacctaggtgatgaggcaagagaagaaagcggaaacgaactcgactactctagtgaagacgatgaggatcctttcagcgatcgcaatcgttggcgccgaggaggcatcatagatcctgacgcgaatgaatggggttcagctgcttcaatgagcttgtcatccagcagcagctcggatagttcgaatctgaaaaatgtgttatctcaggtcatcgcgtctacaccacagggggttcctaatgctgacaaattaaccgacaatcaggtaaaacaagtccagcagacccgtcaaaaccgtgatgatctgtccatggagagcgacgtcgcggtggcgggaacagccggaaaagatcgtgctgcgtcggcgtcccagatcgagggacaagagctgattgagcaacagggacttgcggctgggaaagagacggcttctgctgatgctacatcattgacccagtcggcatccaaaggcgcttccagtcagcagtgtattgaggataccagtaagtccctggagctttcttcgctttcgagcctgtcaagcgtagatgcgacacatttgcaggaaatccaatcgatcgtgtcttcagcaatgggcgccaccaacgaattgtcattgacgaacttagagacaccgggattaccaaagccgagtaccactccacgccaggaagttatggagatcagccttgccttagcgaaggccatcactgcattgggtgagagcactcaggctgccttggaaaattttcagtccactcagagtcagtccgcgaacatgaataagatgagtttggaatcccaaggcttgaaaatcgacaaggagcgtgaagaatttaagaaaatgcaggagattcagcaaaagagcggcacaaattcaaccatggatactgtgaataaagttatgattggcgtgacagtggcaattacagtaatctctgttgtttcagcattgtttacctgcggtttgggcttgattggcacagccgctgcgggtgccacagccgccaccgctggggcaacggccgccgccacgaccgctacctctgtgacgaccacagtcgctacccaggtgacgatgcaagcggtggtccaagtcgttaagcaggctattatccaagcagtaaaacgcgccatcgtccaagcgattaaacaggggattaagcaaggcattaaacaagcgatcaaacaggcagtcaaggcaagcgtgaagacacttgccaaaaatgtaggcaagattttcagcgcaggcaagaacgctgtgagtaagtccttcccaaaattgtctaaggtgattaatacacttggttccaaatgggttactcttggcgtgggggcccttacagcggtgccgcagttagtcagtggcattacctcccttcaattgtctgatatgcaaaaagaacttgcacaaatccaaaaggaagtgggtgcacttacggcgcagagtgagatgatgaaagcgtttacactgttctggcagcaagcttcgaaaatcgcggccaaacaaacggaatcaccttcagagacgcaacaacaggcagctaagaccggcgcccagatcgctaaagcgttgtccgccatttcgggtgctttagctgctgctgcttagctcgag

SEQ ID NO: 80 - protein, 834 aa, synthetic construct
  REGION 1..834: note = synthetic construct
MDNGDRLYRADSRPPDEIKRSGGLMPRGHNEYFDRGTQMNINLYDHARGTQTGFVRYDDGYVSTSLSLRSAHLAGQSILSGYSTYYIYVIATAPNMFNVNDVLGVYSPHPYEQEVSALGGIPYSQIYGWYRVNFGVIDERLHRNREYRDRYYRNLNIAPAEDGYRLAGFPPDHQAWREEPWIHHAPQGCGNSSRMKSERLKKLESELHDLTQWMQLGLVPKKEIERHQEEIRLLESKILEEKERLQLLKESGEIKEYVTPRRTPAKTIYPDGPSVSDVEFVESSDTEVDLDAGDTIEIDLGDEAREESGNELDYSSEDDEDPFSDRNRWRRGGIIDPDANEWGSAASMSLSSSSSSDSSNLKNVLSQVIASTPQGVPNADKLTDNQVKQVQQTRQNRDDLSMESDVAVAGTAGKDRAASASQIEGQELIEQQGLAAGKETASADATSLTQSASKGASSQQCIEDTSKSLELSSLSSLSSVDATHLQEIQSIVSSAMGATNELSLTNLETPGLPKPSTTPRQEVMEISLALAKAITALGESTQAALENFQSTQSQSANMNKMSLESQGLKIDKEREEFKKMQEIQQKSGTNSTMDTVNKVMIGVTVAITVISVVSALFTCGLGLIGTAAAGATAATAGATAAATTATSVTTTVATQVTMQAVVQVVKQAIIQAVKRAIVQAIKQGIKQGIKQAIKQAVKASVKTLAKNVGKIFSAGKNAVSKSFPKLSKVINTLGSKWVTLGVGALTAVPQLVSGITSLQLSDMQKELAQIQKEVGALTAQSEMMKAFTLFWQQASKIAAKQTESPSETQQQAAKTGAQIAKALSAISGALAAAA

SEQ ID NO: 81 - DNA, 749 bp, synthetic construct; identical to SEQ ID NO: 71
  misc_feature 1..749: note = synthetic construct
  source 1..749: mol_type = other DNA

SEQ ID NO: 82 - protein, 246 aa, synthetic construct; identical to SEQ ID NO: 72
  REGION 1..246: note = synthetic construct

SEQ ID NO: 83 - DNA, 666 bp, Chlamydia spp
  source 1..666: mol_type = genomic DNA
atagatcctcttaagctttttccaaattttgatggggataaggagagtgctgcggtgaataaaccttcagcatctcctatgcccagcgaattaagtaaaaatgttgcctcattctctttagggggtggaggtgctgcgttggattcgacagtgtccacagaaaagctatcgttgatggctatgatgcaggataaaaattcgcagttgatcgatcctgagttggaggaagctctgaactctgaagagttacaagagcagatccatttgttaaaaagtcgtttgtgggatgcacaaacgcagatgcaaatgcaagatcccgacaagttggcctctgagcatgtagatgctttaggagtcattgttgatttaatcaatggggattttcaagcgatagctgaacatacacaacagacggtcaagcagggtaatggtgacgaagaaaaatctgttacacgcaagatagtcgattgggtctcttcaggagaagaaattttgaatcgtgctttgttgtatttctccgatcgtaatggagaaagagaaacattagccgatttcttaaaagttcagtatgccgttcaaagagctacacaacgcgccgagttatttgccagtattctaggtgccacggtgagtagtgtaaaaacgattatgacaacccagttaggt

SEQ ID NO: 84 - protein, 222 aa, Chlamydia spp
MIDPLKLFPNFDGDKESAAVNKPSASPMPSELSKNVASFSLGGGGAALDSTVSTEKLSLMAMMQDKNSQLIDPELEEALNSEELQEQIHLLKSRLWDAQTQMQMQDPDKLASEHVDALGVIVDLINGDFQAIAEHTQQTVKQGNGDEEKSVTRKIVDWVSSGEEILNRALLYFSDRNGERETLADFLKVQYAVQRATQRAELFASILGATVSSVKTIMTTQL

SEQ ID NO: 85 - DNA, 2145 bp, synthetic construct
  misc_feature 1..2145: note = synthetic construct
  source 1..2145: mol_type = other DNA
atagatcctcttaagctttttccaaattttgatggggataaggagagtgctgcggtgaataaaccttcagcatctcctatgcccagcgaattaagtaaaaatgttgcctcattctctttagggggtggaggtgctgcgttggattcgacagtgtccacagaaaagctatcgttgatggctatgatgcaggataaaaattcgcagttgatcgatcctgagttggaggaagctctgaactctgaagagttacaagagcagatccatttgttaaaaagtcgtttgtgggatgcacaaacgcagatgcaaatgcaagatcccgacaagttggcctctgagcatgtagatgctttaggagtcattgttgatttaatcaatggggattttcaagcgatagctgaacatacacaacagacggtcaagcagggtaatggtgacgaagaaaaatctgttacacgcaagatagtcgattgggtctcttcaggagaagaaattttgaatcgtgctttgttgtatttctccgatcgtaatggagaaagagaaacattagccgatttcttaaaagttcagtatgccgttcaaagagctacacaacgcgccgagttatttgccagtattctaggtgccacggtgagtagtgtaaaaacgattatgacaacccagttaggtggttcagctgcttcaatgagcttgtcatccagcagcagctcggatagttcgaatctgaaaaatgtgttatctcaggtcatcgcgtctacaccacagggggttcctaatgctgacaaattaaccgacaatcaggtaaaacaagtccagcagacccgtcaaaaccgtgatgatctgtccatggagagcgacgtcgcggtggcgggaacagccggaaaagatcgtgctgcgtcggcgtcccagatcgagggacaagagctgattgagcaacagggacttgcggctgggaaagagacggcttctgctgatgctacatcattgacccagtcggcatccaaaggcgcttccagtcagcagtgtattgaggataccagtaagtccctggagctttcttcgctttcgagcctgtcaagcgtagatgcgacacatttgcaggaaatccaatcgatcgtgtcttcagcaatgggcgccaccaacgaattgtcattgacgaacttagagacaccgggattaccaaagccgagtaccactccacgccaggaagttatggagatcagccttgccttagcgaaggccatcactgcattgggtgagagcactcaggctgccttggaaaattttcagtccactcagagtcagtccgcgaacatgaataagatgagtttggaatcccaaggcttgaaaatcgacaaggagcgtgaagaatttaagaaaatgcaggagattcagcaaaagagcggcacaaattcaaccatggatactgtgaataaagttatgattggcgtgacagtggcaattacagtaatctctgttgtttcagcattgtttacctgcggtttgggcttgattggcacagccgctgcgggtgccacagccgccaccgctggggcaacggccgccgccacgaccgctacctctgtgacgaccacagtcgctacccaggtgacgatgcaagcggtggtccaagtcgttaagcaggctattatccaagcagtaaaacgcgccatcgtccaagcgattaaacaggggattaagcaaggcattaaacaagcgatcaaacaggcagtcaaggcaagcgtgaagacacttgccaaaaatgtaggcaagattttcagcgcaggcaagaacgctgtgagtaagtccttcccaaaattgtctaaggtgattaatacacttggttccaaatgggttactcttggcgtgggggcccttacagcggtgccgcagttagtcagtggcattacctcccttcaattgtctgatatgcaaaaagaacttgcacaaatccaaaaggaagtgggtgcacttacggcgcagagtgagatgatgaaagcgtttacactgttctggcagcaagcttcgaaaatcgcggccaaacaaacggaatcaccttcagagacgcaacaacaggcagctaagaccggcgcccagatcgctaaagcgttgtccgccatttcgggtgctttagctgctgctgcttag

SEQ ID NO: 86 - protein, 715 aa, synthetic construct
  REGION 1..715: note = synthetic construct
MIDPLKLFPNFDGDKESAAVNKPSASPMPSELSKNVASFSLGGGGAALDSTVSTEKLSLMAMMQDKNSQLIDPELEEALNSEELQEQIHLLKSRLWDAQTQMQMQDPDKLASEHVDALGVIVDLINGDFQAIAEHTQQTVKQGNGDEEKSVTRKIVDWVSSGEEILNRALLYFSDRNGERETLADFLKVQYAVQRATQRAELFASILGATVSSVKTIMTTQLGGSAASMSLSSSSSSDSSNLKNVLSQVIASTPQGVPNADKLTDNQVKQVQQTRQNRDDLSMESDVAVAGTAGKDRAASASQIEGQELIEQQGLAAGKETASADATSLTQSASKGASSQQCIEDTSKSLELSSLSSLSSVDATHLQEIQSIVSSAMGATNELSLTNLETPGLPKPSTTPRQEVMEISLALAKAITALGESTQAALENFQSTQSQSANMNKMSLESQGLKIDKEREEFKKMQEIQQKSGTNSTMDTVNKVMIGVTVAITVISVVSALFTCGLGLIGTAAAGATAATAGATAAATTATSVTTTVATQVTMQAVVQVVKQAIIQAVKRAIVQAIKQGIKQGIKQAIKQAVKASVKTLAKNVGKIFSAGKNAVSKSFPKLSKVINTLGSKWVTLGVGALTAVPQLVSGITSLQLSDMQKELAQIQKEVGALTAQSEMMKAFTLFWQQASKIAAKQTESPSETQQQAAKTGAQIAKALSAISGALAAAA

SEQ ID NO: 87 - DNA, 2742 bp, synthetic construct
  misc_feature 1..2742: note = synthetic construct
  source 1..2742: mol_type = other DNA
catatggacaatggcgatcgtttataccgtgccgactcgcgtcccccagatgagattaaacgtagcggtgggttaatgccacgtgggcacaatgagtattttgaccgtggaacacagatgaacattaacctttacgatcatgcccgtgggacccagaccgggtttgtccgttatgatgacgggtatgttagtacgagtttgtccttacgctccgcacaccttgcgggacaaagtattttatcaggctacagcacatattacatttatgtgatcgccactgccccaaacatgttcaatgtgaacgatgtgttgggggtttacagcccccatccatatgaacaagaagtctcggcccttggggggatcccatatagccagatttatggttggtaccgcgtaaattttggtgtgattgatgaacgtttgcatcgtaaccgtgaataccgcgatcgctactaccgtaacttgaacattgcacctgccgaggacggctatcgtttagcgggattcccacccgatcatcaggcgtggcgtgaggaaccgtggatccatcacgcccctcaggggtgcgggaacagtagtcgccatatgatagatcctcttaagctttttccaaattttgatggggataaggagagtgctgcggtgaataaaccttcagcatctcctatgcccagcgaattaagtaaaaatgttgcctcattctctttagggggtggaggtgctgcgttggattcgacagtgtccacagaaaagctatcgttgatggctatgatgcaggataaaaattcgcagttgatcgatcctgagttggaggaagctctgaactctgaagagttacaagagcagatccatttgttaaaaagtcgtttgtgggatgcacaaacgcagatgcaaatgcaagatcccgacaagttggcctctgagcatgtagatgctttaggagtcattgttgatttaatcaatggggattttcaagcgatagctgaacatacacaacagacggtcaagcagggtaatggtgacgaagaaaaatctgttacacgcaagatagtcgattgggtctcttcaggagaagaaattttgaatcgtgctttgttgtatttctccgatcgtaatggagaaagagaaacattagccgatttcttaaaagttcagtatgccgttcaaagagctacacaacgcgccgagttatttgccagtattctaggtgccacggtgagtagtgtaaaaacgattatgacaacccagttaggtggttcagctgcttcaatgagcttgtcatccagcagcagctcggatagttcgaatctgaaaaatgtgttatctcaggtcatcgcgtctacaccacagggggttcctaatgctgacaaattaaccgacaatcaggtaaaacaagtccagcagacccgtcaaaaccgtgatgatctgtccatggagagcgacgtcgcggtggcgggaacagccggaaaagatcgtgctgcgtcggcgtcccagatcgagggacaagagctgattgagcaacagggacttgcggctgggaaagagacggcttctgctgatgctacatcattgacccagtcggcatccaaaggcgcttccagtcagcagtgtattgaggataccagtaagtccctggagctttcttcgctttcgagcctgtcaagcgtagatgcgacacatttgcaggaaatccaatcgatcgtgtcttcagcaatgggcgccaccaacgaattgtcattgacgaacttagagacaccgggattaccaaagccgagtaccactccacgccaggaagttatggagatcagccttgccttagcgaaggccatcactgcattgggtgagagcactcaggctgccttggaaaattttcagtccactcagagtcagtccgcgaacatgaataagatgagtttggaatcccaaggcttgaaaatcgacaaggagcgtgaagaatttaagaaaatgcaggagattcagcaaaagagcggcacaaattcaaccatggatactgtgaataaagttatgattggcgtgacagtggcaattacagtaatctctgttgtttcagcattgtttacctgcggtttgggcttgattggcacagccgctgcgggtgccacagccgccaccgctggggcaacggccgccgccacgaccgctacctctgtgacgaccacagtcgctacccaggtgacgatgcaagcggtggtccaagtcgttaagcaggctattatccaagcagtaaaacgcgccatcgtccaagcgattaaacaggggattaagcaaggcattaaacaagcgatcaaacaggcagtcaaggcaagcgtgaagacacttgccaaaaatgtaggcaagattttcagcgcaggcaagaacgctgtgagtaagtccttcccaaaattgtctaaggtgattaatacacttggttccaaatgggttactcttggcgtgggggcccttacagcggtgccgcagttagtcagtggcattacctcccttcaattgtctgatatgcaaaaagaacttgcacaaatccaaaaggaagtgggtgcacttacggcgcagagtgagatgatgaaagcgtttacactgttctggcagcaagcttcgaaaatcgcggccaaacaaacggaatcaccttcagagacgcaacaacaggcagctaagaccggcgcccagatcgctaaagcgttgtccgccatttcgggtgctttagctgctgctgcttagctcgag

SEQ ID NO: 88 - protein, 909 aa, synthetic construct
  REGION 1..909: note = synthetic construct
MDNGDRLYRADSRPPDEIKRSGGLMPRGHNEYFDRGTQMNINLYDHARGTQTGFVRYDDGYVSTSLSLRSAHLAGQSILSGYSTYYIYVIATAPNMFNVNDVLGVYSPHPYEQEVSALGGIPYSQIYGWYRVNFGVIDERLHRNREYRDRYYRNLNIAPAEDGYRLAGFPPDHQAWREEPWIHHAPQGCGNSSRMIDPLKLFPNFDGDKESAAVNKPSASPMPSELSKNVASFSLGGGGAALDSTVSTEKLSLMAMMQDKNSQLIDPELEEALNSEELQEQIHLLKSRLWDAQTQMQMQDPDKLASEHVDALGVIVDLINGDFQAIAEHTQQTVKQGNGDEEKSVTRKIVDWVSSGEEILNRALLYFSDRNGERETLADFLKVQYAVQRATQRAELFASILGATVSSVKTIMTTQLGGSAASMSLSSSSSSDSSNLKNVLSQVIASTPQGVPNADKLTDNQVKQVQQTRQNRDDLSMESDVAVAGTAGKDRAASASQIEGQELIEQQGLAAGKETASADATSLTQSASKGASSQQCIEDTSKSLELSSLSSLSSVDATHLQEIQSIVSSAMGATNELSLTNLETPGLPKPSTTPRQEVMEISLALAKAITALGESTQAALENFQSTQSQSANMNKMSLESQGLKIDKEREEFKKMQEIQQKSGTNSTMDTVNKVMIGVTVAITVISVVSALFTCGLGLIGTAAAGATAATAGATAAATTATSVTTTVATQVTMQAVVQVVKQAIIQAVKRAIVQAIKQGIKQGIKQAIKQAVKASVKTLAKNVGKIFSAGKNAVSKSFPKLSKVINTLGSKWVTLGVGALTAVPQLVSGITSLQLSDMQKELAQIQKEVGALTAQSEMMKAFTLFWQQASKIAAKQTESPSETQQQAAKTGAQIAKALSAISGALAAAA

SEQ ID NO: 89 - DNA, 647 bp, synthetic construct
  misc_feature 1..647: note = synthetic construct
  source 1..647: mol_type = other DNA
atgggcagcagccatcaccatcatcaccacagccaggatccgatgccaccaagcaagatccaatgtcttgaaacttttgaaagaacttatggacacctttatctacaacatgcgtccctaatgcgtcatttagcctatctactcgataaaattgctcgctcttaccctcatatgtgtccgcttcccgataatatggaagcgtactttgagaattatatccccaataaagatatccctctggacacctatcaaaaaattttcaaactgtcctcagaagatcttgaacaagtctacaaggaaggatacaacgcctatttacaaggagactatgaggaaagttctaccgctttttactggttgattttctttaacccatttgtgtctaaattttggttttcattaggagcttcgctccatatgcgccaaaaatatcaacaagctcttcatgcttatggtgtagctgctttgctaagagaaaaagacccttatcctcattactatgcctacatctgctacaccctgctcaataatcctgaagaagctgaaaaagctcttgatcttgcttggcaaaaagtaaaaacaagctctgcctatagctctttaaaagaagaaattttagcgatcaaatcgtacgcctaagcggccgc

SEQ ID NO: 90 - protein, 212 aa, synthetic construct
  REGION 1..212: note = synthetic construct
MGSSHHHHHHSQDPMPPSKIQCLETFERTYGHLYLQHASLMRHLAYLLDKIARSYPHMCPLPDNMEAYFENYIPNKDIPLDTYQKIFKLSSEDLEQVYKEGYNAYLQGDYEESSTAFYWLIFFNPFVSKFWFSLGASLHMRQKYQQALHAYGVAALLREKDPYPHYYAYICYTLLNNPEEAEKALDLAWQKVKTSSAYSSLKEEILAIKSYA

SEQ ID NO: 91 - DNA, 1521 bp, Chlamydia spp
  source 1..1521: mol_type = genomic DNA
atgagctcttggtttgcacaggcgacggacgtcgctttgagccagacccttgatctgcctgacgcttcattggcggttcaaaccgaaaaatttccatacagctgttcaatctctaaggaatccgccccatcatgtattcgtaaaatcttcgcccatttagcatctcagaaggaaagtgctccgctgtctttttctcgtttacaaccgactactccgaaagaacgcatcctgtttttcgggtcatcgccttcctcccaattgtcctcgactgtccgcaccacaacctcttctccatggaatctttttagcaactcccaggcacgcaactcgacccgtaaattgtcggagaagcttcatttgagctcagagttatccgcccgtgactccactaagccttcgtcgagcgaaccggttaaaccatcggaaaatcttttgcacacccctgagcatcataaggaatccttctcaagtttgaaaaaggataacttatctcctatcatggaggagatcgactcattctctgcagagacagagtcccttgaagagcgtttggtcacccagaaaaaggaggagacggtggcccaggagcaaaagcacccattgctgcgtacatctactccgccatcaaaggccagcggggaatcacaagattctagcgaacacagctcaaaggaagatccttatagtcaacaaccgagccataaaatccaacgccgtaaagagcgtgctaagcgcgtcgtcccaattattactccgccaacggtgggtatctttagtttgagctaccttcttacaaaacaggggatcttagcggatttcagcgcctattcggcatacaaggataatttagaaacaactcagcaagagctgaccatgttgcatcaagaacgtatcgagcaagtccaaaagatcgtggataaaagtaagacaatgcgcttttgggattcattagcatccattgtggccacaatcattccatggatcgaaatgggtgttgcagtaaccatcatcgcactgggaggtggaatcctttcctggtgctctctttttgctgcgcttatcatgattgtaatttcattattggaagcattcgacgggtggcgtgcaatcgctaagcatttaccaggtaacgatcttgaaaagaagatgcgttatttaggttacgtaaagttggccttaactgtgttctcgtgcttactgagtttaagcgccttgtatgtagcaaaattaggaatgagtccgcttttggagggggttgtgaagagtatcgcaccagcattaagtggtatgctgggtttgactcaaggcgtagcactgtatttacaatcttcatcgcaaaagattcgtgcccgctgcactcagatcgacgcacgcattgaattgattaactgggaacgcgatgagtatttcttgcgtgctgaacaacttcttgattcaatgcaaacgtccttcgaacaacttactgaaacattacagttacaacgtgaaattgatcagacatttacagacgctttgcgctag

SEQ ID NO: 92 - protein, 506 aa, Chlamydia spp
MSSWFAQATDVALSQTLDLPDASLAVQTEKFPYSCSISKESAPSCIRKIFAHLASQKESAPLSFSRLQPTTPKERILFFGSSPSSQLSSTVRTTTSSPWNLFSNSQARNSTRKLSEKLHLSSELSARDSTKPSSSEPVKPSENLLHTPEHHKESFSSLKKDNLSPIMEEIDSFSAETESLEERLVTQKKEETVAQEQKHPLLRTSTPPSKASGESQDSSEHSSKEDPYSQQPSHKIQRRKERAKRVVPIITPPTVGIFSLSYLLTKQGILADFSAYSAYKDNLETTQQELTMLHQERIEQVQKIVDKSKTMRFWDSLASIVATIIPWIEMGVAVTIIALGGGILSWCSLFAALIMIVISLLEAFDGWRAIAKHLPGNDLEKKMRYLGYVKLALTVFSCLLSLSALYVAKLGMSPLLEGVVKSIAPALSGMLGLTQGVALYLQSSSQKIRARCTQIDARIELINWERDEYFLRAEQLLDSMQTSFEQLTETLQLQREIDQTFTDALR

SEQ ID NO: 93 - DNA, 1977 bp, synthetic construct
  misc_feature 1..1977: note = synthetic construct
  source 1..1977: mol_type = other DNA
aaaagtgagcgtttaaaaaaattagaatcagagcttcatgatcttacccagtggatgcaacttggccttgttcctaaaaaagaaatcgagagacaccaggaagaaatccgtctgctagaaagcaaaatccttgaagagaaagaacgtctacaacttctcaaagaaagcggtgagatcaaagagtacgtaacccctcgaagaactccagctaaaaccatttacccagatggccccagcgtttcagacgttgagtttgtagaatcctcggatacagaagtggatctcgatgccggtgacacaattgagattgacctaggtgatgaggcaagagaagaaagcggaaacgaactcgactactctagtgaagacgatgaggatcctttcagcgatcgcaatcgttggcgccgaggaggcatcatagatcctgacgcgaatgaatggggttcagctgcttcaatgagctcttggtttgcacaggcgacggacgtcgctttgagccagacccttgatctgcctgacgcttcattggcggttcaaaccgaaaaatttccatacagctgttcaatctctaaggaatccgccccatcatgtattcgtaaaatcttcgcccatttagcatctcagaaggaaagtgctccgctgtctttttctcgtttacaaccgactactccgaaagaacgcatcctgtttttcgggtcatcgccttcctcccaattgtcctcgactgtccgcaccacaacctcttctccatggaatctttttagcaactcccaggcacgcaactcgacccgtaaattgtcggagaagcttcatttgagctcagagttatccgcccgtgactccactaagccttcgtcgagcgaaccggttaaaccatcggaaaatcttttgcacacccctgagcatcataaggaatccttctcaagtttgaaaaaggataacttatctcctatcatggaggagatcgactcattctctgcagagacagagtcccttgaagagcgtttggtcacccagaaaaaggaggagacggtggcccaggagcaaaagcacccattgctgcgtacatctactccgccatcaaaggccagcggggaatcacaagattctagcgaacacagctcaaaggaagatccttatagtcaacaaccgagccataaaatccaacgccgtaaagagcgtgctaagcgcgtcgtcccaattattactccgccaacggtgggtatctttagtttgagctaccttcttacaaaacaggggatcttagcggatttcagcgcctattcggcatacaaggataatttagaaacaactcagcaagagctgaccatgttgcatcaagaacgtatcgagcaagtccaaaagatcgtggataaaagtaagacaatgcgcttttgggattcattagcatccattgtggccacaatcattccatggatcgaaatgggtgttgcagtaaccatcatcgcactgggaggtggaatcctttcctggtgctctctttttgctgcgcttatcatgattgtaatttcattattggaagcattcgacgggtggcgtgcaatcgctaagcatttaccaggtaacgatcttgaaaagaagatgcgttatttaggttacgtaaagttggccttaactgtgttctcgtgcttactgagtttaagcgccttgtatgtagcaaaattaggaatgagtccgcttttggagggggttgtgaagagtatcgcaccagcattaagtggtatgctgggtttgactcaaggcgtagcactgtatttacaatcttcatcgcaaaagattcgtgcccgctgcactcagatcgacgcacgcattgaattgattaactgggaacgcgatgagtatttcttgcgtgctgaacaacttcttgattcaatgcaaacgtccttcgaacaacttactgaaacattacagttacaacgtgaaattgatcagacatttacagacgctttgcgctag

SEQ ID NO: 94 - protein, 659 aa, synthetic construct
  REGION 1..659: note = synthetic construct
MKSERLKKLESELHDLTQWMQLGLVPKKEIERHQEEIRLLESKILEEKERLQLLKESGEIKEYVTPRRTPAKTIYPDGPSVSDVEFVESSDTEVDLDAGDTIEIDLGDEAREESGNELDYSSEDDEDPFSDRNRWRRGGIIDPDANEWGSAASMSSWFAQATDVALSQTLDLPDASLAVQTEKFPYSCSISKESAPSCIRKIFAHLASQKESAPLSFSRLQPTTPKERILFFGSSPSSQLSSTVRTTTSSPWNLFSNSQARNSTRKLSEKLHLSSELSARDSTKPSSSEPVKPSENLLHTPEHHKESFSSLKKDNLSPIMEEIDSFSAETESLEERLVTQKKEETVAQEQKHPLLRTSTPPSKASGESQDSSEHSSKEDPYSQQPSHKIQRRKERAKRVVPIITPPTVGIFSLSYLLTKQGILADFSAYSAYKDNLETTQQELTMLHQERIEQVQKIVDKSKTMRFWDSLASIVATIIPWIEMGVAVTIIALGGGILSWCSLFAALIMIVISLLEAFDGWRAIAKHLPGNDLEKKMRYLGYVKLALTVFSCLLSLSALYVAKLGMSPLLEGVVKSIAPALSGMLGLTQGVALYLQSSSQKIRARCTQIDARIELINWERDEYFLRAEQLLDSMQTSFEQLTETLQLQREIDQTFTDALR

SEQ ID NO: 95 - DNA, 2574 bp, synthetic construct
  misc_feature 1..2574: note = synthetic construct
  source 1..2574: mol_type = other DNA
catatggacaatggcgatcgtttataccgtgccgactcgcgtcccccagatgagattaaacgtagcggtgggttaatgccacgtgggcacaatgagtattttgaccgtggaacacagatgaacattaacctttacgatcatgcccgtgggacccagaccgggtttgtccgttatgatgacgggtatgttagtacgagtttgtccttacgctccgcacaccttgcgggacaaagtattttatcaggctacagcacatattacatttatgtgatcgccactgccccaaacatgttcaatgtgaacgatgtgttgggggtttacagcccccatccatatgaacaagaagtctcggcccttggggggatcccatatagccagatttatggttggtaccgcgtaaattttggtgtgattgatgaacgtttgcatcgtaaccgtgaataccgcgatcgctactaccgtaacttgaacattgcacctgccgaggacggctatcgtttagcgggattcccacccgatcatcaggcgtggcgtgaggaaccgtggatccatcacgcccctcaggggtgcgggaacagtagtcgccatatgaaaagtgagcgtttaaaaaaattagaatcagagcttcatgatcttacccagtggatgcaacttggccttgttcctaaaaaagaaatcgagagacaccaggaagaaatccgtctgctagaaagcaaaatccttgaagagaaagaacgtctacaacttctcaaagaaagcggtgagatcaaagagtacgtaacccctcgaagaactccagctaaaaccatttacccagatggccccagcgtttcagacgttgagtttgtagaatcctcggatacagaagtggatctcgatgccggtgacacaattgagattgacctaggtgatgaggcaagagaagaaagcggaaacgaactcgactactctagtgaagacgatgaggatcctttcagcgatcgcaatcgttggcgccgaggaggcatcatagatcctgacgcgaatgaatggggttcagctgcttcaatgagctcttggtttgcacaggcgacggacgtcgctttgagccagacccttgatctgcctgacgcttcattggcggttcaaaccgaaaaatttccatacagctgttcaatctctaaggaatccgccccatcatgtattcgtaaaatcttcgcccatttagcatctcagaaggaaagtgctccgctgtctttttctcgtttacaaccgactactccgaaagaacgcatcctgtttttcgggtcatcgccttcctcccaattgtcctcgactgtccgcaccacaacctcttctccatggaatctttttagcaactcccaggcacgcaactcgacccgtaaattgtcggagaagcttcatttgagctcagagttatccgcccgtgactccactaagccttcgtcgagcgaaccggttaaaccatcggaaaatcttttgcacacccctgagcatcataaggaatccttctcaagtttgaaaaaggataacttatctcctatcatggaggagatcgactcattctctgcagagacagagtcccttgaagagcgtttggtcacccagaaaaaggaggagacggtggcccaggagcaaaagcacccattgctgcgtacatctactccgccatcaaaggccagcggggaatcacaagattctagcgaacacagctcaaaggaagatccttatagtcaacaaccgagccataaaatccaacgccgtaaagagcgtgctaagcgcgtcgtcccaattattactccgccaacggtgggtatctttagtttgagctaccttcttacaaaacaggggatcttagcggatttcagcgcctattcggcatacaaggataatttagaaacaactcagcaagagctgaccatgttgcatcaagaacgtatcgagcaagtccaaaagatcgtggataaaagtaagacaatgcgcttttgggattcattagcatccattgtggccacaatcattccatggatcgaaatgggtgttgcagtaaccatcatcgcactgggaggtggaatcctttcctggtgctctctttttgctgcgcttatcatgattgtaatttcattattggaagcattcgacgggtggcgtgcaatcgctaagcatttaccaggtaacgatcttgaaaagaagatgcgttatttaggttacgtaaagttggccttaactgtgttctcgtgcttactgagtttaagcgccttgtatgtagcaaaattaggaatgagtccgcttttggagggggttgtgaagagtatcgcaccagcattaagtggtatgctgggtttgactcaaggcgtagcactgtatttacaatcttcatcgcaaaagattcgtgcccgctgcactcagatcgacgcacgcattgaattgattaactgggaacgcgatgagtatttcttgcgtgctgaacaacttcttgattcaatgcaaacgtccttcgaacaacttactgaaacattacagttacaacgtgaaattgatcagacatttacagacgctttgcgctagctcgag

SEQ ID NO: 96 - protein, 853 aa, synthetic construct
  REGION 1..853: note = synthetic construct
MDNGDRLYRADSRPPDEIKRSGGLMPRGHNEYFDRGTQMNINLYDHARGTQTGFVRYDDGYVSTSLSLRSAHLAGQSILSGYSTYYIYVIATAPNMFNVNDVLGVYSPHPYEQEVSALGGIPYSQIYGWYRVNFGVIDERLHRNREYRDRYYRNLNIAPAEDGYRLAGFPPDHQAWREEPWIHHAPQGCGNSSRMKSERLKKLESELHDLTQWMQLGLVPKKEIERHQEEIRLLESKILEEKERLQLLKESGEIKEYVTPRRTPAKTIYPDGPSVSDVEFVESSDTEVDLDAGDTIEIDLGDEAREESGNELDYSSEDDEDPFSDRNRWRRGGIIDPDANEWGSAASMSSWFAQATDVALSQTLDLPDASLAVQTEKFPYSCSISKESAPSCIRKIFAHLASQKESAPLSFSRLQPTTPKERILFFGSSPSSQLSSTVRTTTSSPWNLFSNSQARNSTRKLSEKLHLSSELSARDSTKPSSSEPVKPSENLLHTPEHHKESFSSLKKDNLSPIMEEIDSFSAETESLEERLVTQKKEETVAQEQKHPLLRTSTPPSKASGESQDSSEHSSKEDPYSQQPSHKIQRRKERAKRVVPIITPPTVGIFSLSYLLTKQGILADFSAYSAYKDNLETTQQELTMLHQERIEQVQKIVDKSKTMRFWDSLASIVATIIPWIEMGVAVTIIALGGGILSWCSLFAALIMIVISLLEAFDGWRAIAKHLPGNDLEKKMRYLGYVKLALTVFSCLLSLSALYVAKLGMSPLLEGVVKSIAPALSGMLGLTQGVALYLQSSSQKIRARCTQIDARIELINWERDEYFLRAEQLLDSMQTSFEQLTETLQLQREIDQTFTDALR

SEQ ID NO: 97 - DNA, 647 bp, synthetic construct; identical to SEQ ID NO: 89
  misc_feature 1..647: note = synthetic construct
  source 1..647: mol_type = other DNA

SEQ ID NO: 98 - protein, 212 aa, synthetic construct; identical to SEQ ID NO: 90
  REGION 1..212: note = synthetic construct

SEQ ID NO: 99 - DNA, 1977 bp, synthetic construct; identical to SEQ ID NO: 93
  misc_feature 1..1977: note = synthetic construct
  source 1..1977: mol_type = other DNA

SEQ ID NO: 100 - protein, 659 aa, synthetic construct; identical to SEQ ID NO: 94
  REGION 1..659: note = synthetic construct

SEQ ID NO: 101 - DNA, 2574 bp, synthetic construct; identical to SEQ ID NO: 95
  misc_feature 1..2574: note = synthetic construct
  source 1..2574: mol_type = other DNA

SEQ ID NO: 102 - protein, 853 aa, synthetic construct; identical to SEQ ID NO: 96
  REGION 1..853: note = synthetic construct

SEQ ID NO: 103 - DNA, 810 bp, synthetic construct
  misc_feature 1..810: note = synthetic construct
  source 1..810: mol_type = other DNA
atgcgttgcactcgggcaattcgccaaaccgcaagaacaggctggctgacgtggctggcgattcttgccgtcacggcgcccgtgacttcgccggcatgggccgacgatcctcccgccaccgtataccgctatgactcccgcccgccggaggacgttttccagaacggattcacggcgtggggaaacaacgacaatgtgctcgaccatctgaccggacgttcctgccaggtcggcagcagcaacagcgctttcgtctccaccagcagcagccggcgctataccgaggtctatctcgaacatcgcatgcaggaagcggtcgaggccgaacgcgccggcaggggcaccggccacttcatcggctacatctacgaagtccgcgccgacaacaatttctacggcgccgccagctcgtacttcgaatacgtcgacacttatggcgacaatgccggccgtatcctcgccggcgcgctggccacctaccagagcgaatatctggcacaccggcgcattccgcccgaaaacatccgcagggtaacgcgggtctatcacaacggcatcaccggcgagaccacgaccacggagtattccaacgctcgctacgtcagccagcagactcgcgccaatcccaacccctacacatcgcgaaggtccgtagcgtcgatcgtcggcacattggtgcgcatggcgccggtgataggcgcttgcatggcgcggcaggccgaaagctccgaggccatggcagcctggtccgaacgcgccggcgaggcgatggttctcgtgtactacgaaagcatcgcgtattcgttctag

SEQ ID NO: 104 - protein, 269 aa, synthetic construct
  REGION 1..269: note = synthetic construct
MRCTRAIRQTARTGWLTWLAILAVTAPVTSPAWADDPPATVYRYDSRPPEDVFQNGFTAWGNNDNVLDHLTGRSCQVGSSNSAFVSTSSSRRYTEVYLEHRMQEAVEAERAGRGTGHFIGYIYEVRADNNFYGAASSYFEYVDTYGDNAGRILAGALATYQSEYLAHRRIPPENIRRVTRVYHNGITGETTTTEYSNARYVSQQTRANPNPYTSRRSVASIVGTLVRMAPVIGACMARQAESSEAMAAWSERAGEAMVLVYYESIAYSF

SEQ ID NO: 105 - DNA, 681 bp, synthetic construct
  misc_feature 1..681: note = synthetic construct
  source 1..681: mol_type = other DNA
atgccgatcgaccgcaagacgctctgccatctcctgtccgttctgccgttggccctcctcggatctcacgtggcgcgggcctccacgccaggcatcgtcattccgccgcaggaacagattacccagcatggcagcccctatggacgctgcgcgaacaagacccgtgccctgaccgtggcggaattgcgcggcagcggcgatctgcaggagtacctgcgtcatgtgacgcgcggctggtcaatatttgcgctctacgatggcacctatctcggcggcgaatatggcggcgtgatcaaggacggaacacccggcggcgcattcgacctgaaaacgacgttctgcatcatgaccacgcgcaatacgggtcaacccgcaacggatcactactacagcaacgtcaccgccactcgcctgctctccagcaccaacagcaggctatgcgcggtcttcgtcagaagcgggcaaccggtcattggcgcctgcaccagcccgtatgacggcaagtactggagcatgtacagccggctgcggaaaatgctttacctgatctacgtggccggcatctccgtacgcgtccatgtcagcaaggaagaacagtattacgactatgaggacgcaacgttcgagacttacgcccttaccggcatctccatctgcaatcctggatcatccttatgctga

SEQ ID NO: 106 - protein, 226 aa, synthetic construct
  REGION 1..226: note = synthetic construct
MPIDRKTLCHLLSVLPLALLGSHVARASTPGIVIPPQEQITQHGGPYGRCANKTRALTVAELRGSGDLQEYLRHVTRGWSIFALYDGTYLGGEYGGVIKDGTPGGAFDLKTTFCIMTTRNTGQPATDHYYSNVTATRLLSSTNSRLCAVFVRSGQPVIGACTSPYDGKYWSMYSRLRKMLYLIYVAGISVRVHVSKEEQYYDYEDATFETYALTGISICNPGSSLC

SEQ ID NO: 107 - DNA, 684 bp, synthetic construct
  misc_feature 1..684: note = synthetic construct
  source 1..684: mol_type = other DNA
atgctgatcaacaacaagaagctgcttcatcacattctgcccatcctggtgctcgccctgctgggcatgcgcacggcccaggccgttgcgccaggcatcgtcatcccgccgaaggcactgttcacccaacagggcggcgcctatggacgctgcccgaacggaacccgcgccttgaccgtggccgaactgcgcggcaacgccgaattgcagacgtatttgcgccagataacgcccggctggtccatatacggtctctatgacggtacgtacctgggccaggcgtacggcggcatcatcaaggacgcgccgccaggcgcggggttcatttatcgcgaaactttctgcatcacgaccatatacaagaccgggcaaccggctgcggatcactactacagcaaggtcacggccacgcgcctgctcgccagcaccaacagcaggctgtgcgcggtattcgtcagggacgggcaatcggtcatcggagcctgcgccagcccgtatgaaggcaggtacagagacatgtacgacgcgctgcggcgcctgctgtacatgatctatatgtccggccttgccgtacgcgtccacgtcagcaaggaagagcagtattacgactacgaggacgccacattccagacctatgccctcaccggcatttccctctgcaacccggcagcgtcgatatgctga

SEQ ID NO: 108 - protein, 227 aa, synthetic construct
  REGION 1..227: note = synthetic construct
MLINNKKLLHHILPILVLALLGMRTAQAVAPGIVIPPKALFTQQGGAYGRCPNGTRALTVAELRGNAELQTYLRQITPGWSIYGLYDGTYLGQAYGGIIKDAPPGAGFIYRETFCITTIYKTGQPAADHYYSKVTATRLLASTNSRLCAVFVRDGQSVIGACASPYEGRYRDMYDALRRLLYMIYMSGLAVRVHVSKEEQYYDYEDATFQTYALTGISLCNPAASIC

SEQ ID NO: 109 - DNA, 459 bp, synthetic construct
  misc_feature 1..459: note = synthetic construct
  source 1..459: mol_type = other DNA
atgctgagacgcttccccactcgaaccaccgccccgggacagggcggcgcccggcggtcgcgcgtgcgcgccctggcgtggttgctggcatccggcgcgatgacgcatctttcccccgccctggccgacgttccttatgtgctggtgaagaccaatatggtggtcaccagcgtagccatgaagccgtatgaagtcaccccgacgcgcatgctggtctgcggcatcgccgccaaactgggcgccgcggccagcagcccggacgcgcacgtgccgttctgcttcggcaaggatctcaagcgtcccggcagcagtcccatggaagtcatgttgcgcgccgtcttcatgcaacaacggccgctgcgcatgtttctgggtcccaagcaactcactttcgaaggcaagcccgcgctcgaactgatccggatggtcgaatgcagcggcaagcaggattgcccctga

SEQ ID NO: 110 - protein, 152 aa, synthetic construct
  REGION 1..152: note = synthetic construct
MLRRFPTRTTAPGQGGARRSRVRALAWLLASGAMTHLSPALADVPYVLVKTNMVVTSVAMKPYEVTPTRMLVCGIAAKLGAAASSPDAHVPFCFGKDLKRPGSSPMEVMLRAVFMQQRPLRMFLGPKQLTFEGKPALELIRMVECSGKQDCP

SEQ ID NO: 111 - DNA, 402 bp, synthetic construct
  misc_feature 1..402: note = synthetic construct
  source 1..402: mol_type = other DNA
atgcagcggcaagcaggattgcccctgaaggcgaaccccatgcataccatcgcatccatcctgttgtccgtgctcggcatatacagcccggctgacgtcgccggcttgccgacccatctgtacaagaacttcactgtccaggagctggccttgaaactgaagggcaagaatcaggagttctgcctgaccgccttcatgtcgggcagaagcctggtccgggcgtgcctgtccgacgcgggacacgagcacgacacgtggttcgacaccatgcttggctttgccatatccgcgtatgcgctcaagagccggatcgcgctgacggtggaagactcgccgtatccgggcactcccggcgatctgctcgaactgcagatctgcccgctcaacggatattgcgaatga

SEQ ID NO: 112 - protein, 133 aa, synthetic construct
  REGION 1..133: note = synthetic construct
MQRQAGLPLKANPMHTIASILLSVLGIYSPADVAGLPTHLYKNFTVQELALKLKGKNQEFCLTAFMSGRSLVRACLSDAGHEHDTWFDTMLGFAISAYALKSRIALTVEDSPYPGTPGDLLELQICPLNGYCE

SEQ ID NO: 113 - DNA, 868 bp, synthetic construct
  misc_feature 1..868: note = synthetic construct
  source 1..868: mol_type = other DNA
atgattgacatcatgttgcatataggttagataaaacaagtggttatctttccggattgtcttcttgtatgatatataagttttcctcgatgaaaaatataactttcattttttttattttattagcatcgccattatatgcaaatggcgacagattataccgtgctgactctagacccccagatgaaataaaacgtttccggagtcttatgcccagaggtaatgagtacttcgatagaggaactcaaatgaatattaatctttatgatcacgcgagaggaacacaaaccggctttgtcagatatgatgacggatatgtttccacttctcttagtttgagaagtgctcacttagcaggacagtatatattatcaggatattcacttactatatatatcgttatagcaaatatgtttaatgttaatgatgtaattagcgtatacagccctcacccatatgaacaggaggtttctgcgttaggtggaataccatattctcagatatatggatggtatcgtgttaattttggtgtgattgatgaacgattacatcgtaacagggaatatagagaccggtattacagaaatctgaatatagctccggcagaggatggttacagattagcaggtttcccaccggatcaccaagcttggagagaagaaccctggattcatcatgcaccacaaggttgtggagattcatcaggaacaatcacaggtgatacttgtaatgaggagacccagaatctgagcacaatatatgccagggaatatcaatcaaaagttaagaggcagatattttcagactatcagtcagaggttgacatatataacagaattcgggatgaattatgaataaagtaaaatgt

SEQ ID NO: 114 - DNA, 604 bp, synthetic construct
  misc_feature 1..604: note = synthetic construct
  source 1..604: mol_type = other DNA
gttgacatatataacagaattcgggatgaattatgaataaagtaaaatgttatgttttatttacggcgttactatcctctctatatgcacacggagctccccagactattacagaactatgttcggaatatcgcaacacacaaatatatacgataaatgacaagatactatcatatacggaatcgatggcaggcaaaagagaaatggttatcattacatttaagagcggcgaaacatttcaggtcgaagtcccgggcagtcaacatatagactcccagaaaaaagccattgaaaggatgaaggacacattaagaatcacatatctgaccgagaccaaaattgataaattatgtgtatggaataataaaacccccaattcaattgcggcaatcagtatgaaaaactagtttgctttaaaagcatgtctaatgctaggaacctatataacaactactgtacttatactaatgagccttatgctgcatttgaaaaggcggtagaggaggcaataccgatccttaaactgtaacactataacagcttccactacagggagctgttatagcacacagaaaaaactaagctaggctggaggggcaagctt

SEQ ID NO: 115 - DNA, 777 bp, Vibrio cholerae
  source 1..777: mol_type = genomic DNA
atggtaaagataatatttgtgttttttattttcttatcatcattttcatatgcaaatgatgataagttatatcgggcagattctagacctcctgatgaaataaagcagtcaggtggtcttatgccaagaggacagagtgagtactttgaccgaggtactcaaatgaatatcaacctttatgatcatgcaagaggaactcagacgggatttgttaggcacgatgatggatatgtttccacctcaattagtttgagaagtgcccacttagtgggtcaaactatattgtctggtcattctacttattatatatatgttatagccactgcacccaacatgtttaacgttaatgatgtattaggggcatacagtcctcatccagatgaacaagaagtttctgctttaggtgggattccatactcccaaatatatggatggtatcgagttcattttggggtgcttgatgaacaattacatcgtaataggggctacagagatagatattacagtaacttagatattgctccagcagcagatggttatggattggcaggtttccctccggagcatagagcttggagggaagagccgtggattcatcatgcaccgccgggttgtgggaatgctccaagatcatcgatcagtaatacttgcgatgaaaaaacccaaagtctaggtgtaaaattccttgacgaataccaatctaaagttaaaagacaaatattttcaggctatcaatctgatattgatacacataatagaattaaggatgaattatga

SEQ ID NO: 116 - protein, 70 aa, Vibrio cholerae
HRAWREEPWIHHAPPGCGNAPRSSMSNTCDEKTQSLGVKFLDEYQSKVKRQIFSGYQSDIDTHNRIKDEL

SEQ ID NO: 117 - DNA, 375 bp, Vibrio cholerae
  source 1..375: mol_type = genomic DNA
atgattaaattaaaatttggtgttttttttacagttttactatcttcagcatatgcacatggaacacctcaaaatattactgatttgtgtgcagaataccacaacacacaaatatatacgctaaatgataagatattttcgtatacagaatctctagctggaaaaagagagatggctatcattacttttaagaatggtgcaatttttcaagtagaagtaccaggtagtcaacatatagattcacaaaaaaaagcgattgaaaggatgaaggataccctgaggattgcatatcttactgaagctaaagtcgaaaagttatgtgtatggaataataaaacgcctcatgcgattgccgcaattagtatggcaaattaa

SEQ ID NO: 118 - protein, 124 aa, Vibrio cholerae
MIKLKFGVFFTVLLSSAYAHGTPQNITDLCAEYHNTQIYTLNDKIFSYTESLAGKREMAIITFKNGAIFQVEVPGSQHIDSQKKAIERMKDTLRIAYLTEAKVEKLCVWNNKTPHAIAAISMAN

(19) United States
(12) Patent Application Publication
(10) Pub. No.: US 2025/0277005 A1
(43) Pub. Date: Sep. 4, 2025

(54) METHODS AND COMPOSITIONS RELATED TO THE NEXT GENERATION VACCINE

(71) Applicant: UNIVERSITY OF KANSAS, Lawrence, KS (US)

(72) Inventors: Wendy L. PICKING, Lawrence, KS (US); William D. PICKING, Lawrence, KS (US)

(21) Appl. No.: 19/213,316

(22) Filed: May 20, 2025

Related U.S. Application Data
(62) Division of application No. 17/931,356, filed on Sep. 12, 2022, now Pat. No. 12,331,085.
Application No. 17/931,356 is a continuation of application No. 17/053,544, filed on Nov. 6, 2020, now Pat. No. 11,439,700.
(60) Provisional application No. 62/667,599, filed on May 6, 2018.

(51) Int. Cl. C07K 14/28; A61K 39/02; A61K 39/104; C07K 14/21; C07K 14/235; C07K 14/245

(52) U.S. Cl. CPC C07K 14/28; A61K 39/104; A61K 39/107; C07K 14/21; C07K 14/235; C07K 14/245; C07K 2319/00

(57) ABSTRACT

Disclosed are compositions comprising a Gram negative needle tip protein and a translocator protein and methods of their use.

### I. CROSS-REFERENCE TO RELATED APPLICATIONS

The present application claims the priority benefit of U.S. Provisional Patent Application Ser. No. 62/667,599, filed May 6, 2018, which is incorporated by reference in its entity herein.

### II. BACKGROUND

Bordetella pertussis is a Gram-negative bacterial pathogen that causes pertussis, or whooping cough, a highly contagious, severe respiratory disease that is life threatening for infants and young children. This pathogen colonizes the trachea and secretes toxins that paralyze the cilia, which prevents clearance of mucous. Severe (paroxysmal), non-productive coughing fits are a result and attempts to acquire oxygen are manifested by the characteristic “whoop” upon gasps for air. The majority of deaths associated with pertussis are actually caused by secondary respiratory infections resulting from the inability to clear pulmonary secretions. In the 1940s a whole-cell pertussis (wP) vaccine was introduced that dramatically reduced the mortality caused by pertussis. Due to side effects attributed to the wP vaccine, a new acellular pertussis (aP) vaccine was developed and introduced in the US and other parts of the world in the 1990s. Although the aP vaccine has few side effects, its protective efficacy is lower than that of the wP vaccine. In 2012, which is considered the most recent major epidemic, 48,277 cases of pertussis were reported and, in 2015, 20,762 cases were reported. During the last 15 years, in addition to a greater overall incidence of pertussis, there is growing concern over the increase in the peak number of reported cases for each ensuing epidemic. In 2015, 45% of the 0.5 to 6-year-old children that contracted pertussis had been vaccinated with DTaP at least three times (with five vaccinations being optimal: 2, 4, 6, 15 months and one at 4-6 years). Additionally, there is evidence that selective pressure is causing B. pertussis to eliminate virulence factors that are components of the aP vaccine, further compromising the vaccine's efficacy. Taken together, a better vaccine is needed.

### III. SUMMARY

Disclosed are methods and compositions related to polypeptides comprising a fusion of the needle tip protein and translocator protein of a type III secretion apparatus (T3SA) from a type III secretion system (T3SS) of a Gram negative bacteria.

Disclosed herein are fusion polypeptides comprising a fusion of a needle tip protein (such as, for example, Bsp22, LcrV, BipD, PcrV, CT053, or CT668) or an antigenic fragment thereof and a translocator protein (such as, for example, BopB, YopB, BipB, PopB, CopB, or CopB2) or an antigenic fragment thereof from a Type III secretion system (T3SS) of a Gram negative bacteria (such as, for example, Bordetella spp., Burkholderia spp., Chlamydia spp., Pseudomonas spp., Vibrio spp. or Yersinia spp.); wherein the gram negative bacteria is not a Salmonella enterica or Shigella spp.

In one aspect, disclosed herein are fusion polypeptides, wherein the fusion polypeptide is arranged such that the needle tip protein is 5′ of the translocator protein.

Also disclosed herein are fusion polypeptides of any preceding aspect, wherein the fusion further comprises an adjuvant such as, for example, Cholera Toxin or antigenic fragment thereof (such as, for example, CTA1) or double mutant labile toxin (dmLT) or an antigenic fragment thereof labile toxin (such as, for example, LTA1) from Enterotoxigenic Escherichia coli. In some aspect, the dmLT or fragment thereof can also be fused to the needle tip protein-translocator protein fusion at the 5′ end.

In one aspect, disclosed herein are fusion polypeptides of any preceding aspect, wherein the fusion polypeptide further comprises pertussis toxoid (PTd).

Also disclosed herein are compositions comprising a T3SA needle tip protein (such as, for example, Bsp22, LcrV, BipD, PcrV, or CdsF) or an antigenic fragment thereof from a Gram negative bacteria (such as, for example, Bordetella spp., Burkholderia spp., Chlamydia spp., Pseudomonas spp., Vibrio spp., or Yersinia spp.) and a T3SA first translocator protein (such as, for example, BopB, YopB, BipB, PopB, or CopB/CopB2) or an antigenic fragment thereof from a Gram negative bacteria; wherein the gram negative bacteria is not a Salmonella enterica or Shigella spp. In one aspect, the composition can comprise the needle tip protein or fragment thereof and the translocator protein or fragment thereof as separate components or as a fusion polypeptide. Also disclosed herein are compositions of any preceding aspect, wherein the composition comprises an adjuvant (such as, for example, dmLT, LTA1, cholera toxin, or CTA1) and/or bacterial toxin protein such as a pertussis toxoid (PTd).

In one aspect, disclosed herein are vaccines comprising the fusion polypeptides or compositions of any preceding aspect. In some embodiments, the vaccine can further comprise an acellular gram negative vaccine or active components thereof. In one aspect, the vaccine can comprise pertussis toxoid (PTd).

Also disclosed herein are methods of treating, inhibiting, or preventing an infection of a Gram negative bacteria (such as, for example, Bordetella spp., Burkholderia spp., Chlamydia spp., Pseudomonas spp., Vibrio spp., or Yersinia spp.) in a subject comprising administering to the subject the fusion polypeptide, composition, or vaccine of any preceding aspect.

In one aspect, disclosed herein are methods of treating, inhibiting, or preventing an infection of a Gram negative bacteria of any preceding aspect, wherein the method further inhibits or prevents colony formation of the bacteria and/or transmission of the bacteria to another subject.

Also disclosed herein are methods of eliciting an immune response in a subject to a Gram negative bacteria (such as, for example, Bordetella spp., Burkholderia spp., Chlamydia spp., Pseudomonas spp., Vibrio spp., or Yersinia spp.) comprising administering to the subject the fusion polypeptide, composition, or vaccine of any preceding aspect. For example, disclosed herein are methods of eliciting an immune response against at least one Gram negative bacteria serovar in a subject in need thereof, comprising administering to the subject a composition comprising at least one needle tip protein or an antigenic fragment thereof and/or at least one translocator protein or an antigenic fragment thereof; wherein said composition is administered in an amount sufficient to elicit an immune response to said at least one Gram negative bacteria serovar in said subject; and wherein the Gram negative bacteria is not a Shigella spp. or Salmonella enterica.

In one aspect, disclosed herein are methods of eliciting an immune response in a subject to a Gram negative bacteria of any preceding aspect, wherein the immune response provides sterilizing immunity.

### IV. BRIEF DESCRIPTION OF THE DRAWINGS

The accompanying drawings, which are incorporated in and constitute a part of this specification, illustrate several embodiments and together with the description illustrate the disclosed compositions and methods.

FIG. 1 shows the protective efficacy of intranasally administered 22BF against B. bronchiseptica challenge. Mice (n=10) were vaccinated intranasally biweekly three times with the indicated formulation which contained 10 μg protein±dmLT. Zoetis vaccine was delivered subcutaneously on day 1 and 21 as per manufacturer's directions. Mice were challenged with 1.3×10⁷ B. bronchiseptica on day 56. BopB was not available at day 0. #P<0.05 compared to survival of mice vaccinated with PBS.

FIG. 2 shows the weight gain/loss of vaccinated mice during B. bronchiseptica challenge. Mice (same as above) were weighed daily in p.m. Note that the 22BF+dmLT mice gain weight and have small error bars.

FIG. 3A and FIG. 3B show the protective efficacy of 22BF+dmLT. Mice were vaccinated on days 0, 14, 28 and challenged on day 56 with a sublethal dose of B. bronchiseptica. FIG. 3A shows that on day 7 of the challenge, the CFU/lung were determined. *=P<0.05, **=P<0.01 when compared to dmLT. FIG. 3B shows the decrease in CFU compared to the 22BF average. *=P<0.05, **=P<0.01 when compared to 22BF.

FIGS. 4A, 4B, and 4C show the kinetics of IgG response. Blood was collected on days-1, 13, 27, 41, and 55. The kinetics of anti-Bsp22, -BopB and -dmLT IgG were assessed in all sera and shown in (4A) BopB, (4B) Bsp22, and (4C) dmLT. Typical logarithmic increases were seen. *=P value of <0.05 when comparing to PBS controls.

FIGS. 5A, 5B, 5C, 5D, 5E, 5F, 5G, 5H, and 5I show stimulation of antibody secreting cells from bone marrow, spleen and lungs. Bone marrow, spleens and lungs were collected on day 56. Single cell suspensions from 5 mice per group were stimulated in vitro Bsp22, BopB or dmLT. IgG (black) and IgA (white) ASC were measured by ELISpot. Bars represent mean ASC per 10⁶ cells +SD from replicate wells. Data for bone marrow is shown in (4A), (5D), and (5G) for Bsp22, BopB, and dmLT, respectively. Data for spleen is shown in (5B), (5E), and (5H) for Bsp22, BopB, and dmLT, respectively. Data for lungs is shown in (5C), (5F), and (5I) for Bsp22, BopB, and dmLT, respectively.

FIGS. 6A, 6B, 6C, 6D, 6E, 6F, 6G, 6H, and 6I show Th1 cytokine secretion. Splenocytes were extracted from 5 mice of each group and incubated with Bsp22, BopB or dmLT. After 48 h, supernatants were collected and levels of cytokine secretion in response to specified antigen were then measured (in pg/ml) using an MSD cytokine detection plate. Each bar represents mean of triplicate wells ±S.D. Asterisk specified a P<0.05 when comparing specified groups. Data for IFN-γ is shown in (6A), (6B), and (6C) for Bsp22, BopB, and dmLT, respectively. Data for TNF-α is shown in (6D), (6E), and (6F) for Bsp22, BopB, and dmLT, respectively. Data for IL-1B is shown in (6G), (6H), and (6I) for Bsp22, BopB, and dmLT, respectively.

FIGS. 7A, 7B, 7C, 7D, 7E, 7F, 7G, 7H, and 7I show Th1 cytokine secretion. Splenocytes were extracted from 5 mice of each group and incubated with Bsp22, BopB or dmLT. After 48 h, supernatants were collected and levels of cytokine secretion in response to specified antigen were then measured (in pg/ml) using an MSD cytokine detection plate. Each bar represents mean of triplicate wells ±S.D. Asterisk specified a P<0.05 when comparing specified groups. Data for IL-2 is shown in (7A), (7B), and (7C) for Bsp22, BopB, and dmLT, respectively. Data for IL-10 is shown in (7D), (7E), and (7F) for Bsp22, BopB, and dmLT, respectively. Data for IL-6 is shown in (7G), (7H), and (7I) for Bsp22, BopB, and dmLT, respectively.

FIGS. 8A, 8B, and 8C shows IL-17 secretion. Splenocytes were extracted from 5 mice of each group and incubated with Bsp22, BopB or dmLT. After 48 h, supernatants were collected and levels of IL-17 secretion in response to labeled antigen were then measured by the MSD® U-Plex Platform Multiplex Assay and the data is shown in (8A) BopB, (8B) Bsp22, and (8C) dmLT. Each bar represents the mean of triplicate wells ±S.D. Significance (Asterisk=P<0.05) was calculated for the comparison between labeled groups.

FIGS. 9A, 9B, 9C, 9D, 9E, and 9F show Th2 cytokine secretion. Splenocytes were extracted from 5 mice of each group and incubated with Bsp22, BopB or dmLT. After 48 h, supernatants were collected and levels of cytokine secretion in response to specified antigen were then measured (in pg/ml) using an MSD cytokine detection plate. Each bar represents mean of triplicate wells ±S.D. Asterisk specified a P<0.05 when comparing specified groups. Data for IL-4 is shown in (9A), (9B), and (9C) for Bsp22, BopB, and dmLT, respectively. Data for IL-5 is shown in (9D), (9E), and (9F) for Bsp22, BopB, and dmLT, respectively.

FIG. 10 shows the change in weight in percentage after infection with sublethal dosage of B. pertussis intranasally. There was an observable difference in weight loss between mice vaccinated with the 22BF+dmLT+PTd formulation and those that only received PBS. By Day 7 all mice aside from PBS treated mice had recovered to within 3% of pre-infection weight.

FIGS. 11A, 11B, 11C, and 11D show serum antibody responses to BopB, Bsp22, Pertussis Toxin Mutant, and dmLT. Mice were immunized on days 0, 14, and 28 with 22BF+PTd admixed with dmLT. Serum IgG antibodies specific for BopB, Bsp22, PTd, and dmLT were measured by ELISA and the data is shown in (11A) BopB, (11B) Bsp22, (11C) PTd, and (11D) dmLT. Data are the mean titers (EU ml\hat{ }-1) from group pools of animal samples. An asterisk indicates a P value of 0.05 when comparing vaccinated mice and the PBS controls. No responses were seen in the control mice that received PBS. Mice vaccinated with Infanrix only displayed a response against pertussis toxin mutant, which is part of its formulation.

FIG. 12 shows the Lung Colony forming units (CFU) from mice 3 days post intranasal infection. Mice vaccinated intranasally with 22BF+PTd and dmLT showed statistical (P<0.05) decreases in lung CFUs when compared to PBS treated mice. The mice vaccinated intradermally and mice vaccinated intramuscularly with 22BF+PTd and dmLT either showed sterilizing immunity, or no statistical decrease in lung CFUs. Infanrix appeared to show a decrease in lung CFU, but this was not statistically significant (P>0.05). (*=P<0.05, KW p-value=0.0003).

FIG. 13 shows the Lung Colony forming units (CFU) from mice 7 days post intranasal infection. Mice vaccinated intranasally with 22BF+PTd and dmLT showed statistical (P<0.05) decreases in lung CFUs when compared to PBS treated mice, with 60% of the mice showing sterilizing immunity. The mice vaccinated intramuscularly or intradermally with 22BF+PTd and dmLT showed no statistical decrease in lung CFUs. Infanrix appeared to display sterilizing immunity with CFU measuring below the limit of detection. (*=P<0.05, **P<0.01, KW p-value=0.0001).

FIG. 14 shows the protective efficacy of LTA1-DBF vs DBF+dmLT. Mice were vaccinated intramuscularly on days 0, 14 and 28 with the indicated μg of DBF+0.1 μg dmLT or DBF equivalent of LTA1-DBF. The positive control was DBF+dmLT delivered intranasally. On day 56 the mice were challenged with Shigella flexneri. FIG. 14 indicates the percent survival of mice post infection with Shigella flexneri.

FIGS. 15A, 15B, and 15C show the kinetics of IgG response. Mice from FIG. 14 were bled prior to vaccination and on day 42. Sera were assessed for anti-IpaD, -IpaB and -dmLT IgG, and the data is shown in (15A) IpaD, (15Bb) IpaB, and (15C) dmLT. Differences in the IgG levels in mice vaccinated with dmLT vs. LTA1 are attributed to the recognition of the entire dmLT on the well.

FIGS. 16A, 16B, and 16C show the stimulation of antibody secreting cells from bone marrow. Bone marrow was collected on day 56. Single cell suspensions from 5 mice per group were stimulated in vitro IpaD, IpaB or dmLT. IgG (black) and IgA (white) ASC were measured by ELISpot, and the data is shown in (16A) IpaD, (16B) IpaB, and (16C) dmLT. Bars represent mean ASC per 10⁶ cells +SD from replicate wells.

FIGS. 17A, 17B, and 17C show the stimulation of antibody secreting cells from spleen. Spleens were collected on day 56. Single cell suspensions from 5 mice per group were stimulated in vitro IpaD, IpaB or dmLT. IgG (black) and IgA (white) ASC were measured by ELISpot. Bars represent mean ASC per 10⁶ cells +SD from replicate wells.

FIGS. 18A, 18B, and 18C show the stimulation of antibody secreting cells from lungs. Lungs were collected on day 56. Single cell suspensions from 5 mice per group were stimulated in vitro IpaD, IpaB or dmLT. IgG (black) and IgA (white) ASC were measured by ELISpot, and the data is shown in (18A) IpaD, (18B) IpaB, and (18C) dmLT. Bars represent mean ASC per 10⁶ cells +SD from replicate wells.

FIGS. 19A, 19B, 19C, and 19D show the protective efficacy and IgG response kinetics of LTA1-22BF. Mice were vaccinated on days 0, 14, 28 and challenged on day 56 with a sublethal dose of B. pertussis. On day 7 of the challenge, the CFU/lung were determined. FIG. 19A shows the CFU/lung while FIG. 19B shows the decrease in CFU compared to the PBS average. *=P<0.05 when compared to PBS. FIGS. 19C and 19D how the kinetics of the response of the anti-Bsp22 and anti-BopB IgG, respectively. No difference is seen between the mice vaccinated with 22BF+dmLT and LTA1-22BF.

FIG. 20 shows the ADPr activity of L-antigens. LTA1 was fused to DBF, 22BF or SseB. LTA1, however, must retain its ADP-ribosylation activity to maintain adjuvant activity. The ADPr of NAD+ was biotin conjugated and LTA1 transferred the biotin-ADPr moiety to ARF4. The biotin was then detected with Streptavidin-IR800. Lane 1: LTA1-DBF; 2: LTA1-22BF; 3: LTA1-SseB.

### V. DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. DEFINITIONS

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

The needle tip protein and/or translocator proteins or antigenic portions thereof disclosed herein are used to elicit an immune response in subjects to whom they are administered. By “elicit an immune response”, “induces or enhances an immune response”, or “stimulates an immune response” which are used interchangeably herein, is meant that the subject mounts one or both of an innate and/or an adaptive immune reaction against antigenic determinants of the proteins or antigenic portions thereof that are administered. Preferably a statistically measurable induction or increase in an immune response over a control sample to which the needle tip protein and/or translocator proteins or antigenic portions thereof disclosed herein has not been administered. Preferably the induction or enhancement of the immune response results in a prophylactic or therapeutic response in a subject. In particular, the adaptive immune reaction entails production of e.g. B and T cell lymphocytes and antibodies specific for binding and forming complexes with the antigenic determinants. In some embodiments, the proteins and/or antigenic fragments thereof elicit a protective immune response in the subject, i.e. administration of one or more of the proteins and/or antigenic portions thereof results in an immune response that is protective against later challenge by the disease causing organism itself, either preventing infection altogether, or lessening the impact of infection by decreasing disease symptoms that would otherwise occur, had the subject not been vaccinated as described herein.

“Vaccine” as used herein is a preparation that stimulates an immune response that produces immunity against particular antigens, e.g. Gram negative bacteria. Vaccines may be administered prophylactically (for example, to prevent or inhibit the establishment of an infection) or therapeutically to inhibit, reduce, or treat an established infection, or to ameliorate the effects or symptoms of an infection. Vaccines may contain, but are not limited to, live, attenuated infectious material such as viruses or bacteria, and dead or inactivated organisms or purified products derived therefrom. A vaccine can be administered by injection, orally, or by inhalation. Injections may be, but are not limited to, subcutaneous (sc), intramuscular (im), intraperitoneal (ip), intradermal (id) or intravenous (iv).

“Optional” or “optionally” means that the subsequently described event or circumstance may or may not occur, and that the description includes instances where said event or circumstance occurs and instances where it does not.

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician or veterinarian.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. COMPOSITIONS

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutations of these compounds may not be explicitly disclosed, each is specifically contemplated and described herein. For example, if a particular needle tip protein (such as, for example, IpaD, SipD, SseB, Bsp22, LcrV, BipD, PcrV, CT053, or CT668), translocator protein (such as, for example, IpaB, SipB, SseC, BopB, YopB, BipB, PopB, CopB, or CopB2), or fusion polypeptide thereof (such as, for example, 22BF, BurkF, PaF, YerF, CT053-CopB, CT053-CopB2, CT668-CopB, or CT668-CopB2) is disclosed and discussed and a number of modifications that can be made to a number of molecules including the needle tip protein (such as, for example, IpaD, SipD, SseB, Bsp22, LcrV, BipD, PcrV, CT053, or CT668), translocator protein (such as, for example, IpaB, SipB, SseC, BopB, YopB, BipB, PopB, CopB, or CopB2), or fusion polypeptide thereof (such as, for example, 22BF, BurkF, PaF, YerF, CT053-CopB, CT053-CopB2, CT668-CopB, or CT668-CopB2) are discussed, specifically contemplated is each and every combination and permutation of needle tip protein such as, for example, IpaD, SipD, SseB, Bsp22, LcrV, BipD, PcrV, CT053, or CT668), translocator protein (such as, for example, IpaB, SipB, SseC, BopB, YopB, BipB, PopB, CopB, or CopB2), or fusion polypeptide thereof (such as, for example, DBF, S1, S2, 22BF, BurkF, PaF, YerF, CT053-CopB, CT053-CopB2, CT668-CopB, or CT668-CopB2) and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

To infect a host, B. pertussis uses an arsenal of well-characterized virulence factors. These factors include pertussis toxin (PT), adenylate cyclase toxin (ACT), the type III secretion system (T3SS), tracheal cytotoxin (TCT), dermonecrotic toxin (DNT), filamentous hemagglutinin (FHA), pertactin (PRN), and lipooligosaccharide (LOS). Current aP vaccines are comprised of PT, FHA, PRN, and the fimbrial proteins in varying proportions, but not necessarily all four proteins. Though the aP vaccine causes fewer adverse reactions than the wP vaccine, it is not as efficacious. This same situation exists for other pathogenic Gram negative bacteria. Accordingly, disclosed herein are fusion polypeptides from a Type III secretion system (T3SS) of a Gram negative bacteria (such as, for example, Shigella spp., Salmonella enterica, Bordetella spp. (such as, for example B. pertussis and/or B. bronchiseptica), Burkholderia spp. (such as, for example, B. cepacian, B. mallei, and/or B. pseudomallei), Chlamydia spp. (such as, for example, C. trachomatis), Pseudomonas spp., Vibrio spp. or Yersinia spp.) comprising a polypeptide of needle tip protein (such as, for example, IpaD, SipD, SseB, Bsp22, LcrV, BipD, PcrV, CT053, or CT668) or an antigenic fragment thereof and polypeptides of a translocator protein (such as, for example, IpaB, SipB, SseC, BopB, YopB, BipB, PopB, CopB, or CopB2) or an antigenic fragment thereof. In some aspect, the fusion polypeptide does not comprise a needle tip protein polypeptide or translocator polypeptide from a Shigella spp. (IpaD and IpaB) or a Salmonella spp. (such as, for example, S. enterica) (SipD, SseB, SipB, and SseC). It is recognized and herein contemplated that the disclosed polypeptides can be separate components of a composition or more preferably a fusion construct. By a “fusion polypeptide” is meant a peptide, polypeptide, or protein that is translated from a single, contiguous nucleic acid molecule, and which comprises sequences from at least two different proteins or antigenic regions thereof. Typically, the individual sequences are joined via a linker or spacer sequence of e.g. from about 2 to about 20 amino acids, usually from about 2 to about 10 amino acids. The amino acids in linking sequences are typically uncharged and the linker sequence usually does not exhibit secondary or tertiary structure, but does allow the fused protein/peptide segments to adopt functional secondary, tertiary, etc. conformations. One such exemplary fusion polypeptide includes Bsp22 (as set forth in SEQ ID NO: 4 and encoded by the nucleic acid sequence as set forth in SEQ ID NO: 3) and BopB (as set forth in SEQ ID NO: 6 and encoded by the nucleic acid sequence as set forth in SEQ ID NO: 5). The amino acid sequence of this chimera (i.e., 22BF) is set forth in SEQ ID NO: 2. The chimera may be encoded by any suitable nucleic acid sequence, e.g. the exemplary nucleic acid sequence depicted in SEQ ID NO: 1.

Thus, in one aspect, disclosed herein are fusion polypeptides comprising a fusion of a needle tip protein (such as, for example, IpaD, SipD, SseB, Bsp22, LcrV, BipD, PcrV, CT053, or CT668) or an antigenic fragment thereof and a translocator protein (such as, for example, IpaB, SipB, SseC, BopB, YopB, BipB, PopB, CopB, or CopB2) or an antigenic fragment thereof from a Type III secretion system (T3SS) of a Gram negative bacteria (such as, for example, Bordetella spp., Burkholderia spp., Chlamydia spp., Pseudomonas spp., Vibrio spp. or Yersinia spp. For example, the fusion polypeptide can comprise a fusion of the Shigella spp. needle tip protein (IpaD) and first translocator protein (IpaB) or fragments thereof (the fusion referred to as DBF), Salmonella spp. (such as, for example, S. enterica) SPI-1 needle tip protein (SipD) (as set forth in SEQ ID NO: 52 and encoded by the nucleic acid sequence as set forth in SEQ ID NO: 51) and translocator protein (SipB) (as set forth in SEQ ID NO: 54 and encoded by the nucleic acid sequence as set forth in SEQ ID NO: 53) or fragments thereof (the fusion referred to as S1) (as set forth in SEQ ID NO: 56 and encoded by the nucleic acid sequence as set forth in SEQ ID NO: 55), Salmonella spp. (such as, for example, S. enterica) SPI-2 needle tip protein (SseB) (as set forth in SEQ ID NO: 62 and encoded by the nucleic acid sequence as set forth in SEQ ID NO: 61) and translocator protein (SseC) (as set forth in SEQ ID NO: 64 and encoded by the nucleic acid sequence as set forth in SEQ ID NO: 63) or fragments thereof (the fusion referred to as S2) (as set forth in SEQ ID NO: 66 and encoded by the nucleic acid sequence as set forth in SEQ ID NO: 65), Bordetella spp. needle-tip protein (Bsp22) and translocator protein (BopB), or fragments thereof (the fusion referred to as 22BF); a fusion of the Yersinia spp. needle-tip protein (LerV) (as set forth in SEQ ID NO: 42 and encoded by the nucleic acid sequence as set forth in SEQ ID NO: 41) and translocator protein (YopB) (as set forth in SEQ ID NO: 44 and encoded by the nucleic acid sequence as set forth in SEQ ID NO: 43), or fragments thereof (the fusion referred to as YerF) (as set forth in SEQ ID NO: 46 and encoded by the nucleic acid sequence as set forth in SEQ ID NO: 45); a fusion of the Burkholderia spp. needle-tip protein (BipD) (as set forth in SEQ ID NO: 22 and encoded by the nucleic acid sequence as set forth in SEQ ID NO: 21) and translocator protein (BipB) (as set forth in SEQ ID NO: 24 and encoded by the nucleic acid sequence as set forth in SEQ ID NO: 23), or fragments thereof (the fusion referred to as BurkF) (as set forth in SEQ ID NO: 26 and encoded by the nucleic acid sequence as set forth in SEQ ID NO: 25); a fusion of the Pseudomonas spp. needle-tip protein (PcrV) (as set forth in SEQ ID NO: 32 and encoded by the nucleic acid sequence as set forth in SEQ ID NO: 31) and translocator protein (PopB) (as set forth in SEQ ID NO: 34 and encoded by the nucleic acid sequence as set forth in SEQ ID NO: 33), or fragments thereof (the fusion referred to as PaF) (as set forth in SEQ ID NO: 36 and encoded by the nucleic acid sequence as set forth in SEQ ID NO: 35); and/or a fusion of the Chlamydia spp. needle-tip protein CT053 (as set forth in SEQ ID NO: 74 and encoded by the nucleic acid sequence as set forth in SEQ ID NO: 73) or CT668 (as set forth in SEQ ID NO: 84 and encoded by the nucleic acid sequence as set forth in SEQ ID NO: 83) and translocator protein (CopB (as set forth in SEQ ID NO: 76 and encoded by the nucleic acid sequence as set forth in SEQ ID NO: 75) or CopB2 (as set forth in SEQ ID NO: 92 and encoded by the nucleic acid sequence as set forth in SEQ ID NO: 91)), or fragments thereof such fusions including but not limited to CT053-CopB (the CT053-CopB fusion as set forth in SEQ ID NO: 78 and encoded by the nucleic acid sequence as set forth in SEQ ID NO: 77), CT668-CopB (the CT668-CopB fusion as set forth in SEQ ID NO: 86 and encoded by the nucleic acid sequence as set forth in SEQ ID NO: 85), CT053-CopB2 (the CT053-CopB2 fusion as set forth in SEQ ID NO: 94 and encoded by the nucleic acid sequence as set forth in SEQ ID NO: 93), and CT053-CopB2 (the CT668-CopB2 fusion as set forth in SEQ ID NO: 100 and encoded by the nucleic acid sequence as set forth in SEQ ID NO: 99) and collectively each fusion being referred to as ChlamF. In one aspect, the fusion polypeptide does not comprise any needle tip protein or translocator protein or fragment thereof from a Salmonella spp. or a Shigella spp. Accordingly, disclosed herein are fusion polypeptides comprising a fusion of a needle tip protein (such as, for example, Bsp22, LcrV, BipD, PcrV, CT053, or CT668) or an antigenic fragment thereof and a translocator protein (such as, for example, BopB, YopB, BipB, PopB, CopB, or CopB2) or an antigenic fragment thereof from a Type III secretion system (T3SS) of a Gram negative bacteria (such as, for example, Bordetella spp., Burkholderia spp., Chlamydia spp., Pseudomonas spp., Vibrio spp. or Yersinia spp, wherein the gram negative bacteria is not a Salmonella enterica or Shigella spp.

It is understood and herein contemplated that the arrangement of the polypeptides in a fusion construct can have significant impact on the antigenicity of the fusion construct. Accordingly, in one aspect, disclosed herein are fusion polypeptides, wherein the fusion polypeptide is arranged such that the needle tip protein is 5′ of the translocator protein.

The present invention provides compositions for use in eliciting an immune response and/or vaccinating an individual against Gram negative bacterial infection, and/or against disease symptoms caused by Gram negative bacterial infection. The compositions include one or more substantially purified proteins, polypeptides or antigenic regions thereof as described herein, or substantially purified nucleic acid sequences (e.g. DNA cDNA, RNA, etc.) encoding such proteins, polypeptides or antigenic regions thereof, and a pharmacologically suitable/compatible carrier. By “substantially purified” is meant that the molecule is largely free of other organic molecules, cellular debris, solvents, etc. when tested using standard techniques known to those of skill in the art (e.g. gel electrophoresis, column chromatography, sequencing, mass spectroscopy, etc.). For example, the molecule is generally at least about 50, 55, 60, 65, 70, or 75% pure by wt %, and preferably is at least about 80, 85, 90, 95% or more preferably pure (e.g. 96, 97, 98, 99 or even 100% pure). The preparation of proteins, polypeptides, and peptides as described herein is well-known to those in the art, and includes, for example, recombinant preparation; isolation from a natural source; chemical synthesis; etc. The purification of proteinaceous materials is also known. However, specific exemplary methods for preparing the vaccinating agents utilized in the practice of the invention are described in detail in the Examples section below.

In addition, the composition may contain adjuvants, many of which are known in the art. For example, adjuvants suitable for use in the invention include but are not limited to: bacterial or microbial derivatives such as non-toxic derivatives of enterobacterial lipopolysaccharide (LPS), Lipid A derivatives, immunostimulatory oligonucleotides and ADP-ribosylating toxins and detoxified derivatives thereof. Non-toxic derivatives of LPS include monophosphoryl lipid A (MPL) and 3-O-deacylated MPL (3dMPL). 3dMPL is a mixture of three de-O-acylated monophosphoryl lipid A with 4, 5 or 6 acylated chains. A preferred non-toxic derivative of LPS is 3 De-O-acylated monophosphoryl lipid A. Other non-toxic LPS derivatives include monophosphoryl lipid A mimics, such as aminoalkyl glucosaminide phosphate derivatives, e.g. RC-529.

Lipid A derivatives include derivatives of lipid A from Escherichia coli such as OM-174. Immunostimulatory oligonucleotides suitable for use as adjuvants in the invention include nucleotide sequences containing a CpG motif (a dinucleotide sequence containing an unmethylated cytosine linked by a phosphate bond to a guanosine). Double-stranded RNAs and oligonucleotides containing palindromic or poly(dG) sequences have also been shown to be immunostimulatory. The CpG's can include nucleotide modifications/analogs such as phosphorothioate modifications and can be double-stranded or single-stranded, e.g. replacement of guanosine with 2′-deoxy-7-deazaguanosine. The CpG sequence may include, for example, the motif GTCGTT or TTCGTT. The CpG sequence may be specific for inducing a Th1 immune response, such as a CpG-A ODN, or it may be more specific for inducing a B cell response, such a CpG-B ODN, CpG-A and CpG-B ODNs. Preferably, the CpG is a CpG-A ODN. Preferably, the CpG oligonucleotide is constructed so that the 5′ end is accessible for receptor recognition. Optionally, two CpG oligonucleotide sequences may be attached at their 3′ ends to form “immunomers”.

Bacterial ADP-ribosylating toxins and detoxified derivatives thereof may be used as adjuvants in the invention. Preferably, the protein is derived from E. coli (e.g. E. coli heat labile enterotoxin “LT”), cholera (“CT”)(Table 1), or pertussis (“PT”).

TABLE 1
Cholera Toxin (CTA1) subunits and sequences

| Subunit | DNA sequence | AA sequence |
|---|---|---|
| Subunit | ATGGTAAAGATAATATTTGTGTTTTT | MVKIIFVFFIFL |
| A | TATTTTCTTATCATCATTTTCATATG | SSFSYANDDKLY |
| | CAAATGATGATAAGTTATATCGGGCA | RADSRPPDEIKQ |
| | GATTCTAGACCTCCTGATGAAATAAA | SGGLMPRGQSEY |
| | GCAGTCAGGTGGTCTTATGCCAAGAG | FDRGTQMNINLY |
| | GACAGAGTGAGTACTTTGACCGAGGT | DHARGTQTGFVR |
| | ACTCAAATGAATATCAACCTTTATGA | HDDGYVSTSISL |
| | TCATGCAAGAGGAACTCAGACGGGAT | RSAHLVGQTILS |
| | TTGTTAGGCACGATGATGGATATGTT | GHSTYYIYVIAT |
| | TCCACCTCAATTAGTTTGAGAAGTGC | APNMFNVNDVLG |
| | CCACTTAGTGGGTCAAACTATATTGT | AYSPHPDEQEVS |
| | CTGGTCATTCTACTTATTATATATAT | ALGGIPYSQIYG |
| | GTTATAGCCACTGCACCCAACATGTT | WYRVHFGVLDEQ |
| | TAACGTTAATGATGTATTAGGGGCAT | LHRNRGYRDRYY |
| | ACAGTCCTCATCCAGATGAACAAGAA | SNLDIAPAADGY |
| | GTTTCTGCTTTAGGTGGGATTCCATA | GLAGFPPEHRAW |
| | CTCCCAAATATATGGATGGTATCGAG | REEPWIHHAPPG |
| | TTCATTTTGGGGTGCTTGATGAACAA | CGNAPRSSMSNT |
| | TTACATCGTAATAGGGGCTACAGAGA | CDEKTQSLGVKF |
| | TAGATATTACAGTAACTTAGATATTG | LDEYQSKVKRQI |
| | CTCCAGCAGCAGATGGTTATGGATTG | FSGYQSDIDTHN |
| | GCAGGTTTCCCTCCGGAGCATAGAGC | RIKDEL |
| | TTGGAGGGAAGAGCCGTGGATTCATC | (SEQ ID |
| | ATGCACCGCCGGGTTGTGGGAATGCT | NO: 116) |
| | CCAAGATCATCGATCAGTAATACTTG | |
| | CGATGAAAAAACCCAAAGTCTAGGTG | |
| | TAAAATTCCTTGACGAATACCAATCT | |
| | AAAGTTAAAAGACAAATATTTTCAGG | |
| | CTATCAATCTGATATTGATACACATA | |
| | ATAGAATTAAGGATGAATTATGA | |
| | (SEQ ID NO: 115) | |
| Subunit | ATGATTAAATTAAAATTTGGTGTTTT | MIKLKFGVFFTV |
| B | TTTTACAGTTTTACTATCTTCAGCAT | LLSSAYAHGTPQ |
| | ATGCACATGGAACACCTCAAAATATT | NITDLCAEYHNT |
| | ACTGATTTGTGTGCAGAATACCACAA | QIYTLNDKIFSY |
| | CACACAAATATATACGCTAAATGATA | TESLAGKREMAI |
| | AGATATTTTCGTATACAGAATCTCTA | ITFKNGAIFQVE |
| | GCTGGAAAAAGAGAGATGGCTATCAT | VPGSQHIDSQKK |
| | TACTTTTAAGAATGGTGCAATTTTTC | AIERMKDTLRIA |
| | AAGTAGAAGTACCAGGTAGTCAACAT | YLTEAKVEKLCV |
| | ATAGATTCACAAAAAAAAGCGATTGA | WNNKTPHAIAAI |
| | AAGGATGAAGGATACCCTGAGGATTG | SMAN |
| | CATATCTTACTGAAGCTAAAGTCGAA | (SEQ ID |
| | AAGTTATGTGTATGGAATAATAAAAC | NO: 118) |
| | GCCTCATGCGATTGCCGCAATTAGTA | |
| | TGGCAAATTAA | |
| | (SEQ ID NO: 117) | |

The toxin or toxoid is preferably in the form of a holotoxin, comprising both A and B subunits. Preferably, the A subunit contains a detoxifying mutation; preferably the B subunit is not mutated. More preferably, the adjuvant is a detoxified LT mutant such as LT-5 K63, LT-R72, and LT-G192. The use of ADP-ribosylating toxins and detoxified derivatives thereof, particularly LT-K63 and LT-R72, is known. Such adjuvants are described, for example, in issued U.S. Pat. No. 8,039,007 (the complete contents of which is hereby incorporated by reference in entirety). Various interleukins may also be used as adjuvants to increase the immune response in a subject. In preferred embodiments, the adjuvant is a mucosal adjuvant such as, for example, the double mutant heat-labile toxin (dmLT) as set forth in SEQ ID NOs: 113 and 114) from enterotoxigenic E. coli or the active moiety thereof known as LTA1 (as set forth in SEQ ID NO: 13 and encoded by the nucleic acid sequence as set forth in SEQ ID NO: 12) and encoded by nor cholera toxin or the active moiety thereof known as CTA1. Accordingly, disclosed herein are fusion polypeptides of any preceding aspect, wherein the fusion further comprises an adjuvant such as, for example, double mutant labile toxin (dmLT) or an antigenic fragment thereof (such as, for example, LTA1 or CTA1) from Enterotoxigenic Escherichia coli. In some aspect, the dmLT or fragment thereof can also be fused to the needle tip protein-translocator protein fusion at the 5′ end. For example, specifically disclosed herein are LTA1-DBF, LTA1-S1 (as set forth in SEQ ID NO: 57 and encoded by the nucleic acid sequence as set forth in SEQ ID NO: 58), LTA1-S2 (as set forth in SEQ ID NO: 68 and encoded by the nucleic acid sequence as set forth in SEQ ID NO: 67), LTA1-SseB (as set forth in SEQ ID NO: 70 and encoded by the nucleic acid sequence as set forth in SEQ ID NO: 69), LTA1-22BF (as set forth in SEQ ID NO: 18 and encoded by the nucleic acid sequence as set forth in SEQ ID NO: 17), LTA1-BurkF (as set forth in SEQ ID NO: 28 and encoded by the nucleic acid sequence as set forth in SEQ ID NO: 27), LTA1-CT668-CopB (as set forth in SEQ ID NO: 88 and encoded by the nucleic acid sequence as set forth in SEQ ID NO: 87), LTA1-CT668-CopB2 (as set forth in SEQ ID NO: 102 and encoded by the nucleic acid sequence as set forth in SEQ ID NO: 101), LTA1-CT053-CopB (as set forth in SEQ ID NO: 80 and encoded by the nucleic acid sequence as set forth in SEQ ID NO: 79), LTA1-CT053-CopB2 (as set forth in SEQ ID NO: 96 and encoded by the nucleic acid sequence as set forth in SEQ ID NO: 95), LTA1-PaF (as set forth in SEQ ID NO: 38 and encoded by the nucleic acid sequence as set forth in SEQ ID NO: 37), and LTA1-YerF (as set forth in SEQ ID NO: 48 and encoded by the nucleic acid sequence as set forth in SEQ ID NO: 47).

Whooping cough still causes significant mortality and morbidity in children all over the world. It also continues to be a problem in adults whose immunity has waned. Herein is disclosed a strong candidate for a new protective vaccine based on research on the T3SS proteins and resulting subunit vaccines, including the vaccine against shigellosis. It is demonstrated herein that the vaccine has 100% protective efficacy against B. bronchiseptica using 22BF+dmLT. While this is a remarkable step forward, examined herein is the immune response and the protective efficacy of 22BF+dmLT±PTd against B. pertussis. The vaccine can also be taken a step further by eliciting sterilizing immunity so that the B. pertussis transmission chain can be broken.

Originally, the mechanism of protection against B. pertussis, an extracellular organism, was thought to be the humoral immune response, however, cell-mediated immunity has been found to also be important for protection with bacterial clearance mediated by Th1 and Th17 cells. By measuring cytokines corresponding to specific immune pathways, Ross et al. concluded that the wP vaccine promotes Th1 and Th17 responses while the aP vaccine elicits a mix of Th1 and Th2 responses. These differences likely account for the increased protection seen for the wP vaccine. A study in a baboon model compared wP vaccines with an aP vaccine and confirmed that the wP elicits a Th1/Th17 response while the aP vaccine elicits a Th1/Th2 response. Moreover, these studies found that aP does not prevent colonization or transmission of B. pertussis, even in asymptomatic subjects. Thus, the current pertussis resurgence could be due, in part, to the ability of the aP vaccine to protect the host against the overt symptoms of the disease while not preventing colonization and the resulting transmission of B. pertussis to susceptible children. Furthermore, protection of newborns against pertussis via aP or wP is problematic due not only to possible side effects but also because newborns lack the ability to mount a vaccine-induced Th1 response elicited through the requisite antigen presentation and T-cell activation. Although it has been shown, in some cases, that neonatal immunization can prime the immune system for subsequent booster vaccinations, the development of a protective pertussis vaccine for infants remains a need.

As noted above, the current aP vaccine does not provide sterilizing immunity. That is, the aP vaccine protects the immunized host, but does not stop colonization and transmission of the Bordetella spp. In one aspect, disclosed herein are fusion polypeptides of any preceding aspect, wherein the composition or fusion polypeptide further comprises an acellular Gram negative vaccine component (such as, for example, the acellular pertussis vaccine (aP) component pertussis toxoid (PTd)).

Pertussis toxin (PTX) is produced by Bordetella pertussis, the bacterium responsible for whooping cough. Pertussis toxin is a multi-component protein composed of six non-covalently bound subunits ranging in molecular weight from approximately about 9 kDa to about 28 kDa. These subunits are designated as S1, S2, S3, S4 and S5 and occur in native pertussis toxin in a ratio of 1:1:1:2:1, where the subunit S4 is present in two copies The largest subunit S1, also called the A protomer, is responsible for the ADP-ribosyltransferase activity. List Labs produces Pertussis Toxin Mutant R9K, E129A (both in the S1 subunit), a genetically inactivated mutant of pertussis toxin, which has a modified sequence encoding the enzyme subunit (Table 2). Virulence of this pertussis mutant is reduced relative to that found with the wild type.

TABLE 2
Pertussis Toxic Mutant R9K, E129A

| Subunit | DNA sequence | AA sequence |
|---|---|---|
| Subunit | ATGCGTTGCACTCGGGCAATTCGCCA | MRCTRAIRQTAR |
| 1 | AACCGCAAGAACAGGCTGGCTGACGT | TGWLTWLAILAV |
| | GGCTGGCGATTCTTGCCGTCACGGCG | TAPVTSPAWADD |
| | CCCGTGACTTCGCCGGCATGGGCCGA | PPATVYRYDSRP |
| | CGATCCTCCCGCCACCGTATACCGCT | PEDVFQNGFTAW |
| | ATGACTCCCGCCCGCCGGAGGACGTT | GNNDNVLDHLTG |
| | TTCCAGAACGGATTCACGGCGTGGGG | RSCQVGSSNSAF |
| | AAACAACGACAATGTGCTCGACCATC | VSTSSSRRYTEV |
| | TGACCGGACGTTCCTGCCAGGTCGGC | YLEHRMQEAVEA |
| | AGCAGCAACAGCGCTTTCGTCTCCAC | ERAGRGTGHFIG |
| | CAGCAGCAGCCGGCGCTATACCGAGG | YIYEVRADNNFY |
| | TCTATCTCGAACATCGCATGCAGGAA | GAASSYFEYVDT |
| | GCGGTCGAGGCCGAACGCGCCGGCAG | YGDNAGRILAGA |
| | GGGCACCGGCCACTTCATCGGCTACA | LATYQSEYLAHR |
| | TCTACGAAGTCCGCGCCGACAACAAT | RIPPENIRRVTR |
| | TTCTACGGCGCCGCCAGCTCGTACTT | VYHNGITGETTT |
| | CGAATACGTCGACACTTATGGCGACA | TEYSNARYVSQQ |
| | ATGCCGGCCGTATCCTCGCCGGCGCG | TRANPNPYTSRR |
| | CTGGCCACCTACCAGAGCGAATATCT | SVASIVGTLVRM |
| | GGCACACCGGCGCATTCCGCCCGAAA | APVIGACMARQA |
| | ACATCCGCAGGGTAACGCGGGTCTAT | ESSEAMAAWSER |
| | CACAACGGCATCACCGGCGAGACCAC | AGEAMVLVYYES |
| | GACCACGGAGTATTCCAACGCTCGCT | IAYSF |
| | ACGTCAGCCAGCAGACTCGCGCCAAT | (SEQ ID |
| | CCCAACCCCTACACATCGCGAAGGTC | NO: 104) |
| | CGTAGCGTCGATCGTCGGCACATTGG | |
| | TGCGCATGGCGCCGGTGATAGGCGCT | |
| | TGCATGGCGCGGCAGGCCGAAAGCTC | |
| | CGAGGCCATGGCAGCCTGGTCCGAAC | |
| | GCGCCGGCGAGGCGATGGTTCTCGTG | |
| | TACTACGAAAGCATCGCGTATTCGTT | |
| | CTAG | |
| | (SEQ ID NO: 103) | |
| Subunit | ATGCCGATCGACCGCAAGACGCTCTG | MPIDRKTLCHLL |
| 2 | CCATCTCCTGTCCGTTCTGCCGTTGG | SVLPLALLGSHV |
| | CCCTCCTCGGATCTCACGTGGCGCGG | ARASTPGIVIPP |
| | GCCTCCACGCCAGGCATCGTCATTCC | QEQITQHGGPYG |
| | GCCGCAGGAACAGATTACCCAGCATG | RCANKTRALTVA |
| | GCAGCCCCTATGGACGCTGCGCGAAC | ELRGSGDLQEYL |
| | AAGACCCGTGCCCTGACCGTGGCGGA | RHVTRGWSIFAL |
| | ATTGCGCGGCAGCGGCGATCTGCAGG | YDGTYLGGEYGG |
| | AGTACCTGCGTCATGTGACGCGCGGC | VIKDGTPGGAFD |
| | TGGTCAATATTTGCGCTCTACGATGG | LKTTFCIMTTRN |
| | CACCTATCTCGGCGGCGAATATGGCG | TGQPATDHYYSN |
| | GCGTGATCAAGGACGGAACACCCGGC | VTATRLLSSTNS |
| | GGCGCATTCGACCTGAAAACGACGTT | RLCAVFVRSGQP |
| | CTGCATCATGACCACGCGCAATACGG | VIGACTSPYDGK |
| | GTCAACCCGCAACGGATCACTACTAC | YWSMYSRLRKML |
| | AGCAACGTCACCGCCACTCGCCTGCT | YLIYVAGISVRV |
| | CTCCAGCACCAACAGCAGGCTATGCG | HVSKEEQYYDYE |
| | CGGTCTTCGTCAGAAGCGGGCAACCG | DATFETYALTGI |
| | GTCATTGGCGCCTGCACCAGCCCGTA | SICNPGSSLC |
| | TGACGGCAAGTACTGGAGCATGTACA | (SEQ ID |
| | GCCGGCTGCGGAAAATGCTTTACCTG | NO: 106) |
| | ATCTACGTGGCCGGCATCTCCGTACG | |
| | CGTCCATGTCAGCAAGGAAGAACAGT | |
| | ATTACGACTATGAGGACGCAACGTTC | |
| | GAGACTTACGCCCTTACCGGCATCTC | |
| | CATCTGCAATCCTGGATCATCCTTAT | |
| | GCTGA | |
| | (SEQ ID NO: 105) | |
| Subunit | ATGCTGATCAACAACAAGAAGCTGCT | MLINNKKLLHHI |
| 3 | TCATCACATTCTGCCCATCCTGGTGC | LPILVLALLGMR |
| | TCGCCCTGCTGGGCATGCGCACGGCC | TAQAVAPGIVIP |
| | CAGGCCGTTGCGCCAGGCATCGTCAT | PKALFTQQGGAY |
| | CCCGCCGAAGGCACTGTTCACCCAAC | GRCPNGTRALTV |
| | AGGGCGGCGCCTATGGACGCTGCCCG | AELRGNAELQTY |
| | AACGGAACCCGCGCCTTGACCGTGGC | LRQITPGWSIYG |
| | CGAACTGCGCGGCAACGCCGAATTGC | LYDGTYLGQAYG |
| | AGACGTATTTGCGCCAGATAACGCCC | GIIKDAPPGAGF |
| | GGCTGGTCCATATACGGTCTCTATGA | IYRETFCITTIY |
| | CGGTACGTACCTGGGCCAGGCGTACG | KTGQPAADHYYS |
| | GCGGCATCATCAAGGACGCGCCGCCA | KVTATRLLASTN |
| | GGCGCGGGGTTCATTTATCGCGAAAC | SRLCAVFVRDGQ |
| | TTTCTGCATCACGACCATATACAAGA | SVIGACASPYEG |
| | CCGGGCAACCGGCTGCGGATCACTAC | RYRDMYDALRRL |
| | TACAGCAAGGTCACGGCCACGCGCCT | LYMIYMSGLAVR |
| | GCTCGCCAGCACCAACAGCAGGCTGT | VHVSKEEQYYDY |
| | GCGCGGTATTCGTCAGGGACGGGCAA | EDATFQTYALTG |
| | TCGGTCATCGGAGCCTGCGCCAGCCC | ISLCNPAASIC |
| | GTATGAAGGCAGGTACAGAGACATGT | (SEQ ID |
| | ACGACGCGCTGCGGCGCCTGCTGTAC | NO: 108) |
| | ATGATCTATATGTCCGGCCTTGCCGT | |
| | ACGCGTCCACGTCAGCAAGGAAGAGC | |
| | AGTATTACGACTACGAGGACGCCACA | |
| | TTCCAGACCTATGCCCTCACCGGCAT | |
| | TTCCCTCTGCAACCCGGCAGCGTCGA | |
| | TATGCTGA | |
| | (SEQ ID NO: 107) | |
| Subunit | ATGCTGAGACGCTTCCCCACTCGAAC | MLRRFPTRTTAP |
| 4 | CACCGCCCCGGGACAGGGCGGCGCCC | GQGGARRSRVRA |
| | GGCGGTCGCGCGTGCGCGCCCTGGCG | LAWLLASGAMTH |
| | TGGTTGCTGGCATCCGGCGCGATGAC | LSPALADVPYVL |
| | GCATCTTTCCCCCGCCCTGGCCGACG | VKTNMVVTSVAM |
| | TTCCTTATGTGCTGGTGAAGACCAAT | KPYEVTPTRMLV |
| | ATGGTGGTCACCAGCGTAGCCATGAA | CGIAAKLGAAAS |
| | GCCGTATGAAGTCACCCCGACGCGCA | SPDAHVPFCFGK |
| | TGCTGGTCTGCGGCATCGCCGCCAAA | LKRPGSSPMEVM |
| | CTGGGCGCCGCGGCCAGCAGCCCGGA | LRAVFMQDQRPL |
| | CGCGCACGTGCCGTTCTGCTTCGGCA | RMFLGPKQLTFE |
| | AGGATCTCAAGCGTCCCGGCAGCAGT | GKPALELIRMVE |
| | CCCATGGAAGTCATGTTGCGCGCCGT | CSGKQDCP |
| | CTTCATGCAACAACGGCCGCTGCGCA | (SEQ ID |
| | TGTTTCTGGGTCCCAAGCAACTCACT | NO: 110) |
| | TTCGAAGGCAAGCCCGCGCTCGAACT | |
| | GATCCGGATGGTCGAATGCAGCGGCA | |
| | AGCAGGATTGCCCCTGA | |
| | (SEQ ID NO: 109) | |
| Subunit | ATGCAGCGGCAAGCAGGATTGCCCCT | MQRQAGLPLKAN |
| 5 | GAAGGCGAACCCCATGCATACCATCG | PMHTIASILLSV |
| | CATCCATCCTGTTGTCCGTGCTCGGC | LGIYSPADVAGL |
| | ATATACAGCCCGGCTGACGTCGCCGG | PTHLYKNFTVQE |
| | CTTGCCGACCCATCTGTACAAGAACT | LALKLKGKNQEF |
| | TCACTGTCCAGGAGCTGGCCTTGAAA | CLTAFMSGRSLV |
| | CTGAAGGGCAAGAATCAGGAGTTCTG | RACLSDAGHEHD |
| | CCTGACCGCCTTCATGTCGGGCAGAA | TWFDTMLGFAIS |
| | GCCTGGTCCGGGCGTGCCTGTCCGAC | AYALKSRIALTV |
| | GCGGGACACGAGCACGACACGTGGTT | EDSPYPGTPGDL |
| | CGACACCATGCTTGGCTTTGCCATAT | LELQICPLNGYC |
| | CCGCGTATGCGCTCAAGAGCCGGATC | E |
| | GCGCTGACGGTGGAAGACTCGCCGTA | (SEQ ID |
| | TCCGGGCACTCCCGGCGATCTGCTCG | NO: 112) |
| | AACTGCAGATCTGCCCGCTCAACGGA | |
| | TATTGCGAATGA | |
| | (SEQ ID NO: 111) | |

It is understood and herein contemplated that the disclosed polypeptides, adjuvants, and acellular vaccine components for use in eliciting an immune response or for treating, inhibiting, or preventing a Gram negative bacterial infection can be administered in compositions such as vaccines as individual polypeptides or as a fusion construct or a combination thereof. Thus, in one aspect, disclosed herein are compositions comprising a T3SA needle tip protein (such as, for example, Bsp22, LcrV, BipD, PcrV, CT053, or CT668) or an antigenic fragment thereof from a Gram negative bacteria (such as, for example, Bordetella spp., Burkholderia spp., Chlamydia spp., Pseudomonas spp., Vibrio spp. or Yersinia spp.) and a T3SA translocator protein (such as, for example, BopB, YopB, BipB, PopB, CopB, or CopB2) or an antigenic fragment thereof from a Gram negative bacteria; wherein the gram negative bacteria is not a Salmonella enterica or Shigella spp. In one aspect, the composition can comprise the needle tip protein or fragment thereof and the translocator protein or fragment thereof as separate components or as a fusion polypeptide. Also disclosed herein are compositions of any preceding aspect, wherein the composition comprises an adjuvant (such as, for example, cholera toxin, CTA1, dmLT, or LTA1) and/or bacterial toxin protein, such as a pertussis toxoid (PTd). Thus, in one aspect, disclosed herein are vaccines comprising any of the peptides, polypeptides, proteins, fusion peptides, fusion polypeptides, fusion proteins, or compositions disclosed herein. In some embodiments, the vaccine can further comprise an acellular gram negative vaccine or active components thereof.

### 1. Sequence Similarities

It is understood that as discussed herein the use of the terms homology and identity mean the same thing as similarity. Thus, for example, if the use of the word homology is used between two non-natural sequences it is understood that this is not necessarily indicating an evolutionary relationship between these two sequences, but rather is looking at the similarity or relatedness between their nucleic acid sequences. Many of the methods for determining homology between two evolutionarily related molecules are routinely applied to any two or more nucleic acids or proteins for the purpose of measuring sequence similarity regardless of whether they are evolutionarily related or not.

In general, it is understood that one way to define any known variants and derivatives or those that might arise, of the disclosed genes and proteins herein, is through defining the variants and derivatives in terms of homology to specific known sequences. This identity of particular sequences disclosed herein is also discussed elsewhere herein. In general, variants of genes and proteins herein disclosed (such as, for example, Bsp22, LcrV, BipD, PcrV, CT053, CT668, BopB, YopB, BipB, PopB, CopB, CopB2, 22BF, BurkF, PaF, YerF, CT053-CopB, CT053-CopB2, CT668-CopB, or CT668-CopB2) typically have at least about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent homology to the stated sequence or the native sequence. Those of skill in the art readily understand how to determine the homology of two proteins or nucleic acids, such as genes. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2:482 (1981), by the homology alignment algorithm of Needleman and Wunsch, J. MoL Biol. 48:443 (1970), by the search for similarity method of Pearson and Lipman, Proc. Natl. Acad. Sci. U.S.A. 85:2444 (1988), by computerized implementations of these algorithms (GAP, BESTFIT, FASTA, and TFASTA in the Wisconsin Genetics Software Package, Genetics Computer Group, 575 Science Dr., Madison, WI), or by inspection.

It is understood that any of the methods typically can be used and that in certain instances the results of these various methods may differ, but the skilled artisan understands if identity is found with at least one of these methods, the sequences would be said to have the stated identity, and be disclosed herein.

For example, as used herein, a sequence recited as having a particular percent homology to another sequence refers to sequences that have the recited homology as calculated by any one or more of the calculation methods described above. For example, a first sequence has 80 percent homology, as defined herein, to a second sequence if the first sequence is calculated to have 80 percent homology to the second sequence using the Zuker calculation method even if the first sequence does not have 80 percent homology to the second sequence as calculated by any of the other calculation methods. As another example, a first sequence has 80 percent homology, as defined herein, to a second sequence if the first sequence is calculated to have 80 percent homology to the second sequence using both the Zuker calculation method and the Pearson and Lipman calculation method even if the first sequence does not have 80 percent homology to the second sequence as calculated by the Smith and Waterman calculation method, the Needleman and Wunsch calculation method, the Jaeger calculation methods, or any of the other calculation methods. As yet another example, a first sequence has 80 percent homology, as defined herein, to a second sequence if the first sequence is calculated to have 80 percent homology to the second sequence using each of calculation methods (although, in practice, the different calculation methods will often result in different calculated homology percentages).

### 2. Nucleic Acids

There are a variety of molecules disclosed herein that are nucleic acid based, including for example the nucleic acids that encode, for example Bsp22, LcrV, BipD, PcrV, CT053, CT668, BopB, YopB, BipB, PopB, CopB, CopB2, 22BF, BurkF, PaF, YerF, CT053-CopB, CT053-CopB2, CT668-CopB, or CT668-CopB2 or antigenic fragments thereof, as well as various functional nucleic acids. The disclosed nucleic acids are made up of for example, nucleotides, nucleotide analogs, or nucleotide substitutes. Non-limiting examples of these and other molecules are discussed herein. It is understood that for example, when a vector is expressed in a cell, that the expressed mRNA will typically be made up of A, C, G, and U. Likewise, it is understood that if, for example, an antisense molecule is introduced into a cell or cell environment through for example exogenous delivery, it is advantageous that the antisense molecule be made up of nucleotide analogs that reduce the degradation of the antisense molecule in the cellular environment.

### a) Nucleotides and Related Molecules

A nucleotide is a molecule that contains a base moiety, a sugar moiety and a phosphate moiety. Nucleotides can be linked together through their phosphate moieties and sugar moieties creating an internucleoside linkage. The base moiety of a nucleotide can be adenin-9-yl (A), cytosin-1-yl (C), guanin-9-yl (G), uracil-1-yl (U), and thymin-1-yl (T). The sugar moiety of a nucleotide is a ribose or a deoxyribose. The phosphate moiety of a nucleotide is pentavalent phosphate. An non-limiting example of a nucleotide would be 3′-AMP (3′-adenosine monophosphate) or 5′-GMP (5′-guanosine monophosphate). There are many varieties of these types of molecules available in the art and available herein.

A nucleotide analog is a nucleotide which contains some type of modification to either the base, sugar, or phosphate moieties. Modifications to nucleotides are well known in the art and would include for example, 5-methylcytosine (5-me-C), 5-hydroxymethyl cytosine, xanthine, hypoxanthine, and 2-aminoadenine, as well as modifications at the sugar or phosphate moieties. There are many varieties of these types of molecules available in the art and available herein.

Nucleotide substitutes are molecules having similar functional properties to nucleotides, but which do not contain a phosphate moiety, such as peptide nucleic acid (PNA). Nucleotide substitutes are molecules that will recognize nucleic acids in a Watson-Crick or Hoogsteen manner, but which are linked together through a moiety other than a phosphate moiety. Nucleotide substitutes are able to conform to a double helix type structure when interacting with the appropriate target nucleic acid. There are many varieties of these types of molecules available in the art and available herein.

It is also possible to link other types of molecules (conjugates) to nucleotides or nucleotide analogs to enhance for example, cellular uptake. Conjugates can be chemically linked to the nucleotide or nucleotide analogs. Such conjugates include but are not limited to lipid moieties such as a cholesterol moiety. (Letsinger et al., Proc. Natl. Acad. Sci. USA, 1989, 86, 6553-6556). There are many varieties of these types of molecules available in the art and available herein.

A Watson-Crick interaction is at least one interaction with the Watson-Crick face of a nucleotide, nucleotide analog, or nucleotide substitute. The Watson-Crick face of a nucleotide, nucleotide analog, or nucleotide substitute includes the C2, N1, and C6 positions of a purine based nucleotide, nucleotide analog, or nucleotide substitute and the C2, N3, C4 positions of a pyrimidine based nucleotide, nucleotide analog, or nucleotide substitute.

A Hoogsteen interaction is the interaction that takes place on the Hoogsteen face of a nucleotide or nucleotide analog, which is exposed in the major groove of duplex DNA. The Hoogsteen face includes the N7 position and reactive groups (NH2 or O) at the C6 position of purine nucleotides.

### b) Sequences

There are a variety of sequences related to the protein molecules involved in the signaling pathways disclosed herein, for example Bsp22, LcrV, BipD, PcrV, CT053, CT668, BopB, YopB, BipB, PopB, CopB, CopB2, 22BF, BurkF, PaF, YerF, CT053-CopB, CT053-CopB2, CT668-CopB, or CT668-CopB2, or any of the nucleic acids disclosed herein for making Bsp22, LcrV, BipD, PcrV, CT053, CT668, BopB, YopB, BipB, PopB, CopB, CopB2, 22BF, BurkF, PaF, YerF, CT053-CopB, CT053-CopB2, CT668-CopB, or CT668-CopB2, all of which are encoded by nucleic acids or are nucleic acids. The sequences for the human analogs of these genes, as well as other analogs, and alleles of these genes, and splice variants and other types of variants, are available in a variety of protein and gene databases, including GENBANK®. Those of skill in the art understand how to resolve sequence discrepancies and differences and to adjust the compositions and methods relating to a particular sequence to other related sequences. Primers and/or probes can be designed for any given sequence given the information disclosed herein and known in the art.

### 3. Nucleic Acid Delivery

In the methods described above which include the administration and uptake of exogenous DNA into the cells of a subject (i.e., gene transduction or transfection), the disclosed nucleic acids can be in the form of naked DNA or RNA, or the nucleic acids can be in a vector for delivering the nucleic acids to the cells, whereby the antibody-encoding DNA fragment is under the transcriptional regulation of a promoter, as would be well understood by one of ordinary skill in the art. The vector can be a commercially available preparation, such as an adenovirus vector (Quantum Biotechnologies, Inc. (Laval, Quebec, Canada). Delivery of the nucleic acid or vector to cells can be via a variety of mechanisms. As one example, delivery can be via a liposome, using commercially available liposome preparations such as LIPOFECTIN, LIPOFECTAMINE (GIBCO-BRL, Inc., Gaithersburg, MD), SUPERFECT (Qiagen, Inc. Hilden, Germany) and TRANSFECTAM (Promega Biotec, Inc., Madison, WI), as well as other liposomes developed according to procedures standard in the art. In addition, the disclosed nucleic acid or vector can be delivered in vivo by electroporation, the technology for which is available from Genetronics, Inc. (San Diego, CA) as well as by means of a SONOPORATION machine (ImaRx Pharmaceutical Corp., Tucson, AZ).

As one example, vector delivery can be via a viral system, such as a retroviral vector system which can package a recombinant retroviral genome (see e.g., Pastan et al., Proc. Natl. Acad. Sci. U.S.A. 85:4486, 1988; Miller et al., Mol. Cell. Biol. 6:2895, 1986). The recombinant retrovirus can then be used to infect and thereby deliver to the infected cells nucleic acid encoding a broadly neutralizing antibody (or active fragment thereof). The exact method of introducing the altered nucleic acid into mammalian cells is, of course, not limited to the use of retroviral vectors. Other techniques are widely available for this procedure including the use of adenoviral vectors (Mitani et al., Hum. Gene Ther. 5:941-948, 1994), adeno-associated viral (AAV) vectors (Goodman et al., Blood 84:1492-1500, 1994), lentiviral vectors (Naidini et al., Science 272:263-267, 1996), pseudotyped retroviral vectors (Agrawal et al., Exper. Hematol. 24:738-747, 1996). Physical transduction techniques can also be used, such as liposome delivery and receptor-mediated and other endocytosis mechanisms (see, for example, Schwartzenberger et al., Blood 87:472-478, 1996). This disclosed compositions and methods can be used in conjunction with any of these or other commonly used gene transfer methods.

As one example, if the antibody-encoding nucleic acid is delivered to the cells of a subject in an adenovirus vector, the dosage for administration of adenovirus to humans can range from about 10⁷ to about 10⁹ plaque forming units (pfu) per injection but can be as high as about 10¹² pfu per injection (Crystal, Hum. Gene Ther. 8:985-1001, 1997; Alvarez and Curiel, Hum. Gene Ther. 8:597-613, 1997). A subject can receive a single injection, or, if additional injections are necessary, they can be repeated at six month intervals (or other appropriate time intervals, as determined by the skilled practitioner) for an indefinite period and/or until the efficacy of the treatment has been established.

Parenteral administration of the nucleic acid or vector, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. For additional discussion of suitable formulations and various routes of administration of therapeutic compounds, see, e.g., Remington: The Science and Practice of Pharmacy (19th ed.) ed. A. R. Gennaro, Mack Publishing Company, Easton, PA 1995.

### 4. Delivery of the Compositions to Cells

There are a number of compositions and methods which can be used to deliver nucleic acids to cells, either in vitro or in vivo. These methods and compositions can largely be broken down into two classes: viral based delivery systems and non-viral based delivery systems. For example, the nucleic acids can be delivered through a number of direct delivery systems such as, electroporation, lipofection, calcium phosphate precipitation, plasmids, viral vectors, viral nucleic acids, phage nucleic acids, phages, cosmids, or via transfer of genetic material in cells or carriers such as cationic liposomes. Appropriate means for transfection, including viral vectors, chemical transfectants, or physico-mechanical methods such as electroporation and direct diffusion of DNA, are described by, for example, Wolff, J. A., et al., Science, 247, 1465-1468, (1990); and Wolff, J. A. Nature, 352, 815-818, (1991). Such methods are well known in the art and readily adaptable for use with the compositions and methods described herein. In certain cases, the methods will be modified to specifically function with large DNA molecules. Further, these methods can be used to target certain diseases and cell populations by using the targeting characteristics of the carrier.

### a) Nucleic Acid Based Delivery Systems

Transfer vectors can be any nucleotide construction used to deliver genes into cells (e.g., a plasmid), or as part of a general strategy to deliver genes, e.g., as part of recombinant retrovirus or adenovirus (Ram et al. Cancer Res. 53:83-88, (1993)).

As used herein, plasmid or viral vectors are agents that transport the disclosed nucleic acids, such as 22BF into the cell without degradation and include a promoter yielding expression of the gene in the cells into which it is delivered. Viral vectors are, for example, Adenovirus, Adeno-associated virus, Herpes virus, Vaccinia virus, Polio virus, AIDS virus, neuronal trophic virus, Sindbis and other RNA viruses, including these viruses with the HIV backbone. Also preferred are any viral families which share the properties of these viruses which make them suitable for use as vectors. Retroviruses include Murine Maloney Leukemia virus, MMLV, and retroviruses that express the desirable properties of MMLV as a vector. Retroviral vectors are able to carry a larger genetic payload, i.e., a transgene or marker gene, than other viral vectors, and for this reason are a commonly used vector. However, they are not as useful in non-proliferating cells. Adenovirus vectors are relatively stable and easy to work with, have high titers, and can be delivered in aerosol formulation, and can transfect non-dividing cells. Pox viral vectors are large and have several sites for inserting genes, they are thermostable and can be stored at room temperature. A preferred embodiment is a viral vector which has been engineered so as to suppress the immune response of the host organism, elicited by the viral antigens. Preferred vectors of this type will carry coding regions for Interleukin 8 or 10.

Viral vectors can have higher transaction (ability to introduce genes) abilities than chemical or physical methods to introduce genes into cells. Typically, viral vectors contain, nonstructural early genes, structural late genes, an RNA polymerase III transcript, inverted terminal repeats necessary for replication and encapsidation, and promoters to control the transcription and replication of the viral genome. When engineered as vectors, viruses typically have one or more of the early genes removed and a gene or gene/promotor cassette is inserted into the viral genome in place of the removed viral DNA. Constructs of this type can carry up to about 8 kb of foreign genetic material. The necessary functions of the removed early genes are typically supplied by cell lines which have been engineered to express the gene products of the early genes in trans.

### (1) Retroviral Vectors

A retrovirus is an animal virus belonging to the virus family of Retroviridae, including any types, subfamilies, genus, or tropisms. Retroviral vectors, in general, are described by Verma, I. M., Retroviral vectors for gene transfer.

A retrovirus is essentially a package which has packed into it nucleic acid cargo. The nucleic acid cargo carries with it a packaging signal, which ensures that the replicated daughter molecules will be efficiently packaged within the package coat. In addition to the package signal, there are a number of molecules which are needed in cis, for the replication, and packaging of the replicated virus. Typically a retroviral genome, contains the gag, pol, and env genes which are involved in the making of the protein coat. It is the gag, pol, and env genes which are typically replaced by the foreign DNA that it is to be transferred to the target cell. Retrovirus vectors typically contain a packaging signal for incorporation into the package coat, a sequence which signals the start of the gag transcription unit, elements necessary for reverse transcription, including a primer binding site to bind the tRNA primer of reverse transcription, terminal repeat sequences that guide the switch of RNA strands during DNA synthesis, a purine rich sequence 5′ to the 3′ LTR that serve as the priming site for the synthesis of the second strand of DNA synthesis, and specific sequences near the ends of the LTRs that enable the insertion of the DNA state of the retrovirus to insert into the host genome. The removal of the gag, pol, and env genes allows for about 8 kb of foreign sequence to be inserted into the viral genome, become reverse transcribed, and upon replication be packaged into a new retroviral particle. This amount of nucleic acid is sufficient for the delivery of a one to many genes depending on the size of each transcript. It is preferable to include either positive or negative selectable markers along with other genes in the insert.

Since the replication machinery and packaging proteins in most retroviral vectors have been removed (gag, pol, and env), the vectors are typically generated by placing them into a packaging cell line. A packaging cell line is a cell line which has been transfected or transformed with a retrovirus that contains the replication and packaging machinery, but lacks any packaging signal. When the vector carrying the DNA of choice is transfected into these cell lines, the vector containing the gene of interest is replicated and packaged into new retroviral particles, by the machinery provided in cis by the helper cell. The genomes for the machinery are not packaged because they lack the necessary signals.

### (2) Adenoviral Vectors

The construction of replication-defective adenoviruses has been described (Berkner et al., J. Virology 61:1213-1220 (1987); Massie et al., Mol. Cell. Biol. 6:2872-2883 (1986); Haj-Ahmad et al., J. Virology 57:267-274 (1986); Davidson et al., J. Virology 61:1226-1239 (1987); Zhang “Generation and identification of recombinant adenovirus by liposome-mediated transfection and PCR analysis” BioTechniques 15:868-872 (1993)). The benefit of the use of these viruses as vectors is that they are limited in the extent to which they can spread to other cell types, since they can replicate within an initial infected cell, but are unable to form new infectious viral particles. Recombinant adenoviruses have been shown to achieve high efficiency gene transfer after direct, in vivo delivery to airway epithelium, hepatocytes, vascular endothelium, CNS parenchyma and a number of other tissue sites (Morsy, J. Clin. Invest. 92:1580-1586 (1993); Kirshenbaum, J. Clin. Invest. 92:381-387 (1993); Roessler, J. Clin. Invest. 92:1085-1092 (1993); Moullier, Nature Genetics 4:154-159 (1993); La Salle, Science 259:988-990 (1993); Gomez-Foix, J. Biol. Chem. 267:25129-25134 (1992); Rich, Human Gene Therapy 4:461-476 (1993); Zabner, Nature Genetics 6:75-83 (1994); Guzman, Circulation Research 73:1201-1207 (1993); Bout, Human Gene Therapy 5:3-10 (1994); Zabner, Cell 75:207-216 (1993); Caillaud, Eur. J. Neuroscience 5:1287-1291 (1993); and Ragot, J. Gen. Virology 74:501-507 (1993)). Recombinant adenoviruses achieve gene transduction by binding to specific cell surface receptors, after which the virus is internalized by receptor-mediated endocytosis, in the same manner as wild type or replication-defective adenovirus (Chardonnet and Dales, Virology 40:462-477 (1970); Brown and Burlingham, J. Virology 12:386-396 (1973); Svensson and Persson, J. Virology 55:442-449 (1985); Seth, et al., J. Virol. 51:650-655 (1984); Seth, et al., Mol. Cell. Biol. 4:1528-1533 (1984); Varga et al., J. Virology 65:6061-6070 (1991); Wickham et al., Cell 73:309-319 (1993)).

A viral vector can be one based on an adenovirus which has had the E1 gene removed and these virons are generated in a cell line such as the human 293 cell line. In another preferred embodiment both the E1 and E3 genes are removed from the adenovirus genome.

### 5. Adeno-Associated Viral Vectors

Another type of viral vector is based on an adeno-associated virus (AAV). This defective parvovirus is a preferred vector because it can infect many cell types and is nonpathogenic to humans. AAV type vectors can transport about 4 to 5 kb and wild type AAV is known to stably insert into chromosome 19. Vectors which contain this site specific integration property are preferred. An especially preferred embodiment of this type of vector is the P4.1 C vector produced by Avigen, San Francisco, CA, which can contain the herpes simplex virus thymidine kinase gene, HSV-tk, and/or a marker gene, such as the gene encoding the green fluorescent protein, GFP.

In another type of AAV virus, the AAV contains a pair of inverted terminal repeats (ITRs) which flank at least one cassette containing a promoter which directs cell-specific expression operably linked to a heterologous gene. Heterologous in this context refers to any nucleotide sequence or gene which is not native to the AAV or B19 parvovirus.

Typically the AAV and B19 coding regions have been deleted, resulting in a safe, noncytotoxic vector. The AAV ITRs, or modifications thereof, confer infectivity and site-specific integration, but not cytotoxicity, and the promoter directs cell-specific expression. U.S. Pat. No. 6,261,834 is herein incorporated by reference for material related to the AAV vector.

The disclosed vectors thus provide DNA molecules which are capable of integration into a mammalian chromosome without substantial toxicity.

The inserted genes in viral and retroviral usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

### a) Large Payload Viral Vectors

Molecular genetic experiments with large human herpesviruses have provided a means whereby large heterologous DNA fragments can be cloned, propagated and established in cells permissive for infection with herpesviruses (Sun et al., Nature genetics 8:33-41, 1994; Cotter and Robertson, Curr Opin Mol Ther 5:633-644, 1999). These large DNA viruses (herpes simplex virus (HSV) and Epstein-Barr virus (EBV), have the potential to deliver fragments of human heterologous DNA>150 kb to specific cells. EBV recombinants can maintain large pieces of DNA in the infected B-cells as episomal DNA. Individual clones carried human genomic inserts up to 330 kb appeared genetically stable The maintenance of these episomes requires a specific EBV nuclear protein, EBNA1, constitutively expressed during infection with EBV. Additionally, these vectors can be used for transfection, where large amounts of protein can be generated transiently in vitro. Herpesvirus amplicon systems are also being used to package pieces of DNA>220 kb and to infect cells that can stably maintain DNA as episomes.

Other useful systems include, for example, replicating and host-restricted non-replicating vaccinia virus vectors.

### b) Non-Nucleic Acid Based Systems

The disclosed compositions can be delivered to the target cells in a variety of ways. For example, the compositions can be delivered through electroporation, or through lipofection, or through calcium phosphate precipitation. The delivery mechanism chosen will depend in part on the type of cell targeted and whether the delivery is occurring for example in vivo or in vitro.

Thus, the compositions can comprise, in addition to the disclosed needle tip protein-translocator protein fusion (such as, for example, 22BF) or vectors for example, lipids such as liposomes, such as cationic liposomes (e.g., DOTMA, DOPE, DC-cholesterol) or anionic liposomes. Liposomes can further comprise proteins to facilitate targeting a particular cell, if desired. Administration of a composition comprising a compound and a cationic liposome can be administered to the blood afferent to a target organ or inhaled into the respiratory tract to target cells of the respiratory tract. Regarding liposomes, see, e.g., Brigham et al. Am. J. Resp. Cell. Mol. Biol. 1:95-100 (1989); Felgner et al. Proc. Natl. Acad. Sci USA 84:7413-7417 (1987); U.S. Pat. No. 4,897,355. Furthermore, the compound can be administered as a component of a microcapsule that can be targeted to specific cell types, such as macrophages, or where the diffusion of the compound or delivery of the compound from the microcapsule is designed for a specific rate or dosage.

In the methods described above which include the administration and uptake of exogenous DNA into the cells of a subject (i.e., gene transduction or transfection), delivery of the compositions to cells can be via a variety of mechanisms. As one example, delivery can be via a liposome, using commercially available liposome preparations such as LIPOFECTIN, LIPOFECTAMINE (GIBCO-BRL, Inc., Gaithersburg, MD), SUPERFECT (Qiagen, Inc. Hilden, Germany) and TRANSFECTAM (Promega Biotec, Inc., Madison, WI), as well as other liposomes developed according to procedures standard in the art. In addition, the disclosed nucleic acid or vector can be delivered in vivo by electroporation, the technology for which is available from Genetronics, Inc. (San Diego, CA) as well as by means of a SONOPORATION machine (ImaRx Pharmaceutical Corp., Tucson, AZ).

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and Mckenzie, Immunolog. Reviews, 129:57-80, (1992); and Roffler, et al., Biochem. Pharmacol, 42:2062-2065, (1991)). These techniques can be used for a variety of other specific cell types. Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

Nucleic acids that are delivered to cells which are to be integrated into the host cell genome, typically contain integration sequences. These sequences are often viral related sequences, particularly when viral based systems are used. These viral integration systems can also be incorporated into nucleic acids which are to be delivered using a non-nucleic acid based system of deliver, such as a liposome, so that the nucleic acid contained in the delivery system can become integrated into the host genome.

Other general techniques for integration into the host genome include, for example, systems designed to promote homologous recombination with the host genome. These systems typically rely on sequence flanking the nucleic acid to be expressed that has enough homology with a target sequence within the host cell genome that recombination between the vector nucleic acid and the target nucleic acid takes place, causing the delivered nucleic acid to be integrated into the host genome. These systems and the methods necessary to promote homologous recombination are known to those of skill in the art.

### c) In Vivo/Ex Vivo

As described above, the compositions can be administered in a pharmaceutically acceptable carrier and can be delivered to the subject's cells in vivo and/or ex vivo by a variety of mechanisms well known in the art (e.g., uptake of naked DNA, liposome fusion, intramuscular injection of DNA via a gene gun, endocytosis and the like).

If ex vivo methods are employed, cells or tissues can be removed and maintained outside the body according to standard protocols well known in the art. The compositions can be introduced into the cells via any gene transfer mechanism, such as, for example, calcium phosphate mediated gene delivery, electroporation, microinjection or proteoliposomes. The transduced cells can then be infused (e.g., in a pharmaceutically acceptable carrier) or homotopically transplanted back into the subject per standard methods for the cell or tissue type. Standard methods are known for transplantation or infusion of various cells into a subject.

### 6. Expression Systems

The nucleic acids that are delivered to cells typically contain expression controlling systems. For example, the inserted genes in viral and retroviral systems usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

### a) Viral Promoters and Enhancers

Preferred promoters controlling transcription from vectors in mammalian host cells may be obtained from various sources, for example, the genomes of viruses such as: polyoma, Simian Virus 40 (SV40), adenovirus, retroviruses, hepatitis-B virus and most preferably cytomegalovirus, or from heterologous mammalian promoters, e.g. beta actin promoter. The early and late promoters of the SV40 virus are conveniently obtained as an SV40 restriction fragment which also contains the SV40 viral origin of replication (Fiers et al., Nature, 273:113 (1978)). The immediate early promoter of the human cytomegalovirus is conveniently obtained as a HindIII E restriction fragment (Greenway, P. J. et al., Gene 18:355-360 (1982)). Of course, promoters from the host cell or related species also are useful herein.

Enhancer generally refers to a sequence of DNA that functions at no fixed distance from the transcription start site and can be either 5′ (Laimins, L. et al., Proc. Natl. Acad. Sci. 78:993 (1981)) or 3′ (Lusky, M. L., et al., Mol. Cell Bio. 3:1108 (1983)) to the transcription unit. Furthermore, enhancers can be within an intron (Banerji, J. L. et al., Cell 33:729 (1983)) as well as within the coding sequence itself (Osborne, T. F., et al., Mol. Cell Bio. 4:1293 (1984)). They are usually between 10 and 300 bp in length, and they function in cis. Enhancers function to increase transcription from nearby promoters. Enhancers also often contain response elements that mediate the regulation of transcription. Promoters can also contain response elements that mediate the regulation of transcription. Enhancers often determine the regulation of expression of a gene. While many enhancer sequences are now known from mammalian genes (globin, elastase, albumin, α-fetoprotein and insulin), typically one will use an enhancer from a eukaryotic cell virus for general expression. Preferred examples are the SV40 enhancer on the late side of the replication origin (bp 100-270), the cytomegalovirus early promoter enhancer, the polyoma enhancer on the late side of the replication origin, and adenovirus enhancers.

The promotor and/or enhancer may be specifically activated either by light or specific chemical events which trigger their function. Systems can be regulated by reagents such as tetracycline and dexamethasone. There are also ways to enhance viral vector gene expression by exposure to irradiation, such as gamma irradiation, or alkylating chemotherapy drugs.

In certain embodiments the promoter and/or enhancer region can act as a constitutive promoter and/or enhancer to maximize expression of the region of the transcription unit to be transcribed. In certain constructs the promoter and/or enhancer region be active in all eukaryotic cell types, even if it is only expressed in a particular type of cell at a particular time. A preferred promoter of this type is the CMV promoter (650 bases). Other preferred promoters are SV40 promoters, cytomegalovirus (full length promoter), and retroviral vector LTR.

It has been shown that all specific regulatory elements can be cloned and used to construct expression vectors that are selectively expressed in specific cell types such as melanoma cells. The glial fibrillary acetic protein (GFAP) promoter has been used to selectively express genes in cells of glial origin.

Expression vectors used in eukaryotic host cells (yeast, fungi, insect, plant, animal, human or nucleated cells) may also contain sequences necessary for the termination of transcription which may affect mRNA expression. These regions are transcribed as polyadenylated segments in the untranslated portion of the mRNA encoding tissue factor protein. The 3′ untranslated regions also include transcription termination sites. It is preferred that the transcription unit also contains a polyadenylation region. One benefit of this region is that it increases the likelihood that the transcribed unit will be processed and transported like mRNA. The identification and use of polyadenylation signals in expression constructs is well established. It is preferred that homologous polyadenylation signals be used in the transgene constructs. In certain transcription units, the polyadenylation region is derived from the SV40 early polyadenylation signal and consists of about 400 bases. It is also preferred that the transcribed units contain other standard sequences alone or in combination with the above sequences improve expression from, or stability of, the construct.

### b) Markers

The viral vectors can include nucleic acid sequence encoding a marker product. This marker product is used to determine if the gene has been delivered to the cell and once delivered is being expressed. Preferred marker genes are the E. Coli lacZ gene, which encodes β-galactosidase, and green fluorescent protein.

In some embodiments the marker may be a selectable marker. Examples of suitable selectable markers for mammalian cells are dihydrofolate reductase (DHFR), thymidine kinase, neomycin, neomycin analog G418, hydromycin, and puromycin. When such selectable markers are successfully transferred into a mammalian host cell, the transformed mammalian host cell can survive if placed under selective pressure. There are two widely used distinct categories of selective regimes. The first category is based on a cell's metabolism and the use of a mutant cell line which lacks the ability to grow independent of a supplemented media. Two examples are: CHO DHFR-cells and mouse LTK-cells. These cells lack the ability to grow without the addition of such nutrients as thymidine or hypoxanthine. Because these cells lack certain genes necessary for a complete nucleotide synthesis pathway, they cannot survive unless the missing nucleotides are provided in a supplemented media. An alternative to supplementing the media is to introduce an intact DHFR or TK gene into cells lacking the respective genes, thus altering their growth requirements. Individual cells which were not transformed with the DHFR or TK gene will not be capable of survival in non-supplemented media.

The second category is dominant selection which refers to a selection scheme used in any cell type and does not require the use of a mutant cell line. These schemes typically use a drug to arrest growth of a host cell. Those cells which have a novel gene would express a protein conveying drug resistance and would survive the selection. Examples of such dominant selection use the drugs neomycin, (Southern P. and Berg, P., J. Molec. Appl. Genet. 1:327 (1982)), mycophenolic acid, (Mulligan, R. C. and Berg, P. Science 209:1422 (1980)) or hygromycin, (Sugden, B. et al., Mol. Cell. Biol. 5:410-413 (1985)). The three examples employ bacterial genes under eukaryotic control to convey resistance to the appropriate drug G418 or neomycin (geneticin), xgpt (mycophenolic acid) or hygromycin, respectively. Others include the neomycin analog G418 and puromycin.

### 7. Peptides

### a) Protein Variants

As discussed herein there are numerous variants of the needle tip protein-translocator protein fusion (such as, for example, Bsp22, LcrV, BipD, PcrV, CT053, CT668, BopB, YopB, BipB, PopB, CopB, CopB2, 22BF, BurkF, PaF, YerF, CT053-CopB, CT053-CopB2, CT668-CopB, or CT668-CopB2) that are known and herein contemplated. In addition, to the known functional strain variants there are derivatives of the needle tip protein and translocator protein which also function in the disclosed methods and compositions. Protein variants and derivatives are well understood to those of skill in the art and can involve amino acid sequence modifications. For example, amino acid sequence modifications typically fall into one or more of three classes: substitutional, insertional or deletional variants. Insertions include amino and/or carboxyl terminal fusions as well as intrasequence insertions of single or multiple amino acid residues. Insertions ordinarily will be smaller insertions than those of amino or carboxyl terminal fusions, for example, on the order of one to four residues. Immunogenic fusion protein derivatives, such as those described in the examples, are made by fusing a polypeptide sufficiently large to confer immunogenicity to the target sequence by cross-linking in vitro or by recombinant cell culture transformed with DNA encoding the fusion. Deletions are characterized by the removal of one or more amino acid residues from the protein sequence. Typically, no more than from about 2 to about 6 residues are deleted at any one site within the protein molecule. These variants ordinarily are prepared by site specific mutagenesis of nucleotides in the DNA encoding the protein, thereby producing DNA encoding the variant, and thereafter expressing the DNA in recombinant cell culture. Techniques for making substitution mutations at predetermined sites in DNA having a known sequence are well known, for example M13 primer mutagenesis and PCR mutagenesis. Amino acid substitutions are typically of single residues, but can occur at a number of different locations at once; insertions usually will be on the order of from about 1 to about 10 amino acid residues; and deletions will range from about 1 to about 30 residues. Deletions or insertions preferably are made in adjacent pairs, i.e. a deletion of 2 residues or insertion of 2 residues. Substitutions, deletions, insertions or any combination thereof may be combined to arrive at a final construct. The mutations must not place the sequence out of reading frame and preferably will not create complementary regions that could produce secondary mRNA structure. Substitutional variants are those in which at least one residue has been removed and a different residue inserted in its place. Such substitutions generally are made in accordance with the following Tables 3 and 4 and are referred to as conservative substitutions.

TABLE 3
Amino Acid Abbreviations

| Amino Acid | Abbreviations | |
|---|---|---|
| Alanine | Ala | A |
| Allosoleucine | AIle | |
| Arginine | Arg | R |
| Asparagine | Asn | N |
| Aspartic acid | Asp | D |
| Cysteine | Cys | C |
| Glutamic acid | Glu | E |
| Glutamine | Gln | Q |
| Glycine | Gly | G |
| Histidine | His | H |
| Isolelucine | Ile | I |
| Leucine | Leu | L |
| Lysine | Lys | K |
| Phenylalanine | Phe | F |
| Proline | Pro | P |
| Pyroglutamic acid | pGlu | |
| Serine | Ser | S |
| Threonine | Thr | T |
| Tyrosine | Tyr | Y |
| Tryptophan | Trp | W |
| Valine | Val | V |

TABLE 4
Amino Acid Substitutions

| Original | Exemplary Conservative Substitutions, |
|---|---|
| Residue | others are known in the art. |
| Ala | Ser |
| Arg | Lys; Gln |
| Asn | Gln; His |
| Asp | Glu |
| Cys | Ser |
| Gln | Asn, Lys |
| Glu | Asp |
| Gly | Pro |
| His | Asn; Gln |
| Ile | Leu; Val |
| Leu | Ile; Val |
| Lys | Arg; Gln |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

Substantial changes in function or immunological identity are made by selecting substitutions that are less conservative than those in Table 4, i.e., selecting residues that differ more significantly in their effect on maintaining (a) the structure of the polypeptide backbone in the area of the substitution, for example as a sheet or helical conformation, (b) the charge or hydrophobicity of the molecule at the target site, or (c) the bulk of the side chain. The substitutions which in general are expected to produce the greatest changes in the protein properties will be those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; (d) a residue having a bulky side chain, e.g., phenylalanine, is substituted for (or by) one not having a side chain, e.g., glycine, in this case, or (c) by increasing the number of sites for sulfation and/or glycosylation.

For example, the replacement of one amino acid residue with another that is biologically and/or chemically similar is known to those skilled in the art as a conservative substitution. For example, a conservative substitution would be replacing one hydrophobic residue for another, or one polar residue for another. The substitutions include combinations such as, for example, Gly, Ala; Val, Ile, Leu; Asp, Glu; Asn, Gln; Ser, Thr; Lys, Arg; and Phe, Tyr. Such conservatively substituted variations of each explicitly disclosed sequence are included within the mosaic polypeptides provided herein.

Substitutional or deletional mutagenesis can be employed to insert sites for N-glycosylation (Asn-X-Thr/Ser) or O-glycosylation (Ser or Thr). Deletions of cysteine or other labile residues also may be desirable. Deletions or substitutions of potential proteolysis sites, e.g. Arg, is accomplished for example by deleting one of the basic residues or substituting one by glutaminyl or histidyl residues.

Certain post-translational derivatizations are the result of the action of recombinant host cells on the expressed polypeptide. Glutaminyl and asparaginyl residues are frequently post-translationally deamidated to the corresponding glutamyl and asparyl residues. Alternatively, these residues are deamidated under mildly acidic conditions. Other post-translational modifications include hydroxylation of proline and lysine, phosphorylation of hydroxyl groups of seryl or threonyl residues, methylation of the o-amino groups of lysine, arginine, and histidine side chains (T. E. Creighton, Proteins: Structure and Molecular Properties, W. H. Freeman & Co., San Francisco pp 79-86 [1983]), acetylation of the N-terminal amine and, in some instances, amidation of the C-terminal carboxyl.

It is understood that one way to define the variants and derivatives of the disclosed proteins herein is through defining the variants and derivatives in terms of homology/identity to specific known sequences. For example, SEQ ID NO: 1 sets forth a particular sequence of Bordetella needle tip protein-translocator protein fusion (22BF) and SEQ ID NO: 2 sets forth a particular sequence of a 22BF fusion protein. Specifically disclosed are variants of these and other proteins herein disclosed which have at least, 70% or 75% or 80% or 85% or 90% or 95% homology to the stated sequence. Those of skill in the art readily understand how to determine the homology of two proteins. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2:482 (1981), by the homology alignment algorithm of Needleman and Wunsch, J. MoL Biol. 48:443 (1970), by the search for similarity method of Pearson and Lipman, Proc. Natl. Acad. Sci. U.S.A. 85:2444 (1988), by computerized implementations of these algorithms (GAP, BESTFIT, FASTA, and TFASTA in the Wisconsin Genetics Software Package, Genetics Computer Group, 575 Science Dr., Madison, WI), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989.

It is understood that the description of conservative mutations and homology can be combined together in any combination, such as embodiments that have at least 70% homology to a particular sequence wherein the variants are conservative mutations.

As this specification discusses various proteins and protein sequences it is understood that the nucleic acids that can encode those protein sequences are also disclosed. This would include all degenerate sequences related to a specific protein sequence, i.e. all nucleic acids having a sequence that encodes one particular protein sequence as well as all nucleic acids, including degenerate nucleic acids, encoding the disclosed variants and derivatives of the protein sequences. Thus, while each particular nucleic acid sequence may not be written out herein, it is understood that each and every sequence is in fact disclosed and described herein through the disclosed protein sequence. For example, one of the many nucleic acid sequences that can encode the protein sequence set forth in SEQ ID NO: 2 is set forth in SEQ ID NO: 1. It is understood that for this mutation all of the nucleic acid sequences that encode this particular derivative of the 22BF are also disclosed. It is also understood that while no amino acid sequence indicates what particular DNA sequence encodes that protein within an organism, where particular variants of a disclosed protein are disclosed herein, the known nucleic acid sequence that encodes that protein in the particular needle tip protein-translocator protein fusion (such as, for example, 22BF) from which that protein arises is also known and herein disclosed and described.

It is understood that there are numerous amino acid and peptide analogs which can be incorporated into the disclosed compositions. For example, there are numerous D amino acids or amino acids which have a different functional substituent then the amino acids shown in Table 3 and Table 4. The opposite stereo isomers of naturally occurring peptides are disclosed, as well as the stereo isomers of peptide analogs. These amino acids can readily be incorporated into polypeptide chains by charging tRNA molecules with the amino acid of choice and engineering genetic constructs that utilize, for example, amber codons, to insert the analog amino acid into a peptide chain in a site specific way.

Molecules can be produced that resemble peptides, but which are not connected via a natural peptide linkage. For example, linkages for amino acids or amino acid analogs can include —CH₂NH—, —CH₂S—, —CH₂—CH₂—, —CH═CH— (cis and trans), —COCH₂—, —CH(OH)CH₂—, and —CHH₂SO— (These and others can be found in Spatola, A. F. in Chemistry and Biochemistry of Amino Acids, Peptides, and Proteins, B. Weinstein, eds., Marcel Dekker, New York, p. 267 (1983); Spatola, A. F., Vega Data (March 1983), Vol. 1, Issue 3, Peptide Backbone Modifications (general review); Morley, Trends Pharm Sci (1980) pp. 463-468; Hudson, D. et al., Int J Pept Prot Res 14:177-185 (1979) (—CH₂NH—, —CH₂CH₂—); Spatola et al. Life Sci 38:1243-1249 (1986) (—CH₂—S); Hann J. Chem. Soc Perkin Trans. I 307-314 (1982) (—CH═CH—, cis and trans); Almquist et al. J. Med. Chem. 23:1392-1398 (1980) (—COCH₂—); Jennings-White et al. Tetrahedron Lett 23:2533 (1982) (—COCH₂—); Szelke et al. European Appln, EP 45665 CA (1982): 97:39405 (1982) (—CH(OH)CH₂—); Holladay et al. Tetrahedron. Lett 24:4401-4404 (1983) (—C(OH)CH₂—); and Hruby Life Sci 31:189-199 (1982) (—CH₂—S—); each of which is incorporated herein by reference. A particularly preferred non-peptide linkage is —CH₂NH—. It is understood that peptide analogs can have more than one atom between the bond atoms, such as β-alanine, γ-aminobutyric acid, and the like.

Amino acid analogs and analogs and peptide analogs often have enhanced or desirable properties, such as, more economical production, greater chemical stability, enhanced pharmacological properties (half-life, absorption, potency, efficacy, etc.), altered specificity (e.g., a broad-spectrum of biological activities), reduced antigenicity, and others.

D-amino acids can be used to generate more stable peptides, because D amino acids are not recognized by peptidases and such. Systematic substitution of one or more amino acids of a consensus sequence with a D-amino acid of the same type (e.g., D-lysine in place of L-lysine) can be used to generate more stable peptides. Cysteine residues can be used to cyclize or attach two or more peptides together. This can be beneficial to constrain peptides into particular conformations.

### 8. Pharmaceutical Carriers/Delivery of Pharmaceutical Products

As described above, the compositions can also be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions may be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and Mckenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

### a) Pharmaceutically Acceptable Carriers

The compositions, including antibodies, can be used therapeutically in combination with a pharmaceutically acceptable carrier.

Suitable carriers and their formulations are described in Remington: The Science and Practice of Pharmacy (19th ed.) ed. A. R. Gennaro, Mack Publishing Company, Easton, PA 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, more preferably from about 7 to about 7.6, and most preferably about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, anti-inflammatory agents, anesthetics, and the like.

The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, tri-alkyl and aryl amines and substituted ethanolamines.

### b) Therapeutic Uses

Effective dosages and schedules for administering the compositions may be determined empirically, and making such determinations is within the skill in the art. The dosage ranges for the administration of the compositions are those large enough to produce the desired effect in which the symptoms of the disorder are effected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone might range from about 1 μg/kg to up to 100 mg/kg of body weight or more per day, depending on the factors mentioned above.

In a preferred embodiment, the amount of protein that is administered per dose of vaccine is in the range of from about 0.0001 to about 1000 μg/kg. In one embodiment, the amount is in the range of from about 0.001 to about 1000 μg/kg of body weight of the recipient. In one embodiment, the amount is in the range of from about 0.01 to about 1000 μg/kg of body weight of the recipient. In one embodiment, the amount is in the range of from about 0.01 to about 100 μg/kg of body weight of the recipient. Those of skill in the art will recognize that the precise dosage may vary from situation to situation and from patient to patient, depending on e.g. age, gender, overall health, various genetic factors, and other variables known to those of skill in the art. Dosages are typically determined e.g. in the course of animal and/or human clinical trials as conducted by skilled medical personnel, e.g. physicians or veterinarians.

### C. METHODS OF USING THE COMPOSITIONS

Herein, the protective efficacy of the Bordetella spp. tip/translocator fusion, 22BF, is examined against lethal lung challenge and with complete (sterilizing) clearance of colonizing bacteria. Unlike some components of the current aP vaccine, Bsp22 and BopB are required for infection and are not mutable since they must be retained structurally and functionally within the context of a large nanomachine residing within the Bordetella cell envelope. Furthermore, targeting the Bordetella T3SA renders the pathogen less able to fight off the host innate and adaptive immune responses. Regardless of whether 22BF is protective alone or when used with components of the current aP vaccine, the innovation of this high risk, high reward investigation lies in whether this subunit vaccine can elicit sterilizing immunity and thereby prevent the colonization that results in host to host transmission. It has been reported that Bsp22 (a component of the 22BF fusion vaccine) does not elicit a serum antibody response in humans during the course of natural infection and is not a protective antigen in mice. Nevertheless, as shown herein, protective and sterilizing immunity can be obtained with the compositions disclosed herein.

Thus, in one aspect, disclosed herein are methods of eliciting an immune response in a subject to a Gram negative bacteria (such as, for example, Bordetella spp., Burkholderia spp., Chlamydia spp., Pseudomonas spp., Vibrio spp. Enteropathogenic or Enterohemorrhagic E. coli or Yersinia spp.) comprising administering to the subject the fusion polypeptides, compositions, or vaccines disclosed herein. Accordingly, in one aspect, disclosed herein are methods of eliciting an immune response against at least one Gram negative bacteria serovar in a subject in need thereof, comprising administering to the subject a composition comprising at least one needle tip protein or a fragment thereof and/or at least one translocator protein or a fragment thereof; wherein said composition is administered in an amount sufficient to elicit an immune response to said at least one Gram negative bacteria serovar in said subject; and wherein the Gram negative bacteria is not a Shigella spp. or Salmonella spp. In one aspect, the immune response elicited provides sterilizing immunity to the infectious bacterium.

As can be appreciated by the skilled artisan, the methods of eliciting an immune response can be used for the purpose of treating, inhibiting, or preventing an infection of a Gram negative bacteria (such as, for example, Bordetella spp., Burkholderia spp., Chlamydia spp., Pseudomonas spp., Vibrio spp. Enteropathogenic or Enterohemorrhagic E. coli or Yersinia spp.). Thus, in one aspect, disclosed herein are methods of treating, inhibiting, or preventing an infection of a Gram negative bacteria in a subject comprising administering to the subject a therapeutic amount of any of the fusion polypeptides, compositions, or vaccines disclosed herein. As one goal of any vaccine is not only to prevent infection or reducing the severity of disease in the individual receiving the vaccine, but also to prevent further transmission of the infectious agent (sterilizing immunity), it is understood and herein contemplated that the disclosed methods of treatment, inhibition, or preventing an infection can further comprise inhibiting and/or preventing colony formation of the bacteria and/or transmission of the bacteria to another subject.

The term “therapeutically effective” refers to the amount of the composition used that is of sufficient quantity to ameliorate one or more causes or symptoms of a disease or disorder. Such amelioration only requires a reduction or alteration, not necessarily elimination.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

The term “inhibit” refers to a decrease in an activity, response, condition, disease, or other biological parameter. This can include, but is not limited to, the complete ablation of the activity, response, condition, or disease. This can also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

### D. EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Example 1

a) Use of a T3SS Needle Tip/Translocator Protein Fusion as a Protective Antigen Against B. pertussis:

The dominant antigen eliciting protection against Gram-negative pathogens is LPS, which confers O-antigen serotype specificity. The initial project focused on Shigella, however, there are at least 58 distinct Shigella serotypes. This reduces broad-spectrum efficacy for live, attenuated and whole, killed vaccines, which tend to be somatic antigen driven. IpaD and IpaB are the surface-localized needle tip and first translocator proteins of the T3SA, respectively. They are essential for virulence in all Shigella, are >98% conserved across all Shigella species, and provide serotype-independent protection. When IpaD+IpaB+dmLT was given IN to mice, the formulation was about 80-90% protective against lethal challenge by homologous and heterologous Shigella spp. (Table 5). To reduce the production cost, IpaD and IpaB were genetically fused to make DBF. Not only did the DBF provide protection against lethal challenge, it also unexpectedly increased the cell-mediated immunity, most notably the IL-17 and IFN-γ responses. When the Salmonella enterica tip and first translocator proteins of the T3SSs of SPI-1 and SPI-2 (Salmonella Pathogenicity Islands 1 and 2) were fused, about 70% protection against lethal challenge by two S. enterica serovars was observed when both fusion proteins were administered simultaneously (Table 5).

TABLE 5
Protective efficacy of tip/translocator fusions
against challenge by appropriate pathogen.

| Vaccine | Protection (%) | Pathogen |
|---|---|---|
| DBF + dmLT | 80-90 | S. flexneri, S. sonnei |
| S1S2 + MPL (IM) | 70 | S. Typhimurium, S. Enteritidis |
| 22BF + dmLT | 100 | B. bronchiseptica |

Mice (n = 10) were vaccinated 3 times biweekly with the vaccine and then challenged with indicated pathogen at day 56. Protection is indicated as percent survival after 21 days.

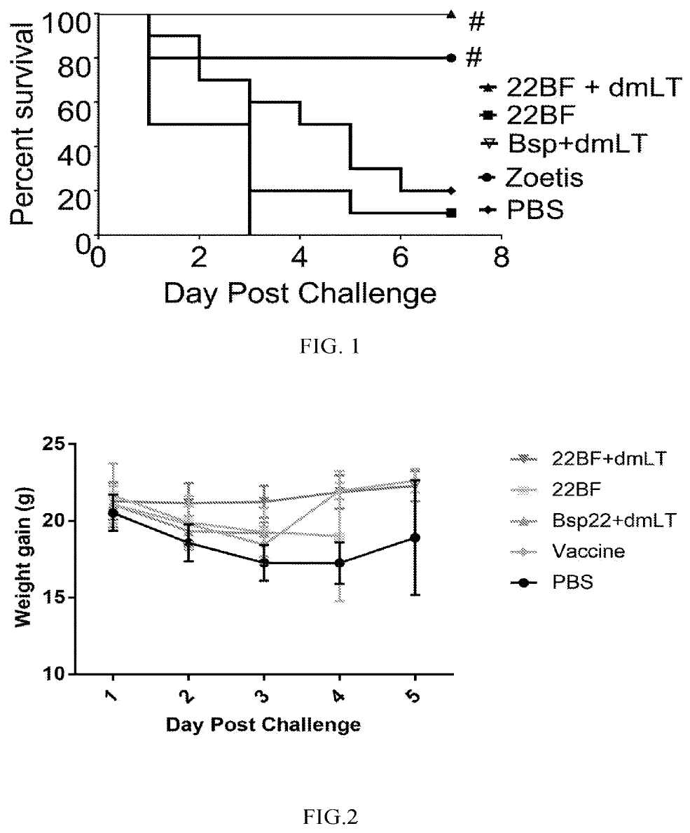

B. bronchiseptica and B. pertussis infections have been shown to also require a T3SS for virulence. To determine if a subunit of the T3SS could be used to confer protection, B. pertussis Bsp22 and BopB, the T3SS needle tip and first translocator proteins, which are 98% conserved with those of B. bronchiseptica were genetically fused. Mice were vaccinated biweekly, three times with 22BF, 22BF+dmLT, Bsp22+dmLT, or PBS. One group was also vaccinated twice with the Zoetis canine vaccine that is a killed cellular extract of B. bronchiseptica. The 22BF+dmLT exhibited 100% protection against B. bronchiseptica in a mouse lethal pulmonary model, while the commercial vaccine provided 80% protection (FIG. 1). Moreover, the mice vaccinated with 22BF+dmLT did not show signs of illness and they actually gained weight throughout the challenge (FIG. 2). In contrast, the mice vaccinated with the Zoetis vaccine lost weight and then regained it, however, they continued to display lower health scores than the mice vaccinated with 22BF+dmLT for the remainder of the experiment (FIG. 2).

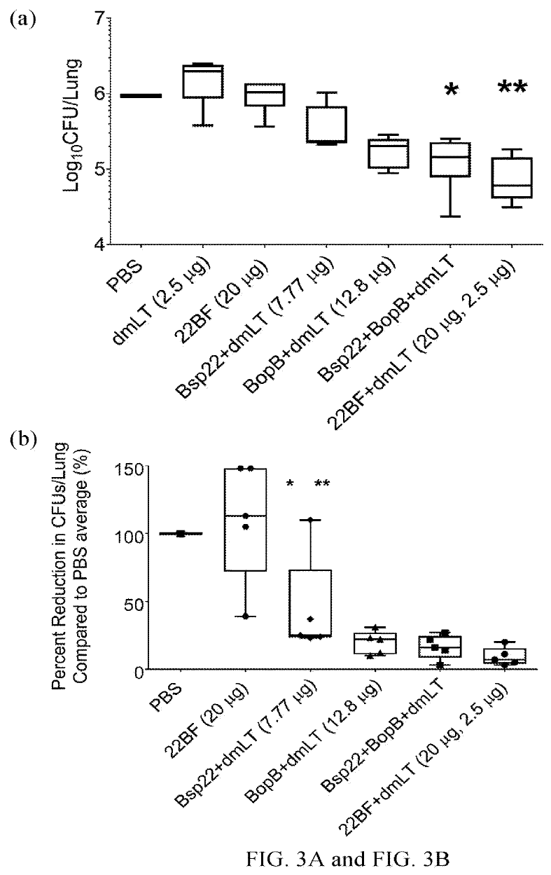

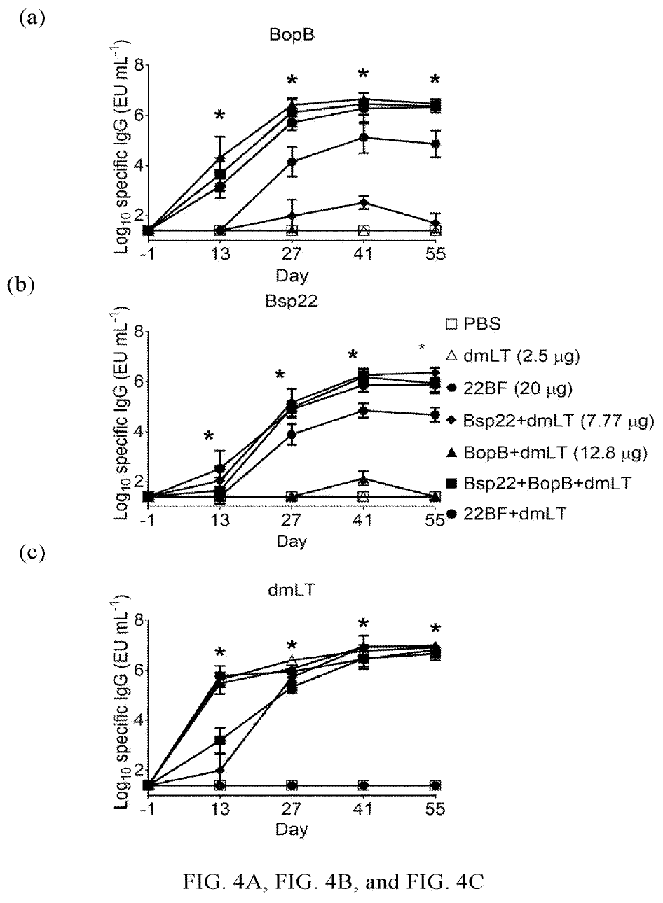

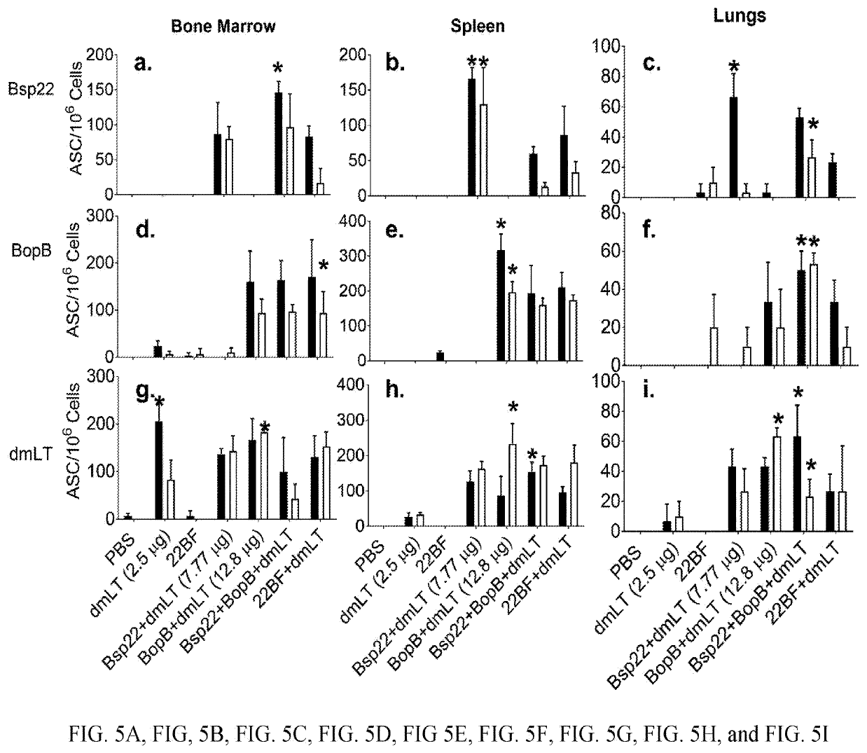

Next, the protective efficacy of the 22BF fusion with and without dmLT and in comparison to the needle tip protein Bsp22 and translocator protein BopB and the effect of fusion of dmLT relative to concurrent administration was examined. Mice were vaccinated IN on days 0, 14, 28 and challenged with Bordetella bronchiseptica on day 56. On day 7 mice were sacrificed and the colony forming units (CFU)/lung were determined (FIG. 3). 22BF+dmLT reduced the B. bronchiseptica CFU lung burden by 96.5%. Blood and fecal samples were collected on days-1, 13, 27, 41, and 55. The kinetics of IgG were assessed. Typical logarithmic increases were seen (FIG. 4) regardless of the antigen on the well. No IgA was detected in fecal samples of any of the mice. Antibody secreting cells in the bone marrow, spleen, and lungs (FIG. 5) were stimulated with Bsp22, BopB and dmLT. All organs from mice that had been vaccinated with the single proteins or double proteins or fusion, as long as the dmLT was present, exhibited some level of IgG or IgA ASCs

While protection is important, the key to a next generation subunit vaccine against Bordetella spp. must be that it exhibits sterilizing immunity (to prevent carriage). The lungs of the mice that survived the challenge of FIG. 2 were examined to look for sterilizing immunity. All of the remaining 22BF and PBS vaccinated mice exhibited CFU/lung. In contrast, the 22BF+dmLT vaccinated mice demonstrated 38% sterilizing immunity while only 12% of the mice vaccinated with the Zoetis vaccine demonstrated sterilizing immunity. Thus, the 22BF represents an innovative new antigen to protect against B. bronchiseptica and B. pertussis due to the conservation of these antigens.

It is shown herein that the use of a broad, serotype-independent subunit vaccine against Shigella spp. and S. enterica serotypes. These vaccines are based on the fusion of the T3SA tip and first translocator proteins, which are highly conserved within a given bacterial genus. The DBF has also been shown to protect monkeys from severe diarrhea and S. enterica S1S2 fusions are protective in a bovine calf model. A genetic fusion of the B. pertussis T3SA tip/translocator system, 22BF, was generated which protects 100% of the mice challenged with a lethal dose of the heterologous B. bronchiseptica. Furthermore, 22BF elicited 38% sterilizing immunity while the commercial vaccine provided 12%. Herein, not only was the B. bronchiseptica experiment repeated, but also it demonstrated that the 22BF+dmLT protects against B. pertussis. B. bronchiseptica was chosen as the first challenge pathogen since it lacks pertussis toxin (PT), which could conceivably compromise adequate assessment of the 22BF. PT is an especially important toxin secreted by B. pertussis that can significantly affect the health of the host. Shiga toxin was also considered when DBF vaccinated mice were challenged with S. dysenteriae, however, DBF protected 40% of those vaccinated mice while the individual proteins did not. Furthermore, due to the disease progression of S. dysenteriae, mice killed by the Shiga toxin versus those succumbing to the bacterial infection (based on health score immediately preceding death) were distinguished. 22BF provides some level of protection against B. pertussis, which can be boosted by the addition of pertussis toxoid (PTd). At the end of this study, it was demonstrated that 22BF protects against both B. bronchiseptica and B. pertussis. Results for two vaccination routes and doses are obtained. PTd is required in the protection against B. pertussis was shown herein. Finally, it is demonstrated that 22BF+dmLT elicits >90, if not 100%, sterilizing immunity.

### b) Cytokine Assay

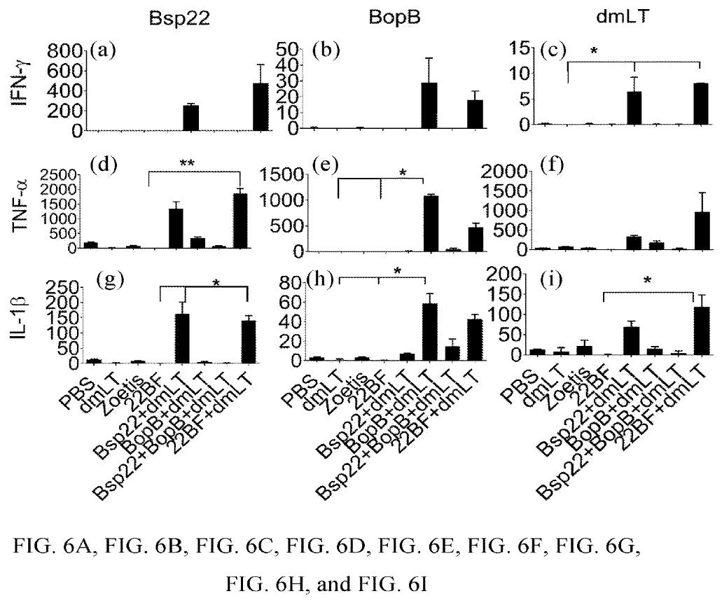

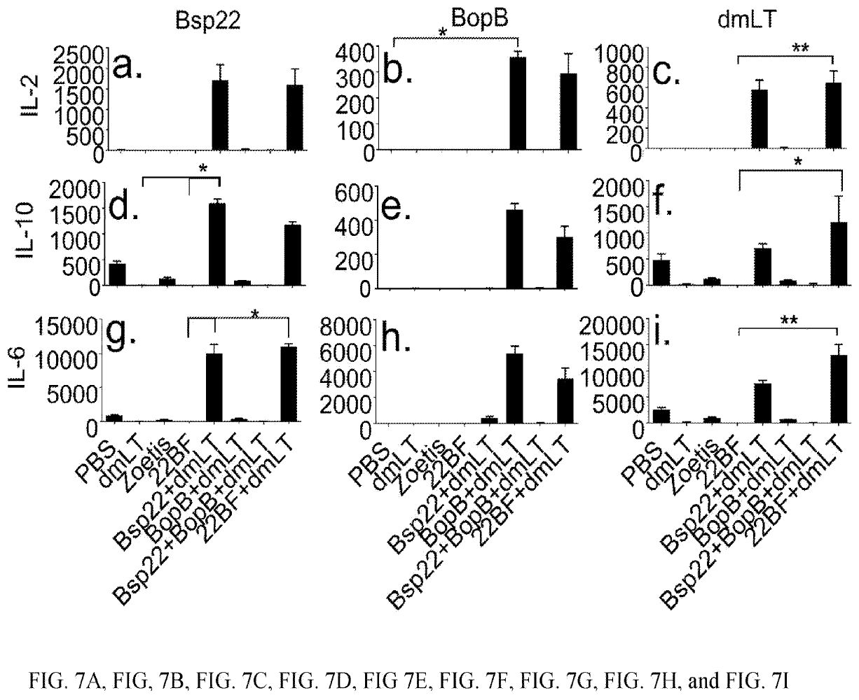

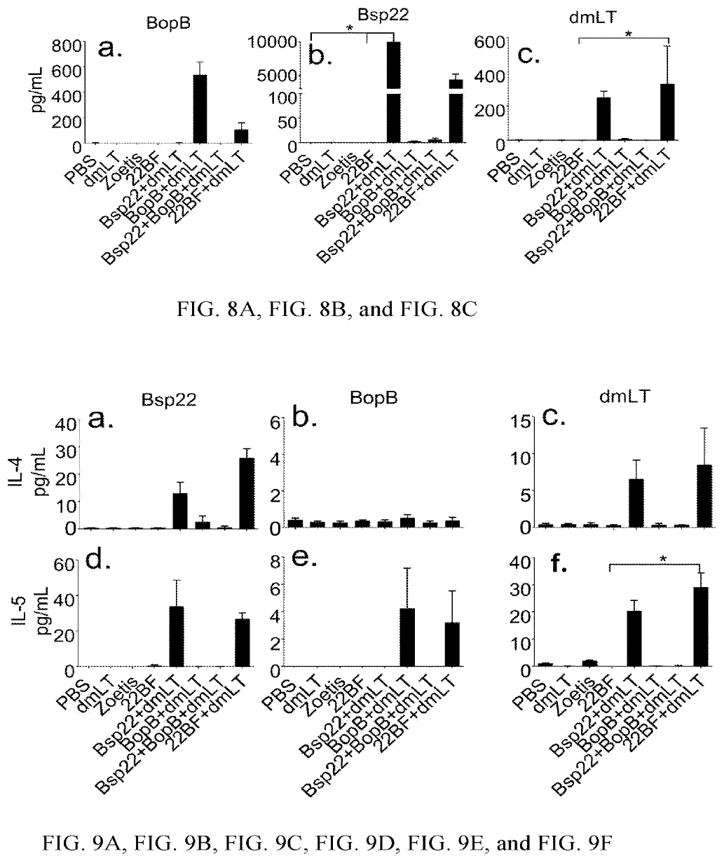

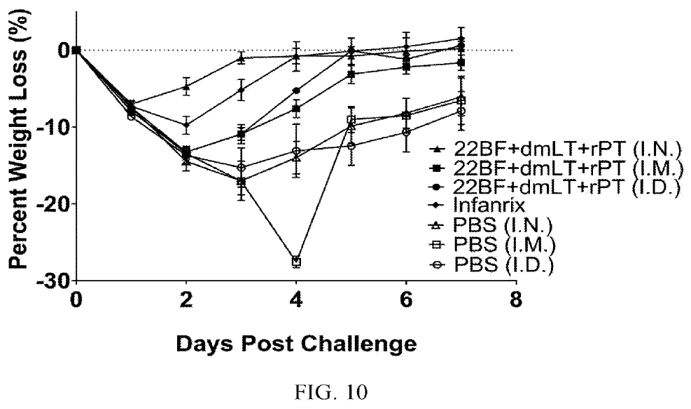

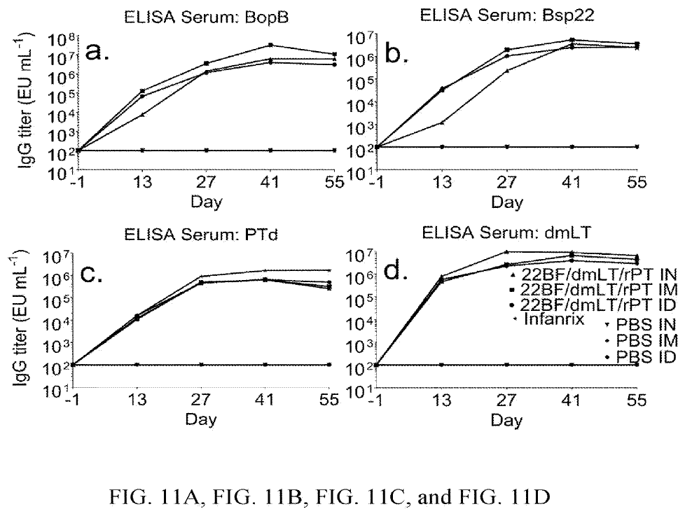

Cytokines were collected from splenocytes collected on Day 55. 100 μL of homogenized splenocytes were seeded at a concentration of 5×10⁶ cells/mL onto a flat-bottom 96-well plate. 100 μL of BopB, Bsp22, or dmLT at a concentration of 20 μg/mL was added onto cells (bringing final protein concentration to 10 μg/mL). Plates were incubated at 37° C. and 5% CO2 for 48 hours. After incubation, plates were centrifuged at 1600 rpm at 4° C. for seven minutes. Supernatants were collected and stored at −20° C. until analysis. Cytokines were analyzed using the MSD® U-Plex Platform Multiplex Assay for the following cytokines for the following immune responses: Th1: IFN-γ, TNF-α, IL-1B, IL-2, IL-6, IL-10; (FIGS. 6 and 7) Th17: IL-17A; (FIG. 8) Th2: IL-4, IL-5 (FIG. 9).

### 2. Example 2: Assess the Respective Humoral and Cell-Mediated Immune Responses Elicited by 22BF+dmLT Delivered IN and IM

IN delivery of 20 μg 22BF+2.5 μg dmLT protects 100% of vaccinated mice from death and provide 38% sterilizing immunity in the lungs following B. bronchiseptica challenge. 50 μg or 15 μg 22BF IN is delivered and then in parallel deliver 100 μg or 20 μg 22BF IM or ID to assess the humoral and cell mediated responses in each case. Based on previous work, it is known that some of these dose/route combinations do not protect mice while others offer protection.

First the effect of the route of administration of 22BF following challenge was assessed. Group 1 is a 22BF+2.5 μg dmLT IN, group 2 is 22BF+2.5 μg dmLT IM, and group 3 is a 22BF+2.5 μg dmLT IN. For each administrative route, PBS vaccinated controls were included (Groups 4, 5, and 6). Group 7 (n=10) is vaccinated subcutaneously on day 1 and 21 with the Zoetis vaccine. After day 56 following initial vaccination, mice were challenged with a sublethal dosage of B. pertussis intranasally. There was an observable difference in weight loss between mice vaccinated with the 22BF+dmLT+PTd formulation and those that only received PBS. By Day 7 all mice aside from PBS treated mice had recovered to within 3% of pre infection weight.

The immune responses can be compared with the protective efficacy and potentially define a protective correlate in the mouse model. The correlate is useful in the development of the 22BF vaccine, making the necessary adjustments when it is translated to humans. The systemic immune response can be assessed by measuring serum IgG against BopB, Bsp22, and dmLT, as well as the mucosal immune response by assessing IgA in fecal pellets. Cytokine secretion is assessed in splenocytes from vaccinated mice.

This experiment can be performed in two parts. The IN route can use a high dose of 50 μg and low dose of 15 μg. The IM route uses a high dose of 100 μg and a low dose of 40 μg. For each route, three groups of female C57BL/6 mice (10/group) are vaccinated on days 0, 14 and 28. Group 1 is a 22BF+2.5 μg dmLT IN, group 2 is 22BF+2.5 μg dmLT IM, and group 3 is a 22BF+2.5 μg dmLT IN. For each administrative route, PBS vaccinated controls were included (Groups 4, 5, and 6). Group 7 (n=10) is vaccinated subcutaneously on day 1 and 21 with the Zoetis vaccine. Blood and fecal pellets are collected on days-1, 13, 27, 41 and 55 to assess mucosal and systemic humoral responses. Individual samples are tested for the presence of anti-Bsp22, -BopB, -dmLT, -PTd IgG and IgA antibodies by ELISA. Mice were immunized on days 0, 14, and 28 with 22BF+PTd admixed with dmLT. Serum IgG antibodies specific for BopB, Bsp22, PTd, and dmLT were measured by ELISA. and IFN-γ/IL-17A secreting cells by ELISpot and cytokine secretion using Multi array assays. BAL is collected to measure IgG and IgA responses. GraphPad Prism 5.04 can be used for graphics and statistical comparisons. Differences were analyzed using t test or ANOVA where appropriate. A P value of less than 0.05 is considered significant for all comparisons.

For these experiments, serum IgG levels are >10⁵ EU/ml, antibody secreting cells at >50 IgG ASC/10⁶ cells or >20 IgA ASC/10⁶ cells, and cytokine secreting cells at >50 IFN-γ/10⁶ cells and IL-17/10⁶ cells. There can also be unique systemic and mucosal humoral immune responses from mice immunized via the IN and IM routes. 50 μg IN was chosen to facilitate an increase in sterilizing immunity. The 100 μg IM dose was based on prior findings. Antibody secreting cells specific for both proteins are detected in both mucosal and memory compartments. Finally, the full profile of cytokine secretion elicited by the vaccine can demonstrate a dose and administration route dependence. Thus, these two routes (each with a high and low dose) are expected to give rise to unique immune response profiles.

### 3. Example 3: Determine the Protective Efficacy of 22BF+dmLT Against B. bronchiseptica and B. pertussis Challenge

As discussed above, it is demonstrated herein that initial protective efficacy of 22BF+dmLT against B. bronchiseptica challenge. Insight is gained into the immune responses elicited by two doses of 22BF delivered IN and IM. Here, mice can be vaccinated and challenged with B. bronchiseptica and B. pertussis. In addition to assessing protective efficacy and sterilizing immunity of the 22BF+dmLT as well as the requirement for PTd, a protective correlate can be established for use. This method was used to identify a protective correlate associated with DBF protection of mice against Shigella. This, however, prior to the present disclosure has never been determined for such a vaccine type against an extracellular pathogen.

a) Assess Protective Efficacy of the 22BF+dmLT Delivered IN and IM Against B. Bronshiseptica Challenge Using the Mouse Lung Model with Two Challenge Doses—a Lethal Dose to Assess Survival and a Sublethal Dose to Assess Sterilizing Immunity.

Protective efficacy of 22BF+dmLT delivered IN, IM, and ID against a B. bronchiseptica challenge can be assessed in the mouse lung model. A high dose of B. bronchiseptica can be administered initially to assess protection via the lethal dose. In a second trial, a lower dose can be used to assess sterilizing immunity.

Experimental Details: Mice (20/group) are vaccinated IN on days 0, 14 and 28. Serum, and stool samples are collected as described above to measure specific antibody responses to confirm that immune responses are comparable to those obtained above. For bacterial challenges, 10 mice are challenged on day 56 with 1×10⁷ B. bronchiseptica (lethal dose) and 10 animals are challenged with 1×10⁶ B. bronchiseptica (sub-lethal dose). The mouse experiment can be repeated with vaccination occurring by the IM route. Survival can be plotted and a Log-rank test used to evaluate the differences. A P value of less than 0.05 is considered significant for all comparisons. Association of protective efficacy and markers of humoral and cellular immunity can be assessed with logistic regression models (see FIG. 1).

With respect to the IN vaccinated mice, at both doses, some level of protection is shown in the lethal lung model. The mice vaccinated with 50 μg are protected with complete sterilizing immunity. The 15 μg dose gives >90% protection with a moderate level of sterilizing immunity. Similarly, 100 μg 22BF+dmLT delivered IM has a high level of protection as well as sterilizing immunity, but perhaps not 100%, but greater than 70% protection. The 40 μg dose shows minimal protection. With these results, a protective correlate for B. bronchiseptica can be predicted, as long as the immune responses were above the levels anticipated.

b) Assess Protective Efficacy of the 22BF+dmLT±PTd Delivered IN, IM, and ID Against B. Pertussis Challenge Using the Mouse Lung Model with Two Challenge Doses—a Lethal Dose to Assess Survival and a Sublethal Dose to Assess Sterilizing Immunity.

The ultimate test of the 22BF formulation is the protective efficacy against B. pertussis. Here, the protective efficacy of 22BF+dmLT is tested with a focus on a B. pertussis challenge using the mouse lung model. Vaccinations occur IN with PTd. Furthermore, a high dose of B. pertussis is used initially to assess protection via the lethal lung model.

Mice (10/group) are vaccinated IN on days 0, 14 and 28. Scrum, and stool samples are collected. as described above to measure specific antibody responses to confirm a comparable immune response. For bacterial challenges, all mice can be challenged on day 56 with 1×10⁷ B. pertussis (lethal dose). The experiment can be repeated using IM route and ID route again with the most protective vaccine and challenge with a lethal dose and a sub-lethal dose to assess protection and sterilizing immunity. Survival can be plotted and a Log-rank test used to evaluate the differences. A P value of less than 0.05 is considered significant for all comparisons.

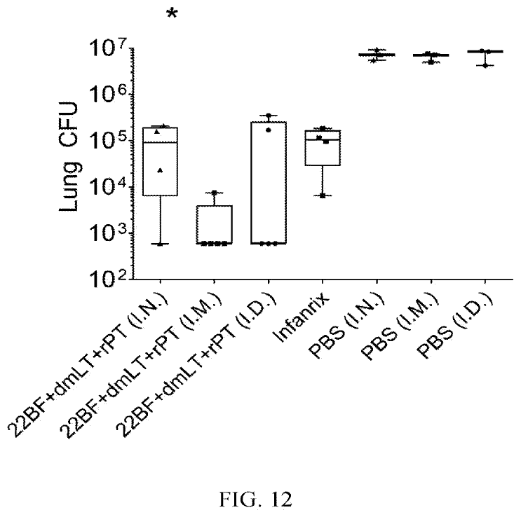

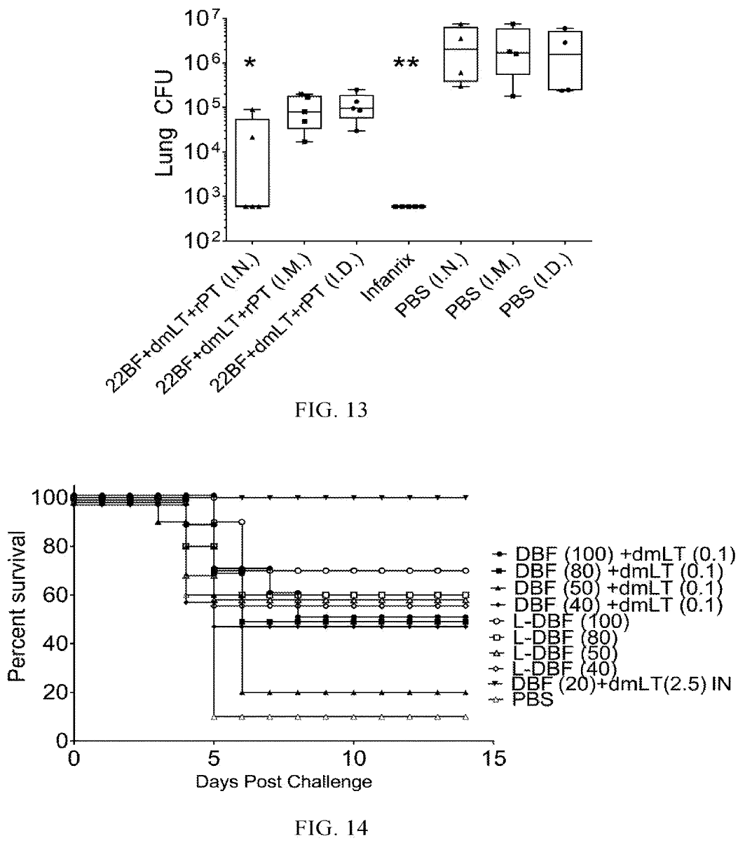

PTd can additionally be administered for protection against B. pertussis and to prevent the cellular damage associated with PT as well as increase sterilizing immunity. Mice can be vaccinated IN, IM, or ID with 22BF+PTd and dmLT and challenged with B. pertussis. Lung CFU were measured at day 3 (FIG. 12) and day 7 (FIG. 13) post challenge. As with the predicted B. bronchiseptica results, 100% protection and sterilizing immunity is obtained with 50 μg 22BF+dmLT+PTd delivered IN with reduced protection for the 15 μg dose delivered IN. Similarly, the 100 μg 22BF+dmLT+PTd delivered IM and ID achieves some significant level of protection, but sterilizing immunity is limited though could be greater at higher dosage by day 7 post challenge. Antibodies are important, but the impact of cytokines cannot be ignored.

### 4. Example 4: LTA-1 Fusion

LTA1 is the active moiety of lethal toxin from Enterotoxigenic E. coli (ETEC). The activity of the LTA1 is required for the adjuvant activity of dmLT. The double mutants are in the region usually targeted by a protease to allow A1 to traffic to the cytoplasm of intestinal cells to cause the secretory diarrhea. Without the protease the LT still has some activation of cAMP. Likewise, LTA1 remains active.

### a) LTA1-Fusions:

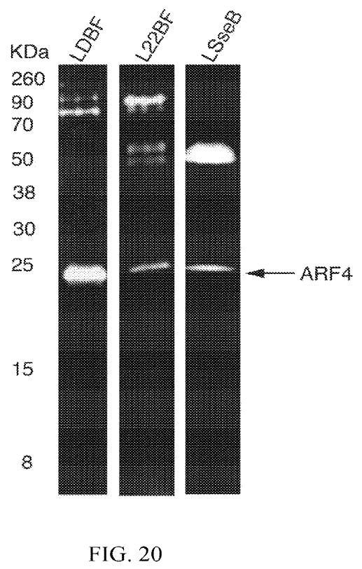

The LTA1-fusions were expressed in a manner similar to the fusion alone. The LTA1 sequence was inserted 5′ to the start of the each fusion. Some of the LTA1-fusions required a small linker between the LTA1 and fusion in order for protein production to occur. LTA1-DBF, LTA1-S1, LTA1-S2, LTA1-SseB, LTA1-22BF, LTA1-BurkF, and LTA1-PaF were produced. One of the assays that appear to be required for adjuvant activity is the ability to ADP ribosylate ARF4. The ADP ribosylation assay was performed with the LTA1-fusions. In the assay, ADPr was biotin conjugated and when mixed with LTA1 and rARF4, the LTA1 transferred the biot-ADPr to rARF4. The biotin was then detected with Streptavidin-IR800 (FIG. 20).

### b) LTA1-Fusion Protective Efficacy:

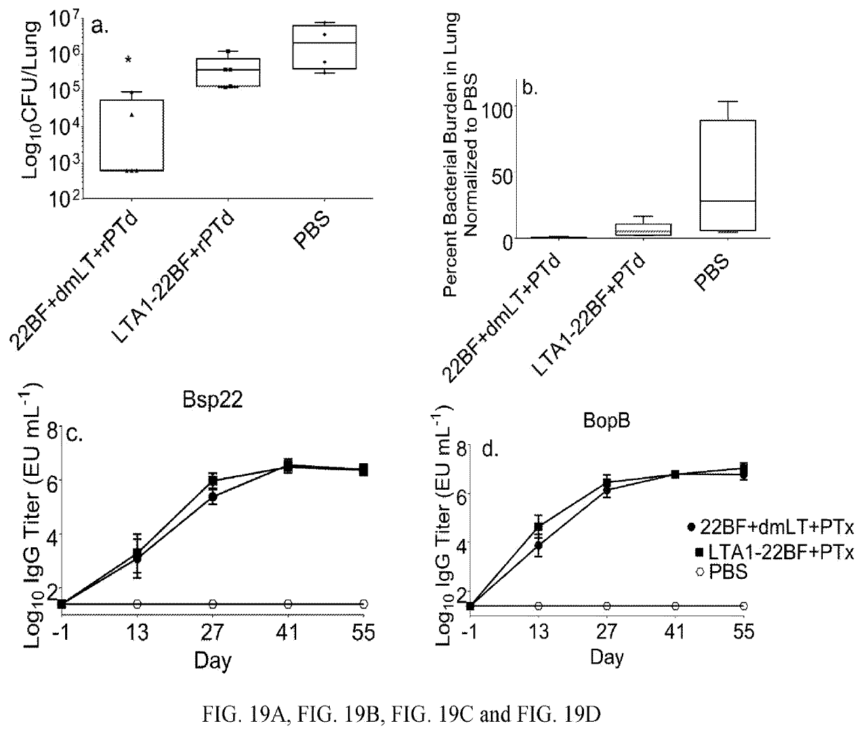

Mice were vaccinated parenterally with LTA1-DBF or DBF+dmLT (FIG. 14). Although the DBF+dmLT delivered IN elicited complete protection, this formulation cannot be given to humans since the dmLT can travel the olfactory nerve to the brain causing Bell's palsy. Thus, parenteral routes or other mucosal routes are required. DBF (100 μg)+dmLT (0.1 μg) delivered IM was only 50% protective while LTA1-DBF at a concentration of 100 μg DBF equivalent was 70% protective. The lower dilutions, regardless of formulation, exhibited less protective efficacy. Similarly, LTA1-22BF+PTd elicited significant protective efficacy at a concentration of 80 μg of 22BF equivalents (FIG. 19A-D)

### c) LTA1-DBF Immune Response:

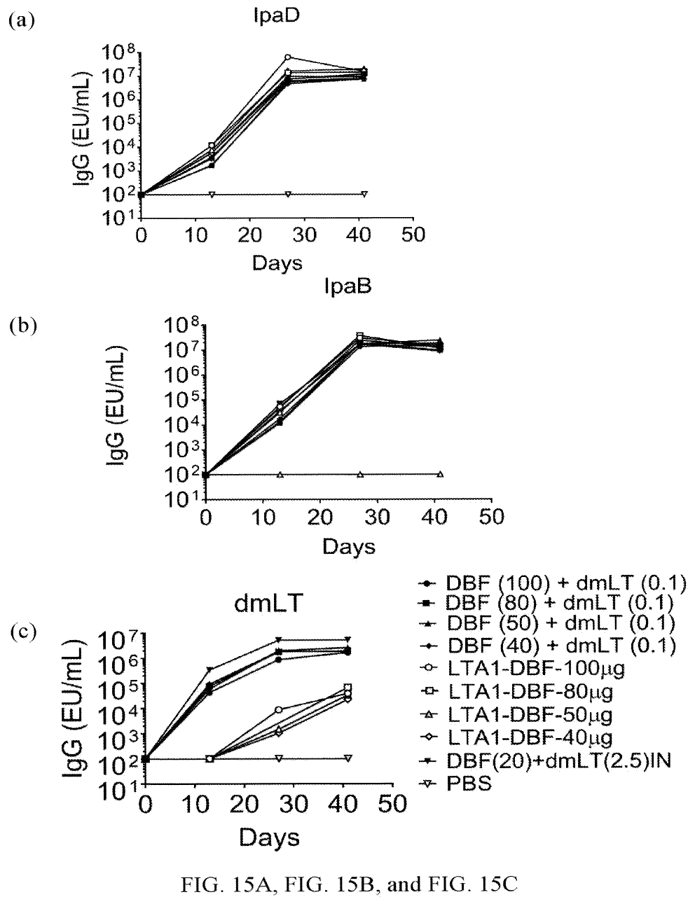

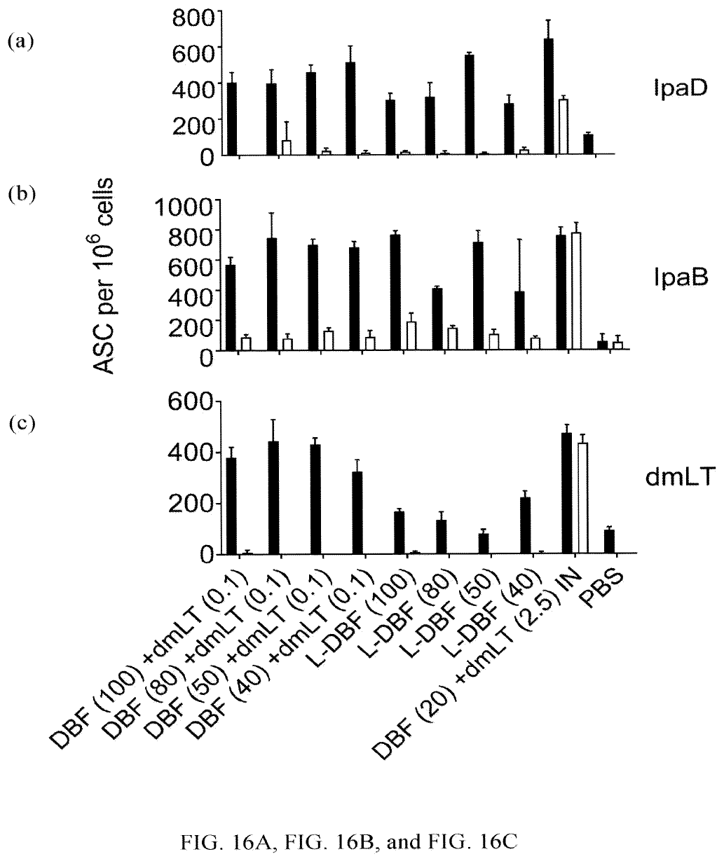

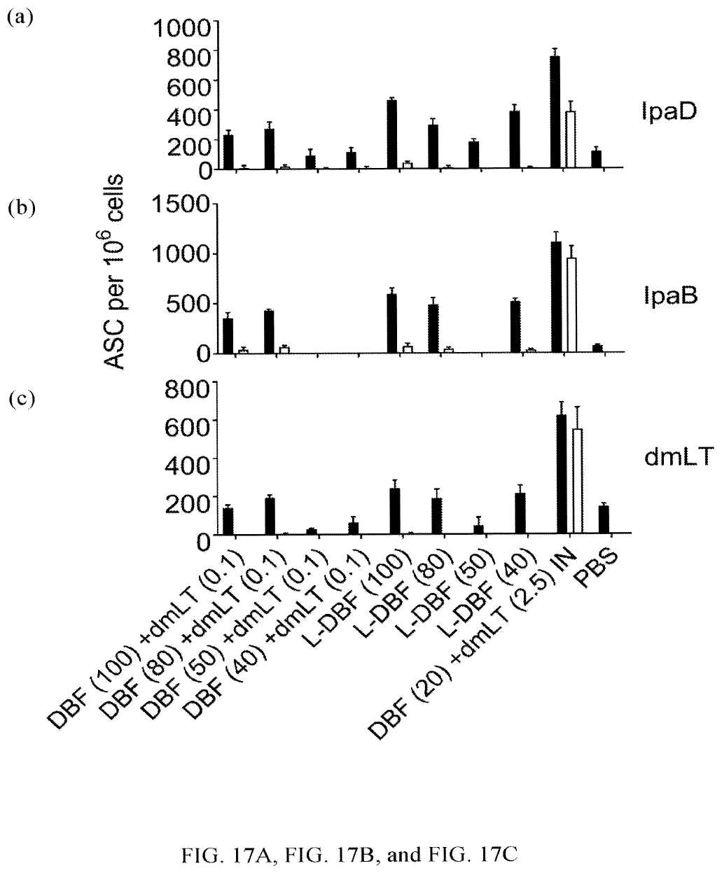

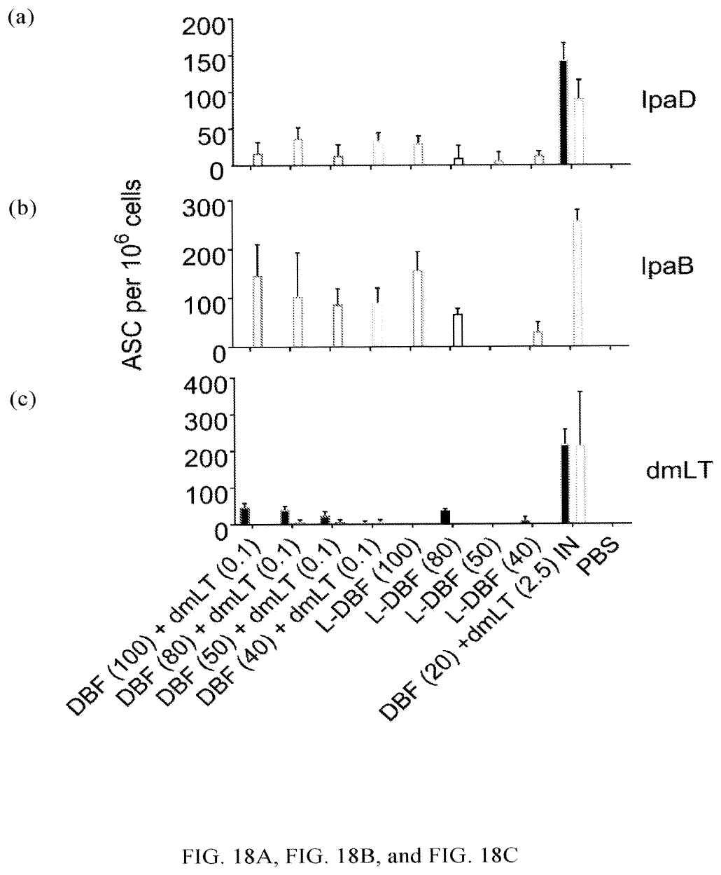

When the kinetics of the IgG titer was examined, responses against IpaD and IpaB were essentially the same. Mice from FIG. 14 were bled prior to vaccination and on day 42. Sera were assessed for anti-IpaD, -IpaB and -dmLT IgG. The lower IgG titers of the LTA1 samples can be attributed to the lower recognition of the dmLT on the well for the LTA1 samples vs the samples from mice vaccinated with dmLT (FIG. 15). No IgA was detected in the fecal samples of the mice vaccinated IM, but was detectable in mice vaccinated IN with DBF+dmLT. Antibody-secreting cells (ASCs) present in the bone marrow (FIG. 16), spleen (FIG. 17), and lungs (FIG. 18) were also stimulated with IpaD, IpaB and dmLT. In each compartment, anti-IpaD, anti-IpaB, and anti-dmLT IgG ASCs could be detected. Interestingly, IgA ASCs could also be detected in the bone marrow against all dilutions of LTA1-DBF and resemble a curve similar to the dose escalation. A similar phenomenon was seen in the IgA ASCs from the lungs, but less pronounced.

### d) LTA1-Dbf Purification.

The yield of LTA1-DBF was very low. Therefore, a linker was inserted in the DNA sequence between LTA1 and DBF to encode GSAAS (Seq. ID No. 14). The mother plasmid was Novagen's pACYCDuet-1. The translocator for each fusion cannot be made without its cognate chaperone. Therefore, the complex of LTA1-DBF/Histag-IpgC (IPG chaperone comprises the nucleic acid sequence as set forth in SEQ ID NO: 10 which encodes the amino acid sequence as set forth in SEQ ID NO: 11) was produced from the plasmid PACYC-His-IpgC-LTA1-GSAAS-DBF where the ipgC gene was inserted into the BamHI/HindIII sites allowing for expression of His-tag IpgC and LTA1-GSAAS-DBF (nucleic acid sequence as set forth in SEQ ID NO: 15 and amino acid sequence as set forth in SEQ ID NO: 16)) was inserted at the NdeI-XhoI site. The DBF sequence had a 3′ stop codon prior to the XhoI restriction site.

pACYC-His-IpgC-LTA1-GSAAS-DBF was transformed into Tuner cells. A small overnight culture of LB+ Chloramphenicol (Cm) that had been inoculated with the freezer stock of the cells was transferred to 8 L TB, and grown at 37° C. until OD=1-1.5, add 0.5 mM IPTG with 20 μg/liter AEBSF, 16 C overnight, harvested at 4000 rpm for 15 min at 4° C., and resuspended in IMAC binding buffer. The cells were frozenat −80° C. until ready for purification. After thawing the suspension was sonicated at 70% amplitude for 3-4 min, 15 s on, 30 s off, clarified by centrifugation at 13000 rpm for 30 min at 4° C. and decanted to obtain supernatant.

IMAC purification with 5 ml NINTA FF crude column on AKTA was as follows: (1) equilibrate column with 5CV binding buffer (20 mM Tris, 500 mM NaCl, 5 mM Imidazole pH 7.9), (2) load supernatant on column, collect FT in outlet1, (3) wash with binding buffer for 30CV, (4) elute with linear 0-60% elution buffer (20 mM Tris, 500 mM NaCl, 500 mM Imidazole pH 7.9) for 10CV, (5) elute with 60% elution buffer for 2CV, (6) wash column with 100% elution buffer for 3CV, (7) re-equilibrate column with 5CV binding buffer for 5CV.

HIC purification of the protein was as follows: Dilute pooled fraction into equal volume of 2×HIC binding buffer (50 mM Sodium Phosphate (dibasic), 1M Ammonium Sulfate, pH 7.0). Purify with 5 ml HIC Phenyl HP column: (1) equilibrate column with 5CV binding buffer, (2) load diluted sample on column, collect FT in outlet1, (3) wash with binding buffer for 5CV, (4) elute with linear 0-100% elution buffer (5 mM Sodium Phosphate (dibasic), pH 7.0) for 40CV, (6) elute with 100% elution buffer for 6CV, (7) wash column with 100% elution buffer for 3CV, (8) reequilibrate column with binding buffer for 5CV.

Pooled fractions were dialyzed in 4 L Q binding buffer for 2 hrs, exchanged buffer, and then dialyzed overnight.

Purification using a 5 mL Q FF columns on AKTA was as follows: (1) equilibrate column with 5CV binding buffer (50 mM Tris, pH 8.0), (2) load dialyzed sample on column, collect FT in outlet1, (3) wash with binding buffer for 5CV, (4) elute with linear 0-30% elution buffer (50 mM Tris, IM NaCl, pH 8.0) for 20CV, (6) elute with 100% elution buffer for 5CV, (7) wash column with 100% elution buffer for 3CV, (8) re-equilibrate column with binding buffer for 5CV.

To facilitate final IMAC purification 8×IMAC binding buffer (NO Imidazole) was added to pooled fractions to obtain 1× and then LDAO to 0.05% was added

Purification by LDAO IMAC using 5 ml NINTA FF was as follows: (1) equilibrate column with 5CV LDAO (20 mM Tris, 500 mM NaCl, 0.05% LDAO pH 7.9) binding buffer, (2) load supernatant on column, fractionate FT, (3) wash with binding buffer for 5CV, fractionate, (4) wash with 3% LDAO elution buffer (20 mM Tris, 500 mM NaCl, 500 mM Imidazole, 0.005% LDAO pH 7.9) for 5CV, fractionate (5) elute with 6% LDAO elution buffer for 6.65CV, fractionate (6) elute with 100% LDAO elution buffer for 5CV, (8) re-equilibrate column with 5CV binding buffer for 5CV.

Pooled samples were dialyzed in 4 L PBS+0.005% LDAO, exchanged buffer after 2 hrs, and then dialyzed overnight.

### e) Protective Efficacy of LTA1-22BF

The initial assessment of the protective efficacy of the LTA1-22BF is presented here and demonstrated that LTA1-22BF+rPT reduced the CFU lung burden by 99.8% while the 22BF+dmLT+rPT reduced it to 99.98% (FIG. 19 (a)). Non-toxic pertussis toxin was added to the groups that prior to challenge by B. pertussis to offset the damage that pertussis toxin would cause and negatively impact the challenge. The kinetics of the IgG response is also shown where no differences are seen between the two groups (FIG. 19 (b))

### f) LTA1-22BF Purification

The mother plasmid was Novagen's pACYCDuet-1. The translocator for each fusion cannot be made without its cognate chaperone. Therefore, the complex of LTA1-22BF/Histag-BcrHI is produced from the plasmid pACYC-His-BcrH1-LTA1-22BF where the brcHI gene (as set forth in SEQ ID NO: 7 with a histidine tag and encodes the amino acid sequence as set forth in SEQ ID NO: 8, the sequence minus the his-tag set forth in SEQ ID NO: 9) is inserted into the BamHI/HindIII sites allowing for expression of His-tag BcrHI and 22BF is inserted at the NdeI-XhoI site. The 22BF sequence has a 3′ stop codon prior to the XhoI restriction site.

pACYC-His-BcrHI-22BF was transformed into Tuner cells. A small overnight culture of LB+Chloramphenicol (Cm) that had been inoculated with the freezer stock of the cells was transferred to 8 L TB, grown at 37° C. until OD=1-1.5, added 0.5 mM IPTG with 20 ug/liter AEBSF, 16° C. overnight, harvested at 4000 rpm for 15 min at 4 C, and resuspended in IMAC binding buffer. The cells were frozen at −80 until ready for purification. After thawing, the suspension was sonicated at 70% amplitude for 3-4 min, 15 s on, 30 s off, clarified by centrifugation at 13000 rpm for 30 min at 4° C., and decanted to obtain supernatant.

IMAC purification with 5 ml NiNTA FF crude column on AKTA was as follows: (1) equilibrate column with 5CV binding buffer (IMAC elution buffer: 20 mM Tris, 500 mM NaCl, 500 mM Imidazole pH 7.9), (2) load supernatant on column, collect FT in outlet1, (3) wash with binding buffer for 30CV, (4) elute with linear 0-60% elution buffer for 10CV, (5) elute with 60% elution buffer for 2CV, (6) wash column with 100% elution buffer for 3CV, (7) re-equilibrate column with 5CV binding buffer (IMAC binding buffer: 20 mM Tris, 500 mM NaCl, 5 mM Imidazole pH 7.9) for 5CV.

Diluted pooled fractions 20× into Q binding buffer Q binding buffer: 50 mM Tris, pH 8.0). Purification with 3×5 mL Q FF columns on AKTA was as follows: (1) equilibrate column with 6CV binding buffer, (2) load dialyzed sample on column, collect FT in outlet1, (3) wash with binding buffer for 12CV, (4) elute with 15% elution buffer (Q elution buffer: 50 mM Tris, 1M NaCl, pH 8.0) for 6CV, (5) elute with linear 15-40% elution buffer for 34CV, (6) elute with 100% elution buffer for 6CV, (7) wash column with 100% elution buffer for 3CV, (8) re-equilibrate column with binding buffer for 6CV.

To facilitate final IMAC purification 8×IMAC binding buffer (NO Imidazole) was added to pooled fractions to obtain 1× and then LDAO to 0.05% was added. Purification by LDAO IMAC using 5 ml NiNTA FF was as follows: (1) equilibrate column with 5CV LDAO binding buffer (LDAO IMAC binding buffer: 20 mM Tris, 500 mM NaCl, 0.05% LDAO pH 7.9), (2) load supernatant on column, fractionate FT, (3) wash with binding buffer for 5CV, fractionate, (4) wash with 3% LDAO elution buffer (LDAO IMAC elution buffer: 20 mM Tris, 500 mM NaCl, 500 mM Imidazole, 0.005% LDAO pH 7.9) for 5CV, fractionate (5) elute with 6% LDAO elution buffer for 6.65CV, fractionate (6) elute with 100% LDAO elution buffer for 5CV, (8) re-equilibrate column with 5CV binding buffer for 5CV.

Pooled samples were dialyzed in 4 L PBS+0.005% LDAO, exchanged buffer after 2 hrs, and then dialyzed overnight.

### g) LTA1-BurkF Purification

The mother plasmid was Novagen's pACYCDuet-1. The translocator for each fusion cannot be made without its cognate chaperone. Therefore, the complex of LTA1-BurkF/Histag-BicA (SEQ ID NOs: 28 and 20) was produced from the plasmid pACYC-His-BicA-LTA1-BurkF (as set forth in SEQ ID NO: 20 and encoded by the nucleic acid sequence as set forth in SEQ ID NO: 19) wherein the bicA gene was inserted into the BamHI/HindIII sites allowing for expression of His-tag BicA and LTA1-BurkF was inserted at the NdeI-XhoI site. The BurkF sequence had a 3′ stop codon prior to the XhoI restriction site.

pACYC-His-BicA-LTA1-BurkF was transformed into Tuner cells. A small overnight culture of LB+Chloramphenicol (Cm) that had been inoculated with the freezer stock of the cells was transferred to 8 L TB, grown at 37° C. until OD=1-1.5, added 0.5 mM IPTG with 20 ug/liter AEBSF, 16° C. overnight, harvested at 4000 rpm for 15 min at 4 C, and resuspended in IMAC binding buffer. The cells were frozen at −80 until ready for purification. After thawing the suspension was sonicated at 70% amplitude for 3-4 min, 15 s on, 30 s off, Clarified by centrifugation at 13000 rpm for 30 min at 4° C., and decanted to obtain supernatant.

IMAC purification with 5 ml NiNTA FF crude column on AKTA was as follows: (1) equilibrate column with 5CV binding buffer (20 mM Tris, 500 mM NaCl, 5 mM Imidazole pH 7.9), (2) load supernatant on column, collect FT in outlet1, (3) wash with binding buffer for 30CV, (4) elute with linear 0-60% elution buffer (20 mM Tris, 500 mM NaCl, 500 mM Imidazole pH 7.9) for 10CV, (5) elute with 60% elution buffer for 2CV, (6) wash column with 100% elution buffer for 3CV, (7) re-equilibrate column with 5CV binding buffer for 5CV.

The pooled fractions were diluted 20× into Q binding buffer (50 mM Tris, pH 8.0). Purification using 3×5 mL Q FF columns on AKTA was as follows: (1) equilibrate column with 6CV binding buffer, (2) load dialyzed sample on column, collect FT in outlet1, (3) wash with binding buffer for 12CV, (4) elute with 15% elution buffer (50 mM Tris, IM NaCl, pH 8.0) for 6CV, (5) elute with linear 15-40% elution buffer for 34CV, (6) elute with 100% elution buffer for 6CV, (7) wash column with 100% elution buffer for 3CV, (8) re-equilibrate column with binding buffer for 6CV.

To facilitate final IMAC purification add 8×IMAC binding buffer (NO Imidazole) to pooled fractions to obtain 1× and then LDAO to 0.05% was added:

Purification by LDAO IMAC using 5 ml NiNTA FF was as follows: (1) equilibrate column with 5CV LDAO binding buffer (20 mM Tris, 500 mM NaCl, 0.05% LDAO pH 7.9), (2) load supernatant on column, fractionate FT, (3) wash with binding buffer for 5CV, fractionate, (4) wash with 3% LDAO elution buffer (20 mM Tris, 500 mM NaCl, 500 mM Imidazole, 0.005% LDAO pH 7.9) for 5CV, fractionate (5) elute with 6% LDAO elution buffer for 6.65CV, fractionate (6) elute with 100% LDAO elution buffer for 5CV, (8) re-equilibrate column with 5CV binding buffer for 5CV.

Pooled samples were dialyzed in 4 L PBS+0.005% LDAO, exchanged buffer after 2 hrs, and then dialyzed overnight.

### h) LTA1-PaF

The PaF+dmLT vaccinated mice exhibited 100% survival with 44% sterilizing immunity against Pa challenge in a mouse lethal pulmonary model, while the PBS vaccinated mice exhibited 60% survival but all had >108-9 CFU/lung.

### i) LTA1-PaF Purification

The mother plasmid is Novagen's pACYCDuet-1. The translocator for each fusion cannot be made without its cognate chaperone. Therefore, the complex of LTA1-PaF/Histag-PcrHI was produced from the plasmid pACYC-His-PcrH1-LTA1-PaF where the brcHI gene was inserted into the BamHI/HindIII sites allowing for expression of His-tag PcrHI (as set forth in SEQ ID NO: 30 and encoded by the nucleic acid sequence as set forth in SEQ ID NO: 29) and LTA1-PaF (as set forth in SEQ ID NO: 38 and encoded by the nucleic acid sequence as set forth in SEQ ID NO: 37) which was inserted at the NdeI-XhoI site. The PaF sequence had a 3′ stop codon prior to the XhoI restriction site. The purification of LTA1-PaF was the same of for LTA1-22BF.

### E. REFERENCES

Amuguni J H, Lee S, Kerstein K O, Brown D W, Belitsky B R, Herrmann J E, Keusch G T, Sonenshein A L, Tzipori S. Sublingually administered Bacillus subtilis cells expressing tetanus toxin C fragment induce protective systemic and mucosal antibodies against tetanus toxin in mice. Vaccine. 2011; 29 (29-30): 4778-84. Epub 2011 May 14. doi: S0264-410X(11)00638-4 [pii]
Bulir D C, Liang S, Lee A, Chong S, Simms E, Stone C, Kaushic C, Ashkar A, Mahony J B. Immunization with chlamydial type III secretion antigens reduces vaginal shedding and prevents fallopian tube pathology following live C. muridarum challenge. Vaccine. 2016; 34 (34): 3979-85. doi: 10.1016/j.vaccine.2016.06.046. PubMed PMID: 27325352.
Chen X, Choudhari S P, Martinez-Becerra F J, Kim J H, Dickenson N E, Toth R Tt, Joshi S B, Greenwood J C, 2nd, Clements J D, Picking W D, Middaugh C R, Picking W L. Impact of Detergent on Biophysical Properties and Immune Response of the IpaDB Fusion Protein, a Candidate Subunit Vaccine against Shigella Species. Infect Immun. 2015; 83 (1): 292-9. doi: 10.1128/IAI.02457-14. PubMed PMID: 25368115.
Fennelly N K, Sisti F, Higgins S C, Ross P J, van der Heide H, Mooi F R, Boyd A, Mills K H. Bordetella pertussis expresses a functional type III secretion system that subverts protective innate and adaptive immune responses. Infect Immun. 2008; 76 (3): 1257-66. doi: 10.1128/IAI.00836-07. PubMed PMID: 18195025; PMCID: PMC2258832.
Galan J E, Wolf-Watz H. Protein delivery into eukaryotic cells by type III secretion machines. Nature. 2006; 444 (7119): 567-73. PubMed PMID: 17136086.
Higgs R, Higgins S C, Ross P J, Mills K H. Immunity to the respiratory pathogen Bordetella pertussis. Mucosal Immunol. 2012; 5 (5): 485-500. doi: 10.1038/mi.2012.54. PubMed PMID: 22718262.
Kilgore P E, Salim A M, Zervos M J, Schmitt H J. Pertussis: Microbiology, Disease, Treatment, and Prevention. Clin Microbiol Rev. 2016; 29 (3): 449-86. doi: 10.1128/CMR.00083-15. PubMed PMID: 27029594; PMCID: PMC4861987.
Martin S W, Pawloski L, Williams M, Weening K, DeBolt C, Qin X, Reynolds L, Kenyon C, Giambrone G, Kudish K, Miller L, Selvage D, Lee A, Skoff T H, Kamiya H, Cassiday P K, Tondella M L, Clark T A. Pertactin-negative Bordetella pertussis strains: evidence for a possible selective advantage. Clin Infect Dis. 2015; 60 (2): 223-7. doi: 10.1093/cid/ciu788. PubMed PMID: 25301209.
Martinez-Becerra F J, Chen X, Dickenson N E, Choudhari S P, Harrison K, Clements J D, Picking W D, Van De Verg L L, Walker R I, Picking W L. Characterization of a novel fusion protein from IpaB and IpaD of Shigella spp. and its potential as a pan-Shigella vaccine. Infect Immun. 2013; 81 (12): 4470-7. doi: 10.1128/IAI.00859-13. PubMed PMID: 24060976; PMCID: 3837967.
Martinez-Becerra F J, Kissmann J M, Diaz-McNair J, Choudhari S P, Quick A M, Mellado-Sanchez G, Clements J D, Pasetti M F, Picking W L. Broadly protective Shigella vaccine based on type III secretion apparatus proteins. Infect Immun. 2012; 80 (3): 1222-31. doi: 10.1128/IAI.06174-11. PubMed PMID: 22202122; PMCID: 3294653.
Mattoo S, Cherry J D. Molecular pathogenesis, epidemiology, and clinical manifestations of respiratory infections due to Bordetella pertussis and other Bordetella subspecies. Clin Microbiol Rev. 2005; 18 (2): 326-82. doi: 10.1128/CMR.18.2.326-382.2005. PubMed PMID: 15831828; PMCID: PMC1082800.
Medhekar B, Shrivastava R, Mattoo S, Gingery M, Miller J F. Bordetella Bsp22 forms a filamentous type III secretion system tip complex and is immunoprotective in vitro and in vivo. Molecular microbiology. 2009; 71 (2): 492-504. PubMed PMID: 19040642.
National Center for Immunization and Respiratory Diseases DoBD, CDC. 2015 Final Pertussis Surveillance Report 2017.
Nogawa H, A. Kuwae, T. Matsuzawa, and A. Abe. The type III secretion protein BopD in Bordetella bronchiseptica is complexed with BopB for pore formation on the host plasma membrane. J Bacteriol. 2004; 186 (12): 3806-13.
Norton E B, Lawson L B, Freytag L C, Clements J D. Characterization of a mutant Escherichia coli heat-labile toxin, LT (R192G/L211A), as a safe and effective oral adjuvant. Clinical and vaccine immunology: CVI. 2011; 18 (4): 546-51. doi: 10.1128/CVI.00538-10. PubMed PMID: 21288994; PMCID: 3122563.
Norton E B, Lawson L B, Mahdi Z, Freytag L C, Clements J D. The A subunit of Escherichia coli heat-labile enterotoxin functions as a mucosal adjuvant and promotes IgG2a, IgA, and Th17 responses to vaccine antigens. Infect Immun. 2012; 80 (7): 2426-35. doi: 10.1128/IAI.00181-12. PubMed PMID: 22526674; PMCID: 3416479.
Qin L, Gilbert P B, Corey L, McElrath M J, Self S G. A framework for assessing immunological correlates of protection in vaccine trials. J Infect Dis. 2007; 196 (9): 1304-12. doi: 10.1086/522428. PubMed PMID: 17922394.
Ross P J, Sutton C E, Higgins S, Allen A C, Walsh K, Misiak A, Lavelle E C, McLoughlin R M, Mills K H. Relative contribution of Th1 and Th17 cells in adaptive immunity to Bordetella pertussis: towards the rational design of an improved acellular pertussis vaccine. PLOS Pathog. 2013; 9 (4): e1003264. doi: 10.1371/journal.ppat.1003264. PubMed PMID: 23592988; PMCID: PMC3617212.
Sealey K L, Belcher T, Preston A. Bordetella pertussis epidemiology and evolution in the light of pertussis resurgence. Infect Genet Evol. 2016; 40:136-43. doi: 10.1016/j.meegid.2016.02.032. PubMed PMID: 26932577.
Siegrist C A. Neonatal and early life vaccinology. Vaccine. 2001; 19 (25-26): 3331-46. PubMed PMID: 11348697.
Villarino Romero R, Bibova I, Cerny O, Vecerek B, Wald T, Benada O, Zavadilova J, Osicka R, Sebo P. The Bordetella pertussis type III secretion system tip complex protein Bsp22 is not a protective antigen and fails to elicit serum antibody responses during infection of humans and mice. Infect Immun. 2013; 81 (8): 2761-7. doi: 10.1128/IAI.00353-13. PubMed PMID: 23690400; PMCID: PMC3719584.
Warfel J M, Zimmerman L I, Merkel T J. Acellular pertussis vaccines protect against disease but fail to prevent infection and transmission in a nonhuman primate model. Proc Natl Acad Sci USA. 2014; 111 (2): 787-92. doi: 10.1073/pnas.1314688110. PubMed PMID: 24277828; PMCID: PMC3896208.
Warfel J M, Zimmerman L I, Merkel T J. Comparison of Three Whole-Cell Pertussis Vaccines in the Baboon Model of Pertussis. Clin Vaccine Immunol. 2015; 23 (1): 47-54. doi: 10.1128/CVI.00449-15. PubMed PMID: 26561389; PMCID: PMC4711092.

### F. SEQUENCES

22BF Nucleotide Sequence
SEQ ID NO: 1
CATatgaccattgatctcggagtttcactcacgtcgcaggccggcggcctgcaaggcatcgacctcaagagcatggata
tccagactctcatggtgtatgtgcagggtcgtcgcgccgaactcctcacggctcaaatgcagacccaggccgaagtggtgcagaagg
ccaatgaacgcatggcgcagctcaacgaggtcctgtccgcgctgtcccgggccaaggccgagtttccgcccaatccgaagccggg
cgacaccatcccgggctgggacaaccagaaggtcagccggatcgaggttcctctcaatgatgcgctgcgcgctgccggcctgacg
ggcatgttcgaagcgcgcgatggccaagtgaccgcccccggcggccggggtacgcaggtcgtgaacggcacgggcgtcatggcc
ggttccacgacctataaggaactcgaaagtgcctacaccaccgtaaaggggatgctggatacggcgtccaatacgcaacagatggac
atgatcaggctgcaggccgccagcaacaagcgcaacgaggctttcgaggtcatgaccaacaccgagaagcggcgcagcgacctg
aacagttccatcaccaacaacatgcgcaagcttatgaccgtcatgagtacgaccatatccacagccccgagcggcgccgcgcttgcg
ccgtctcgcatagatatgcgggcaccggagcccgggagtgccggcgaaggcgccggcatcctggcgccggtgacgacgctggct
ctggcggcgggccggccggcttttccagcgtcaccgtcgctgcgcaccgcgcccgtcctggatccgccagtgcgcgatctcagccc
cgccgacttggccgacctgctgcgcgtcttgcgatccagggcggtggacgggcagttggccacggcgcgcgagaacctgcagga
cgcgcaagtcaaggcgaagcagaacacccaggcccagctcgacaagctggacgcatggtttcggaaggccgaagaggccgaga
gcaagggatggctgagcaaggtgttcggctggatcggcaaggtgctggcggtcgtggcatcggccctggcggtgggctttgccgcc
gtcgccagcgtggccaccggcgcggcggccacacccatgctgctgctcagcggcatggcactggtcagcgccgtgacatcgctgg
ccgaccagatatcgcaagaggcgggaggcccgcctatcagcctgggcgggtttctctccgggctggccggacgtctgctgacagc
gttgggggtggatcagtcgcaggccgaccaaattgccaagatcgtcgccggcctggccgtgcccgtcgtcttgctgatcgaacccca
gatgctgggcgaaatggcgcaaggcgtggccaggctggctggcgccagcgatgccaccgcggggtacatagccatggcgatgtc
catcgtggcggcgatcgcggtcgccgcgatcaatgccgccggtacagccggcgcgggtagcgcttcggcgatcaagggggcctg
ggatcgggccgccgcggtagccacccaggtccttcaagggggtacggcagtggcgcaaggcggcgtcggcgtgtcgatggcagt
cgatcgcaaacaggccgatctcctggtcgccgacaaggcggatctggcggcgagcctgacaaaactgcgggcggccatggagcg
tgaggcggacgatatcaagaagatcctggctcaattcgacgaggcctatcacatgatcgcgaagatgatcagcgatatggcgagtac
gcacagccaggtcagcgccaacctcgggcggcgccaggcggtgtagCTCGAG
22BF Amino Acid Sequence
SEQ ID NO: 2
MTIDLGVSLTSQAGGLQGIDLKSMDIQTLMVYVQGRRAELLTAQMQTQAEV
VQKANERMAQLNEVLSALSRAKAEFPPNPKPGDTIPGWDNQKVSRIEVPLNDALRA
AGLTGMFEARDGQVTAPGGRGTQVVNGTGVMAGSTTYKELESAYTTVKGMLDTA
SNTQQMDMIRLQAASNKRNEAFEVMTNTEKRRSDLNSSITNNMRKLMTVMSTTIST
APSGAALAPSRIDMRAPEPGSAGEGAGILAPVTTLALAAGRPAFPASPSLRTAPVLDP
PVRDLSPADLADLLRVLRSRAVDGQLATARENLQDAQVKAKQNTQAQLDKLDAWF
RKAEEAESKGWLSKVFGWIGKVLAVVASALAVGFAAVASVATGAAATPMLLLSGM
ALVSAVTSLADQISQEAGGPPISLGGFLSGLAGRLLTALGVDQSQADQIAKIVAGLAV
PVVLLIEPQMLGEMAQGVARLAGASDATAGYIAMAMSIVAAIAVAAINAAGTAGAG
SASAIKGAWDRAAAVATQVLQGGTAVAQGGVGVSMAVDRKQADLLVADKADLA
ASLTKLRAAMEREADDIKKILAQFDEAYHMIAKMISDMASTHSQVSANLGRRQAV
Bsp22 Nucleotide Sequence
SEQ ID NO: 3
CATatgaccattgatctcggagtttcactcacgtcgcaggccggcggcctgcaaggcatcgacctcaagagcatggata
tccagactctcatggtgtatgtgcagggtcgtcgcgccgaactcctcacggctcaaatgcagacccaggccgaagtggtgcagaagg
ccaatgaacgcatggcgcagctcaacgaggtcctgtccgcgctgtcccgggccaaggccgagtttccgcccaatccgaagccggg
cgacaccatcccgggctgggacaaccagaaggtcagccggatcgaggttcctctcaatgatgcgctgcgcgctgccggcctgacg
ggcatgttcgaagcgcgcgatggccaagtgaccgcccccggcggccggggtacgcaggtcgtgaacggcacgggcgtcatggcc
ggttccacgacctataaggaactcgaaagtgcctacaccaccgtaaaggggatgctggatacggcgtccaatacgcaacagatggac
atgatcaggctgcaggccgccagcaacaagcgcaacgaggctttcgaggtcatgaccaacaccgagaagcggcgcagcgacctg
aacagttccatcaccaacaacatgcgc
Bsp22 Amino Acid Sequence
SEQ ID NO: 4
MTIDLGVSLTSQAGGLQGIDLKSMDIQTLMVYVQGRRAELLTAQMQTQAEV
VQKANERMAQLNEVLSALSRAKAEFPPNPKPGDTIPGWDNQKVSRIEVPLNDALRA
AGLTGMFEARDGQVTAPGGRGTQVVNGTGVMAGSTTYKELESAYTTVKGMLDTA
SNTQQMDMIRLQAASNKRNEAFEVMTNTEKRRSDLNSSITNNMR
BopB Nucleotide Sequence
SEQ ID NO: 5
Atgaccgtcatgagtacgaccatatccacagccccgagcggcgccgcgcttgcgccgtctcgcatagatatgcgggcac
cggagcccgggagtgccggcgaaggcgccggcatcctggcgccggtgacgacgctggctctggcggcgggccggccggcttttc
cagcgtcaccgtcgctgcgcaccgcgcccgtcctggatccgccagtgcgcgatctcagccccgccgacttggccgacctgctgcgc
gtcttgcgatccagggcggtggacgggcagttggccacggcgcgcgagaacctgcaggacgcgcaagtcaaggcgaagcagaa
cacccaggcccagctcgacaagctggacgcatggtttcggaaggccgaagaggccgagagcaagggatggctgagcaaggtgtt
cggctggatcggcaaggtgctggcggtcgtggcatcggccctggcggtgggctttgccgccgtcgccagcgtggccaccggcgcg
gcggccacacccatgctgctgctcagcggcatggcactggtcagcgccgtgacatcgctggccgaccagatatcgcaagaggcgg
gaggcccgcctatcagcctgggcgggtttctctccgggctggccggacgtctgctgacagcgttgggggtggatcagtcgcaggcc
gaccaaattgccaagatcgtcgccggcctggccgtgcccgtcgtcttgctgatcgaaccccagatgctgggcgaaatggcgcaagg
cgtggccaggctggctggcgccagcgatgccaccgcggggtacatagccatggcgatgtccatcgtggcggcgatcgcggtcgcc
gcgatcaatgccgccggtacagccggcgcgggtagcgcttcggcgatcaagggggcctgggatcgggccgccgcggtagccacc
caggtccttcaagggggtacggcagtggcgcaaggcggcgtcggcgtgtcgatggcagtcgatcgcaaacaggccgatctcctggt
cgccgacaaggcggatctggcggcgagcctgacaaaactgcgggcggccatggagcgtgaggcggacgatatcaagaagatcct
ggctcaattcgacgaggcctatcacatgatcgcgaagatgatcagcgatatggcgagtacgcacagccaggtcagcgccaacctcg
ggcggcgccaggcggtgtagCTCGAG
BopB Amino Acid Sequence
SEQ ID NO: 6
MTVMSTTISTAPSGAALAPSRIDMRAPEPGSAGEGAGILAPVTTLALAAGRPA
FPASPSLRTAPVLDPPVRDLSPADLADLLRVLRSRAVDGQLATARENLQDAQVKAK
QNTQAQLDKLDAWFRKAEEAESKGWLSKVFGWIGKVLAVVASALAVGFAAVASV
ATGAAATPMLLLSGMALVSAVTSLADQISQEAGGPPISLGGFLSGLAGRLLTALGVD
QSQADQIAKIVAGLAVPVVLLIEPQMLGEMAQGVARLAGASDATAGYIAMAMSIVA
AIAVAAINAAGTAGAGSASAIKGAWDRAAAVATQVLQGGTAVAQGGVGVSMAVD
RKQADLLVADKADLAASLTKLRAAMEREADDIKKILAQFDEAYHMIAKMISDMAST
HSQVSANLGRRQAV
His-BcrH1 chaperone with histidine tag nucleotide sequence
SEQ ID NO: 7
ATGGGCAGCAGCCATCACCATCATCACCACAGCCAGGATCCGATGCCAAA
GTCAGCCGAGCAGGGCGGCTCCCCGGCGTCAGCTTCGCATGAGGCGTTGCGCCA
TATTCTCGACGCAGGCGCTTCGATGGGCAGCTTGCAGGGGTTGGACGAGGTGCA
ACAGCAGGCGTTGTACGCGATCGCTCATGGCGCCTACGAACAGGGCCGCTATGC
CGACGCGTTGAAAATGTTCTGCCTGCTGGTCGCGTGCGATCCGCTGGAAGCCCGT
TATCTGCTGGCCCTGGGCGCCGCGGCCCAGGAGCTGGGGCTGTACGAGCATGCC
TTGCAGCAATACGCGGCCGCGGCGGCTTTGCAGTTGGACTCCCCCAGGCCCCTGT
TGCATGGCGCCGAGTGCCTGTATGCGTTGGGTCGTCGCCGCGACGCCCTGGATAC
GCTCGACATGGTGCTTGAGTTGTGCGGGTCGCCGGAGCATGCGGCCCTGCGCGA
ACGGGCCGAGTCGCTGCGCAGGAGCTATGCACGTGCCGACTGAAAGCTT
His-BcrH1 with histidine tag chaperone amino acid sequence
SEQ ID NO: 8
MGSSHHHHHHSQDPMPKSAEQGGSPASASHEALRHILDAGASMGSLQGLDE
VQQQALYAIAHGAYEQGRYADALKMFCLLVACDPLEARYLLALGAAAQELGLYEH
ALQQYAAAAALQLDSPRPLLHGAECLYALGRRRDALDTLDMVLELCGSPEHAALRE
RAESLRRSYARAD
BcrH1 amino acid sequence
SEQ ID NO: 9
MPKSAEQGGSPASASHEALRHILDAGASMGSLQGLDEVQQQALYAIAHGAY
EQGRYADALKMFCLLVACDPLEARYLLALGAAAQELGLYEHALQQYAAAAALQLD
SPRPLLHGAECLYALGRRRDALDTLDMVLELCGSPEHAALRERAESLRRSYARAD
IpgC Chaperone of DBF nucleic acid sequence
SEQ ID NO: 10
CCatgggcagcagccatcatcatcatcatcacagcagcggcctggtgccgcgcggcagccatatgctcgagatgtcttta
aatatcaccgaaaatgaaagcatctctactgcagtaattgatgcaattaactctggcgctacactgaaagatattaatgcaattcctgatga
tatgatggatgacatttattcatatgcttatgacttttacaacaaaggaagaatagaggaagctgaagttttcttcaggtttttatgtatatacg
acttttacaatatagactacattatgggactcgcagctatttatcagataaaagaacagttccaacaagcagcagacctttatgctgtcgct
tttgcattaggaaaaaatgactatacaccagtattccatactggacaatgccagcttcggttgaaagcccccttaaaagctaaagagtgct
tcgaactcgtaattcaacacagcaatgatgaaaaattaaaaataaaagcacaatcatacttggacgcaattcaggatatcaaggagtag
GATCC
IpgC Chaperone of DBF Amino Acid sequence
SEQ ID NO: 11
MSLNITENESISTAVIDAINSGATLKDINAIPDDMMDDIYSYAYDFYNKGRIEE
AEVFFRFLCIYDFYNVDYIMGLAAIYQIKEQFQQAADLYAVAFALGKNDYTPVFHTG
QCQLRLKAPLKAKECFELVIQHSNDEKLKIKAQSYLDAIQDIKE
LTA1 nucleic acid sequence
SEQ ID NO: 12
CATAtggacaatggcgatcgtttataccgtgccgactcgcgtcccccagatgagattaaacgtagcggtgggttaatgc
cacgtgggcacaatgagtattttgaccgtggaacacagatgaacattaacctttacgatcatgcccgtgggacccagaccgggtttgtc
cgttatgatgacgggtatgttagtacgagtttgtccttacgctccgcacaccttgcgggacaaagtattttatcaggctacagcacatatta
catttatgtgatcgccactgccccaaacatgttcaatgtgaacgatgtgttgggggtttacagcccccatccatatgaacaagaagtctcg
gcccttggggggatcccatatagccagatttatggttggtaccgcgtaaattttggtgtgattgatgaacgtttgcatcgtaaccgtgaata
ccgcgatcgctactaccgtaacttgaacattgcacctgccgaggacggctatcgtttagcgggattcccacccgatcatcaggcgtgg
cgtgaggaaccgtggatccatcacgcccctcaggggtgcgggaacagtagtcgc
LTA1 amino acid sequence
SEQ ID NO: 13
MDNGDRLYRADSRPPDEIKRSGGLMPRGHNEYFDRGTQMNINLYDHARGTQ
TGFVRYDDGYVSTSLSLRSAHLAGQSILSGYSTYYIYVIATAPNMFNVNDVLGVYSP
HPYEQEVSALGGIPYSQIYGWYRVNFGVIDERLHRNREYRDRYYRNLNIAPAEDGYR
LAGFPPDHQAWREEPWIHHAPQGCGNSSR
GSAAS Linker amino acid sequence
SEQ ID NO: 14
GSAAS
LTA1-GSAAS-DBF (IpaD-LE-IpaB) nucleic acid sequence
SEQ ID NO: 15
CATAtggacaatggcgatcgtttataccgtgccgactcgcgtcccccagatgagattaaacgtagcggtgggttaatgc
cacgtgggcacaatgagtattttgaccgtggaacacagatgaacattaacctttacgatcatgcccgtgggacccagaccgggtttgtc
cgttatgatgacgggtatgttagtacgagtttgtccttacgctccgcacaccttgcgggacaaagtattttatcaggctacagcacatatta
catttatgtgatcgccactgccccaaacatgttcaatgtgaacgatgtgttgggggtttacagcccccatccatatgaacaagaagtctcg
gcccttggggggatcccatatagccagatttatggttggtaccgcgtaaattttggtgtgattgatgaacgtttgcatcgtaaccgtgaata
ccgcgatcgctactaccgtaacttgaacattgcacctgccgaggacggctatcgtttagcgggattcccacccgatcatcaggcgtgg
cgtgaggaaccgtggatccatcacgcccctcaggggtgcgggaacagtagtcgcgggtccgcggcatccatgaatat
aacaactctg actaatagta tttccacctc atcattcagt ccaaacaata ccaacggttc atcaaccgaa acagttaatt
ctgatataaa aacaacgacc agttctcatc ctgtaagttc ccttactatg ctcaacgaca cccttcataa tatcagaaca
acaaatcagg cattaaagaa agagctttca caaaaaacgt tgactaaaac atcgctagaa gaaatagcat tacattcatc
tcagattagc atggatgtaa ataaatccgc tcaactattg gatattcttt ccaggaacga atatccaatt aataaagacg
caagagaatt attacattca gccccgaaag aagccgagct tgatggagat caaatgatat ctcatagaga actgtgggct
aaaattgcaa actccatcaa tgatattaat gaacagtatc tgaaagtata tgaacatgcc gttagttcat atactcaaat
gtatcaagat tttagcgctg ttctttccag tcttgccggc tggatctctc ccggaggtaa cgacggaaac tccgtgaaat
tacaagtcaa ctcgcttaaa aaggcattgg aagaactcaa ggaaaaatat aaagataaac cgctatatcc agcaaataat
actgttagtc aggaacaagc aaataaatgg cttacagaat taggtggaac aatcggcaag gtatctcaaa aaaacggggg
atatgttgtc agtataaaca tgaccccaat agacaatatg ttaaaaagct tagataatct aggtggaaat ggcgaggttg
tgctagataa tgcaaaatat caggcatgga atgccggatt ctctgccgaa gatgaaacaa tgaaaaataa tcttcaaact
ttagttcaaa aatacagtaa tgccaatagt atttttgata atttagtaaa ggttttgagt agtacaataa gctcatgtac
agatacagat aaactttttc tccatttc CTCGAG atgcataatgta agcaccacaa ccactggttt tcctcttgcc
aaaatattga cttccactgagcttggagac aatactatcc aagctgcaaa tgatgcagct aacaaattat tttctcttacaattgctgat
cttactgcta accaaaatat taatacaact aatgcacact caacttcaaatatattaatc cctgaactta aagcaccaaa
gtcattaaat gcaagttccc aactaacgcttttaattgga aaccttattc aaatactcgg tgaaaaatct ttaactgcat
taacaaataaaattactgct tggaagtccc agcaacaggc aagacagcaa aaaaacctag aattctccgataaaattaac
actcttctat ctgaaactga aggactaacc agagactatg aaaaacaaattaataaacta aaaaacgcag attctaaaat
aaaagaccta gaaaataaaa ttaaccaaattcaaacaaga ttatcgaacc tcgatccaga gtcaccagaa aagaaaaaat
taagccgggaagaaatacaa ctcactatca aaaaagacgc agcagttaaa gacaggacat tgattgagcagaaaaccctg
tcaattcata gcaaacttac agataaatca atgcaactcg aaaaagaaatagactctttt tctgcatttt caaacacagc
atctgctgaa cagctatcaa cccagcagaaatcattaacc ggacttgcca gtgttactca attgatggca acctttattc
aactagttggaaaaaataat gaagaatctt taaaaaatga tctggctcta ttccagtctc tccaagaatcaagaaaaact
gaaatggaga gaaaatctga tgagtatgct gctgaagtac gtaaagcagaagaactcaac agagtaatgg gttgtgttgg
gaaaatactt ggggcacttt taactatcgttagtgttgtt gcagcagctt tttctggagg agcctctcta gcactggcag
ctgttggtttagctcttatg gttacggatg ctatagtaca agcagcgacc ggcaattcct tcatggaacaagccctgaat
ccgatcatga aagcagtcat tgaaccctta atcaaactcc tttcagatgcatttacaaaa atgctcgaag gcttgggcgt
cgactcgaaa aaagccaaaa tgattggctctattctgggg gcaatcgcag gcgctcttgt cctagttgca gcagtcgttc
tcgtagccactgttggtaaa caggcagcag caaaacttgc agaaaatatt ggcaaaataa taggtaaaaccctcacagac
cttataccaa agtttctcaa gaatttttct tctcaactgg acgatttaatcactaatgct gttgccagat taaataaatt
tcttggtgca gcgggtgatg aagtaatatccaaacaaatt atttccaccc atttaaacca agcagtttta ttaggagaaa
gtgttaactctgccacacaa gcgggaggaa gtgtcgcttc tgctgttttc cagaacagcg cgtcgacaaatctagcagac
ctgacattat cgaaatatca agttgaacaa ctgtcaaaat atatcagtgaagcaatagaa aaattcggcc aattgcagga
agtaattgca gatctattag cctcaatgtccaactctcag gctaatagaa ctgatgttgc aaaagcaatt ttgcaacaaa
ctactgcttga GGATCC
LTA1-GSAAS-IpaD-LE-IpaB (DBF) Amino Acid sequence
SEQ ID NO: 16
MDNGDRLYRADSRPPDEIKRSGGLMPRGHNEYFDRGTQMNINLYDHARGTQ
TGFVRYDDGYVSTSLSLRSAHLAGQSILSGYSTYYIYVIATAPNMFNVNDVLGVYSP
HPYEQEVSALGGIPYSQIYGWYRVNFGVIDERLHRNREYRDRYYRNLNIAPAEDGYR
LAGFPPDHQAWREEPWIHHAPQGCGNSSRGSAASMNITTLTNSISTSSFSPNNTNGSS
TETVNSDIKTTTSSHPSSLTMLNDTLHNIRTTNQALKKELSQKTLRNEYPINKDAREL
LHSAPKEAELDGDQMISHRELWAKIANSINDINEQYLKVYEHAVSSYTQMYQDFSA
VLSSLAGWISPGGNDGNSVKLQVNSLKKALEELKEKYKDKPLYPANNTVSQEQANK
WLTELGGTIGKVSQKNGGYVVSINMTPIDNMLKSLDNLGGNGEVVLDNAKYQAWN
GFSAEDETMKNNLQTLVQKYSNANSIFDNLVKVLSSTISSCTDTDKLFLHFLEMHNV
STTTTGFPLAKILTSTELGDNTIQAANDAANKLFSLTIADLTANQNINTTNAHSTSNILI
PELKAPKSLNASSQLTLLIGNLIQILGEKSLTALTNKITAWKSQQQARQQKNLEFSDKI
NTLLSETEGLTRDYEKQINKLKNADSKIKDLENKINQIQTRLSNLDPESPEKKKLSREE
IQLTIKKDAAVKDRTLIEQKTLSIHSKLTDKSMQLEKEIDSFSAFSNTASAEQLSTQQK
SLTGLASVTQLMATFIQLVGKNNEESLKNDLALFQSLQESRKTEMERKSDEYAAEVR
KAEELNRVMGCVGKILGALLTIVSVVAAAFSGGASLALAAVGLALMVTDAIVQAAT
GNSFMEQALNPIMKAVIEPLIKLLSDAFTKMLEGLGVDSKKAKMIGSILGAIAGALVL
VAAVVLVATVGKQAAAKLAENIGKIIGKTLTDLIPKFLKNFSSQLDDLITNAVARLN
KFLGAAGDEVISKQIISTHLNQAVLLGESVNSATQAGGSVASAVFQNSASTNLADLT
LSKYQVEQLSKYISEAIEKFGQLQEVIADLLASMSNSQANRTDVAKAILQQTTA
LTA1-22BF nucleic acid sequence
SEQ ID NO: 17
CATAtggacaatggcgatcgtttataccgtgccgactcgcgtcccccagatgagattaaacgtagcggtgggttaatgc
cacgtgggcacaatgagtattttgaccgtggaacacagatgaacattaacctttacgatcatgcccgtgggacccagaccgggtttgtc
cgttatgatgacgggtatgttagtacgagtttgtccttacgctccgcacaccttgcgggacaaagtattttatcaggctacagcacatatta
catttatgtgatcgccactgccccaaacatgttcaatgtgaacgatgtgttgggggtttacagcccccatccatatgaacaagaagtctcg
gcccttggggggatcccatatagccagatttatggttggtaccgcgtaaattttggtgtgattgatgaacgtttgcatcgtaaccgtgaata
ccgcgatcgctactaccgtaacttgaacattgcacctgccgaggacggctatcgtttagcgggattcccacccgatcatcaggcgtgg
cgtgaggaaccgtggatccatcacgcccctcaggggtgcgggaacagtagtcgcatgaccattgatctcggagtttcactcacgtcgc
aggccggcggcctgcaaggcatcgacctcaagagcatggatatccagactctcatggtgtatgtgcagggtcgtcgcgccgaactcc
tcacggctcaaatgcagacccaggccgaagtggtgcagaaggccaatgaacgcatggcgcagctcaacgaggtcctgtccgcgct
gtcccgggccaaggccgagtttccgcccaatccgaagccgggcgacaccatcccgggctgggacaaccagaaggtcagccggat
cgaggttcctctcaatgatgcgctgcgcgctgccggcctgacgggcatgttcgaagcgcgcgatggccaagtgaccgcccccggcg
gccggggtacgcaggtcgtgaacggcacgggcgtcatggccggttccacgacctataaggaactcgaaagtgcctacaccaccgta
aaggggatgctggatacggcgtccaatacgcaacagatggacatgatcaggctgcaggccgccagcaacaagcgcaacgaggctt
tcgaggtcatgaccaacaccgagaagcggcgcagcgacctgaacagttccatcaccaacaacatgcgcaagcttatgaccgtcatga
gtacgaccatatccacagccccgagcggcgccgcgcttgcgccgtctcgcatagatatgcgggcaccggagcccgggagtgccgg
cgaaggcgccggcatcctggcgccggtgacgacgctggctctggcggcgggccggccggcttttccagcgtcaccgtcgctgcgc
accgcgcccgtcctggatccgccagtgcgcgatctcagccccgccgacttggccgacctgctgcgcgtcttgcgatccagggcggt
ggacgggcagttggccacggcgcgcgagaacctgcaggacgcgcaagtcaaggcgaagcagaacacccaggcccagctcgac
aagctggacgcatggtttcggaaggccgaagaggccgagagcaagggatggctgagcaaggtgttcggctggatcggcaaggtg
ctggcggtcgtggcatcggccctggcggtgggctttgccgccgtcgccagcgtggccaccggcgcggcggccacacccatgctgc
tgctcagcggcatggcactggtcagcgccgtgacatcgctggccgaccagatatcgcaagaggcgggaggcccgcctatcagcct
gggcgggtttctctccgggctggccggacgtctgctgacagcgttgggggtggatcagtcgcaggccgaccaaattgccaagatcgt
cgccggcctggccgtgcccgtcgtcttgctgatcgaaccccagatgctgggcgaaatggcgcaaggcgtggccaggctggctggc
gccagcgatgccaccgcggggtacatagccatggcgatgtccatcgtggcggcgatcgcggtcgccgcgatcaatgccgccggta
cagccggcgcgggtagcgcttcggcgatcaagggggcctgggatcgggccgccgcggtagccacccaggtccttcaagggggta
cggcagtggcgcaaggcggcgtcggcgtgtcgatggcagtcgatcgcaaacaggccgatctcctggtcgccgacaaggcggatct
ggcggcgagcctgacaaaactgcgggcggccatggagcgtgaggcggacgatatcaagaagatcctggctcaattcgacgaggc
ctatcacatgatcgcgaagatgatcagcgatatggcgagtacgcacagccaggtcagcgccaacctcgggcggcgccaggcggtg
tagCTCGAG
LTA1-22BF Amino acid sequence
SEQ ID NO: 18
MDNGDRLYRADSRPPDEIKRSGGLMPRGHNEYFDRGTQMNINLYDHARGTQ
TGFVRYDDGYVSTSLSLRSAHLAGQSILSGYSTYYIYVIATAPNMFNVNDVLGVYSP
HPYEQEVSALGGIPYSQIYGWYRVNFGVIDERLHRNREYRDRYYRNLNIAPAEDGYR
LAGFPPDHQAWREEPWIHHAPQGCGNSSRMTIDLGVSLTSQAGGLQGIDLKSMDIQT
LMVYVQGRRAELLTAQMQTQAEVVQKANERMAQLNEVLSALSRAKAEFPPNPKPG
DTIPGWDNQKVSRIEVPLNDALRAAGLTGMFEARDGQVTAPGGRGTQVVNGTGVM
AGSTTYKELESAYTTVKGMLDTASNTQQMDMIRLQAASNKRNEAFEVMTNTEKRR
SDLNSSITNNMRKLMTVMSTTISTAPSGAALAPSRIDMRAPEPGSAGEGAGILAPVTT
LALAAGRPAFPASPSLRTAPVLDPPVRDLSPADLADLLRVLRSRAVDGQLATARENL
QDAQVKAKQNTQAQLDKLDAWFRKAEEAESKGWLSKVFGWIGKVLAVVASALAV
GFAAVASVATGAAATPMLLLSGMALVSAVTSLADQISQEAGGPPISLGGFLSGLAGR
LLTALGVDQSQADQIAKIVAGLAVPVVLLIEPQMLGEMAQGVARLAGASDATAGYI
AMAMSIVAAIAVAAINAAGTAGAGSASAIKGAWDRAAAVATQVLQGGTAVAQGG
VGVSMAVDRKQADLLVADKADLAASLTKLRAAMEREADDIKKILAQFDEAYHMIA
KMISDMASTHSQVSANLGRRQAV*
His-BicA (Chaperone of BurkF) nucleic acid sequence
SEQ ID NO: 19
ATGGGCAGCAGCCATCACCATCATCACCACAGCCAGGATCCGATGACGCA
ACGCGACGTGAACATAGACGACATCGAGGCGCAGGAAATGGCGGCGGCGCTGCT
GGACGCGGTCCAGAACGGCGCGACGCTGAAGGACCTGCATCAGGTGCCGCAGGA
CCTGATGGACGGCATCTATGCGTTCGCGTACCGCTTCTACCAGCAGGGGCGGCTC
GACGACGCGGAGGTGTTCTTCCGCTTTCTGCGCATCTACGACTTCTACAACGCCG
AATACGCGATGGGGCTCGCGGCGGTGTGCCAGTTGAAGAAGGAGTACGCGCGGG
CGATCGATCTGTATGCACTCGCGTATTCGCTGTCGAAGGACGACCACCGGCCGAT
GTTCCACACCGGCCAATGCCATCTGCTGATGGGCAAGGCGGCGCTCGCGCGGCG
CTGCTTCGGCATCGTCGTCGAGCGCTCGCGCGACGAGCGCCTCGCGCAGAAGGC
GCAGTCCTATCTCGACGGGCTCGACGAAGTGGGCGCCGACGCGGCGCCCGCATC
CGCCGGGAACGACCACTGAGCGGCCGC
His-BicA (Chaperone of BurkF) Amino acid sequence
SEQ ID NO: 20
MGSSHHHHHHSQDPMTQRDVNIDDIEAQEMAAALLDAVQNGATLKDLHQV
PQDLMDGIYAFAYRFYQQGRLDDAEVFFRFLRIYDFYNAEYAMGLAAVCQLKKEY
ARAIDLYALAYSLSKDDHRPMFHTGQCHLLMGKAALARRCFGIVVERSRDERLAQK
AQSYLDGLDEVGADAAPASAGNDH-
BipD nucleic acid sequence
SEQ ID NO: 21
CATATGAACATGCACGTGGACATGGGTCGTGCGCTGACCGTTCGTGATTG
GCCGGCGCTGGAGGCGCTGGCGAAAACCATGCCGGCGGATGCGGGTGCGCGTGC
GATGACCGATGATGACCTGCGTGCGGCGGGTGTGGACCGTCGTGTTCCGGAGCA
GAAGCTGGGTGCGGCGATTGATGAATTCGCGAGCCTGCGTCTGCCGGATCGTATC
GACGGTCGTTTCGTGGATGGCCGTCGTGCGAACCTGACCGTTTTTGATGATGCGC
GTGTTGCGGTTCGTGGTCATGCGCGTGCGCAACGTAACCTGCTGGAGCGTCTGGA
GACCGAACTGCTGGGTGGCACCCTGGATACCGCGGGTGACGAAGGTGGCATTCA
GCCGGACCCGATCCTGCAAGGCCTGGTGGATGTTATCGGTCAGGGCAAAAGCGA
TATTGACGCGTACGCGACCATCGTGGAAGGTCTGACCAAGTATTTTCAAAGCGTG
GCGGACGTTATGAGCAAACTGCAGGATTACATTAGCGCGAAGGATGACAAAAAC
ATGAAGATCGACGGTGGCAAGATCAAAGCGCTGATTCAGCAAGTGATCGACCAC
CTGCCGACCATGCAGCTGCCGAAGGGTGCGGATATTGCGCGTTGGCGTAAAGAG
CTGGGCGACGCGGTTAGCATCAGCGATAGCGGTGTGGTTACCATTAACCCGGAC
AAACTGATCAAGATGCGTGATAGCCTGCCGCCGGATGGCACCGTTTGGGATACC
GCGCGTTACCAAGCGTGGAACACCGCGTTCAGCGGTCAGAAAGGCCAGCATCCG
GAACGTCGTGCGGATGCGCGTCGTAAATATAGCCACCAGAACAGCAACTTTGAT
AACCTGGTGAAGGTTCTGAGCGGTGCGATTAGCACCCTGACCGACACCCAGAGC
TATCTGCAAATC
BipD amino acid sequence
SEQ ID NO: 22
MNMHVDMGRALTVRDWPALEALAKTMPADAGARAMTDDDLRAAGVDRR
VPEQKLGAAIDEFASLRLPDRIDGRFVDGRRANLTVFDDARVAVRGHARAQRNLLE
RLETELLGGTLDTAGDEGGIQPDPILQGLVDVIGQGKSDIDAYATIVEGLTKYFQSVA
DVMSKLQDYISAKDDKNMKIDGGKIKALIQQVIDHLPTMQLPKGADIARWRKELGD
AVSISDSGVVTINPDKLIKMRDSLPPDGTVWDTARYQAWNTAFSGQKGQHPERRAD
ARRKYSHQNSNFDNLVKVLSGAISTLTDTQSYLQI
BipB nucleic acid sequence
SEQ ID NO: 23
ATGAGCAGCGGTGTTCAAGGTGGCCCGGCGGCGAACGCGAACGCGTACC
AGACCCACCCGCTGCGTGATGCGGCGAGCGCGCTGGGCACCCTGAGCCCGCAGG
CGTATGTGGATGTGGTTAGCGCGGCGCAACGTAACTTCCTGGAGCGTATGAGCC
AACTGGCGAGCGAACAGTGCGATGCGCAACCGGCGGCGCATGATGCGCGTCTGG
ATGATCGTCCGGCGCTGCGTGCGCCGCAGGAACGTGACGCGCCGCCGCTGGGTG
CGAGCGATACCGGTAGCCGTGCGAGCGGTGCGGCGAAACTGACCGAGCTGCTGG
GTGTGCTGATGAGCGTTATTAGCGCGAGCAGCCTGGACGAACTGAAGCAACGTA
GCGATATCTGGAACCAGATGAGCAAAGCGGCGCAAGACAACCTGAGCCGTCTGA
GCGATGCGTTTCAGCGTGCGACCGACGAGGCGAAAGCGGCGGCGGATGCGGCGG
AACAGGCGGCGGCGGCGGCGAAGCAAGCGGGTGCGGACGCGAAAGCGGCGGAT
GCGGCGGTGGATGCGGCGCAAAAACGTTACGATGACGCGGTTAAGCAGGGCCTG
CCGGATGACCGTCTGCAAAGCCTGAAAGCGGCGCTGGAGCAGGCGCGTCAGCAA
GCGGGTGATGCGCATGGTCGTGCGGATGCGCTGCAGGCGGATGCGACCAAGAAA
CTGGACGCGGCGAGCGCGCTGGCGACCCAAGCGCGTGCGTGCGAACAGCAAGTG
GATGACGCGGTTAACCAGGCGACCCAGCAATATGGTGCGAGCGCGAGCCTGCGT
ACCCCGCAAAGCCCGCGTCTGAGCGGTGCGGCGGAGCTGACCGCGGTGCTGGGC
AAGCTGCAGGAACTGATTAGCAGCGGCAACGTTAAAGAGCTGGAAAGCAAGCA
GAAACTGTTCACCGAGATGCAAGCGAAGCGTGAGGCGGAACTGCAAAAGAAAA
GCGACGAATATCAGGCGCAAGTGAAGAAAGCGGAGGAAATGCAGAAAACGATG
GGTTGCATCGGCAAGATTGTGGGTTGGGTTATTACCGCGGTTAGCTTTGCGGCGG
CGGCGTTTACCGGTGGCGCGAGCCTGGCGCTGGCGGCGGTGGGCCTGGCGCTGG
CGGTTGGTGACGAGATTAGCCGTGCGACCACCGGTGTGAGCTTCATGGACAAGC
TGATGCAGCCGGTTATGGATGCGATCCTGAAACCGCTGATGGAGATGATTAGCA
GCCTGATCACCAAGGCGCTGGTTGCGTGCGGCGTTGATCAGCAAAAAGCGGAAC
TGGCGGGTGCGATTCTGGGTGCGGTTGTTACCGGTGTGGCGCTGGTTGCGGCGGC
GTTTGTTGGTGCGAGCGCGGTGAAAGCGGTTGCGAGCAAGGTTATCGACGCGAT
GGCGGGTCAGCTGACCAAGCTGATGGATAGCGCGATTGGCAAAATGCTGGTGCA
ACTGATCGAGAAATTCAGCGAAAAGAGCGGTCTGCAGGCGCTGGGTAGCCGTAC
CGCGACCGCGATGACCCGTATGCGTCGTGCGATTGGCGTTGAGGCGAAGGAAGA
CGGTATGCTGCTGGCGAACCGTTTTGAAAAAGCGGGCACCGTGATGAACGTTGG
TAACCAAGTGAGCCAAGCGGCGGGTGGCATTGTGGTTGGCGTTGAGCGTGCGAA
AGCGATGGGTCTGCTGGCGGATGTGAAAGAAGCGATGTATGACATCAAGCTGCT
GGGTGATCTGCTGAAACAGGCGGTGGACGCGTTTGCGGAGCACAACCGTGTTCT
GGCGCAACTGATGCAGCAAATGAGCGATGCGGGCGAAATGCAGACCAGCACCG
GCAAGCTGATCCTGCGTAACGCGCGTGCGGTTTAAGGATCC
BipB amino acid sequence
SEQ ID NO: 24
MSSGVQGGPAANANAYQTHPLRDAASALGTLSPQAYVDVVSAAQRNFLER
MSQLASEQCDAQPAAHDARLDDRPALRAPQERDAPPLGASDTGSRASGAAKLTELL
GVLMSVISASSLDELKQRSDIWNQMSKAAQDNLSRLSDAFQRATDEAKAAADAAEQ
AAAAAKQAGADAKAADAAVDAAQKRYDDAVKQGLPDDRLQSLKAALEQARQQA
GDAHGRADALQADATKKLDAASALATQARACEQQVDDAVNQATQQYGASASLRT
PQSPRLSGAAELTAVLGKLQELISSGNVKELESKQKLFTEMQAKREAELQKKSDEYQ
AQVKKAEEMQKTMGCIGKIVGWVITAVSFAAAAFTGGASLALAAVGLALAVGDEIS
RATTGVSFMDKLMQPVMDAILKPLMEMISSLITKALVACGVDQQKAELAGAILGAV
VTGVALVAAAFVGASAVKAVASKVIDAMAGQLTKLMDSAIGKMLVQLIEKFSEKS
GLQALGSRTATAMTRMRRAIGVEAKEDGMLLANRFEKAGTVMNVGNQVSQAAGG
IVVGVERAKAMGLLADVKEAMYDIKLLGDLLKQAVDAFAEHNRVLAQLMQQMSD
AGEMQTSTGKLILRNARAV
BurkF nucleic acid sequence
SEQ ID NO: 25
CATATGAACATGCACGTGGACATGGGTCGTGCGCTGACCGTTCGTGATTG
GCCGGCGCTGGAGGCGCTGGCGAAAACCATGCCGGCGGATGCGGGTGCGCGTGC
GATGACCGATGATGACCTGCGTGCGGCGGGTGTGGACCGTCGTGTTCCGGAGCA
GAAGCTGGGTGCGGCGATTGATGAATTCGCGAGCCTGCGTCTGCCGGATCGTATC
GACGGTCGTTTCGTGGATGGCCGTCGTGCGAACCTGACCGTTTTTGATGATGCGC
GTGTTGCGGTTCGTGGTCATGCGCGTGCGCAACGTAACCTGCTGGAGCGTCTGGA
GACCGAACTGCTGGGTGGCACCCTGGATACCGCGGGTGACGAAGGTGGCATTCA
GCCGGACCCGATCCTGCAAGGCCTGGTGGATGTTATCGGTCAGGGCAAAAGCGA
TATTGACGCGTACGCGACCATCGTGGAAGGTCTGACCAAGTATTTTCAAAGCGTG
GCGGACGTTATGAGCAAACTGCAGGATTACATTAGCGCGAAGGATGACAAAAAC
ATGAAGATCGACGGTGGCAAGATCAAAGCGCTGATTCAGCAAGTGATCGACCAC
CTGCCGACCATGCAGCTGCCGAAGGGTGCGGATATTGCGCGTTGGCGTAAAGAG
CTGGGCGACGCGGTTAGCATCAGCGATAGCGGTGTGGTTACCATTAACCCGGAC
AAACTGATCAAGATGCGTGATAGCCTGCCGCCGGATGGCACCGTTTGGGATACC
GCGCGTTACCAAGCGTGGAACACCGCGTTCAGCGGTCAGAAAGGCCAGCATCCG
GAACGTCGTGCGGATGCGCGTCGTAAATATAGCCACCAGAACAGCAACTTTGAT
AACCTGGTGAAGGTTCTGAGCGGTGCGATTAGCACCCTGACCGACACCCAGAGC
TATCTGCAAATCAAGCTTATGAGCAGCGGTGTTCAAGGTGGCCCGGCGGCGAAC
GCGAACGCGTACCAGACCCACCCGCTGCGTGATGCGGCGAGCGCGCTGGGCACC
CTGAGCCCGCAGGCGTATGTGGATGTGGTTAGCGCGGCGCAACGTAACTTCCTG
GAGCGTATGAGCCAACTGGCGAGCGAACAGTGCGATGCGCAACCGGCGGCGCAT
GATGCGCGTCTGGATGATCGTCCGGCGCTGCGTGCGCCGCAGGAACGTGACGCG
CCGCCGCTGGGTGCGAGCGATACCGGTAGCCGTGCGAGCGGTGCGGCGAAACTG
ACCGAGCTGCTGGGTGTGCTGATGAGCGTTATTAGCGCGAGCAGCCTGGACGAA
CTGAAGCAACGTAGCGATATCTGGAACCAGATGAGCAAAGCGGCGCAAGACAA
CCTGAGCCGTCTGAGCGATGCGTTTCAGCGTGCGACCGACGAGGCGAAAGCGGC
GGCGGATGCGGCGGAACAGGCGGCGGCGGCGGCGAAGCAAGCGGGTGCGGACG
CGAAAGCGGCGGATGCGGCGGTGGATGCGGCGCAAAAACGTTACGATGACGCG
GTTAAGCAGGGCCTGCCGGATGACCGTCTGCAAAGCCTGAAAGCGGCGCTGGAG
CAGGCGCGTCAGCAAGCGGGTGATGCGCATGGTCGTGCGGATGCGCTGCAGGCG
GATGCGACCAAGAAACTGGACGCGGCGAGCGCGCTGGCGACCCAAGCGCGTGC
GTGCGAACAGCAAGTGGATGACGCGGTTAACCAGGCGACCCAGCAATATGGTGC
GAGCGCGAGCCTGCGTACCCCGCAAAGCCCGCGTCTGAGCGGTGCGGCGGAGCT
GACCGCGGTGCTGGGCAAGCTGCAGGAACTGATTAGCAGCGGCAACGTTAAAGA
GCTGGAAAGCAAGCAGAAACTGTTCACCGAGATGCAAGCGAAGCGTGAGGCGG
AACTGCAAAAGAAAAGCGACGAATATCAGGCGCAAGTGAAGAAAGCGGAGGAA
ATGCAGAAAACGATGGGTTGCATCGGCAAGATTGTGGGTTGGGTTATTACCGCG
GTTAGCTTTGCGGCGGCGGCGTTTACCGGTGGCGCGAGCCTGGCGCTGGCGGCG
GTGGGCCTGGCGCTGGCGGTTGGTGACGAGATTAGCCGTGCGACCACCGGTGTG
AGCTTCATGGACAAGCTGATGCAGCCGGTTATGGATGCGATCCTGAAACCGCTG
ATGGAGATGATTAGCAGCCTGATCACCAAGGCGCTGGTTGCGTGCGGCGTTGAT
CAGCAAAAAGCGGAACTGGCGGGTGCGATTCTGGGTGCGGTTGTTACCGGTGTG
GCGCTGGTTGCGGCGGCGTTTGTTGGTGCGAGCGCGGTGAAAGCGGTTGCGAGC
AAGGTTATCGACGCGATGGCGGGTCAGCTGACCAAGCTGATGGATAGCGCGATT
GGCAAAATGCTGGTGCAACTGATCGAGAAATTCAGCGAAAAGAGCGGTCTGCAG
GCGCTGGGTAGCCGTACCGCGACCGCGATGACCCGTATGCGTCGTGCGATTGGC
GTTGAGGCGAAGGAAGACGGTATGCTGCTGGCGAACCGTTTTGAAAAAGCGGGC
ACCGTGATGAACGTTGGTAACCAAGTGAGCCAAGCGGCGGGTGGCATTGTGGTT
GGCGTTGAGCGTGCGAAAGCGATGGGTCTGCTGGCGGATGTGAAAGAAGCGATG
TATGACATCAAGCTGCTGGGTGATCTGCTGAAACAGGCGGTGGACGCGTTTGCG
GAGCACAACCGTGTTCTGGCGCAACTGATGCAGCAAATGAGCGATGCGGGCGAA
ATGCAGACCAGCACCGGCAAGCTGATCCTGCGTAACGCGCGTGCGGTTTAAGGA
TCC
BurkF amino acid sequence
SEQ ID NO: 26
MNMHVDMGRALTVRDWPALEALAKTMPADAGARAMTDDDLRAAGVDRR
VPEQKLGAAIDEFASLRLPDRIDGRFVDGRRANLTVFDDARVAVRGHARAQRNLLE
RLETELLGGTLDTAGDEGGIQPDPILQGLVDVIGQGKSDIDAYATIVEGLTKYFQSVA
DVMSKLQDYISAKDDKNMKIDGGKIKALIQQVIDHLPTMQLPKGADIARWRKELGD
AVSISDSGVVTINPDKLIKMRDSLPPDGTVWDTARYQAWNTAFSGQKGQHPERRAD
ARRKYSHQNSNFDNLVKVLSGAISTLTDTQSYLQIKLMSSGVQGGPAANANAYQTH
PLRDAASALGTLSPQAYVDVVSAAQRNFLERMSQLASEQCDAQPAAHDARLDDRP
ALRAPQERDAPPLGASDTGSRASGAAKLTELLGVLMSVISASSLDELKQRSDIWNQM
SKAAQDNLSRLSDAFQRATDEAKAAADAAEQAAAAAKQAGADAKAADAAVDAA
QKRYDDAVKQGLPDDRLQSLKAALEQARQQAGDAHGRADALQADATKKLDAASA
LATQARACEQQVDDAVNQATQQYGASASLRTPQSPRLSGAAELTAVLGKLQELISS
GNVKELESKQKLFTEMQAKREAELQKKSDEYQAQVKKAEEMQKTMGCIGKIVGW
VITAVSFAAAAFTGGASLALAAVGLALAVGDEISRATTGVSFMDKLMQPVMDAILK
PLMEMISSLITKALVACGVDQQKAELAGAILGAVVTGVALVAAAFVGASAVKAVAS
KVIDAMAGQLTKLMDSAIGKMLVQLIEKFSEKSGLQALGSRTATAMTRMRRAIGVE
AKEDGMLLANRFEKAGTVMNVGNQVSQAAGGIVVGVERAKAMGLLADVKEAMY
DIKLLGDLLKQAVDAFAEHNRVLAQLMQQMSDAGEMQTSTGKLILRNARAV
LTA1-BurkF nucleic acid sequence
SEQ ID NO: 27
CATatggacaatggcgatcgtttataccgtgccgactcgcgtcccccagatgagattaaacgtagcggtgggttaatgcc
acgtgggcacaatgagtattttgaccgtggaacacagatgaacattaacctttacgatcatgcccgtgggacccagaccgggtttgtcc
gttatgatgacgggtatgttagtacgagtttgtccttacgctccgcacaccttgcgggacaaagtattttatcaggctacagcacatattac
atttatgtgatcgccactgccccaaacatgttcaatgtgaacgatgtgttgggggtttacagcccccatccatatgaacaagaagtctcg
gcccttggggggatcccatatagccagatttatggttggtaccgcgtaaattttggtgtgattgatgaacgtttgcatcgtaaccgtgaata
ccgcgatcgctactaccgtaacttgaacattgcacctgccgaggacggctatcgtttagcgggattcccacccgatcatcaggcgtgg
cgtgaggaaccgtggatccatcacgcccctcaggggtgcgggaacagtagtcgcCATATGAACATGCACGTGGA
CATGGGTCGTGCGCTGACCGTTCGTGATTGGCCGGCGCTGGAGGCGCTGGCGAA
AACCATGCCGGCGGATGCGGGTGCGCGTGCGATGACCGATGATGACCTGCGTGC
GGCGGGTGTGGACCGTCGTGTTCCGGAGCAGAAGCTGGGTGCGGCGATTGATGA
ATTCGCGAGCCTGCGTCTGCCGGATCGTATCGACGGTCGTTTCGTGGATGGCCGT
CGTGCGAACCTGACCGTTTTTGATGATGCGCGTGTTGCGGTTCGTGGTCATGCGC
GTGCGCAACGTAACCTGCTGGAGCGTCTGGAGACCGAACTGCTGGGTGGCACCC
TGGATACCGCGGGTGACGAAGGTGGCATTCAGCCGGACCCGATCCTGCAAGGCC
TGGTGGATGTTATCGGTCAGGGCAAAAGCGATATTGACGCGTACGCGACCATCG
TGGAAGGTCTGACCAAGTATTTTCAAAGCGTGGCGGACGTTATGAGCAAACTGC
AGGATTACATTAGCGCGAAGGATGACAAAAACATGAAGATCGACGGTGGCAAG
ATCAAAGCGCTGATTCAGCAAGTGATCGACCACCTGCCGACCATGCAGCTGCCG
AAGGGTGCGGATATTGCGCGTTGGCGTAAAGAGCTGGGCGACGCGGTTAGCATC
AGCGATAGCGGTGTGGTTACCATTAACCCGGACAAACTGATCAAGATGCGTGAT
AGCCTGCCGCCGGATGGCACCGTTTGGGATACCGCGCGTTACCAAGCGTGGAAC
ACCGCGTTCAGCGGTCAGAAAGGCCAGCATCCGGAACGTCGTGCGGATGCGCGT
CGTAAATATAGCCACCAGAACAGCAACTTTGATAACCTGGTGAAGGTTCTGAGC
GGTGCGATTAGCACCCTGACCGACACCCAGAGCTATCTGCAAATCAAGCTTATG
AGCAGCGGTGTTCAAGGTGGCCCGGCGGCGAACGCGAACGCGTACCAGACCCAC
CCGCTGCGTGATGCGGCGAGCGCGCTGGGCACCCTGAGCCCGCAGGCGTATGTG
GATGTGGTTAGCGCGGCGCAACGTAACTTCCTGGAGCGTATGAGCCAACTGGCG
AGCGAACAGTGCGATGCGCAACCGGCGGCGCATGATGCGCGTCTGGATGATCGT
CCGGCGCTGCGTGCGCCGCAGGAACGTGACGCGCCGCCGCTGGGTGCGAGCGAT
ACCGGTAGCCGTGCGAGCGGTGCGGCGAAACTGACCGAGCTGCTGGGTGTGCTG
ATGAGCGTTATTAGCGCGAGCAGCCTGGACGAACTGAAGCAACGTAGCGATATC
TGGAACCAGATGAGCAAAGCGGCGCAAGACAACCTGAGCCGTCTGAGCGATGCG
TTTCAGCGTGCGACCGACGAGGCGAAAGCGGCGGCGGATGCGGCGGAACAGGC
GGCGGCGGCGGCGAAGCAAGCGGGTGCGGACGCGAAAGCGGCGGATGCGGCGG
TGGATGCGGCGCAAAAACGTTACGATGACGCGGTTAAGCAGGGCCTGCCGGATG
ACCGTCTGCAAAGCCTGAAAGCGGCGCTGGAGCAGGCGCGTCAGCAAGCGGGTG
ATGCGCATGGTCGTGCGGATGCGCTGCAGGCGGATGCGACCAAGAAACTGGACG
CGGCGAGCGCGCTGGCGACCCAAGCGCGTGCGTGCGAACAGCAAGTGGATGACG
CGGTTAACCAGGCGACCCAGCAATATGGTGCGAGCGCGAGCCTGCGTACCCCGC
AAAGCCCGCGTCTGAGCGGTGCGGCGGAGCTGACCGCGGTGCTGGGCAAGCTGC
AGGAACTGATTAGCAGCGGCAACGTTAAAGAGCTGGAAAGCAAGCAGAAACTG
TTCACCGAGATGCAAGCGAAGCGTGAGGCGGAACTGCAAAAGAAAAGCGACGA
ATATCAGGCGCAAGTGAAGAAAGCGGAGGAAATGCAGAAAACGATGGGTTGCA
TCGGCAAGATTGTGGGTTGGGTTATTACCGCGGTTAGCTTTGCGGCGGCGGCGTT
TACCGGTGGCGCGAGCCTGGCGCTGGCGGCGGTGGGCCTGGCGCTGGCGGTTGG
TGACGAGATTAGCCGTGCGACCACCGGTGTGAGCTTCATGGACAAGCTGATGCA
GCCGGTTATGGATGCGATCCTGAAACCGCTGATGGAGATGATTAGCAGCCTGAT
CACCAAGGCGCTGGTTGCGTGCGGCGTTGATCAGCAAAAAGCGGAACTGGCGGG
TGCGATTCTGGGTGCGGTTGTTACCGGTGTGGCGCTGGTTGCGGCGGCGTTTGTT
GGTGCGAGCGCGGTGAAAGCGGTTGCGAGCAAGGTTATCGACGCGATGGCGGGT
CAGCTGACCAAGCTGATGGATAGCGCGATTGGCAAAATGCTGGTGCAACTGATC
GAGAAATTCAGCGAAAAGAGCGGTCTGCAGGCGCTGGGTAGCCGTACCGCGACC
GCGATGACCCGTATGCGTCGTGCGATTGGCGTTGAGGCGAAGGAAGACGGTATG
CTGCTGGCGAACCGTTTTGAAAAAGCGGGCACCGTGATGAACGTTGGTAACCAA
GTGAGCCAAGCGGCGGGTGGCATTGTGGTTGGCGTTGAGCGTGCGAAAGCGATG
GGTCTGCTGGCGGATGTGAAAGAAGCGATGTATGACATCAAGCTGCTGGGTGAT
CTGCTGAAACAGGCGGTGGACGCGTTTGCGGAGCACAACCGTGTTCTGGCGCAA
CTGATGCAGCAAATGAGCGATGCGGGCGAAATGCAGACCAGCACCGGCAAGCTG
ATCCTGCGTAACGCGCGTGCGGTTTAAGGATCC
LTA1-BurkF Amino acid sequence
SEQ ID NO: 28
MDNGDRLYRADSRPPDEIKRSGGLMPRGHNEYFDRGTQMNINLYDHARGTQ
TGFVRYDDGYVSTSLSLRSAHLAGQSILSGYSTYYIYVIATAPNMFNVNDVLGVYSP
HPYEQEVSALGGIPYSQIYGWYRVNFGVIDERLHRNREYRDRYYRNLNIAPAEDGYR
LAGFPPDHQAWREEPWIHHAPQGCGNSSRMNMHVDMGRALTVRDWPALEALAKT
MPADAGARAMTDDDLRAAGVDRRVPEQKLGAAIDEFASLRLPDRIDGRFVDGRRA
NLTVFDDARVAVRGHARAQRNLLERLETELLGGTLDTAGDEGGIQPDPILQGLVDVI
GQGKSDIDAYATIVEGLTKYFQSVADVMSKLQDYISAKDDKNMKIDGGKIKALIQQ
VIDHLPTMQLPKGADIARWRKELGDAVSISDSGVVTINPDKLIKMRDSLPPDGTVWD
TARYQAWNTAFSGQKGQHPERRADARRKYSHQNSNFDNLVKVLSGAISTLTDTQSY
LQIKLMSSGVQGGPAANANAYQTHPLRDAASALGTLSPQAYVDVVSAAQRNFLER
MSQLASEQCDAQPAAHDARLDDRPALRAPQERDAPPLGASDTGSRASGAAKLTELL
GVLMSVISASSLDELKQRSDIWNQMSKAAQDNLSRLSDAFQRATDEAKAAADAAEQ
AAAAAKQAGADAKAADAAVDAAQKRYDDAVKQGLPDDRLQSLKAALEQARQQA
GDAHGRADALQADATKKLDAASALATQARACEQQVDDAVNQATQQYGASASLRT
PQSPRLSGAAELTAVLGKLQELISSGNVKELESKQKLFTEMQAKREAELQKKSDEYQ
AQVKKAEEMQKTMGCIGKIVGWVITAVSFAAAAFTGGASLALAAVGLALAVGDEIS
RATTGVSFMDKLMQPVMDAILKPLMEMISSLITKALVACGVDQQKAELAGAILGAV
VTGVALVAAAFVGASAVKAVASKVIDAMAGQLTKLMDSAIGKMLVQLIEKFSEKS
GLQALGSRTATAMTRMRRAIGVEAKEDGMLLANRFEKAGTVMNVGNQVSQAAGG
IVVGVERAKAMGLLADVKEAMYDIKLLGDLLKQAVDAFAEHNRVLAQLMQQMSD
AGEMQTSTGKLILRNARAV-
His-PcrH (Chaperone of PaF) nucleic acid sequence
SEQ ID NO: 29
ATGGGCAGCAGCCATCACCATCATCACCACAGCCAGGATCCGATGAACCA
GCCGACCCCTTCCGACACCGACCAGCAACAGGCGCTGGAGGCCTTCCTGCGCGA
CGGCGGCACCCTGGCGATGCTTCGCGGACTCAGCGAGGACACCCTGGAGCAGCT
CTATGCGCTGGGCTTCAACCAGTACCAGGCGGGCAAGTGGGACGACGCGCAGAA
GATCTTCCAGGCACTGTGCATGCTCGACCACTACGACGCCCGCTACTTTCTCGGC
CTGGGCGCCTGCCGCCAGTCCCTCGGTCTCTATGAACAGGCCCTGCAGAGCTACA
GCTACGGCGCGCTGATGGACATCAACGAGCCGCGCTTTCCCTTCCATGCCGCCGA
GTGCCACCTGCAACTGGGTGATCTCGACGGAGCCGAGAGTGGCTTCTACTCGGCC
CGGGCCCTGGCCGCGGCACAGCCGGCGCACGAGGCCCTGGCCGCGCGTGCCGGC
GCCATGTTGGAAGCCGTAACCGCGAGAAAGGATCGAGCCTATGAATCCGATAAC
GCTTGAAAGCTT
His-PcrH (Chaperone of PaF) Amino acid sequence
SEQ ID NO: 30
MGSSHHHHHHSQDPMNQPTPSDTDQQQALEAFLRDGGTLAMLRGLSEDTLE
QLYALGFNQYQAGKWDDAQKIFQALCMLDHYDARYFLGLGACRQSLGLYEQALQS
YSYGALMDINEPRFPFHAAECHLQLGDLDGAESGFYSARALAAAQPAHEALAARAG
AMLEAVTARKDRAYESDNA-
PcrV nucleic acid sequence
SEQ ID NO: 31
CATATGGAAGTCAGAAACCTTAATGCCGCTCGCGAGCTGTTCCTGGACGA
GCTCCTGGCCGCGTCGGCGGCGCCTGCCAGTGCCGAGCAGGAGGAACTGCTGGC
CCTGTTGCGCAGCGAGCGGATCGTGCTGGCCCACGCCGGCCAGCCGCTGAGCGA
GGCGCAAGTGCTCAAGGCGCTCGCCTGGTTGCTCGCGGCCAATCCGTCCGCGCCT
CCGGGGCAGGGCCTCGAGGTACTCCGCGAAGTCCTGCAGGCACGTCGGCAGCCC
GGTGCGCAGTGGGATCTGCGTGAGTTCCTGGTGTCGGCCTATTTCAGCCTGCACG
GGCGTCTCGACGAGGATGTCATCGGTGTCTACAAGGATGTCCTGCAGACCCAGG
ACGGCAAGCGCAAGGCGCTGCTCGACGAGCTCAAGGCGCTGACCGCGGAGTTGA
AGGTCTACAGCGTGATCCAGTCGCAGATCAACGCCGCGCTGTCGGCCAGGCAGG
GCATCAGGATCGACGCTGGCGGTATCGATCTGGTCGACCCCACGCTATATGGCTA
TGCCGTCGGCGATCCCAGGTGGAAGGACAGCCCCGAGTATGCGCTGCTGAGCAA
TCTGGATACCTTCAGCGGCAAGCTGTCGATCAAGGATTTTCTCAGCGGCTCGCCG
AAGCAGAGCGGGGAACTCAAGGGCCTCAGCGATGAGTACCCCTTCGAGAAGGAC
AACAACCCGGTCGGCAATTTCGCCACCACGGTGAGCGACCGCTCGCGTCCGCTG
AACGACAAGGTCAACGAGAAGACCACCCTGCTCAACGACACCAGCTCCCGCTAC
AACTCGGCGGTCGAGGCGCTCAACCGCTTCATCCAGAAATACGACAGCGTCCTG
AGCGACATTCTCAGCGCGATC
PcrV amino acid sequence
SEQ ID NO: 32
MEVRNLNAARELFLDELLAASAAPASAEQEELLALLRSERIVLAHAGQPLSEA
QVLKALAWLLAANPSAPPGQGLEVLREVLQARRQPGAQWDLREFLVSAYFSLHGRL
DEDVIGVYKDVLQTQDGKRKALLDELKALTAELKVYSVIQSQINAALSARQGIRIDA
GGIDLVDPTLYGYAVGDPRWKDSPEYALLSNLDTFSGKLSIKDFLSGSPKQSGELKG
LSDEYPFEKDNNPVGNFATTVSDRSRPLNDKVNEKTTLLNDTSSRYNSAVEALNRFI
QKYDSVLSDILSAI
PopB nucleic acid sequence
SEQ ID NO: 33
ATGAACCCGATTACGCTGGAACGTGCTGGTCTGCCGTATGGTGTTGCCGA
TGCTGGTGACATCCCGGCTCTGGGTCGCCCGGTCGCACGTGATGTGGAAAGTCTG
CGTGTTGAACGTCTGGCAGCACCGGCAGCTGCAAGCGCATCTGGCACCGGTGTC
GCTCTGACGCCGCCGTCTGCAGCAAGTCAGCAACGTCTGGAAGTTGCTAACCGC
GCGGAAATTGCCTCACTGGTCCAGGCAGTGGGTGAAGACGTGGGTCTGGCACGT
CAAGTGGTTCTGGCAGGTGCATCGACCCTGCTGAGCGCAGGTCTGATGTCGCCGC
AGGCGTTCGAAATTGAACTGGCCAAAATCACCGGCGAAGTTGAAAATCAGCAGA
AAAAACTGAAACTGACGGAAATCGAACAGGCCCGTAAACAGAACCTGCAAAAA
ATGGAAGATAACCAGCAAAAAATCCGCGAATCGGAAGAAGCTGCGAAAGAAGC
GCAGAAAAGCGGCCTGGCCGCAAAAATTTTTGGTTGGATTTCTGCTATCGCGAGT
ATTATCGTGGGTGCAATCATGGTTGCAACCGGTGTCGGTGCTGCAGCAGGTGCAC
TGATGATTGCTGGCGGTGTCATGGGTGTCGTGAGTCAGTCCGTGCAGCAAGCAGC
TGCGGATGGTCTGATCTCAAAAGAAGTGATGGAAAAACTGGGCCCGGCCCTGAT
GGGTATTGAAATGGCCGTGGCACTGCTGGCCGCAGTTGTCTCCTTTGGTGGTTCA
GCAGTTGGTGGTCTGGCACGTCTGGGTGCAAAAATCGGCGGTAAAGCTGCGGAA
ATGACGGCATCCCTGGCTTCAAAAGTGGCAGACCTGGGCGGTAAATTCGGCTCTC
TGGCGGGCCAGTCACTGTCGCATAGCCTGAAACTGGGTGTGCAAGTTTCTGATCT
GACCCTGGACGTTGCAAACGGCGCCGCACAGGCTACGCACAGTGGTTTTCAAGC
GAAAGCTGCGAATCGTCAGGCCGATGTTCAAGAATCCCGTGCAGACCTGACCAC
GCTGCAGGGTGTCATTGAACGTCTGAAAGAAGAACTGAGCCGCATGCTGGAAGC
CTTTCAGGAAATTATGGAACGCATCTTCGCAATGCTGCAAGCGAAAGGCGAAAC
CCTGCACAATCTGTCTTCCCGTCCGGCGGCTATCTGAGGATCC
PopB amino acid sequence
SEQ ID NO: 34
MNPITLERAGLPYGVADAGDIPALGRPVARDVESLRVERLAAPAAASASGTG
VALTPPSAASQQRLEVANRAEIASLVQAVGEDVGLARQVVLAGASTLLSAGLMSPQ
AFEIELAKITGEVENQQKKLKLTEIEQARKQNLQKMEDNQQKIRESEEAAKEAQKSG
LAAKIFGWISAIASIIVGAIMVATGVGAAAGALMIAGGVMGVVSQSVQQAAADGLIS
KEVMEKLGPALMGIEMAVALLAAVVSFGGSAVGGLARLGAKIGGKAAEMTASLAS
KVADLGGKFGSLAGQSLSHSLKLGVQVSDLTLDVANGAAQATHSGFQAKAANRQA
DVQESRADLTTLQGVIERLKEELSRMLEAFQEIMERIFAMLQAKGETLHNLSSRPAAI
PaF nucleic acid sequence
SEQ ID NO: 35
CATATGGAAGTCAGAAACCTTAATGCCGCTCGCGAGCTGTTCCTGGACGA
GCTCCTGGCCGCGTCGGCGGCGCCTGCCAGTGCCGAGCAGGAGGAACTGCTGGC
CCTGTTGCGCAGCGAGCGGATCGTGCTGGCCCACGCCGGCCAGCCGCTGAGCGA
GGCGCAAGTGCTCAAGGCGCTCGCCTGGTTGCTCGCGGCCAATCCGTCCGCGCCT
CCGGGGCAGGGCCTCGAGGTACTCCGCGAAGTCCTGCAGGCACGTCGGCAGCCC
GGTGCGCAGTGGGATCTGCGTGAGTTCCTGGTGTCGGCCTATTTCAGCCTGCACG
GGCGTCTCGACGAGGATGTCATCGGTGTCTACAAGGATGTCCTGCAGACCCAGG
ACGGCAAGCGCAAGGCGCTGCTCGACGAGCTCAAGGCGCTGACCGCGGAGTTGA
AGGTCTACAGCGTGATCCAGTCGCAGATCAACGCCGCGCTGTCGGCCAGGCAGG
GCATCAGGATCGACGCTGGCGGTATCGATCTGGTCGACCCCACGCTATATGGCTA
TGCCGTCGGCGATCCCAGGTGGAAGGACAGCCCCGAGTATGCGCTGCTGAGCAA
TCTGGATACCTTCAGCGGCAAGCTGTCGATCAAGGATTTTCTCAGCGGCTCGCCG
AAGCAGAGCGGGGAACTCAAGGGCCTCAGCGATGAGTACCCCTTCGAGAAGGAC
AACAACCCGGTCGGCAATTTCGCCACCACGGTGAGCGACCGCTCGCGTCCGCTG
AACGACAAGGTCAACGAGAAGACCACCCTGCTCAACGACACCAGCTCCCGCTAC
AACTCGGCGGTCGAGGCGCTCAACCGCTTCATCCAGAAATACGACAGCGTCCTG
AGCGACATTCTCAGCGCGATCGGATCCATGAACCCGATTACGCTGGAACGTGCT
GGTCTGCCGTATGGTGTTGCCGATGCTGGTGACATCCCGGCTCTGGGTCGCCCGG
TCGCACGTGATGTGGAAAGTCTGCGTGTTGAACGTCTGGCAGCACCGGCAGCTG
CAAGCGCATCTGGCACCGGTGTCGCTCTGACGCCGCCGTCTGCAGCAAGTCAGC
AACGTCTGGAAGTTGCTAACCGCGCGGAAATTGCCTCACTGGTCCAGGCAGTGG
GTGAAGACGTGGGTCTGGCACGTCAAGTGGTTCTGGCAGGTGCATCGACCCTGCT
GAGCGCAGGTCTGATGTCGCCGCAGGCGTTCGAAATTGAACTGGCCAAAATCAC
CGGCGAAGTTGAAAATCAGCAGAAAAAACTGAAACTGACGGAAATCGAACAGG
CCCGTAAACAGAACCTGCAAAAAATGGAAGATAACCAGCAAAAAATCCGCGAA
TCGGAAGAAGCTGCGAAAGAAGCGCAGAAAAGCGGCCTGGCCGCAAAAATTTTT
GGTTGGATTTCTGCTATCGCGAGTATTATCGTGGGTGCAATCATGGTTGCAACCG
GTGTCGGTGCTGCAGCAGGTGCACTGATGATTGCTGGCGGTGTCATGGGTGTCGT
GAGTCAGTCCGTGCAGCAAGCAGCTGCGGATGGTCTGATCTCAAAAGAAGTGAT
GGAAAAACTGGGCCCGGCCCTGATGGGTATTGAAATGGCCGTGGCACTGCTGGC
CGCAGTTGTCTCCTTTGGTGGTTCAGCAGTTGGTGGTCTGGCACGTCTGGGTGCA
AAAATCGGCGGTAAAGCTGCGGAAATGACGGCATCCCTGGCTTCAAAAGTGGCA
GACCTGGGCGGTAAATTCGGCTCTCTGGCGGGCCAGTCACTGTCGCATAGCCTGA
AACTGGGTGTGCAAGTTTCTGATCTGACCCTGGACGTTGCAAACGGCGCCGCACA
GGCTACGCACAGTGGTTTTCAAGCGAAAGCTGCGAATCGTCAGGCCGATGTTCA
AGAATCCCGTGCAGACCTGACCACGCTGCAGGGTGTCATTGAACGTCTGAAAGA
AGAACTGAGCCGCATGCTGGAAGCCTTTCAGGAAATTATGGAACGCATCTTCGC
AATGCTGCAAGCGAAAGGCGAAACCCTGCACAATCTGTCTTCCCGTCCGGCGGC
TATCTGAGGATCC
PaF amino acid sequence
SEQ ID NO: 36
MEVRNLNAARELFLDELLAASAAPASAEQEELLALLRSERIVLAHAGQPLSEA
QVLKALAWLLAANPSAPPGQGLEVLREVLQARRQPGAQWDLREFLVSAYFSLHGRL
DEDVIGVYKDVLQTQDGKRKALLDELKALTAELKVYSVIQSQINAALSARQGIRIDA
GGIDLVDPTLYGYAVGDPRWKDSPEYALLSNLDTFSGKLSIKDFLSGSPKQSGELKG
LSDEYPFEKDNNPVGNFATTVSDRSRPLNDKVNEKTTLLNDTSSRYNSAVEALNRFI
QKYDSVLSDILSAIGSMNPITLERAGLPYGVADAGDIPALGRPVARDVESLRVERLAA
PAAASASGTGVALTPPSAASQQRLEVANRAEIASLVQAVGEDVGLARQVVLAGAST
LLSAGLMSPQAFEIELAKITGEVENQQKKLKLTEIEQARKQNLQKMEDNQQKIRESE
EAAKEAQKSGLAAKIFGWISAIASIIVGAIMVATGVGAAAGALMIAGGVMGVVSQS
VQQAAADGLISKEVMEKLGPALMGIEMAVALLAAVVSFGGSAVGGLARLGAKIGG
KAAEMTASLASKVADLGGKFGSLAGQSLSHSLKLGVQVSDLTLDVANGAAQATHS
GFQAKAANRQADVQESRADLTTLQGVIERLKEELSRMLEAFQEIMERIFAMLQAKG
ETLHNLSSRPAAI
LTA1-PaF nucleic acid sequence
SEQ ID NO: 37
CATatggacaatggcgatcgtttataccgtgccgactcgcgtcccccagatgagattaaacgtagcggtgggttaatgcc
acgtgggcacaatgagtattttgaccgtggaacacagatgaacattaacctttacgatcatgcccgtgggacccagaccgggtttgtcc
gttatgatgacgggtatgttagtacgagtttgtccttacgctccgcacaccttgcgggacaaagtattttatcaggctacagcacatattac
atttatgtgatcgccactgccccaaacatgttcaatgtgaacgatgtgttgggggtttacagcccccatccatatgaacaagaagtctcg
gcccttggggggatcccatatagccagatttatggttggtaccgcgtaaattttggtgtgattgatgaacgtttgcatcgtaaccgtgaata
ccgcgatcgctactaccgtaacttgaacattgcacctgccgaggacggctatcgtttagcgggattcccacccgatcatcaggcgtgg
cgtgaggaaccgtggatccatcacgcccctcaggggtgcgggaacagtagtcgcCATATGGAAGTCAGAAACCT
TAATGCCGCTCGCGAGCTGTTCCTGGACGAGCTCCTGGCCGCGTCGGCGGCGCCT
GCCAGTGCCGAGCAGGAGGAACTGCTGGCCCTGTTGCGCAGCGAGCGGATCGTG
CTGGCCCACGCCGGCCAGCCGCTGAGCGAGGCGCAAGTGCTCAAGGCGCTCGCC
TGGTTGCTCGCGGCCAATCCGTCCGCGCCTCCGGGGCAGGGCCTCGAGGTACTCC
GCGAAGTCCTGCAGGCACGTCGGCAGCCCGGTGCGCAGTGGGATCTGCGTGAGT
TCCTGGTGTCGGCCTATTTCAGCCTGCACGGGCGTCTCGACGAGGATGTCATCGG
TGTCTACAAGGATGTCCTGCAGACCCAGGACGGCAAGCGCAAGGCGCTGCTCGA
CGAGCTCAAGGCGCTGACCGCGGAGTTGAAGGTCTACAGCGTGATCCAGTCGCA
GATCAACGCCGCGCTGTCGGCCAGGCAGGGCATCAGGATCGACGCTGGCGGTAT
CGATCTGGTCGACCCCACGCTATATGGCTATGCCGTCGGCGATCCCAGGTGGAAG
GACAGCCCCGAGTATGCGCTGCTGAGCAATCTGGATACCTTCAGCGGCAAGCTG
TCGATCAAGGATTTTCTCAGCGGCTCGCCGAAGCAGAGCGGGGAACTCAAGGGC
CTCAGCGATGAGTACCCCTTCGAGAAGGACAACAACCCGGTCGGCAATTTCGCC
ACCACGGTGAGCGACCGCTCGCGTCCGCTGAACGACAAGGTCAACGAGAAGACC
ACCCTGCTCAACGACACCAGCTCCCGCTACAACTCGGCGGTCGAGGCGCTCAAC
CGCTTCATCCAGAAATACGACAGCGTCCTGAGCGACATTCTCAGCGCGATCGGAT
CCATGAACCCGATTACGCTGGAACGTGCTGGTCTGCCGTATGGTGTTGCCGATGC
TGGTGACATCCCGGCTCTGGGTCGCCCGGTCGCACGTGATGTGGAAAGTCTGCGT
GTTGAACGTCTGGCAGCACCGGCAGCTGCAAGCGCATCTGGCACCGGTGTCGCT
CTGACGCCGCCGTCTGCAGCAAGTCAGCAACGTCTGGAAGTTGCTAACCGCGCG
GAAATTGCCTCACTGGTCCAGGCAGTGGGTGAAGACGTGGGTCTGGCACGTCAA
GTGGTTCTGGCAGGTGCATCGACCCTGCTGAGCGCAGGTCTGATGTCGCCGCAGG
CGTTCGAAATTGAACTGGCCAAAATCACCGGCGAAGTTGAAAATCAGCAGAAAA
AACTGAAACTGACGGAAATCGAACAGGCCCGTAAACAGAACCTGCAAAAAATG
GAAGATAACCAGCAAAAAATCCGCGAATCGGAAGAAGCTGCGAAAGAAGCGCA
GAAAAGCGGCCTGGCCGCAAAAATTTTTGGTTGGATTTCTGCTATCGCGAGTATT
ATCGTGGGTGCAATCATGGTTGCAACCGGTGTCGGTGCTGCAGCAGGTGCACTG
ATGATTGCTGGCGGTGTCATGGGTGTCGTGAGTCAGTCCGTGCAGCAAGCAGCTG
CGGATGGTCTGATCTCAAAAGAAGTGATGGAAAAACTGGGCCCGGCCCTGATGG
GTATTGAAATGGCCGTGGCACTGCTGGCCGCAGTTGTCTCCTTTGGTGGTTCAGC
AGTTGGTGGTCTGGCACGTCTGGGTGCAAAAATCGGCGGTAAAGCTGCGGAAAT
GACGGCATCCCTGGCTTCAAAAGTGGCAGACCTGGGCGGTAAATTCGGCTCTCTG
GCGGGCCAGTCACTGTCGCATAGCCTGAAACTGGGTGTGCAAGTTTCTGATCTGA
CCCTGGACGTTGCAAACGGCGCCGCACAGGCTACGCACAGTGGTTTTCAAGCGA
AAGCTGCGAATCGTCAGGCCGATGTTCAAGAATCCCGTGCAGACCTGACCACGC
TGCAGGGTGTCATTGAACGTCTGAAAGAAGAACTGAGCCGCATGCTGGAAGCCT
TTCAGGAAATTATGGAACGCATCTTCGCAATGCTGCAAGCGAAAGGCGAAACCC
TGCACAATCTGTCTTCCCGTCCGGCGGCTATCTGAGGATCC
LTA1-PaF Amino acid sequence
SEQ ID NO: 38
MDNGDRLYRADSRPPDEIKRSGGLMPRGHNEYFDRGTQMNINLYDHARGTQ
TGFVRYDDGYVSTSLSLRSAHLAGQSILSGYSTYYIYVIATAPNMFNVNDVLGVYSP
HPYEQEVSALGGIPYSQIYGWYRVNFGVIDERLHRNREYRDRYYRNLNIAPAEDGYR
LAGFPPDHQAWREEPWIHHAPQGCGNSSRMEVRNLNAARELFLDELLAASAAPASA
EQEELLALLRSERIVLAHAGQPLSEAQVLKALAWLLAANPSAPPGQGLEVLREVLQA
RRQPGAQWDLREFLVSAYFSLHGRLDEDVIGVYKDVLQTQDGKRKALLDELKALT
AELKVYSVIQSQINAALSARQGIRIDAGGIDLVDPTLYGYAVGDPRWKDSPEYALLS
NLDTFSGKLSIKDFLSGSPKQSGELKGLSDEYPFEKDNNPVGNFATTVSDRSRPLNDK
VNEKTTLLNDTSSRYNSAVEALNRFIQKYDSVLSDILSAIGSMNPITLERAGLPYGVA
DAGDIPALGRPVARDVESLRVERLAAPAAASASGTGVALTPPSAASQQRLEVANRAE
IASLVQAVGEDVGLARQVVLAGASTLLSAGLMSPQAFEIELAKITGEVENQQKKLKL
TEIEQARKQNLQKMEDNQQKIRESEEAAKEAQKSGLAAKIFGWISAIASIIVGAIMVA
TGVGAAAGALMIAGGVMGVVSQSVQQAAADGLISKEVMEKLGPALMGIEMAVAL
LAAVVSFGGSAVGGLARLGAKIGGKAAEMTASLASKVADLGGKFGSLAGQSLSHSL
KLGVQVSDLTLDVANGAAQATHSGFQAKAANRQADVQESRADLTTLQGVIERLKE
ELSRMLEAFQEIMERIFAMLQAKGETLHNLSSRPAAI
SycD (Chaperone for YerF) nucleic acid sequence
SEQ ID NO: 39
ATGGGCAGCAGCCATCACCATCATCACCACAGCCAGGATCCGatgcaacaagag
acgacagacactcaagaataccagctggcaatggaatccttcctaaaaggagggggaactatcgccatgctcaacgaaatttcaagtg
acactttagagcaactctactctcttgcgtttaaccaataccagtcaggaaaatacgaggatgctcacaaggtctttcaagctctctgtgtg
ctagaccactatgattcacgtttctttttagggctaggcgcttgtcgtcaagccatggggcaatacgacttagcgattcatagctacagcta
tggcgccataatggatataaaagaacctcgttttccgtttcatgctgccgaatgtttactgcaaaagggagagcttgctgaagcagaaag
tggcttgttcttggctcaagagcttatcgcagacaaacctgagtttaaggagctttccacccgagttagctcaatgttagaagcaattaaat
tgaaaaaggagatggaacatgagtgcgttgataacccatgaAAGCTT
SycD (Chaperone for YerF) Amino acid sequence
SEQ ID NO: 40
MGSSHHHHHHSSGLVPRGSHMQQETTDTQEYQLAMESFLKGGGTIAMLNEIS
SDTLEQLYSLAFNQYQSGKYEDAHKVFQALCVLDHYDSRFFLGLGACRQAMGQYD
LAIHSYSYGAIMDIKEPRFPFHAAECLLQKGELAEAESGLFLAQELIADKPEFKELSTR
VSSMLEAIKLKKEMEHECVDNP-
LcrV amino acid sequence
SEQ ID NO: 41
CATATGATTAGAGCCTACGAACAAAACCCACAACATTTTATTGAGGATCT
AGAAAAAGTTAGGGTGGAACAACTTACTGGTCATGGTTCTTCAGTTTTAGAAGA
ATTGGTTCAGTTAGTCAAAGATAAAAATATAGATATTTCCATTAAATATGATCCC
AGAAAAGATTCGGAGGTTTTTGCCAATAGAGTAATTACTGATGATATCGAATTGC
TCAAGAAAATCCTAGCTTATTTTCTACCCGAGGATGCCATTCTTAAAGGCGGTCA
TTATGACAACCAACTGCAAAATGGCATCAAGCGAGTAAAAGAGTTCCTTGAATC
ATCGCCGAATACACAATGGGAATTGCGGGCGTTCATGGCAGTAATGCATTTCTCT
TTAACCGCCGATCGTATCGATGATGATATTTTGAAAGTGATTGTTGATTCAATGA
ATCATCATGGTGATGCCCGTAGCAAGTTGCGTGAAGAATTAGCTGAGCTTACCGC
CGAATTAAAGATTTATTCAGTTATTCAAGCCGAAATTAATAAGCATCTGTCTAGT
AGTGGCACCATAAATATCCATGATAAATCCATTAATCTCATGGATAAAAATTTAT
ATGGTTATACAGATGAAGAGATTTTTAAAGCCAGCGCAGAGTACAAAATTCTCG
AGAAAATGCCTCAAACCACCATTCAGGTGGATGGGAGCGAGAAAAAAATAGTCT
CGATAAAGGACTTTCTTGGAAGTGAGAATAAAAGAACCGGGGCGTTGGGTAATC
TGAAAAACTCATACTCTTATAATAAAGATAATAATGAATTATCTCACTTTGCCAC
CACCTGCTCGGATAAGTCCAGGCCGCTCAACGACTTGGTTAGCCAAAAAACAAC
TCAGCTGTCTGATATTACATCACGTTTTAATTCAGCTATTGAAGCACTGAACCGTT
TCATTCAGAAATATGATTCAGTGATGCAACGTCTGCTAGATGACACGTCTGGTAA
A
LcrV amino acid sequence
SEQ ID NO: 42
MIRAYEQNPQHFIEDLEKVRVEQLTGHGSSVLEELVQLVKDKNIDISIKYDPR
KDSEVFANRVITDDIELLKKILAYFLPEDAILKGGHYDNQLQNGIKRVKEFLESSPNT
QWELRAFMAVMHFSLTADRIDDDILKVIVDSMNHHGDARSKLREELAELTAELKIYS
VIQAEINKHLSSSGTINIHDKSINLMDKNLYGYTDEEIFKASAEYKILEKMPQTTIQVD
GSEKKIVSIKDFLGSENKRTGALGNLKNSYSYNKDNNELSHFATTCSDKSRPLNDLV
SQKTTQLSDITSRFNSAIEALNRFIQKYDSVMQRLLDDTSGK
YopB nucleic acid sequence
SEQ ID NO: 43
ATGAGTGCGTTGATAACCCATGATCGCTCAACGCCAGTAACTGGAAGTCT
ACTTCCCTACGTCGAGACACCAGCGCCCGCCCCCCTTCAGACTCAACAAGTCGCG
GGAGAACTGAAGGATAAAAATGGTGGGGTGAGTTCTCAGGGCGTACAGCTCCCT
GCACCACTAGCAGTGGTTGCCAGCCAAGTCACTGAAGGACAACAGCAAGAAATC
ACTAAATTATTGGAGTCGGTCACCCGCGGCACGGCAGGATCTCAACTGATATCA
AATTATGTTTCAGTGCTAACGAATTTTACGCTCGCTTCACCTGATACATTTGAGAT
TGAGTTAGGTAAGCTAGTTTCTAATTTAGAAGAAGTACGCAAAGACATAAAAAT
CGCTGATATTCAGCGTCTTCATGAACAAAACATGAAGAAAATTGAAGAGAATCA
AGAGAAAATCAAAGAAACAGAAGAGAATGCCAAGCAAGTCAAGAAATCCGGCA
TGGCATCAAAGATTTTTGGCTGGCTCAGCGCCATAGCCTCAGTGGTTATCGGTGC
CATCATGGTGGCCTCAGGGGTAGGAGCCGTTGCCGGTGCAATGATGATTGCCTCA
GGCGTAATTGGGATGGCGAATATGGCTGTGAAACAAGCGGCGGAAGATGGCCTG
ATATCCCAAGAGGCAATGCAAGTATTAGGGCCGATACTCACTGCGATTGAAGTC
GCATTGACTGTAGTTTCAACCGTAATGACCTTTGGCGGTTCGGCACTAAAATGCC
TGGCTGATATTGGCGCAAAACTCGGTGCTAACACCGCAAGTCTTGCTGCTAAAGG
AGCCGAGTTTTCGGCCAAAGTTGCCCAAATTTCGACAGGCATATCAAACACTGTC
GGGAATGCAGTGACTAAATTAGGGGGCAGTTTTGGTAGTTTAACAATGAGCCAT
GTAATCCGTACAGGATCACAGGCAACACAAGTCGCCGTTGGTGTGGGCAGCGGA
ATAACTCAGACCATCAATAATAAAAAACAAGCTGATTTACAACATAATAACGCT
GATTTGGCCTTGAACAAGGCAGACATGGCAGCGTTACAAAGTATTATTGACCGA
CTCAAAGAAGAGTTATCCCATTTGTCAGAGTCACATCAACAAGTGATGGAACTG
ATTTTCCAGATGATTAATGCAAAAGGTGACATGCTGCATAATTTGGCCGGCAGAC
CCCATACTGTTTAAGGTACC
YopB amino acid sequence
SEQ ID NO: 44
MSALITHDRSTPVTGSLLPYVETPAPAPLQTQQVAGELKDKNGGVSSQGVQL
PAPLAVVASQVTEGQQQEITKLLESVTRGTAGSQLISNYVSVLTNFTLASPDTFEIELG
KLVSNLEEVRKDIKIADIQRLHEQNMKKIEENQEKIKETEENAKQVKKSGMASKIFG
WLSAIASVVIGAIMVASGVGAVAGAMMIASGVIGMANMAVKQAAEDGLISQEAMQ
VLGPILTAIEVALTVVSTVMTFGGSALKCLADIGAKLGANTASLAAKGAEFSAKVAQ
ISTGISNTVGNAVTKLGGSFGSLTMSHVIRTGSQATQVAVGVGSGITQTINNKKQADL
QHNNADLALNKADMAALQSIIDRLKEELSHLSESHQQVMELIFQMINAKGDMLHNL
AGRPHTV
YerF nucleic acid sequence
SEQ ID NO: 45
CATATGATTAGAGCCTACGAACAAAACCCACAACATTTTATTGAGGATCT
AGAAAAAGTTAGGGTGGAACAACTTACTGGTCATGGTTCTTCAGTTTTAGAAGA
ATTGGTTCAGTTAGTCAAAGATAAAAATATAGATATTTCCATTAAATATGATCCC
AGAAAAGATTCGGAGGTTTTTGCCAATAGAGTAATTACTGATGATATCGAATTGC
TCAAGAAAATCCTAGCTTATTTTCTACCCGAGGATGCCATTCTTAAAGGCGGTCA
TTATGACAACCAACTGCAAAATGGCATCAAGCGAGTAAAAGAGTTCCTTGAATC
ATCGCCGAATACACAATGGGAATTGCGGGCGTTCATGGCAGTAATGCATTTCTCT
TTAACCGCCGATCGTATCGATGATGATATTTTGAAAGTGATTGTTGATTCAATGA
ATCATCATGGTGATGCCCGTAGCAAGTTGCGTGAAGAATTAGCTGAGCTTACCGC
CGAATTAAAGATTTATTCAGTTATTCAAGCCGAAATTAATAAGCATCTGTCTAGT
AGTGGCACCATAAATATCCATGATAAATCCATTAATCTCATGGATAAAAATTTAT
ATGGTTATACAGATGAAGAGATTTTTAAAGCCAGCGCAGAGTACAAAATTCTCG
AGAAAATGCCTCAAACCACCATTCAGGTGGATGGGAGCGAGAAAAAAATAGTCT
CGATAAAGGACTTTCTTGGAAGTGAGAATAAAAGAACCGGGGCGTTGGGTAATC
TGAAAAACTCATACTCTTATAATAAAGATAATAATGAATTATCTCACTTTGCCAC
CACCTGCTCGGATAAGTCCAGGCCGCTCAACGACTTGGTTAGCCAAAAAACAAC
TCAGCTGTCTGATATTACATCACGTTTTAATTCAGCTATTGAAGCACTGAACCGTT
TCATTCAGAAATATGATTCAGTGATGCAACGTCTGCTAGATGACACGTCTGGTAA
AGGATCCATGAGTGCGTTGATAACCCATGATCGCTCAACGCCAGTAACTGGAAG
TCTACTTCCCTACGTCGAGACACCAGCGCCCGCCCCCCTTCAGACTCAACAAGTC
GCGGGAGAACTGAAGGATAAAAATGGTGGGGTGAGTTCTCAGGGCGTACAGCTC
CCTGCACCACTAGCAGTGGTTGCCAGCCAAGTCACTGAAGGACAACAGCAAGAA
ATCACTAAATTATTGGAGTCGGTCACCCGCGGCACGGCAGGATCTCAACTGATAT
CAAATTATGTTTCAGTGCTAACGAATTTTACGCTCGCTTCACCTGATACATTTGAG
ATTGAGTTAGGTAAGCTAGTTTCTAATTTAGAAGAAGTACGCAAAGACATAAAA
ATCGCTGATATTCAGCGTCTTCATGAACAAAACATGAAGAAAATTGAAGAGAAT
CAAGAGAAAATCAAAGAAACAGAAGAGAATGCCAAGCAAGTCAAGAAATCCGG
CATGGCATCAAAGATTTTTGGCTGGCTCAGCGCCATAGCCTCAGTGGTTATCGGT
GCCATCATGGTGGCCTCAGGGGTAGGAGCCGTTGCCGGTGCAATGATGATTGCCT
CAGGCGTAATTGGGATGGCGAATATGGCTGTGAAACAAGCGGCGGAAGATGGCC
TGATATCCCAAGAGGCAATGCAAGTATTAGGGCCGATACTCACTGCGATTGAAG
TCGCATTGACTGTAGTTTCAACCGTAATGACCTTTGGCGGTTCGGCACTAAAATG
CCTGGCTGATATTGGCGCAAAACTCGGTGCTAACACCGCAAGTCTTGCTGCTAAA
GGAGCCGAGTTTTCGGCCAAAGTTGCCCAAATTTCGACAGGCATATCAAACACT
GTCGGGAATGCAGTGACTAAATTAGGGGGCAGTTTTGGTAGTTTAACAATGAGC
CATGTAATCCGTACAGGATCACAGGCAACACAAGTCGCCGTTGGTGTGGGCAGC
GGAATAACTCAGACCATCAATAATAAAAAACAAGCTGATTTACAACATAATAAC
GCTGATTTGGCCTTGAACAAGGCAGACATGGCAGCGTTACAAAGTATTATTGACC
GACTCAAAGAAGAGTTATCCCATTTGTCAGAGTCACATCAACAAGTGATGGAAC
TGATTTTCCAGATGATTAATGCAAAAGGTGACATGCTGCATAATTTGGCCGGCAG
ACCCCATACTGTTTAAGGTACC
YerF amino acid sequence
SEQ ID NO: 46
MIRAYEQNPQHFIEDLEKVRVEQLTGHGSSVLEELVQLVKDKNIDISIKYDPR
KDSEVFANRVITDDIELLKKILAYFLPEDAILKGGHYDNQLQNGIKRVKEFLESSPNT
QWELRAFMAVMHFSLTADRIDDDILKVIVDSMNHHGDARSKLREELAELTAELKIYS
VIQAEINKHLSSSGTINIHDKSINLMDKNLYGYTDEEIFKASAEYKILEKMPQTTIQVD
GSEKKIVSIKDFLGSENKRTGALGNLKNSYSYNKDNNELSHFATTCSDKSRPLNDLV
SQKTTQLSDITSRFNSAIEALNRFIQKYDSVMQRLLDDTSGKGSMSALITHDRSTPVT
GSLLPYVETPAPAPLQTQQVAGELKDKNGGVSSQGVQLPAPLAVVASQVTEGQQQE
ITKLLESVTRGTAGSQLISNYVSVLTNFTLASPDTFEIELGKLVSNLEEVRKDIKIADIQ
RLHEQNMKKIEENQEKIKETEENAKQVKKSGMASKIFGWLSAIASVVIGAIMVASGV
GAVAGAMMIASGVIGMANMAVKQAAEDGLISQEAMQVLGPILTAIEVALTVVSTV
MTFGGSALKCLADIGAKLGANTASLAAKGAEFSAKVAQISTGISNTVGNAVTKLGGS
FGSLTMSHVIRTGSQATQVAVGVGSGITQTINNKKQADLQHNNADLALNKADMAA
LQSIIDRLKEELSHLSESHQQVMELIFQMINAKGDMLHNLAGRPHTV
LTA1-YerF nucleic acid sequence
SEQ ID NO: 47
CATatggacaatggcgatcgtttataccgtgccgactcgcgtcccccagatgagattaaacgtagcggtgggttaatgcc
acgtgggcacaatgagtattttgaccgtggaacacagatgaacattaacctttacgatcatgcccgtgggacccagaccgggtttgtcc
gttatgatgacgggtatgttagtacgagtttgtccttacgctccgcacaccttgcgggacaaagtattttatcaggctacagcacatattac
atttatgtgatcgccactgccccaaacatgttcaatgtgaacgatgtgttgggggtttacagcccccatccatatgaacaagaagtctcg
gcccttggggggatcccatatagccagatttatggttggtaccgcgtaaattttggtgtgattgatgaacgtttgcatcgtaaccgtgaata
ccgcgatcgctactaccgtaacttgaacattgcacctgccgaggacggctatcgtttagcgggattcccacccgatcatcaggcgtgg
cgtgaggaaccgtggatccatcacgcccctcaggggtgcgggaacagtagtcgcCATATGATTAGAGCCTACGA
ACAAAACCCACAACATTTTATTGAGGATCTAGAAAAAGTTAGGGTGGAACAACT
TACTGGTCATGGTTCTTCAGTTTTAGAAGAATTGGTTCAGTTAGTCAAAGATAAA
AATATAGATATTTCCATTAAATATGATCCCAGAAAAGATTCGGAGGTTTTTGCCA
ATAGAGTAATTACTGATGATATCGAATTGCTCAAGAAAATCCTAGCTTATTTTCT
ACCCGAGGATGCCATTCTTAAAGGCGGTCATTATGACAACCAACTGCAAAATGG
CATCAAGCGAGTAAAAGAGTTCCTTGAATCATCGCCGAATACACAATGGGAATT
GCGGGCGTTCATGGCAGTAATGCATTTCTCTTTAACCGCCGATCGTATCGATGAT
GATATTTTGAAAGTGATTGTTGATTCAATGAATCATCATGGTGATGCCCGTAGCA
AGTTGCGTGAAGAATTAGCTGAGCTTACCGCCGAATTAAAGATTTATTCAGTTAT
TCAAGCCGAAATTAATAAGCATCTGTCTAGTAGTGGCACCATAAATATCCATGAT
AAATCCATTAATCTCATGGATAAAAATTTATATGGTTATACAGATGAAGAGATTT
TTAAAGCCAGCGCAGAGTACAAAATTCTCGAGAAAATGCCTCAAACCACCATTC
AGGTGGATGGGAGCGAGAAAAAAATAGTCTCGATAAAGGACTTTCTTGGAAGTG
AGAATAAAAGAACCGGGGCGTTGGGTAATCTGAAAAACTCATACTCTTATAATA
AAGATAATAATGAATTATCTCACTTTGCCACCACCTGCTCGGATAAGTCCAGGCC
GCTCAACGACTTGGTTAGCCAAAAAACAACTCAGCTGTCTGATATTACATCACGT
TTTAATTCAGCTATTGAAGCACTGAACCGTTTCATTCAGAAATATGATTCAGTGA
TGCAACGTCTGCTAGATGACACGTCTGGTAAAGGATCCATGAGTGCGTTGATAAC
CCATGATCGCTCAACGCCAGTAACTGGAAGTCTACTTCCCTACGTCGAGACACCA
GCGCCCGCCCCCCTTCAGACTCAACAAGTCGCGGGAGAACTGAAGGATAAAAAT
GGTGGGGTGAGTTCTCAGGGCGTACAGCTCCCTGCACCACTAGCAGTGGTTGCCA
GCCAAGTCACTGAAGGACAACAGCAAGAAATCACTAAATTATTGGAGTCGGTCA
CCCGCGGCACGGCAGGATCTCAACTGATATCAAATTATGTTTCAGTGCTAACGAA
TTTTACGCTCGCTTCACCTGATACATTTGAGATTGAGTTAGGTAAGCTAGTTTCTA
ATTTAGAAGAAGTACGCAAAGACATAAAAATCGCTGATATTCAGCGTCTTCATG
AACAAAACATGAAGAAAATTGAAGAGAATCAAGAGAAAATCAAAGAAACAGAA
GAGAATGCCAAGCAAGTCAAGAAATCCGGCATGGCATCAAAGATTTTTGGCTGG
CTCAGCGCCATAGCCTCAGTGGTTATCGGTGCCATCATGGTGGCCTCAGGGGTAG
GAGCCGTTGCCGGTGCAATGATGATTGCCTCAGGCGTAATTGGGATGGCGAATA
TGGCTGTGAAACAAGCGGCGGAAGATGGCCTGATATCCCAAGAGGCAATGCAAG
TATTAGGGCCGATACTCACTGCGATTGAAGTCGCATTGACTGTAGTTTCAACCGT
AATGACCTTTGGCGGTTCGGCACTAAAATGCCTGGCTGATATTGGCGCAAAACTC
GGTGCTAACACCGCAAGTCTTGCTGCTAAAGGAGCCGAGTTTTCGGCCAAAGTTG
CCCAAATTTCGACAGGCATATCAAACACTGTCGGGAATGCAGTGACTAAATTAG
GGGGCAGTTTTGGTAGTTTAACAATGAGCCATGTAATCCGTACAGGATCACAGG
CAACACAAGTCGCCGTTGGTGTGGGCAGCGGAATAACTCAGACCATCAATAATA
AAAAACAAGCTGATTTACAACATAATAACGCTGATTTGGCCTTGAACAAGGCAG
ACATGGCAGCGTTACAAAGTATTATTGACCGACTCAAAGAAGAGTTATCCCATTT
GTCAGAGTCACATCAACAAGTGATGGAACTGATTTTCCAGATGATTAATGCAAA
AGGTGACATGCTGCATAATTTGGCCGGCAGACCCCATACTGTTTAAGGTACC
LTA1-YerF Amino acid sequence
SEQ ID NO: 48
MDNGDRLYRADSRPPDEIKRSGGLMPRGHNEYFDRGTQMNINLYDHARGTQ
TGFVRYDDGYVSTSLSLRSAHLAGQSILSGYSTYYIYVIATAPNMFNVNDVLGVYSP
HPYEQEVSALGGIPYSQIYGWYRVNFGVIDERLHRNREYRDRYYRNLNIAPAEDGYR
LAGFPPDHQAWREEPWIHHAPQGCGNSSRMIRAYEQNPQHFIEDLEKVRVEQLTGH
GSSVLEELVQLVKDKNIDISIKYDPRKDSEVFANRVITDDIELLKKILAYFLPEDAILK
GGHYDNQLQNGIKRVKEFLESSPNTQWELRAFMAVMHFSLTADRIDDDILKVIVDS
MNHHGDARSKLREELAELTAELKIYSVIQAEINKHLSSSGTINIHDKSINLMDKNLYG
YTDEEIFKASAEYKILEKMPQTTIQVDGSEKKIVSIKDFLGSENKRTGALGNLKNSYS
YNKDNNELSHFATTCSDKSRPLNDLVSQKTTQLSDITSRFNSAIEALNRFIQKYDSVM
QRLLDDTSGKGSMSALITHDRSTPVTGSLLPYVETPAPAPLQTQQVAGELKDKNGGV
SSQGVQLPAPLAVVASQVTEGQQQEITKLLESVTRGTAGSQLISNYVSVLTNFTLASP
DTFEIELGKLVSNLEEVRKDIKIADIQRLHEQNMKKIEENQEKIKETEENAKQVKKSG
MASKIFGWLSAIASVVIGAIMVASGVGAVAGAMMIASGVIGMANMAVKQAAEDGL
ISQEAMQVLGPILTAIEVALTVVSTVMTFGGSALKCLADIGAKLGANTASLAAKGAE
FSAKVAQISTGISNTVGNAVTKLGGSFGSLTMSHVIRTGSQATQVAVGVGSGITQTIN
NKKQADLQHNNADLALNKADMAALQSIIDRLKEELSHLSESHQQVMELIFQMINAK
GDMLHNLAGRPHTV-
His-SicA chaperone for S1 nucleic sequence
SEQ ID NO: 49
ATGGGCAGCAGCCATCACCATCATCACCACAGCCAGGATCCGatggactaccaga
acaacgtcagcgaagaacgtgttgcggaaatgatttgggatgccgttagtgaaggcgccacgctaaaagacgttcatggaatccctca
agatatgatggacggtttatatgctcatgcttatgagttttataaccagggacgactggatgaagctgagacgttctttcgtttcttatgcatt
tatgatttttacaatcccgattacaccatgggactggcggcagtatgccaactgaaaaaacaatttcagaaagcatgtgacctttatgcag
tagcgtttacgttacttaaaaatgattatcgccccgttttttttaccgggcagtgtcaattattaatgcgtaaggcagcaaaagccagacagt
gttttgaacttgtcaatgaacgtactgaagatgagtctctgcgggcaaaagcgttggtctatctggaggcgctaaaaacggcggagaca
gagcagcacagcgagcaggagaaggagtaaAAGCTT
His-SicA chaperone for S1 Amino acid sequence
SEQ ID NO: 50
MGSSHHHHHHSQDPMDYQNNVSEERVAEMIWDAVSEGATLKDVHGIPQDM
MDGLYAHAYEFYNQGRLDEAETFFRFLCIYDFYNPDYTMGLAAVCQLKKQFQKAC
DLYAVAFTLLKNDYRPVFFTGQCQLLMRKAAKARQCFELVNERTEDESLRAKALVY
LEALKTAETEQHSEQEKE*
SipD nucleic acid sequence
SEQ ID NO: 51
atgcttaatattcaaaattattccgcttctcctcatccggggatcgttgccgaacggccgcagactccctcggcgagcgagc
acgtcgagactgccgtggtaccgtctaccacagaacatcgcggtacagatatcatttcattatcgcaggcggctactaaaatccaccag
gcacagcagacgctgcagtcaacgccaccgatctctgaagagaataatgacgagcgcacgctggcgcgccagcagttgaccagca
gcctgaatgcgctggcgaagtccggcgtgtcattatccgcagaacaaaatgagaacctgcggagcgcgttttctgcgccgacgtcgg
ccttatttagcgcttcgcctatggcgcagccgagaacaaccatttctgatgctgagatttgggatatggtttcccaaaatatatcggcgata
ggtgacagctatctgggcgtttatgaaaacgttgtcgcagtctataccgatttttatcaggccttcagtgatattctttccaaaatgggagg
ctggttattaccaggtaaggacggtaataccgttaagctagatgttacctcactcaaaaatgatttaaacagtttagtcaataaatataatca
aataaacagtaataccgttttatttccagcgcagtcaggcagcggcgttaaagtagccactgaagcggaagcgagacagtggctcagt
gaattgaatttaccgaatagctgcctgaaatcttatggatccggttatgtcgtcaccgttgatctgacgccattacaaaaaatggttcagga
tattgatggtttaggcgcgccgggaaaagactcaaaactcgaaatggataacgccaaatatcaagcctggcagtcgggttttaaagcg
caggaagaaaatatgaaaaccacattacagacgctgacgcaaaaatatagcaatgccaattcattgtacgacaacctggtaaaagtgct
gagcagtacgataagtagcagcctggaaaccgccaaaagcttcctgcaagga
SipD amino acid sequence
SEQ ID NO: 52
MLNIQNYSASPHPGIVAERPQTPSASEHVETAVVPSTTEHRGTDIISLSQAATKI
HQAQQTLQSTPPISEENNDERTLARQQLTSSLNALAKSGVSLSAEQNENLRSAFSAPT
SALFSASPMAQPRTTISDAEIWDMVSQNISAIGDSYLGVYENVVAVYTDFYQAFSDIL
SKMGGWLLPGKDGNTVKLDVTSLKNDLNSLVNKYNQINSNTVLFPAQSGSGVKVA
TEAEARQWLSELNLPNSCLKSYGSGYVVTVDLTPLQKMVQDIDGLGAPGKDSKLEM
DNAKYQAWQSGFKAQEENMKTTLQTLTQKYSNANSLYDNLVKVLSSTISSSLETAK
SFLQG
SipB nucleic acid sequence
SEQ ID NO: 53
atggtaaatgacgcaagtagcattagccgtagcggatatacccaaaatccgcgcctcgctgaggcggcttttgaaggcgtt
cgtaagaacacggactttttaaaagcggcggataaagcttttaaagatgtggtggcaacgaaagcgggcgaccttaaagccggaaca
aagtccggcgagagcgctattaatacggtgggtctaaagccgcctacggacgccgcccgggaaaaactctccagcgaagggcaatt
gacattactgcttggcaagttaatgaccctactgggcgatgtttcgctgtctcaactggagtctcgtctggcggtatggcaggcgatgatt
gagtcacaaaaagagatggggattcaggtatcgaaagaattccagacggctctgggagaggctcaggaggcgacggatctctatga
agccagtatcaaaaagacggataccgccaagagtgtttatgacgctgcgaccaaaaaactgacgcaggcgcaaaataaattgcaatc
gctggacccggctgaccccggctatgcacaagctgaagccgcggtagaacaggccggaaaagaagcgacagaggcgaaagagg
ccttagataaggccacggatgcgacggttaaagcaggcacagacgccaaagcgaaagccgagaaagcggataacattctgaccaa
attccagggaacggctaatgccgcctctcagaatcaggtttcccagggtgagcaggataatctgtcaaatgtcgcccgcctcactatgc
tcatggccatgtttattgagattgtgggcaaaaatacggaagaaagcctgcaaaacgatcttgcgcttttcaacgccttgcaggaaggg
cgtcaggcggagatggaaaagaaatcggctgaattccaggaagagacgcgcaaagccgaggaaacgaaccgcattatgggatgta
tcgggaaagtcctcggcgcgctgctaaccattgtcagcgttgtggccgctgtttttaccggtggggcgagtctggcgctggctgcggt
gggacttgcggtaatggtggccgatgaaattgtgaaggcggcgacgggagtgtcgtttattcagcaggcgctaaacccgattatgga
gcatgtgctgaagccgttaatggagctgattggcaaggcgattaccaaagcgctggaaggattaggcgtcgataagaaaacggcag
agatggccggcagcattgttggtgcgattgtcgccgctattgccatggtggcggtcattgtggtggtcgcagttgtcgggaaaggcgc
ggcggcgaaactgggtaacgcgctgagcaaaatgatgggcgaaacgattaagaagttggtgcctaacgtgctgaaacagttggcgc
aaaacggcagcaaactctttacccaggggatgcaacgtattactagcggtctgggtaatgtgggtagcaagatgggcctgcaaacga
atgccttaagtaaagagctggtaggtaataccctaaataaagtggcgttgggcatggaagtcacgaataccgcagcccagtcagccg
gtggtgttgccgagggcgtatttattaaaaatgccagcgaggcgcttgctgattttatgctcgcccgttttgccatggatcagattcagca
gtggcttaaacaatccgtagaaatatttggtgaaaaccagaaggtaacggcggaactgcaaaaagccatgtcttctgcggtacagcaa
aatgcggatgcttcgcgttttattctgcgccagagtcgcgcataa
SipB amino acid sequence
SEQ ID NO: 54
MVNDASSISRSGYTQNPRLAEAAFEGVRKNTDFLKAADKAFKDVVATKAGD
LKAGTKSGESAINTVGLKPPTDAAREKLSSEGQLTLLLGKLMTLLGDVSLSQLESRL
AVWQAMIESQKEMGIQVSKEFQTALGEAQEATDLYEASIKKTDTAKSVYDAATKKL
TQAQNKLQSLDPADPGYAQAEAAVEQAGKEATEAKEALDKATDATVKAGTDAKA
KAEKADNILTKFQGTANAASQNQVSQGEQDNLSNVARLTMLMAMFIEIVGKNTEES
LQNDLALFNALQEGRQAEMEKKSAEFQEETRKAEETNRIMGCIGKVLGALLTIVSVV
AAVFTGGASLALAAVGLAVMVADEIVKAATGVSFIQQALNPIMEHVLKPLMELIGK
AITKALEGLGVDKKTAEMAGSIVGAIVAAIAMVAVIVVVAVVGKGAAAKLGNALS
KMMGETIKKLVPNVLKQLAQNGSKLFTQGMQRITSGLGNVGSKMGLQTNALSKEL
VGNTLNKVALGMEVTNTAAQSAGGVAEGVFIKNASEALADFMLARFAMDQIQQWL
KQSVEIFGENQKVTAELQKAMSSAVQQNADASRFILRQSRA
S1 nucleic acid sequence
SEQ ID NO: 55
atgcttaatattcaaaattattccgcttctcctcatccggggatcgttgccgaacggccgcagactccctcggcgagcgagc
acgtcgagactgccgtggtaccgtctaccacagaacatcgcggtacagatatcatttcattatcgcaggcggctactaaaatccaccag
gcacagcagacgctgcagtcaacgccaccgatctctgaagagaataatgacgagcgcacgctggcgcgccagcagttgaccagca
gcctgaatgcgctggcgaagtccggcgtgtcattatccgcagaacaaaatgagaacctgcggagcgcgttttctgcgccgacgtcgg
ccttatttagcgcttcgcctatggcgcagccgagaacaaccatttctgatgctgagatttgggatatggtttcccaaaatatatcggcgata
ggtgacagctatctgggcgtttatgaaaacgttgtcgcagtctataccgatttttatcaggccttcagtgatattctttccaaaatgggagg
ctggttattaccaggtaaggacggtaataccgttaagctagatgttacctcactcaaaaatgatttaaacagtttagtcaataaatataatca
aataaacagtaataccgttttatttccagcgcagtcaggcagcggcgttaaagtagccactgaagcggaagcgagacagtggctcagt
gaattgaatttaccgaatagctgcctgaaatcttatggatccggttatgtcgtcaccgttgatctgacgccattacaaaaaatggttcagga
tattgatggtttaggcgcgccgggaaaagactcaaaactcgaaatggataacgccaaatatcaagcctggcagtcgggttttaaagcg
caggaagaaaatatgaaaaccacattacagacgctgacgcaaaaatatagcaatgccaattcattgtacgacaacctggtaaaagtgct
gagcagtacgataagtagcagcctggaaaccgccaaaagcttcctgcaaggagtcgacatggtaaatgacgcaagtagcattagcc
gtagcggatatacccaaaatccgcgcctcgctgaggcggcttttgaaggcgttcgtaagaacacggactttttaaaagcggcggataa
agcttttaaagatgtggtggcaacgaaagcgggcgaccttaaagccggaacaaagtccggcgagagcgctattaatacggtgggtct
aaagccgcctacggacgccgcccgggaaaaactctccagcgaagggcaattgacattactgcttggcaagttaatgaccctactggg
cgatgtttcgctgtctcaactggagtctcgtctggcggtatggcaggcgatgattgagtcacaaaaagagatggggattcaggtatcga
aagaattccagacggctctgggagaggctcaggaggcgacggatctctatgaagccagtatcaaaaagacggataccgccaagagt
gtttatgacgctgcgaccaaaaaactgacgcaggcgcaaaataaattgcaatcgctggacccggctgaccccggctatgcacaagct
gaagccgcggtagaacaggccggaaaagaagcgacagaggcgaaagaggccttagataaggccacggatgcgacggttaaagc
aggcacagacgccaaagcgaaagccgagaaagcggataacattctgaccaaattccagggaacggctaatgccgcctctcagaatc
aggtttcccagggtgagcaggataatctgtcaaatgtcgcccgcctcactatgctcatggccatgtttattgagattgtgggcaaaaatac
ggaagaaagcctgcaaaacgatcttgcgcttttcaacgccttgcaggaagggcgtcaggcggagatggaaaagaaatcggctgaatt
ccaggaagagacgcgcaaagccgaggaaacgaaccgcattatgggatgtatcgggaaagtcctcggcgcgctgctaaccattgtca
gcgttgtggccgctgtttttaccggtggggcgagtctggcgctggctgcggtgggacttgcggtaatggtggccgatgaaattgtgaa
ggcggcgacgggagtgtcgtttattcagcaggcgctaaacccgattatggagcatgtgctgaagccgttaatggagctgattggcaag
gcgattaccaaagcgctggaaggattaggcgtcgataagaaaacggcagagatggccggcagcattgttggtgcgattgtcgccgct
attgccatggtggcggtcattgtggtggtcgcagttgtcgggaaaggcgcggcggcgaaactgggtaacgcgctgagcaaaatgat
gggcgaaacgattaagaagttggtgcctaacgtgctgaaacagttggcgcaaaacggcagcaaactctttacccaggggatgcaac
gtattactagcggtctgggtaatgtgggtagcaagatgggcctgcaaacgaatgccttaagtaaagagctggtaggtaataccctaaat
aaagtggcgttgggcatggaagtcacgaataccgcagcccagtcagccggtggtgttgccgagggcgtatttattaaaaatgccagc
gaggcgcttgctgattttatgctcgcccgttttgccatggatcagattcagcagtggcttaaacaatccgtagaaatatttggtgaaaacc
agaaggtaacggcggaactgcaaaaagccatgtcttctgcggtacagcaaaatgcggatgcttcgcgttttattctgcgccagagtcg
cgcataa
S1 amino acid sequence
SEQ ID NO: 56
MLNIQNYSASPHPGIVAERPQTPSASEHVETAVVPSTTEHRGTDIISLSQAATKI
HQAQQTLQSTPPISEENNDERTLARQQLTSSLNALAKSGVSLSAEQNENLRSAFSAPT
SALFSASPMAQPRTTISDAEIWDMVSQNISAIGDSYLGVYENVVAVYTDFYQAFSDIL
SKMGGWLLPGKDGNTVKLDVTSLKNDLNSLVNKYNQINSNTVLFPAQSGSGVKVA
TEAEARQWLSELNLPNSCLKSYGSGYVVTVDLTPLQKMVQDIDGLGAPGKDSKLEM
DNAKYQAWQSGFKAQEENMKTTLQTLTQKYSNANSLYDNLVKVLSSTISSSLETAK
SFLQGVDMVNDASSISRSGYTQNPRLAEAAFEGVRKNTDFLKAADKAFKDVVATKA
GDLKAGTKSGESAINTVGLKPPTDAAREKLSSEGQLTLLLGKLMTLLGDVSLSQLES
RLAVWQAMIESQKEMGIQVSKEFQTALGEAQEATDLYEASIKKTDTAKSVYDAATK
KLTQAQNKLQSLDPADPGYAQAEAAVEQAGKEATEAKEALDKATDATVKAGTDA
KAKAEKADNILTKFQGTANAASQNQVSQGEQDNLSNVARLTMLMAMFIEIVGKNTE
ESLQNDLALFNALQEGRQAEMEKKSAEFQEETRKAEETNRIMGCIGKVLGALLTIVS
VVAAVFTGGASLALAAVGLAVMVADEIVKAATGVSFIQQALNPIMEHVLKPLMELI
GKAITKALEGLGVDKKTAEMAGSIVGAIVAAIAMVAVIVVVAVVGKGAAAKLGNA
LSKMMGETIKKLVPNVLKQLAQNGSKLFTQGMQRITSGLGNVGSKMGLQTNALSKE
LVGNTLNKVALGMEVTNTAAQSAGGVAEGVFIKNASEALADFMLARFAMDQIQQW
LKQSVEIFGENQKVTAELQKAMSSAVQQNADASRFILRQSRA
LTA1-GSAAS-S1 Nucleic acid sequence
SEQ ID NO: 57
CATatggacaatggcgatcgtttataccgtgccgactcgcgtcccccagatgagattaaacgtagcggtgggttaatgcc
acgtgggcacaatgagtattttgaccgtggaacacagatgaacattaacctttacgatcatgcccgtgggacccagaccgggtttgtcc
gttatgatgacgggtatgttagtacgagtttgtccttacgctccgcacaccttgcgggacaaagtattttatcaggctacagcacatattac
atttatgtgatcgccactgccccaaacatgttcaatgtgaacgatgtgttgggggtttacagcccccatccatatgaacaagaagtctcg
gcccttggggggatcccatatagccagatttatggttggtaccgcgtaaattttggtgtgattgatgaacgtttgcatcgtaaccgtgaata
ccgcgatcgctactaccgtaacttgaacattgcacctgccgaggacggctatcgtttagcgggattcccacccgatcatcaggcgtgg
cgtgaggaaccgtggatccatcacgcccctcaggggtgcgggaacagtagtcgcgggtccgcggcatccatgcttaatattcaaaatt
attccgcttctcctcatccggggatcgttgccgaacggccgcagactccctcggcgagcgagcacgtcgagactgccgtggtaccgt
ctaccacagaacatcgcggtacagatatcatttcattatcgcaggcggctactaaaatccaccaggcacagcagacgctgcagtcaac
gccaccgatctctgaagagaataatgacgagcgcacgctggcgcgccagcagttgaccagcagcctgaatgcgctggcgaagtcc
ggcgtgtcattatccgcagaacaaaatgagaacctgcggagcgcgttttctgcgccgacgtcggccttatttagcgcttcgcctatggc
gcagccgagaacaaccatttctgatgctgagatttgggatatggtttcccaaaatatatcggcgataggtgacagctatctgggcgtttat
gaaaacgttgtcgcagtctataccgatttttatcaggccttcagtgatattctttccaaaatgggaggctggttattaccaggtaaggacgg
taataccgttaagctagatgttacctcactcaaaaatgatttaaacagtttagtcaataaatataatcaaataaacagtaataccgttttatttc
cagcgcagtcaggcagcggcgttaaagtagccactgaagcggaagcgagacagtggctcagtgaattgaatttaccgaatagctgc
ctgaaatcttatggatccggttatgtcgtcaccgttgatctgacgccattacaaaaaatggttcaggatattgatggtttaggcgcgccgg
gaaaagactcaaaactcgaaatggataacgccaaatatcaagcctggcagtcgggttttaaagcgcaggaagaaaatatgaaaacca
cattacagacgctgacgcaaaaatatagcaatgccaattcattgtacgacaacctggtaaaagtgctgagcagtacgataagtagcagc
ctggaaaccgccaaaagcttcctgcaaggagtcgacatggtaaatgacgcaagtagcattagccgtagcggatatacccaaaatccg
cgcctcgctgaggcggcttttgaaggcgttcgtaagaacacggactttttaaaagcggcggataaagcttttaaagatgtggtggcaac
gaaagcgggcgaccttaaagccggaacaaagtccggcgagagcgctattaatacggtgggtctaaagccgcctacggacgccgcc
cgggaaaaactctccagcgaagggcaattgacattactgcttggcaagttaatgaccctactgggcgatgtttcgctgtctcaactgga
gtctcgtctggcggtatggcaggcgatgattgagtcacaaaaagagatggggattcaggtatcgaaagaattccagacggctctggg
agaggctcaggaggcgacggatctctatgaagccagtatcaaaaagacggataccgccaagagtgtttatgacgctgcgaccaaaa
aactgacgcaggcgcaaaataaattgcaatcgctggacccggctgaccccggctatgcacaagctgaagccgcggtagaacaggc
cggaaaagaagcgacagaggcgaaagaggccttagataaggccacggatgcgacggttaaagcaggcacagacgccaaagcga
aagccgagaaagcggataacattctgaccaaattccagggaacggctaatgccgcctctcagaatcaggtttcccagggtgagcagg
ataatctgtcaaatgtcgcccgcctcactatgctcatggccatgtttattgagattgtgggcaaaaatacggaagaaagcctgcaaaacg
atcttgcgcttttcaacgccttgcaggaagggcgtcaggcggagatggaaaagaaatcggctgaattccaggaagagacgcgcaaa
gccgaggaaacgaaccgcattatgggatgtatcgggaaagtcctcggcgcgctgctaaccattgtcagcgttgtggccgctgtttttac
cggtggggcgagtctggcgctggctgcggtgggacttgcggtaatggtggccgatgaaattgtgaaggcggcgacgggagtgtcgt
ttattcagcaggcgctaaacccgattatggagcatgtgctgaagccgttaatggagctgattggcaaggcgattaccaaagcgctgga
aggattaggcgtcgataagaaaacggcagagatggccggcagcattgttggtgcgattgtcgccgctattgccatggtggcggtcatt
gtggtggtcgcagttgtcgggaaaggcgcggcggcgaaactgggtaacgcgctgagcaaaatgatgggcgaaacgattaagaagt
tggtgcctaacgtgctgaaacagttggcgcaaaacggcagcaaactctttacccaggggatgcaacgtattactagcggtctgggtaa
tgtgggtagcaagatgggcctgcaaacgaatgccttaagtaaagagctggtaggtaataccctaaataaagtggcgttgggcatggaa
gtcacgaataccgcagcccagtcagccggtggtgttgccgagggcgtatttattaaaaatgccagcgaggcgcttgctgattttatgct
cgcccgttttgccatggatcagattcagcagtggcttaaacaatccgtagaaatatttggtgaaaaccagaaggtaacggcggaactgc
aaaaagccatgtcttctgcggtacagcaaaatgcggatgcttcgcgttttattctgcgccagagtcgcgcataaCTCGAG
LTA1-GSAAS-S1 Amino acid sequence
SEQ ID NO: 58
MDNGDRLYRADSRPPDEIKRSGGLMPRGHNEYFDRGTQMNINLYDHARGTQ
TGFVRYDDGYVSTSLSLRSAHLAGQSILSGYSTYYIYVIATAPNMFNVNDVLGVYSP
HPYEQEVSALGGIPYSQIYGWYRVNFGVIDERLHRNREYRDRYYRNLNIAPAEDGYR
LAGFPPDHQAWREEPWIHHAPQGCGNSSRGSAASMLNIQNYSASPHPGIVAERPQTP
SASEHVETAVVPSTTEHRGTDIISLSQAATKIHQAQQTLQSTPPISEENNDERTLARQQ
LTSSLNALAKSGVSLSAEQNENLRSAFSAPTSALFSASPMAQPRTTISDAEIWDMVSQ
NISAIGDSYLGVYENVVAVYTDFYQAFSDILSKMGGWLLPGKDGNTVKLDVTSLKN
DLNSLVNKYNQINSNTVLFPAQSGSGVKVATEAEARQWLSELNLPNSCLKSYGSGY
VVTVDLTPLQKMVQDIDGLGAPGKDSKLEMDNAKYQAWQSGFKAQEENMKTTLQ
TLTQKYSNANSLYDNLVKVLSSTISSSLETAKSFLQGVDMVNDASSISRSGYTQNPRL
AEAAFEGVRKNTDFLKAADKAFKDVVATKAGDLKAGTKSGESAINTVGLKPPTDA
AREKLSSEGQLTLLLGKLMTLLGDVSLSQLESRLAVWQAMIESQKEMGIQVSKEFQT
ALGEAQEATDLYEASIKKTDTAKSVYDAATKKLTQAQNKLQSLDPADPGYAQAEA
AVEQAGKEATEAKEALDKATDATVKAGTDAKAKAEKADNILTKFQGTANAASQNQ
VSQGEQDNLSNVARLTMLMAMFIEIVGKNTEESLQNDLALFNALQEGRQAEMEKKS
AEFQEETRKAEETNRIMGCIGKVLGALLTIVSVVAAVFTGGASLALAAVGLAVMVA
DEIVKAATGVSFIQQALNPIMEHVLKPLMELIGKAITKALEGLGVDKKTAEMAGSIV
GAIVAAIAMVAVIVVVAVVGKGAAAKLGNALSKMMGETIKKLVPNVLKQLAQNGS
KLFTQGMQRITSGLGNVGSKMGLQTNALSKELVGNTLNKVALGMEVTNTAAQSAG
GVAEGVFIKNASEALADFMLARFAMDQIQQWLKQSVEIFGENQKVTAELQKAMSSA
VQQNADASRFILRQSRA*
His-SscA chaperone for S2 nucleic acid sequence
SEQ ID NO: 59
ATGGGCAGCAGCCATCACCATCATCACCACAGCCAGGATCCGatgaaaaaagac
ccgaccctacaacaggcacatgacacgatgcggtttttccggcgtggcggctcgctgcgtatgttgttggatgacgatgttacacagcc
gcttaatactctgtatcgctatgccacgcagcttatggaggtaaaagaattcgccggcgcagcgcgactttttcaattgctgacgatatat
gatgcctggtcatttgactactggtttcggttaggggaatgctgccaggctcaaaaacattggggggaagcgatatacgcttatggacg
cgcggcacaaattaagattgatgcgccgcaggcgccatgggccgcagcggaatgctatctcgcgtgtgataacgtctgttatgcaatc
aaagcgttaaaggccgtggtgcgtatttgcggcgaggtcagtgaacatcaaattctccgacagcgtgcagaaaagatgttacagcaac
tttctgacaggagctaaAAGCTT
His-SscA chaperone for S2 Amino acid sequence
SEQ ID NO: 60
MGSSHHHHHHSQDPMKKDPTLQQAHDTMRFFRRGGSLRMLLDDDVTQPLN
TLYRYATQLMEVKEFAGAARLFQLLTIYDAWSFDYWFRLGECCQAQKHWGEAIYA
YGRAAQIKIDAPQAPWAAAECYLACDNVCYAIKALKAVVRICGEVSEHQILRQRAE
KMLQQLSDRS*
SseB nucleic acid sequence
SEQ ID NO: 61
Atgtcttcaggaaacatcttatggggaagtcaaaaccctattgtgtttaaaaatagcttcggcgtcagcaacgctgataccgg
gagccaggatgacttatcccagcaaaatccgtttgccgaagggtatggtgttttgcttattctccttatggttattcaggctatcgcaaataa
taaatttattgaagtccagaagaacgctgaacgtgccagaaatacccaggaaaagtcaaatgagatggatgaggtgattgctaaagca
gccaaaggggatgctaaaaccaaagaggaggtgcctgaggatgtaattaaatacatgcgtgataatggtattctcatcgatggtatgac
cattgatgattatatggctaaatatggcgatcatgggaagctggataaaggtggcctacaggcgatcaaagcggctttggataatgacg
ccaaccggaataccgatcttatgagtcaggggcagataacaattcaaaaaatgtctcaggagcttaacgctgtccttacccaactgaca
gggcttatcagtaagtggggggaaatttccagtatgatagcgcagaaaacgtactca
SseB amino acid sequence
SEQ ID NO: 62
MSSGNILWGSQNPIVFKNSFGVSNADTGSQDDLSQQNPFAEGYGVLLILLMVI
QAIANNKFIEVQKNAERARNTQEKSNEMDEVIAKAAKGDAKTKEEVPEDVIKYMRD
NGILIDGMTIDDYMAKYGDHGKLDKGGLQAIKAALDNDANRNTDLMSQGQITIQK
MSQELNAVLTQLTGLISKWGEISSMIAQKTYS
SseC nucleic acid sequence
SEQ ID NO: 63
atgaatcgaattcacagtaatagcgacagcgccgcaggagtaaccgccttaacacatcatcacttaagcaatgtcagttgcg
tttcctcgggttcgctgggaaagcgccagcatcgtgtgaattctacttttggcgatggcaacgccgcgtgtctgctatccgggaaaatta
gtcttcaggaggcaagcaatgcgttgaagcaactgcttgatgccgtacccggaaatcataagcgtccatcattgcctgactttttgcaga
ccaatcccgcggttttatcaatgatgatgacgtcattaatactcaacgtctttggtaataacgctcaatcgttatgccaacagcttgagcgg
gcaactgaggtgcaaaatgcattacgtaataagcaggtaaaggagtatcaggagcagatccagaaagcgatagagcaggaggataa
agcgcgtaaagcgggtatttttggcgctatttttgactggattaccggcatatttgaaaccgtgattggcgccttaaaagttgtggaaggtt
ttctgtccggaaatcccgcagaaatggctagcggcgtagcttatatggccgcaggttgtgcaggaatggttaaagccggagccgaaa
cggcaatgatgtgcggtgctgaccacgatacctgtcaggcaattattgacgtgacaagtaagattcaatttggttgtgaagccgtcgcg
ctggcactggatgttttccagattggccgtgcttttatggcgacgagaggtttatctggcgcagctgcaaaagtgcttgactccggttttg
gcgaggaagtggttgagcgtatggtaggtgcaggggaagcagaaatagaggagttggctgaaaagtttggcgaagaagtgagcga
aagtttttccaaacaatttgagccgcttgaacgtgaaatggctatggcgaatgagatggcagaggaggctgccgagttttctcgtaacgt
agaaaataatatgacgcgaagcgcgggaaaaagctttacgaaagagggggtgaaagcaatggcaaaagaagcggcaaaagaagc
cctggaaaaatgtgtgcaagaaggtggaaagttcctgttaaaaaaattccgtaataaagttctcttcaatatgttcaaaaaaatcctgtatg
ccttactgagggattgttcatttaaaggcttacaggctatcagatgtgcaaccgagggcgccagtcagatgaatactggcatggttaaca
cagaaaaagcgaagatcgaaaagaaaatagagcaattaataactcagcaacggtttctggatttcataatgcaacaaacagaaaacca
gaaaaagatagaacaaaaacgcttagaggagctttataaggggagcggtgccgcgcttagagatgtattagataccattgatcactata
gtagcgttcaggcgagaatagctggctatcgcgcttaa
SseC amino acid sequence
SEQ ID NO: 64
MNRIHSNSDSAAGVTALTHHHLSNVSCVSSGSLGKRQHRVNSTFGDGNAAC
LLSGKISLQEASNALKQLLDAVPGNHKRPSLPDFLQTNPAVLSMMMTSLILNVFGNN
AQSLCQQLERATEVQNALRNKQVKEYQEQIQKAIEQEDKARKAGIFGAIFDWITGIFE
TVIGALKVVEGFLSGNPAEMASGVAYMAAGCAGMVKAGAETAMMCGADHDTCQ
AIIDVTSKIQFGCEAVALALDVFQIGRAFMATRGLSGAAAKVLDSGFGEEVVERMVG
AGEAEIEELAEKFGEEVSESFSKQFEPLEREMAMANEMAEEAAEFSRNVENNMTRSA
GKSFTKEGVKAMAKEAAKEALEKCVQEGGKFLLKKFRNKVLFNMFKKILYALLRD
CSFKGLQAIRCATEGASQMNTGMVNTEKAKIEKKIEQLITQQRFLDFIMQQTENQKKI
EQKRLEELYKGSGAALRDVLDTIDHYSSVQARIAGYRA
S2 nucleic acid sequence
SEQ ID NO: 65
atgtcttcaggaaacatcttatggggaagtcaaaaccctattgtgtttaaaaatagcttcggcgtcagcaacgctgataccgg
gagccaggatgacttatcccagcaaaatccgtttgccgaagggtatggtgttttgcttattctccttatggttattcaggctatcgcaaataa
taaatttattgaagtccagaagaacgctgaacgtgccagaaatacccaggaaaagtcaaatgagatggatgaggtgattgctaaagca
gccaaaggggatgctaaaaccaaagaggaggtgcctgaggatgtaattaaatacatgcgtgataatggtattctcatcgatggtatgac
cattgatgattatatggctaaatatggcgatcatgggaagctggataaaggtggcctacaggcgatcaaagcggctttggataatgacg
ccaaccggaataccgatcttatgagtcaggggcagataacaattcaaaaaatgtctcaggagcttaacgctgtccttacccaactgaca
gggcttatcagtaagtggggggaaatttccagtatgatagcgcagaaaacgtactcaGAGCTCatgaatcgaattcacagtaata
gcgacagcgccgcaggagtaaccgccttaacacatcatcacttaagcaatgtcagttgcgtttcctcgggttcgctgggaaagcgcca
gcatcgtgtgaattctacttttggcgatggcaacgccgcgtgtctgctatccgggaaaattagtcttcaggaggcaagcaatgcgttgaa
gcaactgcttgatgccgtacccggaaatcataagcgtccatcattgcctgactttttgcagaccaatcccgcggttttatcaatgatgatg
acgtcattaatactcaacgtctttggtaataacgctcaatcgttatgccaacagcttgagcgggcaactgaggtgcaaaatgcattacgta
ataagcaggtaaaggagtatcaggagcagatccagaaagcgatagagcaggaggataaagcgcgtaaagcgggtatttttggcgct
atttttgactggattaccggcatatttgaaaccgtgattggcgccttaaaagttgtggaaggttttctgtccggaaatcccgcagaaatggc
tagcggcgtagcttatatggccgcaggttgtgcaggaatggttaaagccggagccgaaacggcaatgatgtgcggtgctgaccacga
tacctgtcaggcaattattgacgtgacaagtaagattcaatttggttgtgaagccgtcgcgctggcactggatgttttccagattggccgt
gcttttatggcgacgagaggtttatctggcgcagctgcaaaagtgcttgactccggttttggcgaggaagtggttgagcgtatggtaggt
gcaggggaagcagaaatagaggagttggctgaaaagtttggcgaagaagtgagcgaaagtttttccaaacaatttgagccgcttgaa
cgtgaaatggctatggcgaatgagatggcagaggaggctgccgagttttctcgtaacgtagaaaataatatgacgcgaagcgcggga
aaaagctttacgaaagagggggtgaaagcaatggcaaaagaagcggcaaaagaagccctggaaaaatgtgtgcaagaaggtgga
aagttcctgttaaaaaaattccgtaataaagttctcttcaatatgttcaaaaaaatcctgtatgccttactgagggattgttcatttaaaggctt
acaggctatcagatgtgcaaccgagggcgccagtcagatgaatactggcatggttaacacagaaaaagcgaagatcgaaaagaaaa
tagagcaattaataactcagcaacggtttctggatttcataatgcaacaaacagaaaaccagaaaaagatagaacaaaaacgcttagag
gagctttataaggggagcggtgccgcgcttagagatgtattagataccattgatcactatagtagcgttcaggcgagaatagctggctat
cgcgcttaa
S2 amino acid sequence
SEQ ID NO: 66
MSSGNILWGSQNPIVFKNSFGVSNADTGSQDDLSQQNPFAEGYGVLLILLMVI
QAIANNKFIEVQKNAERARNTQEKSNEMDEVIAKAAKGDAKTKEEVPEDVIKYMRD
NGILIDGMTIDDYMAKYGDHGKLDKGGLQAIKAALDNDANRNTDLMSQGQITIQK
MSQELNAVLTQLTGLISKWGEISSMIAQKTYSELMNRIHSNSDSAAGVTALTHHHLS
NVSCVSSGSLGKRQHRVNSTFGDGNAACLLSGKISLQEASNALKQLLDAVPGNHKR
PSLPDFLQTNPAVLSMMMTSLILNVFGNNAQSLCQQLERATEVQNALRNKQVKEYQ
EQIQKAIEQEDKARKAGIFGAIFDWITGIFETVIGALKVVEGFLSGNPAEMASGVAYM
AAGCAGMVKAGAETAMMCGADHDTCQAIIDVTSKIQFGCEAVALALDVFQIGRAF
MATRGLSGAAAKVLDSGFGEEVVERMVGAGEAEIEELAEKFGEEVSESFSKQFEPLE
REMAMANEMAEEAAEFSRNVENNMTRSAGKSFTKEGVKAMAKEAAKEALEKCVQ
EGGKFLLKKFRNKVLFNMFKKILYALLRDCSFKGLQAIRCATEGASQMNTGMVNTE
KAKIEKKIEQLITQQRFLDFIMQQTENQKKIEQKRLEELYKGSGAALRDVLDTIDHYS
SVQARIAGYRA
LTA1-GSAAS-S2 nucleic acid sequence
SEQ ID NO: 67
CATatggacaatggcgatcgtttataccgtgccgactcgcgtcccccagatgagattaaacgtagcggtgggttaatgcc
acgtgggcacaatgagtattttgaccgtggaacacagatgaacattaacctttacgatcatgcccgtgggacccagaccgggtttgtcc
gttatgatgacgggtatgttagtacgagtttgtccttacgctccgcacaccttgcgggacaaagtattttatcaggctacagcacatattac
atttatgtgatcgccactgccccaaacatgttcaatgtgaacgatgtgttgggggtttacagcccccatccatatgaacaagaagtctcg
gcccttggggggatcccatatagccagatttatggttggtaccgcgtaaattttggtgtgattgatgaacgtttgcatcgtaaccgtgaata
ccgcgatcgctactaccgtaacttgaacattgcacctgccgaggacggctatcgtttagcgggattcccacccgatcatcaggcgtgg
cgtgaggaaccgtggatccatcacgcccctcaggggtgcgggaacagtagtcgcgggtccgcggcatccatgtcttcaggaaacat
cttatggggaagtcaaaaccctattgtgtttaaaaatagcttcggcgtcagcaacgctgataccgggagccaggatgacttatcccagc
aaaatccgtttgccgaagggtatggtgttttgcttattctccttatggttattcaggctatcgcaaataataaatttattgaagtccagaagaa
cgctgaacgtgccagaaatacccaggaaaagtcaaatgagatggatgaggtgattgctaaagcagccaaaggggatgctaaaacca
aagaggaggtgcctgaggatgtaattaaatacatgcgtgataatggtattctcatcgatggtatgaccattgatgattatatggctaaatat
ggcgatcatgggaagctggataaaggtggcctacaggcgatcaaagcggctttggataatgacgccaaccggaataccgatcttatg
agtcaggggcagataacaattcaaaaaatgtctcaggagcttaacgctgtccttacccaactgacagggcttatcagtaagtgggggg
aaatttccagtatgatagcgcagaaaacgtactcaGAGCTCatgaatcgaattcacagtaatagcgacagcgccgcaggagtaa
ccgccttaacacatcatcacttaagcaatgtcagttgcgtttcctcgggttcgctgggaaagcgccagcatcgtgtgaattctacttttggc
gatggcaacgccgcgtgtctgctatccgggaaaattagtcttcaggaggcaagcaatgcgttgaagcaactgcttgatgccgtacccg
gaaatcataagcgtccatcattgcctgactttttgcagaccaatcccgcggttttatcaatgatgatgacgtcattaatactcaacgtctttg
gtaataacgctcaatcgttatgccaacagcttgagcgggcaactgaggtgcaaaatgcattacgtaataagcaggtaaaggagtatca
ggagcagatccagaaagcgatagagcaggaggataaagcgcgtaaagcgggtatttttggcgctatttttgactggattaccggcata
tttgaaaccgtgattggcgccttaaaagttgtggaaggttttctgtccggaaatcccgcagaaatggctagcggcgtagcttatatggcc
gcaggttgtgcaggaatggttaaagccggagccgaaacggcaatgatgtgcggtgctgaccacgatacctgtcaggcaattattgac
gtgacaagtaagattcaatttggttgtgaagccgtcgcgctggcactggatgttttccagattggccgtgcttttatggcgacgagaggtt
tatctggcgcagctgcaaaagtgcttgactccggttttggcgaggaagtggttgagcgtatggtaggtgcaggggaagcagaaatag
aggagttggctgaaaagtttggcgaagaagtgagcgaaagtttttccaaacaatttgagccgcttgaacgtgaaatggctatggcgaat
gagatggcagaggaggctgccgagttttctcgtaacgtagaaaataatatgacgcgaagcgcgggaaaaagctttacgaaagaggg
ggtgaaagcaatggcaaaagaagcggcaaaagaagccctggaaaaatgtgtgcaagaaggtggaaagttcctgttaaaaaaattcc
gtaataaagttctcttcaatatgttcaaaaaaatcctgtatgccttactgagggattgttcatttaaaggcttacaggctatcagatgtgcaac
cgagggcgccagtcagatgaatactggcatggttaacacagaaaaagcgaagatcgaaaagaaaatagagcaattaataactcagc
aacggtttctggatttcataatgcaacaaacagaaaaccagaaaaagatagaacaaaaacgcttagaggagctttataaggggagcgg
tgccgcgcttagagatgtattagataccattgatcactatagtagcgttcaggcgagaatagctggctatcgcgcttaaCTCGAG
LTA1-GSAAS-S2 Amino acid sequence
SEQ ID NO: 68
MDNGDRLYRADSRPPDEIKRSGGLMPRGHNEYFDRGTQMNINLYDHARGTQ
TGFVRYDDGYVSTSLSLRSAHLAGQSILSGYSTYYIYVIATAPNMFNVNDVLGVYSP
HPYEQEVSALGGIPYSQIYGWYRVNFGVIDERLHRNREYRDRYYRNLNIAPAEDGYR
LAGFPPDHQAWREEPWIHHAPQGCGNSSRGSAASMSSGNILWGSQNPIVFKNSFGVS
NADTGSQDDLSQQNPFAEGYGVLLILLMVIQAIANNKFIEVQKNAERARNTQEKSNE
MDEVIAKAAKGDAKTKEEVPEDVIKYMRDNGILIDGMTIDDYMAKYGDHGKLDKG
GLQAIKAALDNDANRNTDLMSQGQITIQKMSQELNAVLTQLTGLISKWGEISSMIAQ
KTYSELMNRIHSNSDSAAGVTALTHHHLSNVSCVSSGSLGKRQHRVNSTFGDGNAA
CLLSGKISLQEASNALKQLLDAVPGNHKRPSLPDFLQTNPAVLSMMMTSLILNVFGN
NAQSLCQQLERATEVQNALRNKQVKEYQEQIQKAIEQEDKARKAGIFGAIFDWITGI
FETVIGALKVVEGFLSGNPAEMASGVAYMAAGCAGMVKAGAETAMMCGADHDTC
QAIIDVTSKIQFGCEAVALALDVFQIGRAFMATRGLSGAAAKVLDSGFGEEVVERMV
GAGEAEIEELAEKFGEEVSESFSKQFEPLEREMAMANEMAEEAAEFSRNVENNMTRS
AGKSFTKEGVKAMAKEAAKEALEKCVQEGGKFLLKKFRNKVLFNMFKKILYALLR
DCSFKGLQAIRCATEGASQMNTGMVNTEKAKIEKKIEQLITQQRFLDFIMQQTENQK
KIEQKRLEELYKGSGAALRDVLDTIDHYSSVQARIAGYRA*
LTA1-SseB Nucleic acid sequence
SEQ ID NO: 69
ATGGGCAGCAGCCATCATCATCATCATCACAGCAGCGGCCTGGTGCCGCG
CGGCAGCCATatggacaatggcgatcgtttataccgtgccgactcgcgtcccccagatgagattaaacgtagcggtgggtta
atgccacgtgggcacaatgagtattttgaccgtggaacacagatgaacattaacctttacgatcatgcccgtgggacccagaccgggtt
tgtccgttatgatgacgggtatgttagtacgagtttgtccttacgctccgcacaccttgcgggacaaagtattttatcaggctacagcacat
attacatttatgtgatcgccactgccccaaacatgttcaatgtgaacgatgtgttgggggtttacagcccccatccatatgaacaagaagt
ctcggcccttggggggatcccatatagccagatttatggttggtaccgcgtaaattttggtgtgattgatgaacgtttgcatcgtaaccgtg
aataccgcgatcgctactaccgtaacttgaacattgcacctgccgaggacggctatcgtttagcgggattcccacccgatcatcaggcg
tggcgtgaggaaccgtggatccatcacgcccctcaggggtgcgggaacagtagtcgcgggtccgcggcatccatgtcttcaggaaa
catcttatggggaagtcaaaaccctattgtgtttaaaaatagcttcggcgtcagcaacgctgataccgggagccaggatgacttatccca
gcaaaatccgtttgccgaagggtatggtgttttgcttattctccttatggttattcaggctatcgcaaataataaatttattgaagtccagaag
aacgctgaacgtgccagaaatacccaggaaaagtcaaatgagatggatgaggtgattgctaaagcagccaaaggggatgctaaaac
caaagaggaggtgcctgaggatgtaattaaatacatgcgtgataatggtattctcatcgatggtatgaccattgatgattatatggctaaat
atggcgatcatgggaagctggataaaggtggcctacaggcgatcaaagcggctttggataatgacgccaaccggaataccgatcttat
gagtcaggggcagataacaattcaaaaaatgtctcaggagcttaacgctgtccttacccaactgacagggcttatcagtaagtggggg
gaaatttccagtatgatagcgcagaaaacgtactcataaGGATCC
LTA1-SseB Amino acid sequence
SEQ ID NO: 70
MGSSHHHHHHSSGLVPRGSHMDNGDRLYRADSRPPDEIKRSGGLMPRGHNE
YFDRGTQMNINLYDHARGTQTGFVRYDDGYVSTSLSLRSAHLAGQSILSGYSTYYIY
VIATAPNMFNVNDVLGVYSPHPYEQEVSALGGIPYSQIYGWYRVNFGVIDERLHRNR
EYRDRYYRNLNIAPAEDGYRLAGFPPDHQAWREEPWIHHAPQGCGNSSRGSAASMS
SGNILWGSQNPIVFKNSFGVSNADTGSQDDLSQQNPFAEGYGVLLILLMVIQAIANN
KFIEVQKNAERARNTQEKSNEMDEVIAKAAKGDAKTKEEVPEDVIKYMRDNGILID
GMTIDDYMAKYGDHGKLDKGGLQAIKAALDNDANRNTDLMSQGQITIQKMSQELN
AVLTQLTGLISKWGEISSMIAQKTYS*
HisScc2 chaperone for LTA1-CT053-CopB nucleic acid sequence
SEQ ID NO: 71
ATGGGCAGCAGCCATCACCATCATCACCACAGCCAGGATCCGatgagcactccat
cttctaataattctaaaaaaccttcggcctcttttaataaaaaatcacgtagccgcttggccgagattgctgcacaaaaaaaagcaaaagc
tgaggatttggaacaaaaatatcctgttcctacggaagaggagacaaaacaagttctcatggacatcctacaggggttaagcaacgga
ttaactcttcagcaaattttaggtctctccgacgtcctccttgaagagatctacaccgtagcatataccttctactcccaagggaaatatcg
ggaagctatcggtcttttccaaatcttaacagcctccaaacctcaatgctacaaatacatcttaggtcttagctcttgctatcaccagctaaa
aatgtatgatgaagccgcttttggtttcttcctagctttcgatgctcaacccgaaaaccccatccctccttactacatcgccgatagcttgat
gaagctaaaccaacccgaagaatctcaagacttcctcgatattacgatcgatatgtgtaagaacaagccggaatataaagttcttaaaga
tcgctgcagcattatgaagcaatctttagatgccgtgctgaaaaaagagaaatctgcaaaaggctctgaaacacaagcctcctctcctaa
aaacacaaaagctaaaaaagctgcttctaacaagaaaaaagcaaagtaaGCGGCCGC
HisScc2 chaperone for LTA1-CT053-CopB Amino acid sequence
SEQ ID NO: 72
MGSSHHHHHHSQDPMSTPSSNNSKKPSASFNKKSRSRLAEIAAQKKAKAEDL
EQKYPVPTEEETKQVLMDILQGLSNGLTLQQILGLSDVLLEEIYTVAYTFYSQGKYRE
AIGLFQILTASKPQCYKYILGLSSCYHQLKMYDEAAFGFFLAFDAQPENPIPPYYIADS
LMKLNQPEESQDFLDITIDMCKNKPEYKVLKDRCSIMKQSLDAVLKKEKSAKGSETQ
ASSPKNTKAKKAASNKKKAK*
CT053 nucleic acid sequence
SEQ ID NO: 73
Aaaagtgagcgtttaaaaaaattagaatcagagcttcatgatcttacccagtggatgcaacttggccttgttcctaaaaaaga
aatcgagagacaccaggaagaaatccgtctgctagaaagcaaaatccttgaagagaaagaacgtctacaacttctcaaagaaagcgg
tgagatcaaagagtacgtaacccctcgaagaactccagctaaaaccatttacccagatggccccagcgtttcagacgttgagtttgtag
aatcctcggatacagaagtggatctcgatgccggtgacacaattgagattgacctaggtgatgaggcaagagaagaaagcggaaac
gaactcgactactctagtgaagacgatgaggatcctttcagcgatcgcaatcgttggcgccgaggaggcatcatagatcctgacgcga
atgaatgg
CT053 amino acid sequence
SEQ ID NO: 74
MKSERLKKLESELHDLTQWMQLGLVPKKEIERHQEEIRLLESKILEEKERLQL
LKESGEIKEYVTPRRTPAKTIYPDGPSVSDVEFVESSDTEVDLDAGDTIEIDLGDEARE
ESGNELDYSSEDDEDPFSDRNRWRRGGIIDPDANEW
CopB nucleic acid sequence
SEQ ID NO: 75
atgagcttgtcatccagcagcagctcggatagttcgaatctgaaaaatgtgttatctcaggtcatcgcgtctacaccAcagg
gggttcctaatgctgacaaattaaccgacaatcaggtaaaacaagtccagcagacccgtcaaaaccgtgatgatctgtccatggagag
cgacgtcgcggtggcgggaacagccggaaaagatcgtgctgcgtcggcgtcccagatcgagggacaagagctgattgagcaaca
gggacttgcggctgggaaagagacggcttctgctgatgctacatcattgacccagtcggcatccaaaggcgcttccagtcagcagtgt
attgaggataccagtaagtccctggagctttcttcgctttcgagcctgtcaagcgtagatgcgacacatttgcaggaaatccaatcgatc
gtgtcttcagcaatgggcgccaccaacgaattgtcattgacgaacttagagacaccgggattaccaaagccgagtaccactccAcgc
caggaagttatggagatcagccttgccttagcgaaggccatcactgcattgggtgagagcactcaggctgccttggaaaattttcagtc
cactcagagtcagtccgcgaacatgaataagatgagtttggaatcccaaggcttgaaaatcgacaaggagcgtgaagaatttaagaaa
atgcaggagattcagcaaaagagcggcacaaattcaaccatggatactgtgaataaagttatgattggcgtgacagtggcaattacagt
aatctctgttgtttcagcattgtttacctgcggtttgggcttgattggcacagccgctgcgggtgccacagccgccaccgctggggcaac
ggccgccgccacgaccgctacctctgtgacgaccacagtcgctacccaggtgacgatgcaagcggtggtccaagtcgttaagcagg
ctattatccaagcagtaaaacgcgccatcgtccaagcgattaaacaggggattaagcaaggcattaaacaagcgatcaaacaggcag
tcaaggcaagcgtgaagacacttgccaaaaatgtaggcaagattttcagcgcaggcaagaacgctgtgagtaagtccttcccAaaatt
gtctaaggtgattaatacacttggttccaaatgggttactcttggcgtgggggcccttacagcggtgccgcagttagtcagtggcattac
ctcccttcaattgtctgatatgcaaaaagaacttgcacaaatccaaaaggaagtgggtgcacttacggcgcagagtgagatgatgaaa
gcgtttacactgttctggcagcaagcttcgaaaatcgcggccaaacaaacggaatcaccttcagagacgcaacaacaggcagctaag
accggcgcccagatcgctaaagcgttgtccgccatttcgggtgctttagctgctgctgctTAG
CopB amino acid sequence
SEQ ID NO: 76
MSLSSSSSSDSSNLKNVLSQVIASTPQGVPNADKLTDNQVKQVQQTRQNRDD
LSMESDVAVAGTAGKDRAASASQIEGQELIEQQGLAAGKETASADATSLTQSASKG
ASSQQCIEDTSKSLELSSLSSLSSVDATHLQEIQSIVSSAMGATNELSLTNLETPGLPKP
STTPRQEVMEISLALAKAITALGESTQAALENFQSTQSQSANMNKMSLESQGLKIDK
EREEFKKMQEIQQKSGTNSTMDTVNKVMIGVTVAITVISVVSALFTCGLGLIGTAAA
GATAATAGATAAATTATSVTTTVATQVTMQAVVQVVKQAIIQAVKRAIVQAIKQGI
KQGIKQAIKQAVKASVKTLAKNVGKIFSAGKNAVSKSFPKLSKVINTLGSKWVTLGV
GALTAVPQLVSGITSLQLSDMQKELAQIQKEVGALTAQSEMMKAFTLFWQQASKIA
AKQTESPSETQQQAAKTGAQIAKALSAISGALAAAA
CT053-CopB nucleic acid sequence
SEQ ID NO: 77
aaaagtgagcgtttaaaaaaattagaatcagagcttcatgatcttacccagtggatgcaacttggccttgttcctaaaaaagaa
atcgagagacaccaggaagaaatccgtctgctagaaagcaaaatccttgaagagaaagaacgtctacaacttctcaaagaaagcggt
gagatcaaagagtacgtaacccctcgaagaactccagctaaaaccatttacccagatggccccagcgtttcagacgttgagtttgtaga
atcctcggatacagaagtggatctcgatgccggtgacacaattgagattgacctaggtgatgaggcaagagaagaaagcggaaacg
aactcgactactctagtgaagacgatgaggatcctttcagcgatcgcaatcgttggcgccgaggaggcatcatagatcctgacgcgaa
tgaatggGGTTCAGCTGCTTCAatgagcttgtcatccagcagcagctcggatagttcgaatctgaaaaatgtgttatctca
ggtcatcgcgtctacaccAcagggggttcctaatgctgacaaattaaccgacaatcaggtaaaacaagtccagcagacccgtcaaaa
ccgtgatgatctgtccatggagagcgacgtcgcggtggcgggaacagccggaaaagatcgtgctgcgtcggcgtcccagatcgag
ggacaagagctgattgagcaacagggacttgcggctgggaaagagacggcttctgctgatgctacatcattgacccagtcggcatcc
aaaggcgcttccagtcagcagtgtattgaggataccagtaagtccctggagctttcttcgctttcgagcctgtcaagcgtagatgcgaca
catttgcaggaaatccaatcgatcgtgtcttcagcaatgggcgccaccaacgaattgtcattgacgaacttagagacaccgggattacc
aaagccgagtaccactccAcgccaggaagttatggagatcagccttgccttagcgaaggccatcactgcattgggtgagagcactca
ggctgccttggaaaattttcagtccactcagagtcagtccgcgaacatgaataagatgagtttggaatcccaaggcttgaaaatcgaca
aggagcgtgaagaatttaagaaaatgcaggagattcagcaaaagagcggcacaaattcaaccatggatactgtgaataaagttatgat
tggcgtgacagtggcaattacagtaatctctgttgtttcagcattgtttacctgcggtttgggcttgattggcacagccgctgcgggtgcc
acagccgccaccgctggggcaacggccgccgccacgaccgctacctctgtgacgaccacagtcgctacccaggtgacgatgcaag
cggtggtccaagtcgttaagcaggctattatccaagcagtaaaacgcgccatcgtccaagcgattaaacaggggattaagcaaggcat
taaacaagcgatcaaacaggcagtcaaggcaagcgtgaagacacttgccaaaaatgtaggcaagattttcagcgcaggcaagaacg
ctgtgagtaagtccttcccAaaattgtctaaggtgattaatacacttggttccaaatgggttactcttggcgtgggggcccttacagcggt
gccgcagttagtcagtggcattacctcccttcaattgtctgatatgcaaaaagaacttgcacaaatccaaaaggaagtgggtgcacttac
ggcgcagagtgagatgatgaaagcgtttacactgttctggcagcaagcttcgaaaatcgcggccaaacaaacggaatcaccttcaga
gacgcaacaacaggcagctaagaccggcgcccagatcgctaaagcgttgtccgccatttcgggtgctttagctgctgctgctTAG
CT053-CopB amino acid sequence
SEQ ID NO: 78
MKSERLKKLESELHDLTQWMQLGLVPKKEIERHQEEIRLLESKILEEKERLQL
LKESGEIKEYVTPRRTPAKTIYPDGPSVSDVEFVESSDTEVDLDAGDTIEIDLGDEARE
ESGNELDYSSEDDEDPFSDRNRWRRGGIIDPDANEWGSAASMSLSSSSSSDSSNLKN
VLSQVIASTPQGVPNADKLTDNQVKQVQQTRQNRDDLSMESDVAVAGTAGKDRAA
SASQIEGQELIEQQGLAAGKETASADATSLTQSASKGASSQQCIEDTSKSLELSSLSSL
SSVDATHLQEIQSIVSSAMGATNELSLTNLETPGLPKPSTTPRQEVMEISLALAKAITA
LGESTQAALENFQSTQSQSANMNKMSLESQGLKIDKEREEFKKMQEIQQKSGTNST
MDTVNKVMIGVTVAITVISVVSALFTCGLGLIGTAAAGATAATAGATAAATTATSVT
TTVATQVTMQAVVQVVKQAIIQAVKRAIVQAIKQGIKQGIKQAIKQAVKASVKTLA
KNVGKIFSAGKNAVSKSFPKLSKVINTLGSKWVTLGVGALTAVPQLVSGITSLQLSD
MQKELAQIQKEVGALTAQSEMMKAFTLFWQQASKIAAKQTESPSETQQQAAKTGA
QIAKALSAISGALAAAA
LTA1-CT053-CopB nucleic acid sequence
SEQ ID NO: 79
CATatggacaatggcgatcgtttataccgtgccgactcgcgtcccccagatgagattaaacgtagcggtgggttaatgcc
acgtgggcacaatgagtattttgaccgtggaacacagatgaacattaacctttacgatcatgcccgtgggacccagaccgggtttgtcc
gttatgatgacgggtatgttagtacgagtttgtccttacgctccgcacaccttgcgggacaaagtattttatcaggctacagcacatattac
atttatgtgatcgccactgccccaaacatgttcaatgtgaacgatgtgttgggggtttacagcccccatccatatgaacaagaagtctcg
gcccttggggggatcccatatagccagatttatggttggtaccgcgtaaattttggtgtgattgatgaacgtttgcatcgtaaccgtgaata
ccgcgatcgctactaccgtaacttgaacattgcacctgccgaggacggctatcgtttagcgggattcccacccgatcatcaggcgtgg
cgtgaggaaccgtggatccatcacgcccctcaggggtgcgggaacagtagtcgcCATatgaaaagtgagcgtttaaaaaaattag
aatcagagcttcatgatcttacccagtggatgcaacttggccttgttcctaaaaaagaaatcgagagacaccaggaagaaatccgtctg
ctagaaagcaaaatccttgaagagaaagaacgtctacaacttctcaaagaaagcggtgagatcaaagagtacgtaacccctcgaaga
actccagctaaaaccatttacccagatggccccagcgtttcagacgttgagtttgtagaatcctcggatacagaagtggatctcgatgcc
ggtgacacaattgagattgacctaggtgatgaggcaagagaagaaagcggaaacgaactcgactactctagtgaagacgatgagga
tcctttcagcgatcgcaatcgttggcgccgaggaggcatcatagatcctgacgcgaatgaatggGGTTCAGCTGCTTCA
atgagcttgtcatccagcagcagctcggatagttcgaatctgaaaaatgtgttatctcaggtcatcgcgtctacaccAcagggggttcct
aatgctgacaaattaaccgacaatcaggtaaaacaagtccagcagacccgtcaaaaccgtgatgatctgtccatggagagcgacgtc
gcggtggcgggaacagccggaaaagatcgtgctgcgtcggcgtcccagatcgagggacaagagctgattgagcaacagggactt
gcggctgggaaagagacggcttctgctgatgctacatcattgacccagtcggcatccaaaggcgcttccagtcagcagtgtattgagg
ataccagtaagtccctggagctttcttcgctttcgagcctgtcaagcgtagatgcgacacatttgcaggaaatccaatcgatcgtgtcttc
agcaatgggcgccaccaacgaattgtcattgacgaacttagagacaccgggattaccaaagccgagtaccactccAcgccaggaa
gttatggagatcagccttgccttagcgaaggccatcactgcattgggtgagagcactcaggctgccttggaaaattttcagtccactcag
agtcagtccgcgaacatgaataagatgagtttggaatcccaaggcttgaaaatcgacaaggagcgtgaagaatttaagaaaatgcag
gagattcagcaaaagagcggcacaaattcaaccatggatactgtgaataaagttatgattggcgtgacagtggcaattacagtaatctct
gttgtttcagcattgtttacctgcggtttgggcttgattggcacagccgctgcgggtgccacagccgccaccgctggggcaacggccg
ccgccacgaccgctacctctgtgacgaccacagtcgctacccaggtgacgatgcaagcggtggtccaagtcgttaagcaggctattat
ccaagcagtaaaacgcgccatcgtccaagcgattaaacaggggattaagcaaggcattaaacaagcgatcaaacaggcagtcaagg
caagcgtgaagacacttgccaaaaatgtaggcaagattttcagcgcaggcaagaacgctgtgagtaagtccttcccAaaattgtctaa
ggtgattaatacacttggttccaaatgggttactcttggcgtgggggcccttacagcggtgccgcagttagtcagtggcattacctccctt
caattgtctgatatgcaaaaagaacttgcacaaatccaaaaggaagtgggtgcacttacggcgcagagtgagatgatgaaagcgttta
cactgttctggcagcaagcttcgaaaatcgcggccaaacaaacggaatcaccttcagagacgcaacaacaggcagctaagaccggc
gcccagatcgctaaagcgttgtccgccatttcgggtgctttagctgctgctgctTAGCTCGAG
LTA1-CT053-CopB Amino acid sequence
SEQ ID NO: 80
MDNGDRLYRADSRPPDEIKRSGGLMPRGHNEYFDRGTQMNINLYDHARGTQ
TGFVRYDDGYVSTSLSLRSAHLAGQSILSGYSTYYIYVIATAPNMFNVNDVLGVYSP
HPYEQEVSALGGIPYSQIYGWYRVNFGVIDERLHRNREYRDRYYRNLNIAPAEDGYR
LAGFPPDHQAWREEPWIHHAPQGCGNSSRMKSERLKKLESELHDLTQWMQLGLVP
KKEIERHQEEIRLLESKILEEKERLQLLKESGEIKEYVTPRRTPAKTIYPDGPSVSDVEF
VESSDTEVDLDAGDTIEIDLGDEAREESGNELDYSSEDDEDPFSDRNRWRRGGIIDPD
ANEWGSAASMSLSSSSSSDSSNLKNVLSQVIASTPQGVPNADKLTDNQVKQVQQTR
QNRDDLSMESDVAVAGTAGKDRAASASQIEGQELIEQQGLAAGKETASADATSLTQ
SASKGASSQQCIEDTSKSLELSSLSSLSSVDATHLQEIQSIVSSAMGATNELSLTNLETP
GLPKPSTTPRQEVMEISLALAKAITALGESTQAALENFQSTQSQSANMNKMSLESQG
LKIDKEREEFKKMQEIQQKSGTNSTMDTVNKVMIGVTVAITVISVVSALFTCGLGLIG
TAAAGATAATAGATAAATTATSVTTTVATQVTMQAVVQVVKQAIIQAVKRAIVQAI
KQGIKQGIKQAIKQAVKASVKTLAKNVGKIFSAGKNAVSKSFPKLSKVINTLGSKWV
TLGVGALTAVPQLVSGITSLQLSDMQKELAQIQKEVGALTAQSEMMKAFTLFWQQA
SKIAAKQTESPSETQQQAAKTGAQIAKALSAISGALAAAA*
HisScc2 chaperone for LTA1-CT668-CopB nucleic acid sequence
SEQ ID NO: 81
ATGGGCAGCAGCCATCACCATCATCACCACAGCCAGGATCCGatgagcactccat
cttctaataattctaaaaaaccttcggcctcttttaataaaaaatcacgtagccgcttggccgagattgctgcacaaaaaaaagcaaaagc
tgaggatttggaacaaaaatatcctgttcctacggaagaggagacaaaacaagttctcatggacatcctacaggggttaagcaacgga
ttaactcttcagcaaattttaggtctctccgacgtcctccttgaagagatctacaccgtagcatataccttctactcccaagggaaatatcg
ggaagctatcggtcttttccaaatcttaacagcctccaaacctcaatgctacaaatacatcttaggtcttagctcttgctatcaccagctaaa
aatgtatgatgaagccgcttttggtttcttcctagctttcgatgctcaacccgaaaaccccatccctccttactacatcgccgatagcttgat
gaagctaaaccaacccgaagaatctcaagacttcctcgatattacgatcgatatgtgtaagaacaagccggaatataaagttcttaaaga
tcgctgcagcattatgaagcaatctttagatgccgtgctgaaaaaagagaaatctgcaaaaggctctgaaacacaagcctcctctcctaa
aaacacaaaagctaaaaaagctgcttctaacaagaaaaaagcaaagtaaGCGGCCGC
HisScc2 chaperone for LTA1-CT668-CopB Amino acid sequence
SEQ ID NO: 82
MGSSHHHHHHSQDPMSTPSSNNSKKPSASFNKKSRSRLAEIAAQKKAKAEDL
EQKYPVPTEEETKQVLMDILQGLSNGLTLQQILGLSDVLLEEIYTVAYTFYSQGKYRE
AIGLFQILTASKPQCYKYILGLSSCYHQLKMYDEAAFGFFLAFDAQPENPIPPYYIADS
LMKLNQPEESQDFLDITIDMCKNKPEYKVLKDRCSIMKQSLDAVLKKEKSAKGSETQ
ASSPKNTKAKKAASNKKKAK*
CT668 nucleic acid sequence
SEQ ID NO: 83
Atagatcctcttaagctttttccaaattttgatggggataaggagagtgctgcggtgaataaaccttcagcatctcctatgccc
agcgaattaagtaaaaatgttgcctcattctctttagggggtggaggtgctgcgttggattcgacagtgtccacagaaaagctatcgttga
tggctatgatgcaggataaaaattcgcagttgatcgatcctgagttggaggaagctctgaactctgaagagttacaagagcagatccatt
tgttaaaaagtcgtttgtgggatgcacaaacgcagatgcaaatgcaagatcccgacaagttggcctctgagcatgtagatgctttagga
gtcattgttgatttaatcaatggggattttcaagcgatagctgaacatacacaacagacggtcaagcagggtaatggtgacgaagaaaa
atctgttacacgcaagatagtcgattgggtctcttcaggagaagaaattttgaatcgtgctttgttgtatttctccgatcgtaatggagaaag
agaaacattagccgatttcttaaaagttcagtatgccgttcaaagagctacacaacgcgccgagttatttgccagtattctaggtgccacg
gtgagtagtgtaaaaacgattatgacaacccagttaggt
CT668 amino acid sequence
SEQ ID NO: 84
MIDPLKLFPNFDGDKESAAVNKPSASPMPSELSKNVASFSLGGGGAALDSTVS
TEKLSLMAMMQDKNSQLIDPELEEALNSEELQEQIHLLKSRLWDAQTQMQMQDPD
KLASEHVDALGVIVDLINGDFQAIAEHTQQTVKQGNGDEEKSVTRKIVDWVSSGEEI
LNRALLYFSDRNGERETLADFLKVQYAVQRATQRAELFASILGATVSSVKTIMTTQL
CT668-CopB nucleic acid sequence
SEQ ID NO: 85
atagatcctcttaagctttttccaaattttgatggggataaggagagtgctgcggtgaataaaccttcagcatctcctatgccca
gcgaattaagtaaaaatgttgcctcattctctttagggggtggaggtgctgcgttggattcgacagtgtccacagaaaagctatcgttgat
ggctatgatgcaggataaaaattcgcagttgatcgatcctgagttggaggaagctctgaactctgaagagttacaagagcagatccattt
gttaaaaagtcgtttgtgggatgcacaaacgcagatgcaaatgcaagatcccgacaagttggcctctgagcatgtagatgctttaggag
tcattgttgatttaatcaatggggattttcaagcgatagctgaacatacacaacagacggtcaagcagggtaatggtgacgaagaaaaat
ctgttacacgcaagatagtcgattgggtctcttcaggagaagaaattttgaatcgtgctttgttgtatttctccgatcgtaatggagaaaga
gaaacattagccgatttcttaaaagttcagtatgccgttcaaagagctacacaacgcgccgagttatttgccagtattctaggtgccacgg
tgagtagtgtaaaaacgattatgacaacccagttaggtGGTTCAGCTGCTTCAatgagcttgtcatccagcagcagctcg
gatagttcgaatctgaaaaatgtgttatctcaggtcatcgcgtctacaccAcagggggttcctaatgctgacaaattaaccgacaatcag
gtaaaacaagtccagcagacccgtcaaaaccgtgatgatctgtccatggagagcgacgtcgcggtggcgggaacagccggaaaag
atcgtgctgcgtcggcgtcccagatcgagggacaagagctgattgagcaacagggacttgcggctgggaaagagacggcttctgct
gatgctacatcattgacccagtcggcatccaaaggcgcttccagtcagcagtgtattgaggataccagtaagtccctggagctttcttcg
ctttcgagcctgtcaagcgtagatgcgacacatttgcaggaaatccaatcgatcgtgtcttcagcaatgggcgccaccaacgaattgtc
attgacgaacttagagacaccgggattaccaaagccgagtaccactccAcgccaggaagttatggagatcagccttgccttagcgaa
ggccatcactgcattgggtgagagcactcaggctgccttggaaaattttcagtccactcagagtcagtccgcgaacatgaataagatga
gtttggaatcccaaggcttgaaaatcgacaaggagcgtgaagaatttaagaaaatgcaggagattcagcaaaagagcggcacaaatt
caaccatggatactgtgaataaagttatgattggcgtgacagtggcaattacagtaatctctgttgtttcagcattgtttacctgcggtttgg
gcttgattggcacagccgctgcgggtgccacagccgccaccgctggggcaacggccgccgccacgaccgctacctctgtgacgac
cacagtcgctacccaggtgacgatgcaagcggtggtccaagtcgttaagcaggctattatccaagcagtaaaacgcgccatcgtcca
agcgattaaacaggggattaagcaaggcattaaacaagcgatcaaacaggcagtcaaggcaagcgtgaagacacttgccaaaaatg
taggcaagattttcagcgcaggcaagaacgctgtgagtaagtccttcccAaaattgtctaaggtgattaatacacttggttccaaatggg
ttactcttggcgtgggggcccttacagcggtgccgcagttagtcagtggcattacctcccttcaattgtctgatatgcaaaaagaacttgc
acaaatccaaaaggaagtgggtgcacttacggcgcagagtgagatgatgaaagcgtttacactgttctggcagcaagcttcgaaaatc
gcggccaaacaaacggaatcaccttcagagacgcaacaacaggcagctaagaccggcgcccagatcgctaaagcgttgtccgcca
tttcgggtgctttagctgctgctgctTAG
CT668-CopB amino acid sequence
SEQ ID NO: 86
MIDPLKLFPNFDGDKESAAVNKPSASPMPSELSKNVASFSLGGGGAALDSTVS
TEKLSLMAMMQDKNSQLIDPELEEALNSEELQEQIHLLKSRLWDAQTQMQMQDPD
KLASEHVDALGVIVDLINGDFQAIAEHTQQTVKQGNGDEEKSVTRKIVDWVSSGEEI
LNRALLYFSDRNGERETLADFLKVQYAVQRATQRAELFASILGATVSSVKTIMTTQL
GGSAASMSLSSSSSSDSSNLKNVLSQVIASTPQGVPNADKLTDNQVKQVQQTRQNRD
DLSMESDVAVAGTAGKDRAASASQIEGQELIEQQGLAAGKETASADATSLTQSASK
GASSQQCIEDTSKSLELSSLSSLSSVDATHLQEIQSIVSSAMGATNELSLTNLETPGLPK
PSTTPRQEVMEISLALAKAITALGESTQAALENFQSTQSQSANMNKMSLESQGLKIDK
EREEFKKMQEIQQKSGTNSTMDTVNKVMIGVTVAITVISVVSALFTCGLGLIGTAAA
GATAATAGATAAATTATSVTTTVATQVTMQAVVQVVKQAIIQAVKRAIVQAIKQGI
KQGIKQAIKQAVKASVKTLAKNVGKIFSAGKNAVSKSFPKLSKVINTLGSKWVTLGV
GALTAVPQLVSGITSLQLSDMQKELAQIQKEVGALTAQSEMMKAFTLFWQQASKIA
AKQTESPSETQQQAAKTGAQIAKALSAISGALAAAA
LTA1-CT668-CopB nucleic acid sequence
SEQ ID NO: 87
CATatggacaatggcgatcgtttataccgtgccgactcgcgtcccccagatgagattaaacgtagcggtgggttaatgcc
acgtgggcacaatgagtattttgaccgtggaacacagatgaacattaacctttacgatcatgcccgtgggacccagaccgggtttgtcc
gttatgatgacgggtatgttagtacgagtttgtccttacgctccgcacaccttgcgggacaaagtattttatcaggctacagcacatattac
atttatgtgatcgccactgccccaaacatgttcaatgtgaacgatgtgttgggggtttacagcccccatccatatgaacaagaagtctcg
gcccttggggggatcccatatagccagatttatggttggtaccgcgtaaattttggtgtgattgatgaacgtttgcatcgtaaccgtgaata
ccgcgatcgctactaccgtaacttgaacattgcacctgccgaggacggctatcgtttagcgggattcccacccgatcatcaggcgtgg
cgtgaggaaccgtggatccatcacgcccctcaggggtgcgggaacagtagtcgcCATatgatagatcctcttaagctttttccaaatt
ttgatggggataaggagagtgctgcggtgaataaaccttcagcatctcctatgcccagcgaattaagtaaaaatgttgcctcattctcttta
gggggtggaggtgctgcgttggattcgacagtgtccacagaaaagctatcgttgatggctatgatgcaggataaaaattcgcagttgat
cgatcctgagttggaggaagctctgaactctgaagagttacaagagcagatccatttgttaaaaagtcgtttgtgggatgcacaaacgc
agatgcaaatgcaagatcccgacaagttggcctctgagcatgtagatgctttaggagtcattgttgatttaatcaatggggattttcaagc
gatagctgaacatacacaacagacggtcaagcagggtaatggtgacgaagaaaaatctgttacacgcaagatagtcgattgggtctct
tcaggagaagaaattttgaatcgtgctttgttgtatttctccgatcgtaatggagaaagagaaacattagccgatttcttaaaagttcagtat
gccgttcaaagagctacacaacgcgccgagttatttgccagtattctaggtgccacggtgagtagtgtaaaaacgattatgacaaccca
gttaggtGGTTCAGCTGCTTCAatgagcttgtcatccagcagcagctcggatagttcgaatctgaaaaatgtgttatctcag
gtcatcgcgtctacaccAcagggggttcctaatgctgacaaattaaccgacaatcaggtaaaacaagtccagcagacccgtcaaaac
cgtgatgatctgtccatggagagcgacgtcgcggtggcgggaacagccggaaaagatcgtgctgcgtcggcgtcccagatcgagg
gacaagagctgattgagcaacagggacttgcggctgggaaagagacggcttctgctgatgctacatcattgacccagtcggcatcca
aaggcgcttccagtcagcagtgtattgaggataccagtaagtccctggagctttcttcgctttcgagcctgtcaagcgtagatgcgacac
atttgcaggaaatccaatcgatcgtgtcttcagcaatgggcgccaccaacgaattgtcattgacgaacttagagacaccgggattacca
aagccgagtaccactccAcgccaggaagttatggagatcagccttgccttagcgaaggccatcactgcattgggtgagagcactca
ggctgccttggaaaattttcagtccactcagagtcagtccgcgaacatgaataagatgagtttggaatcccaaggcttgaaaatcgaca
aggagcgtgaagaatttaagaaaatgcaggagattcagcaaaagagcggcacaaattcaaccatggatactgtgaataaagttatgat
tggcgtgacagtggcaattacagtaatctctgttgtttcagcattgtttacctgcggtttgggcttgattggcacagccgctgcgggtgcc
acagccgccaccgctggggcaacggccgccgccacgaccgctacctctgtgacgaccacagtcgctacccaggtgacgatgcaag
cggtggtccaagtcgttaagcaggctattatccaagcagtaaaacgcgccatcgtccaagcgattaaacaggggattaagcaaggcat
taaacaagcgatcaaacaggcagtcaaggcaagcgtgaagacacttgccaaaaatgtaggcaagattttcagcgcaggcaagaacg
ctgtgagtaagtccttcccAaaattgtctaaggtgattaatacacttggttccaaatgggttactcttggcgtgggggcccttacagcggt
gccgcagttagtcagtggcattacctcccttcaattgtctgatatgcaaaaagaacttgcacaaatccaaaaggaagtgggtgcacttac
ggcgcagagtgagatgatgaaagcgtttacactgttctggcagcaagcttcgaaaatcgcggccaaacaaacggaatcaccttcaga
gacgcaacaacaggcagctaagaccggcgcccagatcgctaaagcgttgtccgccatttcgggtgctttagctgctgctgctTAG
CTCGAG
LTA1-CT668-CopB Amino acid sequence
SEQ ID NO: 88
MDNGDRLYRADSRPPDEIKRSGGLMPRGHNEYFDRGTQMNINLYDHARGTQ
TGFVRYDDGYVSTSLSLRSAHLAGQSILSGYSTYYIYVIATAPNMFNVNDVLGVYSP
HPYEQEVSALGGIPYSQIYGWYRVNFGVIDERLHRNREYRDRYYRNLNIAPAEDGYR
LAGFPPDHQAWREEPWIHHAPQGCGNSSRMIDPLKLFPNFDGDKESAAVNKPSASP
MPSELSKNVASFSLGGGGAALDSTVSTEKLSLMAMMQDKNSQLIDPELEEALNSEEL
QEQIHLLKSRLWDAQTQMQMQDPDKLASEHVDALGVIVDLINGDFQAIAEHTQQTV
KQGNGDEEKSVTRKIVDWVSSGEEILNRALLYFSDRNGERETLADFLKVQYAVQRA
TQRAELFASILGATVSSVKTIMTTQLGGSAASMSLSSSSSSDSSNLKNVLSQVIASTPQ
GVPNADKLTDNQVKQVQQTRQNRDDLSMESDVAVAGTAGKDRAASASQIEGQELI
EQQGLAAGKETASADATSLTQSASKGASSQQCIEDTSKSLELSSLSSLSSVDATHLQEI
QSIVSSAMGATNELSLTNLETPGLPKPSTTPRQEVMEISLALAKAITALGESTQAALEN
FQSTQSQSANMNKMSLESQGLKIDKEREEFKKMQEIQQKSGTNSTMDTVNKVMIGV
TVAITVISVVSALFTCGLGLIGTAAAGATAATAGATAAATTATSVTTTVATQVTMQA
VVQVVKQAIIQAVKRAIVQAIKQGIKQGIKQAIKQAVKASVKTLAKNVGKIFSAGKN
AVSKSFPKLSKVINTLGSKWVTLGVGALTAVPQLVSGITSLQLSDMQKELAQIQKEV
GALTAQSEMMKAFTLFWQQASKIAAKQTESPSETQQQAAKTGAQIAKALSAISGAL
AAAA*
HisScc3 chaperone for CT053-CopB2 Nucleic acid sequence
SEQ ID NO: 89
ATGGGCAGCAGCCATCACCATCATCACCACAGCCAGGATCCGatgccaccaagc
aagatccaatgtcttgaaacttttgaaagaacttatggacacctttatctacaacatgcgtccctaatgcgtcatttagcctatctactcgata
aaattgctcgctcttaccctcatatgtgtccgcttcccgataatatggaagcgtactttgagaattatatccccaataaagatatccctctgg
acacctatcaaaaaattttcaaactgtcctcagaagatcttgaacaagtctacaaggaaggatacaacgcctatttacaaggagactatg
aggaaagttctaccgctttttactggttgattttctttaacccatttgtgtctaaattttggttttcattaggagcttcgctccatatgcgccaaaa
atatcaacaagctcttcatgcttatggtgtagctgctttgctaagagaaaaagacccttatcctcattactatgcctacatctgctacaccct
gctcaataatcctgaagaagctgaaaaagctcttgatcttgcttggcaaaaagtaaaaacaagctctgcctatagctctttaaaagaaga
aattttagcgatcaaatcgtacgcctaaGCGGCCGC
HisScc3 chaperone for CT053-CopB2 amino acid sequence
SEQ ID NO: 90
MGSSHHHHHHSQDPMPPSKIQCLETFERTYGHLYLQHASLMRHLAYLLDKIA
RSYPHMCPLPDNMEAYFENYIPNKDIPLDTYQKIFKLSSEDLEQVYKEGYNAYLQGD
YEESSTAFYWLIFFNPFVSKFWFSLGASLHMRQKYQQALHAYGVAALLREKDPYPH
YYAYICYTLLNNPEEAEKALDLAWQKVKTSSAYSSLKEEILAIKSYA*
CopB2 nucleic acid sequence
SEQ ID NO: 91
atgagctcttggtttgcacaggcgacggacgtcgctttgagccagacccttgatctgcctgacgcttcattggcggttcaaac
cgaaaaatttccAtacagctgttcaatctctaaggaatccgccccAtcatgtattcgtaaaatcttcgcccatttagcatctcagaaggaa
agtgctccgctgtctttttctcgtttacaaccgactactccgaaagaacgcatcctgtttttcgggtcatcgccttcctcccaattgtcctcga
ctgtccgcaccacaacctcttctccatggaatctttttagcaactcccaggcacgcaactcgacccgtaaattgtcggagaagcttcattt
gagctcagagttatccgcccgtgactccactaagccttcgtcgagcgaaccggttaaaccatcggaaaatcttttgcacacccctgagc
atcataaggaatccttctcaagtttgaaaaaggataacttatctcctatcatggaggagatcgactcattctctgcagagacagagtccctt
gaagagcgtttggtcacccagaaaaaggaggagacggtggcccaggagcaaaagcacccAttgctgcgtacatctactccgccat
caaaggccagcggggaatcacaagattctagcgaacacagctcaaaggaagatccttatagtcaacaaccgagccataaaatccaac
gccgtaaagagcgtgctaagcgcgtcgtcccAattattactccgccaacggtgggtatctttagtttgagctaccttcttacaaaacagg
ggatcttagcggatttcagcgcctattcggcatacaaggataatttagaaacaactcagcaagagctgaccatgttgcatcaagaacgta
tcgagcaagtccaaaaGatcgtggataaaagtaagacaatgcgcttttgggattcattagcatccattgtggccacaatcattccatgga
tcgaaatgggtgttgcagtaaccatcatcgcactgggaggtggaatcctttcctggtgctctctttttgctgcgcttatcatgattgtaatttc
attattggaagcattcgacgggtggcgtgcaatcgctaagcatttaccaggtaacgatcttgaaaagaagatgcgttatttaggttacgta
aagttggccttaactgtgttctcgtgcttactgagtttaagcgccttgtatgtagcaaaattaggaatgagtccgcttttggagggggttgtg
aagagtatcgcaccAgcattaagtggtatgctgggtttgactcaaggcgtagcactgtatttacaatcttcatcgcaaaagattcgtgcc
cgctgcactcagatcgacgcacgcattgaattgattaactgggaacgcgatgagtatttcttgcgtgctgaacaacttcttgattcaatgc
aaacgtccttcgaacaacttactgaaacattacagttacaacgtgaaattgatcagacatttacagacgctttgcgcTAG
CopB2 amino acid sequence
SEQ ID NO: 92
MSSWFAQATDVALSQTLDLPDASLAVQTEKFPYSCSISKESAPSCIRKIFAHLA
SQKESAPLSFSRLQPTTPKERILFFGSSPSSQLSSTVRTTTSSPWNLFSNSQARNSTRKL
SEKLHLSSELSARDSTKPSSSEPVKPSENLLHTPEHHKESFSSLKKDNLSPIMEEIDSFS
AETESLEERLVTQKKEETVAQEQKHPLLRTSTPPSKASGESQDSSEHSSKEDPYSQQP
SHKIQRRKERAKRVVPIITPPTVGIFSLSYLLTKQGILADFSAYSAYKDNLETTQQELT
MLHQERIEQVQKIVDKSKTMRFWDSLASIVATIIPWIEMGVAVTIIALGGGILSWCSLF
AALIMIVISLLEAFDGWRAIAKHLPGNDLEKKMRYLGYVKLALTVFSCLLSLSALYV
AKLGMSPLLEGVVKSIAPALSGMLGLTQGVALYLQSSSQKIRARCTQIDARIELINWE
RDEYFLRAEQLLDSMQTSFEQLTETLQLQREIDQTFTDALR
CT053-CopB2 nucleic acid sequence
SEQ ID NO: 93
aaaagtgagcgtttaaaaaaattagaatcagagcttcatgatcttacccagtggatgcaacttggccttgttcctaaaaaagaa
atcgagagacaccaggaagaaatccgtctgctagaaagcaaaatccttgaagagaaagaacgtctacaacttctcaaagaaagcggt
gagatcaaagagtacgtaacccctcgaagaactccagctaaaaccatttacccagatggccccagcgtttcagacgttgagtttgtaga
atcctcggatacagaagtggatctcgatgccggtgacacaattgagattgacctaggtgatgaggcaagagaagaaagcggaaacg
aactcgactactctagtgaagacgatgaggatcctttcagcgatcgcaatcgttggcgccgaggaggcatcatagatcctgacgcgaa
tgaatggGGTTCAGCTGCTTCAatgagctcttggtttgcacaggcgacggacgtcgctttgagccagacccttgatctgc
ctgacgcttcattggcggttcaaaccgaaaaatttccAtacagctgttcaatctctaaggaatccgccccAtcatgtattcgtaaaatctt
cgcccatttagcatctcagaaggaaagtgctccgctgtctttttctcgtttacaaccgactactccgaaagaacgcatcctgtttttcgggt
catcgccttcctcccaattgtcctcgactgtccgcaccacaacctcttctccatggaatctttttagcaactcccaggcacgcaactcgac
ccgtaaattgtcggagaagcttcatttgagctcagagttatccgcccgtgactccactaagccttcgtcgagcgaaccggttaaaccatc
ggaaaatcttttgcacacccctgagcatcataaggaatccttctcaagtttgaaaaaggataacttatctcctatcatggaggagatcgac
tcattctctgcagagacagagtcccttgaagagcgtttggtcacccagaaaaaggaggagacggtggcccaggagcaaaagcaccc
Attgctgcgtacatctactccgccatcaaaggccagcggggaatcacaagattctagcgaacacagctcaaaggaagatccttatagt
caacaaccgagccataaaatccaacgccgtaaagagcgtgctaagcgcgtcgtcccAattattactccgccaacggtgggtatcttta
gtttgagctaccttcttacaaaacaggggatcttagcggatttcagcgcctattcggcatacaaggataatttagaaacaactcagcaag
agctgaccatgttgcatcaagaacgtatcgagcaagtccaaaaGatcgtggataaaagtaagacaatgcgcttttgggattcattagca
tccattgtggccacaatcattccatggatcgaaatgggtgttgcagtaaccatcatcgcactgggaggtggaatcctttcctggtgctctc
tttttgctgcgcttatcatgattgtaatttcattattggaagcattcgacgggtggcgtgcaatcgctaagcatttaccaggtaacgatcttga
aaagaagatgcgttatttaggttacgtaaagttggccttaactgtgttctcgtgcttactgagtttaagcgccttgtatgtagcaaaattagg
aatgagtccgcttttggagggggttgtgaagagtatcgcaccAgcattaagtggtatgctgggtttgactcaaggcgtagcactgtattt
acaatcttcatcgcaaaagattcgtgcccgctgcactcagatcgacgcacgcattgaattgattaactgggaacgcgatgagtatttcttg
cgtgctgaacaacttcttgattcaatgcaaacgtccttcgaacaacttactgaaacattacagttacaacgtgaaattgatcagacatttac
agacgctttgcgcTAG
CT053-CopB2 amino acid sequence
SEQ ID NO: 94
MKSERLKKLESELHDLTQWMQLGLVPKKEIERHQEEIRLLESKILEEKERLQL
LKESGEIKEYVTPRRTPAKTIYPDGPSVSDVEFVESSDTEVDLDAGDTIEIDLGDEARE
ESGNELDYSSEDDEDPFSDRNRWRRGGIIDPDANEWGSAASMSSWFAQATDVALSQ
TLDLPDASLAVQTEKFPYSCSISKESAPSCIRKIFAHLASQKESAPLSFSRLQPTTPKERI
LFFGSSPSSQLSSTVRTTTSSPWNLFSNSQARNSTRKLSEKLHLSSELSARDSTKPSSSE
PVKPSENLLHTPEHHKESFSSLKKDNLSPIMEEIDSFSAETESLEERLVTQKKEETVAQ
EQKHPLLRTSTPPSKASGESQDSSEHSSKEDPYSQQPSHKIQRRKERAKRVVPIITPPT
VGIFSLSYLLTKQGILADFSAYSAYKDNLETTQQELTMLHQERIEQVQKIVDKSKTM
RFWDSLASIVATIIPWIEMGVAVTIIALGGGILSWCSLFAALIMIVISLLEAFDGWRAIA
KHLPGNDLEKKMRYLGYVKLALTVFSCLLSLSALYVAKLGMSPLLEGVVKSIAPALS
GMLGLTQGVALYLQSSSQKIRARCTQIDARIELINWERDEYFLRAEQLLDSMQTSFE
QLTETLQLQREIDQTFTDALR
LTA1-CT053-CopB2 nucleic acid sequence
SEQ ID NO: 95
CATatggacaatggcgatcgtttataccgtgccgactcgcgtcccccagatgagattaaacgtagcggtgggttaatgcc
acgtgggcacaatgagtattttgaccgtggaacacagatgaacattaacctttacgatcatgcccgtgggacccagaccgggtttgtcc
gttatgatgacgggtatgttagtacgagtttgtccttacgctccgcacaccttgcgggacaaagtattttatcaggctacagcacatattac
atttatgtgatcgccactgccccaaacatgttcaatgtgaacgatgtgttgggggtttacagcccccatccatatgaacaagaagtctcg
gcccttggggggatcccatatagccagatttatggttggtaccgcgtaaattttggtgtgattgatgaacgtttgcatcgtaaccgtgaata
ccgcgatcgctactaccgtaacttgaacattgcacctgccgaggacggctatcgtttagcgggattcccacccgatcatcaggcgtgg
cgtgaggaaccgtggatccatcacgcccctcaggggtgcgggaacagtagtcgcCATatgaaaagtgagcgtttaaaaaaattag
aatcagagcttcatgatcttacccagtggatgcaacttggccttgttcctaaaaaagaaatcgagagacaccaggaagaaatccgtctg
ctagaaagcaaaatccttgaagagaaagaacgtctacaacttctcaaagaaagcggtgagatcaaagagtacgtaacccctcgaaga
actccagctaaaaccatttacccagatggccccagcgtttcagacgttgagtttgtagaatcctcggatacagaagtggatctcgatgcc
ggtgacacaattgagattgacctaggtgatgaggcaagagaagaaagcggaaacgaactcgactactctagtgaagacgatgagga
tcctttcagcgatcgcaatcgttggcgccgaggaggcatcatagatcctgacgcgaatgaatggGGTTCAGCTGCTTCA
atgagctcttggtttgcacaggcgacggacgtcgctttgagccagacccttgatctgcctgacgcttcattggcggttcaaaccgaaaa
atttccAtacagctgttcaatctctaaggaatccgccccAtcatgtattcgtaaaatcttcgcccatttagcatctcagaaggaaagtgct
ccgctgtctttttctcgtttacaaccgactactccgaaagaacgcatcctgtttttcgggtcatcgccttcctcccaattgtcctcgactgtcc
gcaccacaacctcttctccatggaatctttttagcaactcccaggcacgcaactcgacccgtaaattgtcggagaagcttcatttgagctc
agagttatccgcccgtgactccactaagccttcgtcgagcgaaccggttaaaccatcggaaaatcttttgcacacccctgagcatcataa
ggaatccttctcaagtttgaaaaaggataacttatctcctatcatggaggagatcgactcattctctgcagagacagagtcccttgaagag
cgtttggtcacccagaaaaaggaggagacggtggcccaggagcaaaagcacccAttgctgcgtacatctactccgccatcaaaggc
cagcggggaatcacaagattctagcgaacacagctcaaaggaagatccttatagtcaacaaccgagccataaaatccaacgccgtaa
agagcgtgctaagcgcgtcgtcccAattattactccgccaacggtgggtatctttagtttgagctaccttcttacaaaacaggggatctta
gcggatttcagcgcctattcggcatacaaggataatttagaaacaactcagcaagagctgaccatgttgcatcaagaacgtatcgagca
agtccaaaaGatcgtggataaaagtaagacaatgcgcttttgggattcattagcatccattgtggccacaatcattccatggatcgaaat
gggtgttgcagtaaccatcatcgcactgggaggtggaatcctttcctggtgctctctttttgctgcgcttatcatgattgtaatttcattattgg
aagcattcgacgggtggcgtgcaatcgctaagcatttaccaggtaacgatcttgaaaagaagatgcgttatttaggttacgtaaagttgg
ccttaactgtgttctcgtgcttactgagtttaagcgccttgtatgtagcaaaattaggaatgagtccgcttttggagggggttgtgaagagt
atcgcaccAgcattaagtggtatgctgggtttgactcaaggcgtagcactgtatttacaatcttcatcgcaaaagattcgtgcccgctgc
actcagatcgacgcacgcattgaattgattaactgggaacgcgatgagtatttcttgcgtgctgaacaacttcttgattcaatgcaaacgt
ccttcgaacaacttactgaaacattacagttacaacgtgaaattgatcagacatttacagacgctttgcgcTAGCTCGAG
LTA1-CT053-CopB2 Amino acid sequence
SEQ ID NO: 96
MDNGDRLYRADSRPPDEIKRSGGLMPRGHNEYFDRGTQMNINLYDHARGTQ
TGFVRYDDGYVSTSLSLRSAHLAGQSILSGYSTYYIYVIATAPNMFNVNDVLGVYSP
HPYEQEVSALGGIPYSQIYGWYRVNFGVIDERLHRNREYRDRYYRNLNIAPAEDGYR
LAGFPPDHQAWREEPWIHHAPQGCGNSSRMKSERLKKLESELHDLTQWMQLGLVP
KKEIERHQEEIRLLESKILEEKERLQLLKESGEIKEYVTPRRTPAKTIYPDGPSVSDVEF
VESSDTEVDLDAGDTIEIDLGDEAREESGNELDYSSEDDEDPFSDRNRWRRGGIIDPD
ANEWGSAASMSSWFAQATDVALSQTLDLPDASLAVQTEKFPYSCSISKESAPSCIRKI
FAHLASQKESAPLSFSRLQPTTPKERILFFGSSPSSQLSSTVRTTTSSPWNLFSNSQARN
STRKLSEKLHLSSELSARDSTKPSSSEPVKPSENLLHTPEHHKESFSSLKKDNLSPIMEE
IDSFSAETESLEERLVTQKKEETVAQEQKHPLLRTSTPPSKASGESQDSSEHSSKEDPY
SQQPSHKIQRRKERAKRVVPIITPPTVGIFSLSYLLTKQGILADFSAYSAYKDNLETTQ
QELTMLHQERIEQVQKIVDKSKTMRFWDSLASIVATIIPWIEMGVAVTIIALGGGILS
WCSLFAALIMIVISLLEAFDGWRAIAKHLPGNDLEKKMRYLGYVKLALTVFSCLLSL
SALYVAKLGMSPLLEGVVKSIAPALSGMLGLTQGVALYLQSSSQKIRARCTQIDARIE
LINWERDEYFLRAEQLLDSMQTSFEQLTETLQLQREIDQTFTDALR*
HisScc3 chaperone for CT668-CopB2 nucleic acid sequence
SEQ ID NO: 97
ATGGGCAGCAGCCATCACCATCATCACCACAGCCAGGATCCGatgccaccaagc
aagatccaatgtcttgaaacttttgaaagaacttatggacacctttatctacaacatgcgtccctaatgcgtcatttagcctatctactcgata
aaattgctcgctcttaccctcatatgtgtccgcttcccgataatatggaagcgtactttgagaattatatccccaataaagatatccctctgg
acacctatcaaaaaattttcaaactgtcctcagaagatcttgaacaagtctacaaggaaggatacaacgcctatttacaaggagactatg
aggaaagttctaccgctttttactggttgattttctttaacccatttgtgtctaaattttggttttcattaggagcttcgctccatatgcgccaaaa
atatcaacaagctcttcatgcttatggtgtagctgctttgctaagagaaaaagacccttatcctcattactatgcctacatctgctacaccct
gctcaataatcctgaagaagctgaaaaagctcttgatcttgcttggcaaaaagtaaaaacaagctctgcctatagctctttaaaagaaga
aattttagcgatcaaatcgtacgcctaaGCGGCCGC
HisScc3 chaperone for CT668-CopB2 Amino acid sequence
SEQ ID NO: 98
MGSSHHHHHHSQDPMPPSKIQCLETFERTYGHLYLQHASLMRHLAYLLDKIA
RSYPHMCPLPDNMEAYFENYIPNKDIPLDTYQKIFKLSSEDLEQVYKEGYNAYLQGD
YEESSTAFYWLIFFNPFVSKFWFSLGASLHMRQKYQQALHAYGVAALLREKDPYPH
YYAYICYTLLNNPEEAEKALDLAWQKVKTSSAYSSLKEEILAIKSYA*
CT668-CopB2 nucleic acid sequence
SEQ ID NO: 99
aaaagtgagcgtttaaaaaaattagaatcagagcttcatgatcttacccagtggatgcaacttggccttgttcctaaaaaagaa
atcgagagacaccaggaagaaatccgtctgctagaaagcaaaatccttgaagagaaagaacgtctacaacttctcaaagaaagcggt
gagatcaaagagtacgtaacccctcgaagaactccagctaaaaccatttacccagatggccccagcgtttcagacgttgagtttgtaga
atcctcggatacagaagtggatctcgatgccggtgacacaattgagattgacctaggtgatgaggcaagagaagaaagcggaaacg
aactcgactactctagtgaagacgatgaggatcctttcagcgatcgcaatcgttggcgccgaggaggcatcatagatcctgacgcgaa
tgaatggGGTTCAGCTGCTTCAatgagctcttggtttgcacaggcgacggacgtcgctttgagccagacccttgatctgc
ctgacgcttcattggcggttcaaaccgaaaaatttccAtacagctgttcaatctctaaggaatccgccccAtcatgtattcgtaaaatctt
cgcccatttagcatctcagaaggaaagtgctccgctgtctttttctcgtttacaaccgactactccgaaagaacgcatcctgtttttcgggt
catcgccttcctcccaattgtcctcgactgtccgcaccacaacctcttctccatggaatctttttagcaactcccaggcacgcaactcgac
ccgtaaattgtcggagaagcttcatttgagctcagagttatccgcccgtgactccactaagccttcgtcgagcgaaccggttaaaccatc
ggaaaatcttttgcacacccctgagcatcataaggaatccttctcaagtttgaaaaaggataacttatctcctatcatggaggagatcgac
tcattctctgcagagacagagtcccttgaagagcgtttggtcacccagaaaaaggaggagacggtggcccaggagcaaaagcaccc
Attgctgcgtacatctactccgccatcaaaggccagcggggaatcacaagattctagcgaacacagctcaaaggaagatccttatagt
caacaaccgagccataaaatccaacgccgtaaagagcgtgctaagcgcgtcgtcccAattattactccgccaacggtgggtatcttta
gtttgagctaccttcttacaaaacaggggatcttagcggatttcagcgcctattcggcatacaaggataatttagaaacaactcagcaag
agctgaccatgttgcatcaagaacgtatcgagcaagtccaaaaGatcgtggataaaagtaagacaatgcgcttttgggattcattagca
tccattgtggccacaatcattccatggatcgaaatgggtgttgcagtaaccatcatcgcactgggaggtggaatcctttcctggtgctctc
tttttgctgcgcttatcatgattgtaatttcattattggaagcattcgacgggtggcgtgcaatcgctaagcatttaccaggtaacgatcttga
aaagaagatgcgttatttaggttacgtaaagttggccttaactgtgttctcgtgcttactgagtttaagcgccttgtatgtagcaaaattagg
aatgagtccgcttttggagggggttgtgaagagtatcgcaccAgcattaagtggtatgctgggtttgactcaaggcgtagcactgtattt
acaatcttcatcgcaaaagattcgtgcccgctgcactcagatcgacgcacgcattgaattgattaactgggaacgcgatgagtatttcttg
cgtgctgaacaacttcttgattcaatgcaaacgtccttcgaacaacttactgaaacattacagttacaacgtgaaattgatcagacatttac
agacgctttgcgcTAG
CT668-CopB2 amino acid sequence
SEQ ID NO: 100
MKSERLKKLESELHDLTQWMQLGLVPKKEIERHQEEIRLLESKILEEKERLQL
LKESGEIKEYVTPRRTPAKTIYPDGPSVSDVEFVESSDTEVDLDAGDTIEIDLGDEARE
ESGNELDYSSEDDEDPFSDRNRWRRGGIIDPDANEWGSAASMSSWFAQATDVALSQ
TLDLPDASLAVQTEKFPYSCSISKESAPSCIRKIFAHLASQKESAPLSFSRLQPTTPKERI
LFFGSSPSSQLSSTVRTTTSSPWNLFSNSQARNSTRKLSEKLHLSSELSARDSTKPSSSE
PVKPSENLLHTPEHHKESFSSLKKDNLSPIMEEIDSFSAETESLEERLVTQKKEETVAQ
EQKHPLLRTSTPPSKASGESQDSSEHSSKEDPYSQQPSHKIQRRKERAKRVVPIITPPT
VGIFSLSYLLTKQGILADFSAYSAYKDNLETTQQELTMLHQERIEQVQKIVDKSKTM
RFWDSLASIVATIIPWIEMGVAVTIIALGGGILSWCSLFAALIMIVISLLEAFDGWRAIA
KHLPGNDLEKKMRYLGYVKLALTVFSCLLSLSALYVAKLGMSPLLEGVVKSIAPALS
GMLGLTQGVALYLQSSSQKIRARCTQIDARIELINWERDEYFLRAEQLLDSMQTSFE
QLTETLQLQREIDQTFTDALR
LTA1-CT668-CopB2 nucleic acid sequence
SEQ ID NO: 101
CATatggacaatggcgatcgtttataccgtgccgactcgcgtcccccagatgagattaaacgtagcggtgggttaatgcc
acgtgggcacaatgagtattttgaccgtggaacacagatgaacattaacctttacgatcatgcccgtgggacccagaccgggtttgtcc
gttatgatgacgggtatgttagtacgagtttgtccttacgctccgcacaccttgcgggacaaagtattttatcaggctacagcacatattac
atttatgtgatcgccactgccccaaacatgttcaatgtgaacgatgtgttgggggtttacagcccccatccatatgaacaagaagtctcg
gcccttggggggatcccatatagccagatttatggttggtaccgcgtaaattttggtgtgattgatgaacgtttgcatcgtaaccgtgaata
ccgcgatcgctactaccgtaacttgaacattgcacctgccgaggacggctatcgtttagcgggattcccacccgatcatcaggcgtgg
cgtgaggaaccgtggatccatcacgcccctcaggggtgcgggaacagtagtcgcCATatgaaaagtgagcgtttaaaaaaattag
aatcagagcttcatgatcttacccagtggatgcaacttggccttgttcctaaaaaagaaatcgagagacaccaggaagaaatccgtctg
ctagaaagcaaaatccttgaagagaaagaacgtctacaacttctcaaagaaagcggtgagatcaaagagtacgtaacccctcgaaga
actccagctaaaaccatttacccagatggccccagcgtttcagacgttgagtttgtagaatcctcggatacagaagtggatctcgatgcc
ggtgacacaattgagattgacctaggtgatgaggcaagagaagaaagcggaaacgaactcgactactctagtgaagacgatgagga
tcctttcagcgatcgcaatcgttggcgccgaggaggcatcatagatcctgacgcgaatgaatggGGTTCAGCTGCTTCA
atgagctcttggtttgcacaggcgacggacgtcgctttgagccagacccttgatctgcctgacgcttcattggcggttcaaaccgaaaa
atttccAtacagctgttcaatctctaaggaatccgccccAtcatgtattcgtaaaatcttcgcccatttagcatctcagaaggaaagtgct
ccgctgtctttttctcgtttacaaccgactactccgaaagaacgcatcctgtttttcgggtcatcgccttcctcccaattgtcctcgactgtcc
gcaccacaacctcttctccatggaatctttttagcaactcccaggcacgcaactcgacccgtaaattgtcggagaagcttcatttgagctc
agagttatccgcccgtgactccactaagccttcgtcgagcgaaccggttaaaccatcggaaaatcttttgcacacccctgagcatcataa
ggaatccttctcaagtttgaaaaaggataacttatctcctatcatggaggagatcgactcattctctgcagagacagagtcccttgaagag
cgtttggtcacccagaaaaaggaggagacggtggcccaggagcaaaagcacccAttgctgcgtacatctactccgccatcaaaggc
cagcggggaatcacaagattctagcgaacacagctcaaaggaagatccttatagtcaacaaccgagccataaaatccaacgccgtaa
agagcgtgctaagcgcgtcgtcccAattattactccgccaacggtgggtatctttagtttgagctaccttcttacaaaacaggggatctta
gcggatttcagcgcctattcggcatacaaggataatttagaaacaactcagcaagagctgaccatgttgcatcaagaacgtatcgagca
agtccaaaaGatcgtggataaaagtaagacaatgcgcttttgggattcattagcatccattgtggccacaatcattccatggatcgaaat
gggtgttgcagtaaccatcatcgcactgggaggtggaatcctttcctggtgctctctttttgctgcgcttatcatgattgtaatttcattattgg
aagcattcgacgggtggcgtgcaatcgctaagcatttaccaggtaacgatcttgaaaagaagatgcgttatttaggttacgtaaagttgg
ccttaactgtgttctcgtgcttactgagtttaagcgccttgtatgtagcaaaattaggaatgagtccgcttttggagggggttgtgaagagt
atcgcaccAgcattaagtggtatgctgggtttgactcaaggcgtagcactgtatttacaatcttcatcgcaaaagattcgtgcccgctgc
actcagatcgacgcacgcattgaattgattaactgggaacgcgatgagtatttcttgcgtgctgaacaacttcttgattcaatgcaaacgt
ccttcgaacaacttactgaaacattacagttacaacgtgaaattgatcagacatttacagacgctttgcgcTAGCTCGAG
LTA1-CT668-CopB2Amino acid sequence
SEQ ID NO: 102
MDNGDRLYRADSRPPDEIKRSGGLMPRGHNEYFDRGTQMNINLYDHARGTQ
TGFVRYDDGYVSTSLSLRSAHLAGQSILSGYSTYYIYVIATAPNMFNVNDVLGVYSP
HPYEQEVSALGGIPYSQIYGWYRVNFGVIDERLHRNREYRDRYYRNLNIAPAEDGYR
LAGFPPDHQAWREEPWIHHAPQGCGNSSRMKSERLKKLESELHDLTQWMQLGLVP
KKEIERHQEEIRLLESKILEEKERLQLLKESGEIKEYVTPRRTPAKTIYPDGPSVSDVEF
VESSDTEVDLDAGDTIEIDLGDEAREESGNELDYSSEDDEDPFSDRNRWRRGGIIDPD
ANEWGSAASMSSWFAQATDVALSQTLDLPDASLAVQTEKFPYSCSISKESAPSCIRKI
FAHLASQKESAPLSFSRLQPTTPKERILFFGSSPSSQLSSTVRTTTSSPWNLFSNSQARN
STRKLSEKLHLSSELSARDSTKPSSSEPVKPSENLLHTPEHHKESFSSLKKDNLSPIMEE
IDSFSAETESLEERLVTQKKEETVAQEQKHPLLRTSTPPSKASGESQDSSEHSSKEDPY
SQQPSHKIQRRKERAKRVVPIITPPTVGIFSLSYLLTKQGILADFSAYSAYKDNLETTQ
QELTMLHQERIEQVQKIVDKSKTMRFWDSLASIVATIIPWIEMGVAVTIIALGGGILS
WCSLFAALIMIVISLLEAFDGWRAIAKHLPGNDLEKKMRYLGYVKLALTVFSCLLSL
SALYVAKLGMSPLLEGVVKSIAPALSGMLGLTQGVALYLQSSSQKIRARCTQIDARIE
LINWERDEYFLRAEQLLDSMQTSFEQLTETLQLQREIDQTFTDALR
dmLT eltA (LTa) nucleic acid sequence
SEQ ID NO: 113
atgattgaca tcatgttgca tataggttag ataaaacaag tggttatctt tccggattgt cttcttgtat gatatataag ttttcctcga
tgaaaaatat aactttcatt ttttttattt tattagcatc gccattatat gcaaatggcg acagattata ccgtgctgac tctagacccc
cagatgaaat aaaacgtttc cggagtctta tgcccagagg taatgagtac ttcgatagag gaactcaaat gaatattaat
ctttatgatc acgcgagagg aacacaaacc ggctttgtca gatatgatga cggatatgtt tccacttctc ttagtttgag
aagtgctcac ttagcaggac agtatatatt atcaggatat tcacttacta tatatatcgt tatagcaaat atgtttaatg ttaatgatgt
aattagcgta tacagccctc acccatatga acaggaggtt tctgcgttag gtggaatacc atattctcag atatatggat
ggtatcgtgt taattttggt gtgattgatg aacgattaca tcgtaacagg gaatatagag accggtatta cagaaatctg
aatatagctc cggcagagga tggttacaga ttagcaggtt tcccaccgga tcaccaagct tggagagaag aaccctggat
tcatcatgca ccacaaggtt gtggagattc atcaGgaaca atcacaggtg atacttgtaa tgaggagacc cagaatctga
gcacaatata tGCcagggaa tatcaatcaa aagttaagag gcagatattt tcagactatc agtcagaggt tgacatatat
aacagaattc gggatgaatt atgaataaag taaaatgt
eltB (LTb) nucleic acid sequence
SEQ ID NO: 114
gttgacatat ataacagaat tcgggatgaa ttatgaataa agtaaaatgt tatgttttat ttacggcgtt actatcctct ctatatgcac
acggagctcc ccagactatt acagaactat gttcggaata tcgcaacaca caaatatata cgataaatga caagatacta
tcatatacgg aatcgatggc aggcaaaaga gaaatggtta tcattacatt taagagcggc gaaacatttc aggtcgaagt
cccgggcagt caacatatag actcccagaa aaaagccatt gaaaggatga aggacacatt aagaatcaca tatctgaccg
agaccaaaat tgataaatta tgtgtatgga ataataaaac ccccaattca attgcggcaa tcagtatgaa aaactagttt
gctttaaaag catgtctaat gctaggaacc tatataacaa ctactgtact tatactaatg agccttatgc tgcatttgaa
aaggcggtag aggaggcaat accgatcctt aaactgtaac actataacag cttccactac agggagctgt tatagcacac
agaaaaaact aagctaggct ggaggggcaa gctt

What is claimed is:

1. A fusion polypeptide comprising a fusion of a needle tip protein or an antigenic fragment thereof and a translocator protein or an antigenic fragment thereof from a Type III secretion system (T3SS) of a Gram negative bacteria; wherein the gram negative bacteria is not a Salmonella enterica. or Shigella spp.

2. The polypeptide of claim 1, wherein the fusion polypeptide is arranged so that the needle tip protein is 5′ of the translocator protein.

3. The polypeptide of claim 1, wherein the gram negative bacteria comprises Bordetella spp., Burkholderia spp., Chlamydia spp., Pseudomonas spp., Vibrio spp., or Yersinia spp.

4. The polypeptide of claim 1, wherein the needle tip protein comprises Bsp22, LcrV, BipD, PcrV, CT053, or CT668.

5. The polypeptide of claim 1, wherein the translocator protein comprises BopB, YopB, BipB, PopB, CopB, or CopB2.

6. The polypeptide of claim 1, wherein the fusion comprises the Bordetella spp. needle-tip protein (Bsp22) and translocator protein (BopB), or antigenic fragments thereof.

7. The polypeptide of claim 1, wherein the fusion comprises the Yersinia spp. needle-tip protein (LcrV) and translocator protein (YopB), or antigenic fragments thereof.

8. The polypeptide of claim 1, wherein the fusion comprises the Burkholderia spp. needle-tip protein (BipD) and translocator protein (BipB), or antigenic fragments thereof.

9. The polypeptide of claim 1, wherein the fusion comprises the Pseudomonas spp. needle-tip protein (PcrV) and translocator protein (PopB), or antigenic fragments thereof.

10. The polypeptide of claim 1, wherein the fusion comprises the Chlamydia spp. needle-tip protein (CT053 or CT668) and translocator protein (CopB or CopB2), or antigenic fragments thereof.

11. The polypeptide of claim 1, wherein the fusion further comprises double mutant labile toxin (dmLT) or an antigenic fragment thereof from Enterotoxigenic Escherichia coli or cholera toxin or an antigenic fragment thereof.

12. The polypeptide of claim 1, wherein the dmLT comprises the active moiety LTA1.

13. The polypeptide of claim 1, wherein the dmLT retains its ADP ribosylation activity.

14. The polypeptide of claim 1, wherein the dmLT is 5′ of the needle tip protein and translocator protein fusion.

15. A vaccine comprising one or more of the fusion polypeptides of any of claims 1-14.

16. The vaccine of claim 15, further comprising one or more components of an acellular pertussis vaccine.

17. The vaccine of claim 15, further comprising pertussis toxoid (PTd).

18. A method of treating, inhibiting, or preventing an infection of a Gram negative bacteria in a subject comprising administering to the subject the fusion polypeptide of any of claims 1-14 or vaccine of claims 15-17.

19. The method of claim 18, wherein the method further inhibits or prevents colony formation of the bacteria and/or transmission of the bacteria to another subject.

20. A method of eliciting an immune response in a subject to a Gram negative bacteria comprising administering to the subject the fusion polypeptide of any of claims 1-14 or vaccine of claims 15-17.

21. The method of claim 20, wherein the immune response comprises a sterilizing immune response.

22. The method of claims 18-21, wherein the bacteria comprises Bordetella spp., Burkholderia spp., Chlamydia spp., Pseudomonas spp., Vibrio spp., or Yersinia spp.

23. A method of eliciting an immune response against at least one Gram negative bacteria serovar in a subject in need thereof, comprising administering to the subject a composition comprising at least one needle tip protein or an antigenic fragment thereof and/or at least one translocator protein or an antigenic fragment thereof; wherein said composition is administered in an amount sufficient to elicit an immune response to said at least one Gram negative bacteria serovar in said subject; and wherein the Gram negative bacteria is not a Shigella spp. or Salmonella enterica.

24. The method of claim 23, wherein the bacteria comprises Bordetella spp., Burkholderia spp., Chlamydia spp., Pseudomonas spp., Vibrio spp., or Yersinia spp.
